(19) United States
(12) Patent Application Publication
(10) Pub. No.: US 2005/0080031 A1
(43) Pub. Date: Apr. 14, 2005

(54) Nucleic acid treatment of diseases or conditions related to levels of Ras, HER2 and HIV

(75) Inventor: James McSwiggen, Boulder, CO (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Sirna Therapeutics, Inc., Boulder, CO (US)

(21) Appl. No.: 10/724,270

(22) Filed: Nov. 26, 2003

Related U.S. Application Data
(63) Continuation-in-part of application No. PCT/US02/16840, filed on May 29, 2002.
(63) Continuation-in-part of application No. 10/157,580, filed on May 29, 2002.
(63) Continuation-in-part of application No. 10/163,552, filed on Jun. 6, 2002.
(63) Continuation-in-part of application No. 10/238,700, filed on Sep. 10, 2002.
(63) Continuation-in-part of application No. 10/693,059, filed on Oct. 23, 2003.
Application No. 10/693,059 is a continuation-in-part of application No. 10/444,853, filed on May 23, 2003.
Application No. 10/444,853 is a continuation-in-part of application No. 10/417,012, filed on Apr. 16, 2003, now abandoned.
(63) Continuation-in-part of application No. 10/652,791, filed on Aug. 29, 2003.
(63) Continuation-in-part of application No. 10/422,704, filed on Apr. 24, 2003, now abandoned.
Application No. 10/693,059 is a continuation-in-part of application No. 10/427,160, filed on Apr. 30, 2003.
Application No. 10/693,059 is a continuation-in-part of application No. PCT/US02/15876, filed on May 20, 2002.
(63) Continuation-in-part of application No. PCT/US03/05346, filed on Feb. 20, 2003.
(63) Continuation-in-part of application No. PCT/US03/05028, filed on Feb. 20, 2003.
(60) Provisional application No. 60/294,140, filed on May 29, 2001.
(60) Provisional application No. 60/296,249, filed on Jun. 6, 2001.
(60) Provisional application No. 60/318,471, filed on Sep. 10, 2001.
(60) Provisional application No. 60/292,217, filed on May 18, 2001.
(60) Provisional application No. 60/306,883, filed on Jul. 20, 2001.
(60) Provisional application No. 60/311,865, filed on Aug. 13, 2001.
(60) Provisional application No. 60/362,016, filed on Mar. 6, 2002.
(60) Provisional application No. 60/358,580, filed on Feb. 20, 2002.
(60) Provisional application No. 60/363,124, filed on Mar. 11, 2002.
(60) Provisional application No. 60/386,782, filed on Jun. 6, 2002.
(60) Provisional application No. 60/406,784, filed on Aug. 29, 2002.
(60) Provisional application No. 60/408,378, filed on Sep. 5, 2002.
(60) Provisional application No. 60/409,293, filed on Sep. 9, 2002.
(60) Provisional application No. 60/440,129, filed on Jan. 15, 2003.

(51) Int. Cl. A61K 48/00; C07H 21/02

(52) U.S. Cl. 514/44; 536/23.1

(57) ABSTRACT

The present invention relates to nucleic acid molecules, including enzymatic nucleic acid molecules, such as DNAzymes (e.g. DNA enzymes, catalytic DNA), siRNA, aptamers, and antisense that modulate the expression of Ras genes such as K-Ras, H-Ras, and/or N-Ras, HIV genes such as HIV-1, and HER2 genes.

This application is a continuation-in-part of International Application No. PCT/US02/16840, filed May 29, 2002, which claims the benefit of U.S. Provisional Application No. 60/294,140, filed May 29, 2001, U.S. Provisional Application No. 60/296,249, filed Jun. 6, 2001, and U.S. Provisional Application No. 60/318,471, filed Sep. 10, 2001; this application is also a continuation-in-part of application Ser. No. 10/157,580, filed May 29, 2002, and is also a continuation-in-part of application Ser. No. 10/163,552, filed Jun. 6, 2002, and is also a continuation-in-part of application Ser. No. 10/238,700, filed Sep. 10, 2002; this application is also a continuation-in-part of application Ser. No. 10/693,059, filed Oct. 23, 2002, which is a continuation-in-part of application Ser. No. 10/444,853, filed May 23, 2003, which is a continuation-in part of U.S. patent application Ser. No. 10/417,012, filed Apr. 16, 2003; and application Ser. No. 10/693,059 is also a continuation-in-part of application Ser. No. 10/427,160, filed Apr. 30, 2003, and International Application No. PCT/US02/15876, filed May 17, 2002, which claims the benefit of U.S. Provisional Application No. 60/292,217, filed May 18, 2001, U.S. Provisional Application No. 60/306,883, filed Jul. 20, 2001, U.S. Provisional Application No. 60/311,865, filed Aug. 13, 2001, and U.S. Provisional Application No. 60/362,016, filed Mar. 6, 2002; this application is also a continuation-in-part of U.S. application Ser. No. 10/652,791, filed Aug. 29, 2003, and also a continuation-in-part of U.S. application Ser. No. 10/422,704, filed Apr. 24, 2003; this application is also a continuation-in-part of International Patent Application No. PCT/US03/05346, filed Feb. 20, 2003, and a continuation-in-part of International Patent Application No. PCT/US03/05028, filed Feb. 20, 2003, both of which claim the benefit of U.S. Provisional Application No. 60/358,580, filed Feb. 20, 2002, U.S. Provisional Application No. 60/363,124, filed Mar. 11, 2002, U.S. Provisional Application No. 60/386,782, filed Jun. 6, 2002, U.S. Provisional Application No. 60/406,784, filed Aug. 29, 2002, U.S. Provisional Application No. 60/408,378, filed Sep. 5, 2002, U.S. Provisional Application No. 60/409,293, filed Sep. 9, 2002, and U.S. Provisional Application No. 60/440,129, filed Jan. 15, 2003. The instant application claims the benefit of all the listed applications, which are hereby incorporated by reference herein in their entireties, including the drawings.

### TECHNICAL FIELD OF THE INVENTION

The present invention relates to novel nucleic acid compounds and methods for the treatment or diagnosis of diseases or conditions related to levels of Ras gene expression, such as K-Ras, H-Ras, and/or N-Ras expression, HIV infection such as HIV-1, and HER2 gene expression.

### BACKGROUND OF THE INVENTION

Transformation is a cumulative process whereby normal control of cell growth and differentiation is interrupted, usually through the accumulation of mutations affecting the expression of genes that regulate cell growth and differentiation.

The platelet derived growth factor (PDGF) system has served as a prototype for identification of substrates of the receptor tyrosine kinases. Certain enzymes become activated by the PDGF receptor kinase, including phospholipase C and phosphatidylinositol 3′ kinase, Ras guanosine triphosphate (GTPase) activating protein (GAP) and src-like tyrosine kinases. GAP regulates the function of the Ras protein by stimulating the GTPase activity of the 21 kD Ras protein. Barbacid, 56 Ann. Rev. Biochem. 779, 1987. Microinjection of oncogenically activated Ras into NIH 3T3 cells has been shown to induce DNA synthesis. Mutations that cause oncogenic activation of Ras lead to accumulation of Ras bound to GTP, the active form of the molecule. These mutations block the ability of GAP to convert Ras to the inactive form. Mutations that impair the interactions of Ras with GAP also block the biological function of Ras.

While a number of Ras alleles exist (N-Ras, K-Ras, H-Ras) which have been implicated in carcinogenesis, the type most often associated with colon and pancreatic carcinomas is K-Ras. Enzymatic nucleic acid molecules which are targeted to certain regions of the K-Ras allelic mRNAs may also prove inhibitory to the function of the other allelic mRNAs of the N-Ras and H-Ras genes.

Scanlon, International PCT Publication Nos. WO 91/18625, WO 91/18624, and WO 91/18913 describes a ribozyme effective to cleave oncogene RNA from the H-Ras gene. This ribozyme is said to inhibit H-ras expression in response to exogenous stimuli. Reddy WO92/00080 describes the use of ribozymes as therapeutic agents for leukemias, such as chronic myelogenous leukemia (CML) by targeting specific portions of the BCR-ABL gene transcript.

Thompson et al., International PCT publication No. WO 99/54459, describe nucleic acid molecules that modulate gene expression, including Ras gene expression.

Zhang et al., 2000, Gene Ther., 7, 2041; Takunaga et al., 2000, Br. J. Cancer., 83, 833; Zhang et al., 2000, Mol. Biotechnol., 15, 39; Irie et al., 2000, Mol. Urol. 4, 61; Kijima and Scanlon, 2000, Mol. Biotechnol., 14, 59; Funato et al., 2000, Cancer Gene Ther., 7, 495; Tsuchida et al., 2000, Cancer Gene Ther., 7, 373; Zhang et al., 2000, Methods Mol. Med., 35, 261; Irie et al., 1999, Antisense Nucleic Acid Drug Dev., 9, 341; Giannini et al., 1999, Nucleic Acids Res., 27, 2737; Fang et al., 1999, J. Med. Coll. PLA, 14, 25; Tong et al., 1998, Methods Mol. Med., 11, 209; Ohkawa and Kashani-Sabet, 1998, Methods Mol. Med., 11, 153; Scherr et al., 1999, Gene Ther., 6, 152; Tsuchida et al., 1998, Biochem. Biophys. Res. Commun., 252, 368; Scherr et al., 1998, Gene Ther., 5, 1227; Uhlmann et al., European Patent Application EP 808898; Scherr et al., 1997, J. Biol. Chem., 272, 14304; Chang et al., 1997, J. Cancer Res. Clin. Oncol., 123, 91; Ohta et al., 1996, Nucleic Acids Res., 24, 938; Ohta et al., 1994, Ann. N.Y. Acad. Sci., 716, 242; and Funato et al., 1994, Biochem. Pharmacol., 48, 1471 all describe specific ribozymes targeting certain K-Ras, H-Ras, or N-Ras RNA sequences.

Todd, International PCT Publication Nos. WO 01/49877, WO 99/50452, and WO 99/45146 describes specific DNAzymes targeting K-Ras for diagnostic applications.

Acquired immunodeficiency syndrome (AIDS) is thought to be caused by infection with the human immunodeficiency virus, for example HIV-1. Draper et al., U.S. Pat. Nos. 6,159,692, 5,972,704, 5,693,535, and International PCT Publication Nos. WO WO 93/23569, WO 95/04818, describe enzymatic nucleic acid molecules targeting HIV. Todd et al., International PCT Publication No. WO 99/50452, describe methods for using specific DNAzyme motifs for detecting the presence of certain HIV RNAs. Sriram and Banerjea, 2000, Biochem J., 352, 667-673, describe specific RNA cleaving DNA enzymes targeting HIV-1. Zhang et al., 1999, FEBS Lett., 458, 151-156, describe specific RNA cleaving DNA enzymes used in the inhibition of HIV-1 infection.

HER2 (also known as neu, erbB2 and c-erbB2) is an oncogene that encodes a 185-kDa transmembrane tyrosine kinase receptor. HER2 is a member of the epidermal growth factor receptor (EGFR) family and shares partial homology with other family members. In normal adult tissues HER2 expression is low. However, HER2 is overexpressed in at least 25-30% of breast (McGuire, H. C. and Greene, M. I. (1989) The neu (c-erbB-2) oncogene. Semin. Oncol. 16: 148-155) and ovarian cancers (Berchuck, A. Kamel, A., Whitaker, R. et al. (1990)). Overexpression of her-2/neu is associated with poor survival in advanced epithelial ovarian cancer. Cancer Research 50: 4087-4091). Furthermore, overexpression of HER2 in malignant breast tumors has been correlated with increased metastasis, chemoresistance and poor survival rates (Slamon et al., 1987 Science 235: 177-182). Because HER2 expression is high in aggressive human breast and ovarian cancers, but low in normal adult tissues, it is an attractive target for enzymatic nucleic acid-mediated therapy. McSwiggen et al., International PCT Publication No. WO 01/16312 and Beigelman et al., International PCT Publication No. WO 99/55857 describe enzymatic nucleic acid molecules targeting HER2. Thompson and Draper, U.S. Pat. No. 5,599,704, describes enzymatic nucleic acid molecules targeting HER2 (erbB2/neu) gene expression.

### SUMMARY OF THE INVENTION

The present invention features nucleic acid molecules, including, for example, antisense oligonucleotides, siRNA, aptamers, decoys and enzymatic nucleic acid molecules such as DNAzyme enzymatic nucleic acid molecules, which modulate expression of nucleic acid molecules encoding Ras oncogenes, such as K-Ras, H-Ras, and N-Ras. In one embodiment, the invention features an enzymatic nucleic acid molecule comprising a sequence selected from the group consisting of SEQ ID NOs: 2329-4655.

In another embodiment, the invention features an enzymatic nucleic acid molecule comprising at least one binding arm having a sequence complementary to a sequence selected from the group consisting of SEQ ID NOs: 1-2328.

In another embodiment, the invention features a siRNA molecule having complementarity to a sequence selected from the group consisting of SEQ ID NOs: 1-2328.

In another embodiment, the invention features an antisense molecule having complementarity to a sequence selected from the group consisting of SEQ ID NOs: 1-2328.

In another aspect of the invention, the nucleic acid of the invention is adapted to treat cancer.

In one embodiment, the enzymatic nucleic acid molecule of the invention has an endonuclease activity to cleave RNA having a K-Ras sequence.

In another embodiment, the enzymatic nucleic acid molecule of the invention has an endonuclease activity to cleave RNA having an H-Ras sequence.

In another embodiment, the enzymatic nucleic acid molecule of the invention has an endonuclease activity to cleave RNA having an N-Ras sequence.

In one embodiment, the siRNA molecule of the invention has RNA interference activity to K-Ras expression.

In another embodiment, the siRNA molecule of the invention has RNA interference activity to H-Ras expression.

In another embodiment, the siRNA molecule of the invention has RNA interference activity to N-Ras expression.

In one embodiment, a siRNA molecule of the invention comprises a double stranded RNA wherein one strand of the RNA is complementary to the RNA of K-Ras, H-Ras, and/or N-Ras gene. In another embodiment, a siRNA molecule of the invention comprises a double stranded RNA wherein one strand of the RNA comprises a portion of a sequence of RNA of K-Ras, H-Ras, and/or N-Ras gene sequence. In yet another embodiment, a siRNA molecule of the invention comprises a double stranded RNA wherein both strands of RNA are connected by a non-nucleotide linker. Alternately, a siRNA molecule of the invention comprises a double stranded RNA wherein both strands of RNA are connected by a nucleotide linker, such as a loop or stem loop structure.

In one embodiment, a single strand component of a siRNA molecule of the invention is from about 14 to about 50 nucleotides in length. In another embodiment, a single strand component of a siRNA molecule of the invention is about 14, 15, 16, 17, 18, 19, 20, 21, 22, 23, 24, 25, 26, 27, or 28 nucleotides in length. In yet another embodiment, a single strand component of a siRNA molecule of the invention is about 23 nucleotides in length. In one embodiment, a siRNA molecule of the invention is from about 28 to about 56 nucleotides in length. In another embodiment, a siRNA molecule of the invention is about 40, 41, 42, 43, 44, 45, 46, 47, 48, 49, 50, 51, or 52 nucleotides in length. In yet another embodiment, a siRNA molecule of the invention is about 46 nucleotides in length.

In one embodiment, the DNAzyme molecule of the invention is in a “10-23” configuration (see for example Santoro et al., 1997, PNAS, 94, 4262 and Joyce et al., U.S. Pat. No. 5,807,718). In another embodiment, the DNAzyme comprises a sequence complementary to a sequence selected from the group consisting of SEQ ID NOs: 1-2328. In yet another embodiment, the DNAzyme comprises a sequence selected from the group consisting of SEQ ID NOs: 2329-4655.

In another embodiment, the nucleic acid molecule of the invention comprises between 12 and 100 bases complementary to a nucleic acid molecule having a K-Ras sequence. In yet another embodiment, the enzymatic nucleic acid comprises between 14 and 24 bases complementary to a nucleic acid molecule having a K-Ras sequence.

In another embodiment, the nucleic acid molecule of the invention comprises between 12 and 100 bases complementary to a nucleic acid molecule having an H-Ras sequence. In yet another embodiment, the nucleic acid molecule of the invention comprises between 14 and 24 bases complementary to a nucleic acid molecule having an H-Ras sequence.

In another embodiment, the nucleic acid molecule of the invention comprises between 12 and 100 bases complementary to a nucleic acid molecule having an N-Ras sequence. In yet another embodiment, the nucleic acid molecule of the invention comprises between 14 and 24 bases complementary to a nucleic acid molecule having an N-Ras sequence.

In yet another embodiment, the nucleic acid molecule of the invention is chemically synthesized. The nucleic acid molecule can comprise at least one 2′-sugar modification, at least one nucleic acid base modification, and/or at least one phosphate backbone modification.

In one embodiment, the invention features a mammalian cell comprising the nucleic acid molecule of the invention. In another embodiment, the mammalian cell of the invention is a human cell.

In another embodiment, the invention features a method of modulating K-Ras activity in a cell, comprising contacting the cell with the nucleic acid molecule of the invention, under conditions suitable for the modulation of K-Ras activity.

In another embodiment, the invention features a method of modulating H-Ras activity in a cell, comprising contacting the cell with the nucleic acid molecule of the invention, under conditions suitable for the modulation of H-Ras activity.

In another embodiment, the invention features a method of modulating N-Ras activity in a cell, comprising contacting the cell with the nucleic acid molecule of the invention, under conditions suitable for the modulation of N-Ras activity.

In another embodiment, the invention features a method of treatment of a subject having a condition associated with the level of K-Ras, comprising contacting cells of the subject with the nucleic acid molecule of the invention, under conditions suitable for the treatment.

In another embodiment, the invention features a method of treatment of a subject having a condition associated with the level of H-Ras, comprising contacting cells of the subject with the nucleic acid molecule of the invention, under conditions suitable for the treatment.

In another embodiment, the invention features a method of treatment of a subject having a condition associated with the level of N-Ras, comprising contacting cells of the subject with the nucleic acid molecule of the invention, under conditions suitable for the treatment.

In one embodiment, a method of treatment of the invention further comprises the use of one or more drug therapies under conditions suitable for the treatment.

In another embodiment, the invention features a method of cleaving RNA having a K-Ras sequence comprising contacting the K-Ras RNA with the enzymatic nucleic acid molecule of the invention under conditions suitable for the cleavage, for example, where the cleavage is carried out in the presence of a divalent cation, such as Mg2+.

In another embodiment, the invention features a method of cleaving RNA having a H-Ras sequence comprising contacting the H-Ras RNA with the enzymatic nucleic acid molecule of the invention under conditions suitable for the cleavage, for example, where the cleavage is carried out in the presence of a divalent cation, such as Mg2+.

In another embodiment, the invention features a method of cleaving RNA having an N-Ras sequence comprising contacting the N-Ras RNA with the enzymatic nucleic acid molecule of the invention under conditions suitable for the cleavage, for example, where the cleavage is carried out in the presence of a divalent cation, such as Mg2+.

In one embodiment, the nucleic acid molecule of the invention comprises a cap structure, for example, a 3′,3′-linked or 5′,5′-linked deoxyabasic ribose derivative, wherein the cap structure is at the 5′-end, 3′-end, or both the 5′-end and the 3′-end of the nucleic acid molecule.

In another embodiment, the invention features an expression vector comprising a nucleic acid sequence encoding at least one nucleic acid molecule of the invention in a manner that allows expression of the nucleic acid molecule. For example, the invention features an expression vector comprising a nucleic acid encoding a DNAzyme in a manner that allows expression of the DNAzyme.

In yet another embodiment, the invention features a mammalian cell, for example a human cell, comprising an expression vector of the invention.

In another embodiment, the expression vector of the invention further comprises a sequence for a nucleic acid molecule complementary to an RNA having K-Ras sequence.

In another embodiment, the expression vector of the invention further comprises a sequence for a nucleic acid molecule complementary to an RNA having H-Ras sequence.

In another embodiment, the expression vector of the invention further comprises a sequence for a nucleic acid molecule complementary to an RNA having N-Ras sequence.

In one embodiment, an expression vector of the invention comprises a nucleic acid sequence encoding two or more nucleic acid molecules of the invention, which can be the same or different. In another embodiment, an expression vector of the invention further comprises a sequence encoding an antisense nucleic acid molecule complementary to an RNA having a K-Ras, H-Ras or N-Ras sequence.

In another embodiment, the invention features a method for treating cancer, for example colorectal cancer, bladder cancer, lung cancer, pancreatic cancer, breast cancer, or prostate cancer, comprising administering to a subject a nucleic acid molecule of the invention under conditions suitable for the treatment. A method of treatment of cancer of the invention can further comprise administering to a patient one or more other therapies, for example, monoclonal antibody therapy, such as Herceptin (trastuzumab); chemotherapy, such as paclitaxel (Taxol), docetaxel, cisplatin, methotrexate, cyclophosphamide, doxorubin, fluorouracil carboplatin, Leucovorin, Irinotecan (CAMPTOSAR® or CPT-11 or Camptothecin-11 or Campto), Carboplatin, edatrexate, gemcitabine, or vinorelbine; radiation therapy, or analgesic therapy and/or any combination thereof.

In another embodiment, the invention features a composition comprising a nucleic acid molecule of the invention in a pharmaceutically acceptable carrier.

In one embodiment, the invention features a method of administering to a cell, for example a mammalian cell or human cell, the nucleic acid molecule of the invention comprising contacting the cell with the nucleic acid molecule under conditions suitable for administration.

The method of administration can be in the presence of a delivery reagent, for example a lipid, cationic lipid, phospholipid, or liposome.

The present invention features an enzymatic nucleic acid molecule which modulates expression of a nucleic acid molecule encoding a human immunodeficiency virus (HIV), for example HIV-1, HIV-2, and related viruses such as FIV-1 and SIV-1, or a HIV gene, for example LTR, nef, vif, tat, or rev, wherein the enzymatic nucleic acid molecule comprises a DNAzyme configuration.

The invention also features an enzymatic nucleic acid molecule which modulates expression of a nucleic acid molecule encoding HIV or a component of HIV such as net, vif, tat, or rev, wherein the enzymatic nucleic acid molecule is in a Inozyme, G-cleaver, Zinzyme, DNAzyme or Amberzyme configuration.

The present invention also features a siRNA molecule which modulates expression of a nucleic acid molecule encoding a human immunodeficiency virus (HIV), for example HIV-1, HIV-2, and related viruses such as FIV-1 and SIV-1, or a HIV gene, for example LTR, nef, vif, tat, or rev.

The present invention features an enzymatic nucleic acid molecule comprising a sequence selected from the group consisting of SEQ ID NOs. 6727-6799. The invention also features an enzymatic nucleic acid molecule comprising at least one binding arm wherein one or more of said binding arms comprises a sequence complementary to a sequence selected from the group consisting of SEQ ID NOs. 6642-6726. In addition, the present invention features a siRNA nucleic acid molecule comprising sequence complementary to a sequence selected from the group consisting of SEQ ID NOs. 1-76 and 140-148.

In another embodiment, the siRNA molecule of the invention has RNA interference activity to HIV-1 expression and/or replication.

In one embodiment, a siRNA molecule of the invention comprises a double stranded RNA wherein one strand of the RNA is complementary to the RNA of HIV- 1 genome or genes. In another embodiment, a siRNA molecule of the invention comprises a double stranded RNA wherein one strand of the RNA comprises a portion of a sequence of HIV-1 genome or gene sequence. In yet another embodiment, a siRNA molecule of the invention comprises a double stranded RNA wherein both strands of RNA are connected by a non-nucleotide linker. Alternately, a siRNA molecule of the invention comprises a double stranded RNA wherein both strands of RNA are connected by a nucleotide linker, such as a loop or stem loop structure.

In one embodiment, a single strand component of a siRNA molecule of the invention is from about 14 to about 50 nucleotides in length. In another embodiment, a single strand component of a siRNA molecule of the invention is about 14, 15, 16, 17, 18, 19, 20, 21, 22, 23, 24, 25, 26, 27, or 28 nucleotides in length. In yet another embodiment, a single strand component of a siRNA molecule of the invention is about 23 nucleotides in length. In one embodiment, a siRNA molecule of the invention is from about 28 to about 56 nucleotides in length. In another embodiment, a siRNA molecule of the invention is about 40, 41, 42, 43, 44, 45, 46, 47, 48, 49, 50, 51, or 52 nucleotides in length. In yet another embodiment, a siRNA molecule of the invention is about 46 nucleotides in length.

In one embodiment, a nucleic acid molecule of the invention is adapted to treat HIV infection or acquired immunodeficiency syndrome (AIDS).

In another embodiment, the enzymatic nucleic acid molecule of the invention has an endonuclease activity to cleave RNA having HIV sequence.

In yet another embodiment, the enzymatic nucleic acid molecule of the invention is in an Inozyme, Zinzyme, G-cleaver, Amberzyme, DNAzyme or Hammerhead configuration.

In another embodiment, the Inozyme of the invention comprises a sequence complementary to a sequence selected from the group consisting of SEQ ID NOs. 6648-6655, or comprises a sequence selected from the group consisting of SEQ ID NOs. 6733-6740.

In another embodiment, the Zinzyme of the invention comprises a sequence complementary to a sequence selected from the group consisting of SEQ ID NOs. 6656-6663 and 6723-6726, or comprises a sequence selected from the group consisting of SEQ ID NOs 6741-6748 and 6795-6799.

In another embodiment, the Amberzyme of the invention comprises a sequence complementary to a sequence selected from the group consisting of SEQ ID NOs. 6656-6688, or comprises a sequence selected from the group consisting of SEQ ID NOs. 6762-6789.

In another embodiment, the DNAzyme of the invention comprises a sequence complementary to a sequence selected from the group consisting of SEQ ID NOs. 6656-6668 and 6718-6722, or comprises a sequence selected from the group consisting of SEQ ID NOs. 6749-6761 and 6790-6794.

In another embodiment, the Hammerhead of the invention comprises a sequence complementary to a sequence selected from the group consisting of SEQ ID NOs. 6642-6647, or comprises a sequence selected from the group consisting of SEQ ID NOs 6727-6732.

In one embodiment, a nucleic acid molecule of the invention comprises between 12 and 100 bases complementary to a RNA sequence encoding HIV genome, RNA, and/or proteins. In another embodiment, a nucleic acid molecule of the invention comprises between 14 and 24 bases complementary to a RNA sequence encoding HIV genome, RNA, and/or proteins.

In yet another embodiment, a nucleic acid molecule of the invention is chemically synthesized. A nucleic acid molecule of the invention can comprise at least one 2′-sugar modification, at least one nucleic acid base modification, and/or at least one phosphate backbone modification.

The present invention features a mammalian cell including a nucleic acid molecule of the invention. In one embodiment, the mammalian cell of the invention is a human cell.

The invention features a method of reducing HIV activity in a cell, comprising contacting the cell with a nucleic acid molecule of the invention, under conditions suitable for the reduction of HIV activity.

The invention also features a method of treating a subject having a condition associated with the level of HIV, comprising contacting cells of the subject with a nucleic acid molecule of the invention, under conditions suitable for the treatment.

In one embodiment, methods of treatment contemplated by the invention comprise the use of one or more drug therapies under conditions suitable for the treatment.

The invention features a method of cleaving RNA comprising a HIV nucleic acid sequence comprising contacting an enzymatic nucleic acid molecule of the invention with the RNA under conditions suitable for the cleavage. In one embodiment, the cleavage contemplated by the invention is carried out in the presence of a divalent cation, for example Mg²⁺.

The present invention features a method for treatment of acquired immunodeficiency syndrome (AIDS) or an AIDS related condition, for example Kaposi's sarcoma, lymphoma, cervical cancer, squamous cell carcinoma, cardiac myopathy, rheumatic disease, or opportunistic infection, comprising administering to a subject a nucleic acid molecule of the invention under conditions suitable for the treatment.

In one embodiment, nucleic acid molecule of the invention comprises at least five ribose residues, at least ten 2′-O-methyl modifications, and a 3′-end modification, for example a 3′-3′ inverted abasic moiety.

In another embodiment, a nucleic acid molecule of the invention further comprises phosphorothioate linkages on at least three of the 5′ terminal nucleotides.

In yet another embodiment, a DNAzyme of the invention comprises at least ten 2′-O-methyl modifications and a 3′-end modification, for example a 3′-3′ inverted abasic moiety. In a further embodiment, the DNAzyme of the invention further comprises phosphorothioate linkages on at least three of the 5′ terminal nucleotides.

In another embodiment, other drug therapies of the invention comprise antiviral therapy, monoclonal antibody therapy, chemotherapy, radiation therapy, analgesic therapy, or anti-inflammatory therapy.

In yet another embodiment, antiviral therapy of the invention comprises treatment with AZT, ddC, ddI, d4T, 3TC, Ribavirin, delvaridine, nevirapine, efravirenz, ritonavir, saquinivir, indinavir, amprenivir, nelfinavir, or lopinavir.

The invention features a composition comprising a nucleic acid molecule of the invention in a pharmaceutically acceptable carrier.

In one embodiment, the invention features a method of administering to a cell, for example a mammalian cell or human cell, an enzymatic nucleic acid molecule of the invention comprising contacting the cell with the enzymatic nucleic acid molecule under conditions suitable for the administration. The method of administration can be in the presence of a delivery reagent, for example a lipid, cationic lipid, phospholipid, or liposome.

The present invention features enzymatic nucleic acid molecules which modulate expression of nucleic acid molecules encoding HER2. The present invention also features siRNA molecules which modulate the expression of nucleic acid molecules encoding HER2.

In another embodiment, the invention features a siRNA molecule having complementarity to a sequence selected from the group consisting of SEQ ID NOs: 4656-5643 and 6632-6636.

In one embodiment, the invention features an enzymatic nucleic acid molecule comprising a sequence selected from the group consisting of SEQ ID NOs: 5644-6631 and 6637-6641.

In another embodiment, the invention features an enzymatic nucleic acid molecule comprising at least one binding arm having a sequence complementary to a sequence selected from the group consisting of SEQ ID NOs: 4656-5643 and 6632-6636.

In yet another embodiment, a nucleic acid of the invention is adapted to treat cancer.

In another embodiment, an enzymatic nucleic acid molecule of the invention has an endonuclease activity to cleave RNA having HER2 sequence.

In another embodiment, the siRNA molecule of the invention has RNA interference activity to N-Ras gene expression.

In one embodiment, a siRNA molecule of the invention comprises a double stranded RNA wherein one strand of the RNA is complementary to the RNA of HER2 gene. In another embodiment, a siRNA molecule of the invention comprises a double stranded RNA wherein one strand of the RNA comprises a portion of a sequence of RNA having of HER2 gene sequence. In yet another embodiment, a siRNA molecule of the invention comprises a double stranded RNA wherein both strands of RNA are connected by a non-nucleotide linker. Alternately, a siRNA molecule of the invention comprises a double stranded RNA wherein both strands of RNA are connected by a nucleotide linker, such as a loop or stem loop structure.

In one embodiment, a single strand component of a siRNA molecule of the invention is from about 14 to about 50 nucleotides in length. In another embodiment, a single strand component of a siRNA molecule of the invention is about 14, 15, 16, 17, 18, 19, 20, 21, 22, 23, 24, 25, 26, 27, or 28 nucleotides in length. In yet another embodiment, a single strand component of a siRNA molecule of the invention is about 23 nucleotides in length. In one embodiment, a siRNA molecule of the invention is from about 28 to about 56 nucleotides in length. In another embodiment, a siRNA molecule of the invention is about 40, 41, 42, 43, 44, 45, 46, 47, 48, 49, 50, 51, or 52 nucleotides in length. In yet another embodiment, a siRNA molecule of the invention is about 46 nucleotides in length.

In one embodiment, a DNAzyme molecule of the invention is in a “10-23” configuration. In another embodiment, a DNAzyme of the invention comprises a sequence complementary to a sequence having SEQ ID NOs: 4656-5643 and 6632-6636. In yet another embodiment, a DNAzyme molecule of the invention comprises a sequence having SEQ ID NOs: 5644-6631 and 6637-6641.

In another embodiment, a nucleic acid molecule of the invention comprises between 12 and 100 bases complementary to a nucleic acid molecule having HER2 sequence. In yet another embodiment, a nucleic acid molecule of the invention comprises between 14 and 24 bases complementary to a nucleic acid molecule having HER2 sequence.

In yet another embodiment, a nucleic acid molecule of the invention is chemically synthesized. A nucleic acid molecule of the invention can comprise at least one 2′-sugar modification, at least one nucleic acid base modification, and/or at least one phosphate backbone modification.

In one embodiment, the invention features a mammalian cell comprising a nucleic acid molecule of the invention. In another embodiment, the mammalian cell of the invention is a human cell.

In another embodiment, the invention features a method of reducing HER2 activity in a cell, comprising contacting the cell with the nucleic acid molecule of the invention, under conditions suitable for the reduction of HER2 activity.

In another embodiment, the invention features a method of treatment of a subject having a condition associated with the level of HER2, comprising contacting cells of the subject with the nucleic acid molecule of the invention, under conditions suitable for the treatment.

In one embodiment, a method of treatment of the invention further comprises the use of one or more drug therapies under conditions suitable for the treatment.

In another embodiment, the invention features a method of cleaving RNA having HER2 sequence comprising contacting an enzymatic nucleic acid molecule of the invention with the RNA under conditions suitable for the cleavage, for example, where the cleavage is carried out in the presence of a divalent cation, such as Mg2+.

In one embodiment, a nucleic acid molecule of the invention comprises a cap structure, for example a 3′,3′-linked or 5′,5′-linked deoxyabasic ribose derivative, wherein the cap structure is at the 5′-end, 3′-end, or both the 5′-end and the 3′-end of the enzymatic nucleic acid molecule.

In another embodiment, the invention features an expression vector comprising a nucleic acid sequence encoding at least one nucleic acid molecule of the invention, for example a DNAzyme or siRNA molecule, in a manner that allows expression of the nucleic acid molecule.

In yet another embodiment, the invention features a mammalian cell, for example a human cell, comprising an expression vector of the invention.

In another embodiment, an expression vector of the invention further comprises a sequence for a nucleic acid molecule complementary to a nucleic acid molecule having HER2 sequence.

In one embodiment, an expression vector of the invention comprises a nucleic acid sequence encoding two or more nucleic acid molecules, which can be the same or different. In another embodiment, an expression vector of the invention further comprises a sequence encoding an antisense nucleic acid molecule complementary to a nucleic acid molecule having a HER2 sequence.

In another embodiment, the invention features a method for treating cancer, for example breast cancer or ovarian cancer, comprising administering to a subject a nucleic acid molecule of the invention under conditions suitable for the treatment. A method of treatment of cancer of the invention can further comprise administering to a patient one or more other therapies, for example, monoclonal antibody therapy, such as Herceptin (trastuzumab); chemotherapy, such as paclitaxel (Taxol), docetaxel, cisplatin, methotrexate, cyclophosphamide, doxorubin, fluorouracil carboplatin, Leucovorin, Irinotecan (CAMPTOSAR® or CPT-11 or Camptothecin-11 or Campto), Carboplatin, edatrexate, gemcitabine, or vinorelbine; radiation therapy, or analgesic therapy and/or any combination thereof.

In another embodiment, the invention features a composition comprising a nucleic acid molecule of the invention in a pharmaceutically acceptable carrier.

In one embodiment, the invention features a method of administering to a cell, for example a mammalian cell or human cell, a nucleic acid molecule of the invention comprising contacting the cell with the nucleic acid molecule under conditions suitable for administration. The method of administration can be in the presence of a delivery reagent, for example a lipid, cationic lipid, phospholipid, or liposome.

### DETAILED DESCRIPTION OF THE INVENTION

First the drawings will be described briefly.

Drawings

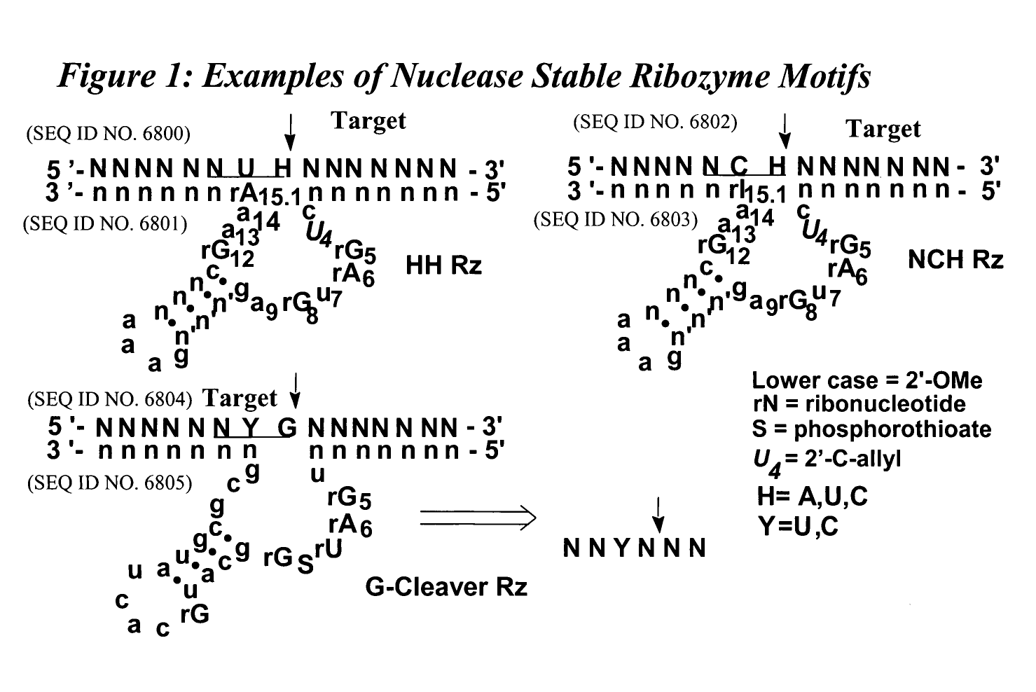

FIG. 1 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig et al., International PCT Publication No. WO 98/58058 and U.S. patent application Ser. No. 08/878,640); G-Cleaver, represents G-cleaver ribozyme motif (Kore et al., 1998, Nucleic Acids Research 26, 4116-4120, Eckstein et al., U.S. Pat. No. 6,127,173). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

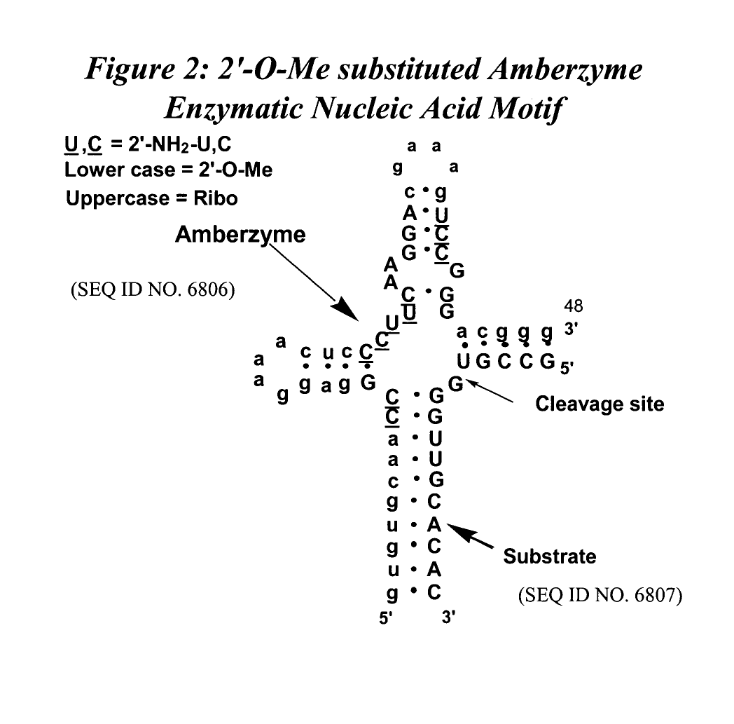

FIG. 2 shows an example of the Amberzyme ribozyme motif that is chemically stabilized (see for example Beigelman et al., International PCT publication No. WO 99/55857 and U.S. patent application Ser. No. 09/476,387.).

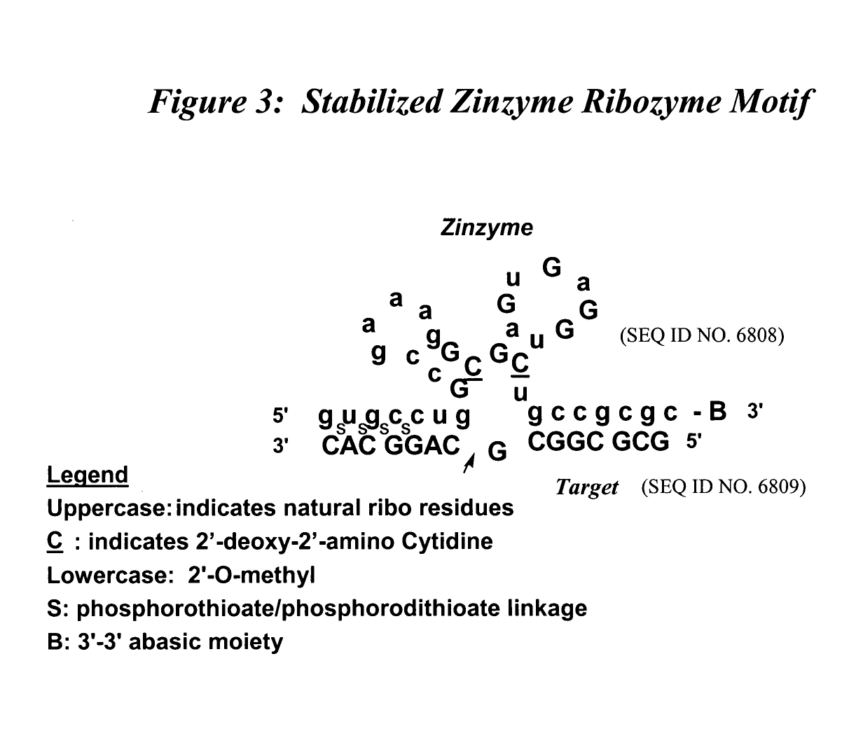

FIG. 3 shows an example of a Zinzyme A ribozyme motif that is chemically stabilized (see for example Beigelman et al., International PCT publication No. WO 99/55857 and U.S. patent application Ser. No. 09/918,728).

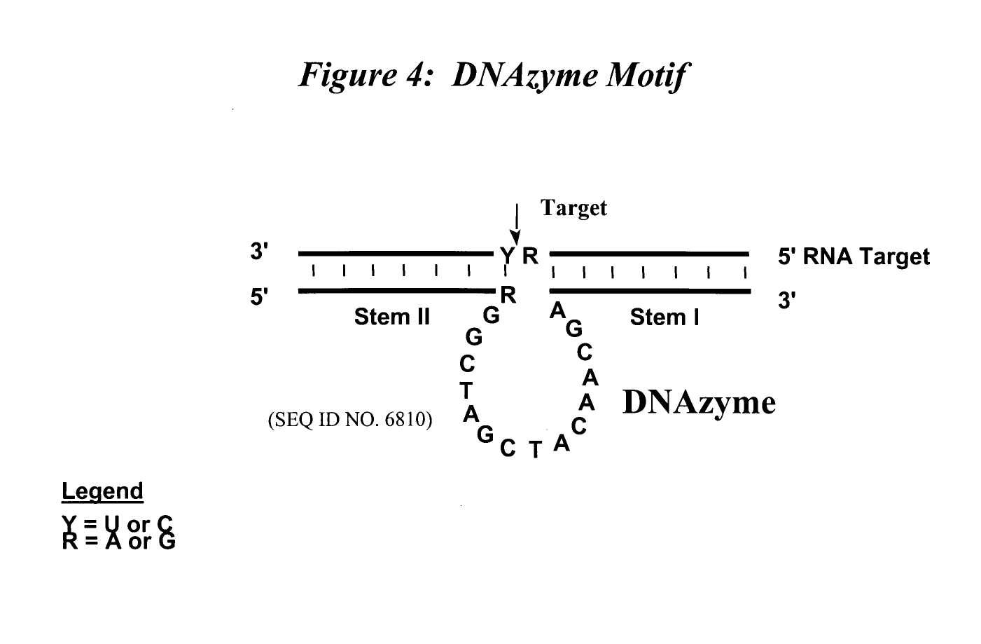

FIG. 4 shows an example of a DNAzyme motif described by Santoro et al., 1997, PNAS, 94, 4262 and Joyce et al., U.S. Pat. No. 5,807,718 .

The invention features novel nucleic acid molecules, including antisense oligonucleotides, siRNA and enzymatic nucleic acid molecules, and methods to modulate gene expression, for example, genes encoding K-Ras, H-Ras and/or N-Ras. In particular, the instant invention features nucleic-acid based molecules and methods to down-regulate the expression of K-Ras, H-Ras and/or N-Ras gene sequences.

The invention features one or more nucleic acid-based molecules and methods that independently or in combination modulate the expression of a gene or genes encoding Ras proteins. In particular embodiments, the invention features nucleic acid-based molecules and methods that modulate the expression of K-Ras gene, for example, Genbank Accession No. NM_{—}004985; H-Ras gene, for example, Genbank Accession No. NM_{—}005343; and/or N-Ras gene, for example, Genbank Accession No. NM_{—}002524.

The description below of the various aspects and embodiments is provided with reference to exemplary K-Ras, H-Ras, and N-Ras genes, referred to hereinafter collectively as Ras. However, the various aspects and embodiments are directed to equivalent sequences and also to other genes which encode K-Ras, H-Ras and/or N-Ras proteins and similar proteins to K-Ras, H-Ras and/or N-Ras. For example, the invention relates to genes with homology to genes that encode K-Ras, H-Ras and/or N-Ras and genes that encode proteins with similar function to K-Ras, H-Ras, and N-Ras proteins. Those additional genes can be analyzed for target sites using the methods described herein. Thus, the modulation and the effects of such modulation of the other genes can be determined as described herein.

In one embodiment, the invention features the use of an enzymatic nucleic acid molecule, preferably in the hammerhead, NCH, G-cleaver, amberzyme, zinzyme and/or DNAzyme motif, to modulate the expression of a Ras gene or inhibit Ras activity. In one embodiment, the invention features the use of these enzymatic nucleic acid molecules to down-regulate the expression of a Ras gene or inhibit Ras activity. In another embodiment, the invention features the use of an antisense oligonucleotide molecule to modulate, for example, down-regulate, the expression of a Ras gene or inhibit Ras activity.

The invention features novel enzymatic nucleic acid molecules, siRNA molecules, and methods to modulate expression and/or activity of human immunodeficiency virus (HIV), for example HIV-1, HIV-2, and related viruses such as FIV-1 and SIV-1, or a HIV gene, for example LTR, nef, vif tat, or rev. In particular, the instant invention features nucleic-acid based molecules and methods to inhibit the replication of a HIV or related virus.

The invention features one or more nucleic acid-based molecules and methods that independently or in combination modulate the expression of gene(s) encoded by HIV and/or inhibit the replication of HIV. In particular embodiments, the invention features nucleic acid-based molecules and methods that modulate the expression of HIV-1 encoded genes, for example (Genbank Accession No. AJ302647); HIV-2 gene, for example (Genbank Accession No. NC_{—}001722), FIV-1, for example (Genbank Accession No. NC_{—}001482), SIV-1, for example (Genbank Accession No. M66437), LTR, for example included in (Genbank Accession No. AJ302647), nef, for example included in (Genbank Accession No. AJ302647), vif for example included in (Genbank Accession No. AJ302647), tat, for example included in (Genbank Accession No. AJ302647), and rev, for example included in (Genbank Accession No. AJ302647).

The description below of the various aspects and embodiments is provided with reference to the exemplary HIV-1 gene, referred to herein as HIV. However, the various aspects and embodiments are also directed to other genes which encode HIV proteins and similar viruses to HIV. Those additional genes can be analyzed for target sites using the methods described for HIV. Thus, the inhibition and the effects of such inhibition of the other genes can be performed as described herein.

Due to the high sequence variability of the HIV genome, selection of nucleic acid molecules for broad therapeutic applications would likely involve the conserved regions of the HIV genome. Specifically, the present invention describes nucleic acid molecules that cleave the conserved regions of the HIV genome. Therefore, one nucleic acid molecule can be designed to cleave all the different isolates of HIV. Nucleic acid molecules designed against conserved regions of various HIV isolates can enable efficient inhibition of HIV replication in diverse subject populations and can ensure the effectiveness of the nucleic acid molecules against HIV quasi species which evolve due to mutations in the non-conserved regions of the HIV genome.

In one embodiment, the invention features the use of an enzymatic nucleic acid molecule, preferably in the hammerhead, NCH, G-cleaver, amberzyme, zinzyme and/or DNAzyme motif, to down-regulate the expression of HIV genes or inhibit the replication of HIV.

The invention features novel nucleic acid molecules, siRNA molecules and methods to modulate gene expression, for example, genes encoding HER2. In particular, the instant invention features nucleic-acid based molecules and methods to inhibit the expression of HER2.

The invention features one or more nucleic acid-based molecules and methods that independently or in combination modulate the expression of a gene or genes encoding HER2. In particular embodiments, the invention features nucleic acid-based molecules and methods that modulate the expression of HER2 gene, for example, Genbank Accession No. NM_{—}004448.

The description below of the various aspects and embodiments is provided with reference to an exemplary HER2 gene, referred to herein as HER2 but also known as ERB2, ERB-B2, NEU, NGL, and v-ERB-B2. However, the various aspects and embodiments are also directed to other genes which encode HER2 proteins and similar proteins to HER2. Those additional genes can be analyzed for target sites using the methods described for HER2. Thus, the inhibition and the effects of such inhibition of the other genes can be performed as described herein.

In one embodiment, the invention features the use of an enzymatic nucleic acid molecule, preferably in the hammerhead, NCH, G-cleaver, amberzyme, zinzyme and/or DNAzyme motif, to down-regulate the expression of HER2 genes or inhibit HER2 activity.

By “modulate” is meant that the expression of the gene, or level of RNAs or equivalent RNAs encoding one or more protein subunits or components, or activity of one or more proteins is up-regulated or down-regulated, such that the expression, level, or activity is greater than or less than that observed in the absence of the nucleic acid molecules of the invention.

By “inhibit” or “down-regulate” it is meant that the expression of the gene, or level of RNAs or equivalent RNAs encoding one or more protein subunits or components, or activity of one or more protein subunits or components, such as Ras, HIV, and/or HER2 protein or proteins, is reduced below that observed in the absence of the nucleic acid molecules of the invention. In one embodiment, inhibition or down-regulation with the enzymatic nucleic acid molecule preferably is below that level observed in the presence of an enzymatically inactive or attenuated enzymatic nucleic acid molecule that is able to bind to the same site on the target RNA, but is unable to cleave that RNA. In another embodiment, inhibition or down-regulation with an antisense oligonucleotide is preferably below that level observed in the presence of, for example, an oligonucleotide with scrambled sequence or with mismatches. In another embodiment, inhibition or down-regulation with an siRNA molecule is preferably below that level observed in the presence of, for example, an oligonucleotide with scrambled sequence or with mismatches. In another embodiment, inhibition or down-regulation of Ras, HIV, or HER2 expression and/or activity with the nucleic acid molecule of the instant invention is greater in the presence of the nucleic acid molecule than in its absence.

By “up-regulate” is meant that the expression of the gene, or level of RNAs or equivalent RNAs encoding one or more protein subunits or components, or activity of one or more protein subunits or components, such as Ras, HIV, or HER2 protein or proteins, is greater than that observed in the absence of the nucleic acid molecules of the invention. For example, the expression of a gene, such as Ras, HIV, or HER2 gene, can be increased in order to treat, prevent, ameliorate, or modulate a pathological condition caused or exacerbated by an absence or low level of gene expression.

By “enzymatic nucleic acid molecule” as used herein, is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term DNAzyme-based enzymatic nucleic acid is used interchangeably with phrases such as catalytic DNA, aptazyme or aptamer-binding DNAzyme, regulatable DNAzyme, catalytic oligonucleotides, nucleozyme, DNAzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving and/or ligation activity to the molecule.

By “nucleic acid molecule” as used herein is meant a molecule having nucleotides. The nucleic acid can be single, double, or multiple stranded and can comprise modified or unmodified nucleotides or non-nucleotides or various mixtures and combinations thereof

By “enzymatic portion” or “catalytic domain” is meant that portion/region of the enzymatic nucleic acid molecule essential for cleavage of a nucleic acid substrate (for example see FIGS. 1-4).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-3. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and can be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra, Hampel et al., EP0360257; Berzal-Herranz et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif or configuration is meant, an enzymatic nucleic acid molecule comprising a motif as is generally described as NCH Rz in FIG. 1 and in Ludwig et al., International PCT Publication No. WO 98/58058 and U.S. patent application Ser. No. 08/878,640. Inozymes possess endonuclease activity to cleave nucleic acid substrates having a cleavage triplet NCH/, where N is a nucleotide, C is cytidine and H is adenosine, uridine or cytidine, and “/” represents the cleavage site. H is used interchangeably with X. Inozymes can also possess endonuclease activity to cleave nucleic acid substrates having a cleavage triplet NCN/, where N is a nucleotide, C is cytidine, and “/” represents the cleavage site. “I” in FIG. 1 represents an Inosine nucleotide, preferably a ribo-Inosine or xylo-Inosine nucleoside.

By “G-cleaver” motif or configuration is meant, an enzymatic nucleic acid molecule comprising a motif as is generally described as G-cleaver Rz in FIG. 1 and in Eckstein et al., U.S. Pat. No. 6,127,173. G-cleavers possess endonuclease activity to cleave nucleic acid substrates having a cleavage triplet NYN/, where N is a nucleotide, Y is uridine or cytidine and “/” represents the cleavage site. G-cleavers can be chemically modified as is generally shown in FIG. 1.

By “amberzyme” motif or configuration is meant, an enzymatic nucleic acid molecule comprising a motif as is generally described in FIG. 2 and in Beigelman et al., International 30 PCT publication No. WO 99/55857 and U.S. patent application Ser. No. 09/476,387. Amberzymes possess endonuclease activity to cleave nucleic acid substrates having a cleavage triplet NG/N, where N is a nucleotide, G is guanosine, and “/” represents the cleavage site. Amberzymes can be chemically modified to increase nuclease stability through substitutions as are generally shown in FIG. 2. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

By “zinzyme” motif or configuration is meant, an enzymatic nucleic acid molecule comprising a motif as is generally described in FIG. 3 and in Beigelman et al., International PCT publication No. WO 99/55857 and U.S. patent application Ser. No. 09/918,728. Zinzymes possess endonuclease activity to cleave nucleic acid substrates having a cleavage triplet including but not limited to YG/Y, where Y is uridine or cytidine, and G is guanosine and “/” represents the cleavage site. Zinzymes can be chemically modified to increase nuclease stability through substitutions as are generally shown in FIG. 3, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group within its own nucleic acid sequence for activity. In particular embodiments the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 4 and is generally reviewed in Usman et al., U.S. Pat. No. 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection, see Santoro et al., supra and as generally described in Joyce et al., U.S. Pat. No. 5,807,718. Additional DNAzyme motifs can be selected by using techniques similar to those described in these references, and hence, are within the scope of the present invention. DNAzymes of the invention can comprise nucleotides modified at the nucleic acid base, sugar, or phosphate backbone. Non-limiting examples of sugar modifications that can be used in DNAzymes of the invention include 2′-O-alkyl modifications such as 2′-O-methyl or 2′-O-allyl, 2′-C-alkyl modifications such as 2′-C-allyl, 2′-deoxy-2′-amino, 2′-halo modifications such as 2′-fluoro, 2′-chloro, or 2′-bromo, isomeric modifications such as arabinofuranose or xylofuranose based nucleic acids, and other sugar modifications such as 4′-thio or 4′-carbocyclic nucleic acids. Non-limiting examples of nucleic acid based modifications that can be used in DNAzymes of the invention include modified purine heterocycles, G-clamp heterocycles, and various modified pyrimidine cycles. Non-limiting examples of backbone modifications that can be used in DNAzymes of the invention include phosphorothioate, phosphorodithioate, phosphoramidate, and methylphosphonate internucleotide linkages. DNAzymes of the invention can comprise naturally occurring nucleic acids, chimeras of chemically modified and naturally occurring nucleic acids, or completely modified nucleic acids.

In general, enzymatic nucleic acids act by first binding to a target RNA. Such binding occurs through the target binding portion of a enzymatic nucleic acid that is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single enzymatic nucleic acid molecule is able to cleave many molecules of target RNA. In addition, the enzymatic nucleic acid molecule is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of an enzymatic nucleic acid molecule.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of oligonucleotides with target nucleic acid molecules (e.g., by forming hydrogen bonds with complementary nucleotides in the target under physiological conditions) that is sufficient to the intended purpose (e.g., cleavage of target RNA by an enzyme).

By “equivalent” RNA to Ras is meant to include those naturally occurring RNA molecules having homology (partial or complete) to Ras nucleic acids or encoding for proteins with similar function as Ras proteins in various organisms, including humans, rodents, primates, rabbits, pigs, protozoans, fungi, plants, and other microorganisms and parasites. The equivalent RNA sequence can also include, in addition to the coding region, regions such as a 5′-untranslated region, a 3′-untranslated region, introns, a intron-exon junction and the like.

By “equivalent” RNA to HIV is meant to include those naturally occurring RNA molecules having homology (partial or complete) to HIV nucleic acids or encoding for proteins with similar function as HIV proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “equivalent” RNA to HER2 is meant to include those naturally occurring RNA molecules having homology (partial or complete) to HER2 nucleic acids or encoding for proteins with similar function as HER2 proteins in various organisms, including humans, rodents, primates, rabbits, pigs, protozoans, fungi, plants, and other microorganisms and parasites. The equivalent RNA sequence also includes, in addition to the coding region, regions such as a 5′-untranslated region, a 3′-untranslated region, introns, a intron-exon junction and the like.

By “homology” is meant the nucleotide sequence of two or more nucleic acid molecules is partially or completely identical.

By “component” of HIV is meant a peptide or protein expressed from an HIV gene, for example nef vif tat, or rev viral gene products.

By “component” of HER2 is meant a peptide or protein subunit expressed from a HER2 gene.

By “component” of Ras is meant a peptide or protein subunit expressed from a Ras gene.

By “gene” it is meant a nucleic acid that encodes an RNA, for example, nucleic acid sequences including but not limited to structural genes encoding a polypeptide.

“Complementarity” refers to the ability of a nucleic acid to form hydrogen bond or bonds with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety. By “decoy” is meant a nucleic acid molecule, for example RNA or DNA, or aptamer that is designed to preferentially bind to a predetermined ligand. Such binding can result in the inhibition or activation of a target molecule. A decoy or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy can be designed to bind to Ras and block the binding of Ras or a decoy can be designed to bind to Ras and prevent interaction with the Ras protein.

By “aptamer” or “nucleic acid aptamer” as used herein is meant a nucleic acid molecule that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand binding domain of a protein, thereby preventing interaction of the naturally occurring ligand with the protein. Similarly, the nucleic acid molecules of the instant invention can bind to RAS, Her-2 or HIV encoded RNA or proteins receptors to block activity of the activity of target protein or nucleic acid. This is a non-limiting example and those in the art will recognize that other embodiments can be readily generated using techniques generally known in the art, see for example Gold et al., U.S. Pat. Nos. 5,475,096 and 5,270,163; Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

The term “short interfering RNA” or “siRNA” as used herein refers to a double stranded nucleic acid molecule capable of RNA interference “RNAi”, see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. As used herein, siRNA molecules need not be limited to those molecules containing only RNA, but further encompasses chemically modified nucleotides and non-nucleotides.

Nucleic acid molecules that modulate expression of Ras-specific RNAs represent a therapeutic approach to treat cancer, including, but not limited to colorectal cancer, bladder cancer, lung cancer, pancreatic cancer, breast cancer, or prostate cancer and any other cancer, disease or condition that responds to the modulation of Ras expression.

Nucleic acid molecules that modulate expression of HIV-specific RNAs also represent a therapeutic approach to treat acquired immunodeficiency syndrome (AIDS) and/or any other disease, condition, or syndrome which respond to the modulation of HIV expression.

Nucleic acid molecules that modulate expression of HER2-specific RNAs represent a therapeutic approach to treat cancer, including, but not limited to breast and ovarian cancer and any other cancer, disease or condition that responds to the modulation of HER2 expression.

In one embodiment of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 2; Beigelman et al., U.S. Ser. No. 09/301,511) and Zinzyme (FIG. 3) (Beigelman et al., U.S. Ser. No. 09/301,511), all included by reference herein including drawings, can also be used in the present invention. These specific motifs or configurations are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In one embodiment of the present invention, a nucleic acid molecule of the instant invention can be between about 10 and 100 nucleotides in length. Exemplary enzymatic nucleic acid molecules of the invention are shown in the Tables herein. For example, enzymatic nucleic acid molecules of the invention are preferably between about 15 and 50 nucleotides in length, more preferably between about 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between about 15 and 40 nucleotides in length, more preferably between about 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between about 15 and 75 nucleotides in length, more preferably between about 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between about 10 and 40 nucleotides in length, more preferably between about 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for a nucleic acid molecule to be of length and conformation sufficient and suitable for the nucleic acid molecule to interact with its target and/or catalyze a reaction contemplated herein. The length of nucleic acid molecules of the instant invention are not limiting within the general limits stated.

Preferably, a nucleic acid molecule that modulates, for example, down-regulates Ras, HIV, and/or HER2 expression and/or activity, comprises between 12 and 100 bases complementary to a RNA molecule of Ras, HIV, and/or HER2 respectively. Even more preferably, a nucleic acid molecule that modulates Ras, HIV, and/or HER2 expression comprises between 14 and 24 bases complementary to a RNA molecule of Ras, HIV, and/or HER2 respectively.

The invention provides a method for producing a class of nucleic acid-based gene modulating agents that exhibit a high degree of specificity for RNA of a desired target. For example, an enzymatic nucleic acid molecule is preferably targeted to a highly conserved sequence region of target RNAs encoding Ras (and specifically a Ras gene) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., enzymatic nucleic acid molecules, siRNA, antisense, and/or DNAzymes) can be expressed from DNA and/or RNA vectors that are delivered to specific cells.

As used herein “cell” is used in its usual biological sense, and does not refer to an entire multicellular organism. A cell can, for example, be in vitro, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

By “Ras proteins” is meant, a peptide or protein comprising Ras tyrosine kinase-type cell surface receptor or a peptide or protein encoded by a Ras gene, such as K-Ras, H-Ras, or N-Ras.

By “HIV proteins” is meant, a peptide or protein comprising a component of HIV or a peptide or protein encoded by a HIV gene.

By “HER2 proteins” is meant, a peptide or protein comprising HER2/ERB2/NEU tyrosine kinase-type cell surface receptor or a peptide or protein encoded by a HER2/ERB2/NEU gene.

By “highly conserved sequence region” is meant, a nucleotide sequence of one or more regions in a target gene that does not vary significantly from one generation to the other or from one biological system to the other.

Nucleic acid-based modulators, including inhibitors, of Ras expression are useful for the prevention and/or treatment of cancer, including but not limited to breast cancer and ovarian cancer and any other disease or condition that respond to the modulation of Ras expression.

Nucleic acid-based inhibitors of HIV expression are useful for the prevention and/or treatment of acquired immunodeficiency disease (AIDS) and related diseases and conditions, including but not limited to Kaposi's sarcoma, lymphoma, cervical cancer, squamous cell carcinoma, cardiac myopathy, rheumatic diseases, and opportunistic infection, for example Pneumocystis carinii, Cytomegalovirus, Herpes simplex, Mycobacteria, Cryptococcus, Toxoplasma, Progressive multifocal leucoencepalopathy (Papovavirus), Mycobacteria, Aspergillus, Cryptococcus, Candida, Cryptosporidium, Isospora belli, Microsporidia and any other disease or condition which respond to the modulation of HIV expression.

Nucleic acid-based inhibitors of HER2 expression are useful for the prevention and/or treatment of cancer, including but not limited to breast cancer and ovarian cancer and any other disease or condition that respond to the modulation of HER2 expression.

By “related” is meant that the reduction of RAS, HIV, or HER2 expression (specifically RAS, HIV, or HER2 genes respectively) RNA levels and thus reduction in the level of the respective protein relieves, to some extent, the symptoms of the disease or condition.

The nucleic acid-based molecules of the invention can be added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers. In certain embodiments, the enzymatic nucleic acid molecules comprise sequences that are complementary to the substrate sequences in the Tables herein. Examples of such enzymatic nucleic acid molecules also are shown in the Tables herein. Examples of such enzymatic nucleic acid molecules consist essentially of sequences defined in these tables.

In another embodiment, the invention features siRNA, antisense nucleic acid molecules and 2-5A chimeras comprising sequences complementary to the substrate sequences shown in the Tables herein. Such nucleic acid molecules can comprise sequences as shown for the binding arms of the enzymatic nucleic acid molecules in the Tables. Similarly, triplex molecules can be targeted to corresponding DNA target regions; such molecules can comprise the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to a substrate such that the substrate molecule forms a loop, and/or an antisense molecule can bind such that the antisense molecule forms a loop. Thus, the antisense molecule can be complementary to two or more non-contiguous substrate sequences. In addition, two or more non-contiguous sequence portions of an antisense molecule can be complementary to a target sequence.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present that do not interfere with such cleavage. Thus, a core region of an enzymatic nucleic acid molecule can, for example, include one or more loop, stem-loop structure, or linker that does not prevent enzymatic activity. Thus, various regions in the sequences in the Tables can be such a loop, stem-loop, nucleotide linker, and/or non-nucleotide linker and can be represented generally as sequence “X”. The nucleic acid molecules of the instant invention, such as Hammerhead, Inozyme, G-cleaver, amberzyme, zinzyme, DNAzyme, antisense, 2-5A antisense, triplex forming nucleic acid, and decoy nucleic acids, can contain other sequences or non-nucleotide linkers that do not interfere with the function of the nucleic acid molecule.

Sequence X can be a linker of ≧2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, sequence X can be a non-nucleotide linker. In yet another embodiment, the nucleotide linker X can be a nucleic acid aptamer, such as an ATP aptamer, Ras Rev aptamer (RRE), Ras Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, a non-nucleotide linker X is as defined herein. Non-nucleotides as can include abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109;

Ma et al, Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, enzymatic nucleic acid molecules, siRNA molecules or antisense molecules that interact with target RNA molecules and modulate gene expression activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, adeno-associated virus, retrovirus, adenovirus, or alphavirus as well as others known in the art. Preferably, recombinant vectors capable of expressing enzymatic nucleic acid molecules or antisense are delivered as described below, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to target RNA and modulate its function or expression. Delivery of enzymatic nucleic acid molecule or antisense expressing vectors can be systemic, such as by intravenous or intramuscular administration, by administration to target cells ex-planted from the patient followed by reintroduction into the patient, or by any other means that allows for introduction into a desired target cell. Antisense DNA and DNAzymes can be expressed via the use of a single stranded DNA intracellular expression vector.

By “vectors” is meant any nucleic acid- and/or viral-based technique used to deliver a desired nucleic acid.

By “subject” or “patient” is meant an organism that is a donor or recipient of explanted cells or the cells of the organism. “Subject” or “patient” also refers to an organism to which the nucleic acid molecules of the invention can be administered. Preferably, a subject or patient is a mammal or mammalian cells. More preferably, a subject or patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme, for example, with a nucleic acid molecule comprising chemical modifications. In some cases, the activity or stability of the nucleic acid molecule can be decreased (i.e., less than ten-fold), but the overall activity of the nucleic acid molecule is enhanced, in vivo.

Nucleic acid molecules of the instant invention, individually, or in combination or in conjunction with other drugs, can be used to treat diseases or conditions discussed above. For example, to treat a disease or condition associated with the levels of Ras, HIV, or HER2, a subject can be treated, or other appropriate cells can be treated, as is evident to those skilled in the art, individually or in combination with one or more drugs under conditions suitable for the treatment.

In a further embodiment, the described molecules, such as antisense, siRNA, or enzymatic nucleic acid molecules, can be used in combination with other known treatments to treat conditions or diseases discussed above. For example, the described molecules can be used in combination with one or more known therapeutic agents to treat cancer, for example colorectal cancer, bladder cancer, lung cancer, pancreatic cancer, breast cancer, or prostate cancer, and any other disease or condition that respond to the modulation of Ras expression.

In another embodiment, the invention features nucleic acid-based inhibitors (e.g., enzymatic nucleic acid molecules, (including DNAzymes), siRNA and methods for their use to down regulate or inhibit the expression of genes (e.g., Ras genes) capable of progression and/or maintenance of cancer and/or other disease states that respond to the modulation of Ras expression.

In a further embodiment, the described molecules, such as antisense, siRNA, or enzymatic nucleic acids, can be used in combination with other known treatments to treat conditions or diseases discussed above. For example, the described molecules can be used in combination with one or more known therapeutic agents to treat acquired immunodeficiency disease (AIDS) and related diseases and conditions, including but not limited to Kaposi's sarcoma, lymphoma, cervical cancer, squamous cell carcinoma, cardiac myopathy, rheumatic diseases, and opportunistic infection, for example Pneumocystis carinii, Cytomegalovirus, Herpes simplex, Mycobacteria, Cryptococcus, Toxoplasma, Progressive multifocal leucoencepalopathy (Papovavirus), Mycobacteria, Aspergillus, Cryptococcus, Candida, Cryptosporidium, Isospora belli, Microsporidia and any other disease or condition which respond to the modulation of HIV expression.

Nucleic acid molecules of the instant invention, individually, or in combination or in conjunction with other drugs, can be used to treat diseases or conditions discussed above. For example, to treat a disease or condition associated with the levels of HER2, a patient can be treated, or other appropriate cells can be treated, as is evident to those skilled in the art, individually or in combination with one or more drugs under conditions suitable for the treatment.

In a further embodiment, the described molecules, such as antisense, siRNA or enzymatic nucleic acid molecules, can be used in combination with other known treatments to treat conditions or diseases discussed above. For example, the described molecules can be used in combination with one or more known therapeutic agents to treat cancer, for example ovarian cancer and/or breast cancer, and any other disease or condition that respond to the modulation of HER2 expression.

In another embodiment, the invention features nucleic acid-based inhibitors (e.g., enzymatic nucleic acid molecules, (including ribozymes, antisense nucleic acids, 2-5A antisense chimeras, triplex DNA, antisense nucleic acids containing RNA cleaving chemical groups), siRNA and methods for their use to down regulate or inhibit the expression of genes (e.g., HER2 genes) capable of progression and/or maintenance of cancer and/or other disease states that respond to the modulation of HER2 expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”.

Other features and advantages of the invention will be apparent from the following description of the preferred embodiments thereof, and from the claims.

Mechanism of Action of Nucleic Acid Molecules of the Invention as is Know in the Art

Antisense: Antisense molecules can be modified or unmodified RNA, DNA, or mixed polymer oligonucleotides and primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). Backbone modified DNA chemistry which have been thus far been shown to act as substrates for RNase H are phosphorothioates, phosphorodithioates, and borontrifluoridates. In addition, 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., USSN 60/101,174, filed on Sep. 21, 1998). All of these references are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

RNA interference: RNA interference refers to the process of sequence specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease m enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., 2001, Genes Dev., 15, 188).

Short interfering RNA mediated RNAi has been studied in a variety of systems. Fire et al., 1998, Nature, 391, 806, were the first to observe RNAi in C. Elegans. Wianny and Goetz, 1999, Nature Cell Biol., 2, 70, describes RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J, 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309), however siRNA molecules lacking a 5′-phosphate are active when introduced exogenously, suggesting that 5′-phosphorylation of siRNA constructs may occur in vivo.

Enzvmatic Nucleic Acid: Several varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention can modulate, e.g., down-regulate, Ras protein expression and can be used to treat disease or diagnose disease associated with the levels of Ras, HIV and/or HER2. Enzymatic nucleic acid sequences targeting Ras, HIV and/or HER2 RNA and sequences that can be targeted with nucleic acid molecules of the invention to down-regulate Ras expression are shown in the Tables herein.

The enzymatic nature of an enzymatic nucleic acid molecule allows the concentration of enzymatic nucleic acid molecule necessary to affect a therapeutic treatment to be lower than a nucleic acid molecule lacking enzymatic activity. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a enzymatic nucleic acid molecule.

Nucleic acid molecules having an endonuclease enzymatic activity are able to repeatedly cleave other separate RNA molecules in a nucleotide base sequence-specific manner. With proper design and construction, such enzymatic nucleic acid molecules can be targeted to virtually any RNA transcript, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to modulate, including down-regulate, Ras, HIV and/or HER2 expression. These allosteric enzymatic nucleic acids or allozymes (see for example George et al., U.S. Pat. Nos. 5,834,186 and 5,741,679, Shih et al., U.S. Pat. No. 5,589,332, Nathan et al., US Patent No 5,871,914, Nathan and Ellington, International PCT publication No. WO 00/24931, Breaker et al., International PCT Publication Nos. WO 00/26226 and 98/27104, and Sullenger et al, International PCT publication No. WO 99/29842) are designed to respond to a signaling agent, for example, mutant Ras, HIV and/or HER2 protein, wild-type Ras, HIV and/or HER2 protein, mutant Ras, HIV and/or HER2 RNA, wild-type Ras, HIV and/or HER2 RNA, other proteins and/or RNAs involved in Ras, HIV and/or HER2 activity, compounds, metals, polymers, molecules and/or drugs that are targeted to Ras, HIV and/or HER2 expressing cells etc., which, in turn, modulate the activity of the enzymatic nucleic acid molecule. In response to interaction with a predetermined signaling agent, the activity of the allosteric enzymatic nucleic acid molecule is activated or inhibited such that the expression of a particular target is selectively regulated, including down-regulated. The target can comprise wild-type Ras, HIV and/or HER2, mutant Ras, HIV and/or HER2, a component of Ras, HIV and/or HER2, and/or a predetermined cellular component that modulates Ras, HIV and/or HER2 activity. For example, allosteric enzymatic nucleic acid molecules that are activated by interaction with a RNA encoding Ras, HIV and/or HER2 protein can be used as therapeutic agents in vivo. The presence of RNA encoding the Ras, HIV and/or HER2 protein activates the allosteric enzymatic nucleic acid molecule that subsequently cleaves the RNA encoding Ras, HIV and/or HER2 protein, resulting in the inhibition of Ras, HIV and/or HER2 protein expression. In this manner, cells that express the Ras, HIV and/or HER2 protein are selectively targeted.

In another non-limiting example, an allozyme can be activated by a Ras, HIV and/or HER2 protein, peptide, or mutant polypeptide that causes the allozyme to inhibit the expression of Ras, HIV and/or HER2 gene, by, for example, cleaving RNA encoded by Ras, HIV and/or HER2 gene. In this non-limiting example, the allozyme acts as a decoy to inhibit the function of Ras, HIV and/or HER2 and also inhibit the expression of Ras, HIV and/or HER2 once activated by the Ras, HIV and/or HER2 protein.

Target sites

Targets for useful enzymatic nucleic acid molecules and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific non-limiting examples of such methods. Enzymatic nucleic acid molecules to such targets are designed as described in the above applications and synthesized to be tested in vitro and in vivo, as also described. The sequences of human K-Ras, H-Ras, HIV-1 and HER2 RNAs were screened for optimal enzymatic nucleic acid target sites using a computer-folding algorithm. Nucleic acid molecule binding/cleavage sites were identified. These sites are shown in the Tables (all sequences are 5′ to 3′ in the tables). The nucleotide base position is noted in the Tables as that site to be cleaved by the designated type of enzymatic nucleic acid molecule. Human sequences can be screened and enzymatic nucleic acid molecule and/or antisense thereafter designed, as discussed in Stinchcomb et al., WO 95/23225. In addition, mouse targeted nucleic acid molecules can be used to test efficacy of action of the enzymatic nucleic acid molecule, siRNA and/or antisense prior to testing in humans.

In addition, enzymatic nucleic acid, siRNA, and antisense nucleic acid molecule binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions, such as between, for example the binding arms and the catalytic core of an enzymatic nucleic acid, are eliminated from consideration. Varying binding arm lengths can be chosen to optimize activity.

Antisense, hammerhead, DNAzyme, NCH, amberzyme, zinzyme or G-Cleaver enzymatic nucleic acid molecule, siRNA, and antisense nucleic acid binding/cleavage sites were identified and were designed to anneal to various sites in the RNA target. The enzymatic nucleic acid binding arms or siRNA and antisense nucleic acid sequences are complementary to the target site sequences described above. The nucleic acid molecules are chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic acid Molecules

Synthesis of nucleic acids greater than 100 nucleotides in length can be difficult using automated methods, and the therapeutic cost of such molecules can be prohibitive. In this invention, small nucleic acid motifs (“small” refers to nucleic acid motifs less than about 100 nucleotides in length, preferably less than about 80 nucleotides in length, and more preferably less than about 50 nucleotides in length; e.g., DNAzymes) are preferably used for exogenous delivery. The simple structure of these molecules increases the ability of the nucleic acid to invade targeted regions of RNA structure. Exemplary molecules of the instant invention are chemically synthesized as described herein, and others can similarly be synthesized.

Oligonucleotides (e.g., DNAzymes, antisense) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table I outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNAzymes is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for RNA and chemically modified RNA or DNA, including certain enzymatic nucleic acid molecules and siRNA molecules, follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table I outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA·3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive nucleic acid molecules or binding attenuated control (BAC) oligonucleotides can be synthesized by substituting one or more nucleotides in the nucleic acid molecule to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Enzymatic nucleic acid molecules are purified by gel electrophoresis using known methods or are purified by high pressure liquid chromatography (HPLC; See Wincott et al., Supra, the totality of which is hereby incorporated herein by reference) and are re-suspended in water.

The sequences of the nucleic acid molecules, including enzymatic nucleic acid molecules and antisense, that are chemically synthesized, are shown in the Tables herein. These sequences are representative only of many more such sequences where the enzymatic portion of the enzymatic nucleic acid molecule (all but the binding arms) is modified to affect activity. For example, the enzymatic nucleic acid sequences listed in the Tables can be formed of deoxyribonucleotides or other nucleotides or non-nucleotides. Such enzymatic nucleic acid molecules with enzymatic activity are equivalent to the enzymatic nucleic acid molecules described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra, all of which are hereby incorporated by reference in their entirety). All of the above references describe various chemical modifications that can be made to the base, phosphate and/or sugar moieties of the nucleic acid molecules described herein. Modifications which enhance their efficacy in cells, and removal of bases from nucleic acid molecules to shorten oligonucleotide synthesis times and reduce chemical requirements are desired.

There are several examples of sugar, base and phosphate modifications that can be introduced into nucleic acid molecules with significant enhancement in their nuclease stability and efficacy. For example, oligonucleotides can be modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry , 35, 14090). Sugar modification of nucleic acid molecules are also known to increase efficacy (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No.5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., USSN 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). The publications describe general methods and strategies to determine the location of incorporation of sugar, base and/or phosphate modifications and the like into enzymatic nucleic acid molecules without inhibiting catalysis. Similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, excessive modifications can cause some toxicity. Therefore, when designing nucleic acid molecules, the amount of these internucleotide linkages should be minimized. The reduction in the concentration of these linkages can lower toxicity, resulting in increased efficacy and higher specificity of the therapeutic nucleic acid molecules.

Nucleic acid molecules having chemical modifications that maintain or enhance activity are provided. Such nucleic acid molecules are also generally more resistant to nucleases than unmodified nucleic acid molecules. Thus, the in vitro and/or in vivo activity should not be significantly lowered. Therapeutic nucleic acid molecules delivered exogenously are optimally stable within cells until translation of the target RNA has been inhibited long enough to reduce the levels of the undesirable protein. This period of time varies between hours to days, depending upon the disease state. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability as described above.

In one embodiment, nucleic acid molecules of the invention include one or more G-clamp nucleotides. A G-clamp nucleotide is a modified cytosine analog wherein modifications result in the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substation within an oligonucleotide can result in substantially enhanced helical thermal stability and mismatch discrimination when hybridized to complementary oligonucleotides. The inclusion of such nucleotides in nucleic acid molecules of the invention can enable both enhanced affinity and specificity to nucleic acid targets.

In another embodiment, the invention features conjugates and/or complexes of nucleic acid molecules targeting Ras genes such as K-Ras, H-Ras, and/or N-Ras. Compositions and conjugates are used to facilitate delivery of molecules into a biological system, such as cells. The conjugates provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel agents for the delivery of molecules, including but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example, enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus containing group and saturated or unsaturated alkyl group, optionally substituted with OH, COOH, oxo, amine, or substituted or unsubstituted aryl groups.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of subjects with nucleic acid molecules can also include combinations of different types of nucleic acid molecules.

In the case that down-regulation of the target is desired, therapeutic nucleic acid molecules (e.g., DNAzymes) delivered exogenously are optimally stable within cells until translation of the target RNA has been inhibited long enough to reduce the levels of the targeted protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and others known in the art have expanded the ability to modify nucleic acid molecules by introducing nucleotide modifications to enhance their nuclease stability as described above.

In another embodiment, nucleic acid catalysts having chemical modifications that maintain or enhance enzymatic activity are provided. Such nucleic acids are also generally more resistant to nucleases than unmodified nucleic acid. Thus, the in vitro and/or in vivo the activity of the nucleic acid should not be significantly lowered. As exemplified herein, such enzymatic nucleic acids are useful for in vitro and/or in vivo techniques even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment, the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

The term “alkyl” as used herein refers to a saturated aliphatic hydrocarbon, including straight-chain, branched-chain “isoalkyl”, and cyclic alkyl groups. The term “alkyl” also comprises alkoxy, alkyl-thio, alkyl-thio-alkyl, alkoxyalkyl, alkylamino, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Preferably, the alkyl group has 1 to 12 carbons. More preferably it is a lower alkyl of from about 1 to 7 carbons, more preferably about 1 to 4 carbons. The alkyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkenyl groups containing at least one carbon-carbon double bond, including straight-chain, branched-chain, and cyclic groups. Preferably, the alkenyl group has about 2 to 12 carbons. More preferably it is a lower alkenyl of from about 2 to 7 carbons, more preferably about 2 to 4 carbons. The alkenyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups.

The term “alkyl” also includes alkynyl groups containing at least one carbon-carbon triple bond, including straight-chain, branched-chain, and cyclic groups. Preferably, the alkynyl group has about 2 to 12 carbons. More preferably it is a lower alkynyl of from about 2 to 7 carbons, more preferably about 2 to 4 carbons. The alkynyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Alkyl groups or moieties of the invention can also include aryl, alkylaryl, carbocyclic aryl, heterocyclic aryl, amide and ester groups. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from about 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide”refers to an —C(O)—NH—R, where R is either alkyl, aryl, alkylaryl or hydrogen. An “ester” refers to an —C(O)—OR′, where R is either alkyl, aryl, alkylaryl or hydrogen.

The term “alkoxyalkyl” as used herein refers to an alkyl-O-alkyl ether, for example, methoxyethyl or ethoxymethyl.

The term “alkyl-thio-alkyl” as used herein refers to an alkyl-S-alkyl thioether, for example, methylthiomethyl or methylthioethyl.

The term “amino” as used herein refers to a nitrogen containing group as is known in the art derived from ammonia by the replacement of one or more hydrogen radicals by organic radicals. For example, the terms “aminoacyl” and “aminoalkyl” refer to specific N-substituted organic radicals with acyl and alkyl substituent groups respectively.

The term “amination” as used herein refers to a process in which an amino group or substituted amine is introduced into an organic molecule.

The term “exocyclic amine protecting moiety” as used herein refers to a nucleobase amino protecting group compatible with oligonucleotide synthesis, for example, an acyl or amide group.

The term “alkenyl” as used herein refers to a straight or branched hydrocarbon of a designed number of carbon atoms containing at least one carbon-carbon double bond. Examples of “alkenyl” include vinyl, allyl, and 2-methyl-3-heptene.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkenyl” as used herein refers to a C3-C8 cyclic hydrocarbon containing at least one carbon-carbon double bond. Examples of cycloalkenyl include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloalkyl” as used herein refers to a C3-C8 cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C3-C7 cycloalkyl group attached to the parent molecular moiety through an alkyl group, as defined above. Examples of cycloalkylalkyl groups include cyclopropylmethyl and cyclopentylethyl.

The terms “halogen” or “halo” as used herein refers to indicate fluorine, chlorine, bromine, and iodine.

The term “heterocycloalkyl,” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “C1-C6 hydrocarbyl” as used herein refers to straight, branched, or cyclic alkyl groups having 1-6 carbon atoms, optionally containing one or more carbon-carbon double or triple bonds. Examples of hydrocarbyl groups include, for example, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, vinyl, 2-pentene, cyclopropylmethyl, cyclopropyl, cyclohexylmethyl, cyclohexyl and propargyl. When reference is made herein to C1-C6 hydrocarbyl containing one or two double or triple bonds it is understood that at least two carbons are present in the alkyl for one double or triple bond, and at least four carbons for two double or triple bonds.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein. There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et aL., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5 -methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′—NH₂ or 2′—O—NH_{2,} which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., DNAzyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of subjects with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995, which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819, all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a subject.

The negatively charged polynucleotides of the invention can be administered (e.g., RNA, DNA or protein) and introduced into a subject by any standard means described herein and known in the art, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for exaple the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes, which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example, sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example, ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example, sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient or subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient or subject in combination with other therapeutic compounds to increase the overall therapeutic effect. The use of multiple compounds to treat an indication can increase the beneficial effects while reducing the presence of side effects.

In another aspect of the invention, nucleic acid molecules of the present invention are preferably expressed from transcription units (see for example Couture et al., 1996, TIG., 12, 510, Skillern et al., International PCT Publication No. WO 00/22113, Conrad, International PCT Publication No. WO 00/22114, and Conrad, U.S. Pat. No. 6,054,299) inserted into DNA or RNA vectors. The recombinant vectors are preferably DNA plasmids or viral vectors. Enzymatic nucleic acid expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intra-muscular administration, by administration to target cells ex-planted from the subject followed by reintroduction into the subject, or by any other means that would allow for introduction into the desired target cell (for a review see Couture et al., 1996, TIG., 12, 510).

One aspect of the invention features an expression vector comprising a nucleic acid sequence encoding at least one of the nucleic acid molecules of the instant invention. The nucleic acid sequence encoding the nucleic acid molecule of the instant invention is operably linked in a manner that allows expression of that nucleic acid molecule.

Another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner that allows expression and/or delivery of said nucleic acid molecule.

In another embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

### Example 1

### Identification of Potential Target Sites in Human Ras RNA

The sequence of human Ras genes were screened for accessible sites using a computer-folding algorithm. Regions of the RNA that do not form secondary folding structures and contain potential enzymatic nucleic acid molecule and/or antisense binding/cleavage sites were identified. The sequences of K-Ras and H-Ras binding/cleavage sites are shown in Tables II and III.

### Example 2

### Selection of Enzymatic Nucleic Acid Cleavage Sites in Human Ras RNA

Enzymatic nucleic acid molecule target sites were chosen by analyzing sequences of Human K-Ras and H-Ras (for example, Genbank accession Nos: NM_{—}004985 and NM_{—}005343 respectively) and prioritizing the sites on the basis of folding. Enzymatic nucleic acid molecules were designed that can bind each target and were individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Enzymatic Nucleic Acid Molecules for Efficient Cleavage and/or Blocking of Ras RNA

DNAzyme molecules are designed to anneal to various sites in the RNA message. The binding arms of the DNAzyme molecules are complementary to the target site sequences described above. The DNAzymes were chemically synthesized. The method of synthesis used followed the procedure for nucleic acid synthesis as described herein and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%. The sequences of the chemically synthesized DNAzyme molecules used in this study are shown below in Tables II and III.

### Example 4

### DNAzyme Cleavage of Ras RNA Target in vitro

DNAzymes targeted to the human K-Ras and H-Ras RNA are designed and synthesized as described above. These enzymatic nucleic acid molecules can be tested for cleavage activity in vitro, for example, using the following procedure. The target sequences and the nucleotide location within the K-Ras and H-Ras RNA are given in Tables II and III respectively.

Cleavage Reactions:

DNAzymes and substrates were synthesized in 96-well format using 0.2 μmol scale. Substrates were 5′-³²P labeled and gel purified using 7.5% polyacrylamide gels, and eluting into water. Assays were done by combining trace substrate with 500 nM DNAzyme or greater, and initiated by adding final concentrations of 40 mM Mg⁺², and 50 mM Tris-Cl pH 8.0. For each DNAzyme/substrate combination a control reaction was done to ensure cleavage was not the result of non-specific substrate degradation. A single three hour time point was taken and run on a 15% polyacrylamide gel to asses cleavage activity. Gels were dried and scanned using a Molecular Dynamics Phosphorimager and quantified using Molecular Dynamics ImageQuant software. Percent cleaved was determined by dividing values for cleaved substrate bands by full-length (uncleaved) values plus cleaved values and multiplying by 100 (%cleaved=[C/(U+C)]*100).

### Example 5

### DNAzyme Cleavage of Ras RNA Target in vivo

Cell Culture

Wickstrom, 2001, Mol. Biotechnol., 18, 35-35, describes a cell culture system in which antisense oligonucleotides targeting H-Ras were studied in transformed mouse cells that form solid tumors. Treatment of cells with antisense targeting H-Ras resulted in the sequence specific and dose dependent inhibition of H-Ras expression. In this study, it was determined that antisense targeting the first intron region of H-Ras were more effective than antisense targeting the initiation codon region.

Kita et al., 1999, Int. J. Cancer, 80, 553-558, describes the growth inhibition of human pancreatic cancer cell lines by antisense oligonucleotides specific to mutated K-Ras genes. Antisense oligonucleotides were transfected to the transformed cells using liposomes. Cellular proliferation, K-Ras mRNA expression, and K-Ras protein synthesis were all evaluated as endpoints. Sato et al., 2000, Cancer Lett., 155, 153-161, describes another human pancreatic cancer cell line, HOR-P1, that is characterized by high angiogenic activity and metastatic potential. Genetic and molecular analysis of this cell line revealed both increased telomerase activity and a mutation in the K-Ras oncogene.

A variety of endpoints have been used in cell culture models to look at Ras-mediated effects after treatment with anti-Ras agents. Phenotypic endpoints include inhibition of cell proliferation, RNA expression, and reduction of Ras protein expression. Because Ras oncogene mutations are directly associated with increased proliferation of cetain tumor cells, a proliferation endpoint for cell culture assays is preferably used as the primary screen. There are several methods by which this endpoint can be measured. Following treatment of cells with DNAzymes, cells are allowed to grow (typically 5 days) after which either the cell viability, the incorporation of [³H] thymidine into cellular DNA and/or the cell density can be measured. The assay of cell density is done in a 96-well format using commercially available fluorescent nucleic acid stains (such as Syto® 13 or CyQuant®). As a secondary, confirmatory endpoint a DNAzyme-mediated decrease in the level of Ras protein expression is evaluated using a Ras-specific ELISA.

Animal Models

Evaluating the efficacy of anti-Ras agents in animal models is an important prerequisite to human clinical trials. As in cell culture models, the most Ras sensitive mouse tumor xenografts are those derived from cancer cells that express mutant Ras proteins. Nude mice bearing H-Ras transformed bladder cancer cell xenografts were sensitive to an anti-Ras antisense nucleic acid, resulting in an 80% inhibition of tumor growth after a 31 day treatment period (Wickstrom, 2001, Mol. Biotechnol., 18, 35-35). Zhang et al., 2000, Gene Ther., 7, 2041, describes an anti-K-Ras ribozyme adenoviral vector (KRbz-ADV) targeting a K-Ras mutant (K-Ras codon 12 GGT to GTT; H441 and H1725 cells respectively). Non-small cell lung cancer cells (NSCLC H441 and H1725 cells) that express the mutant K-Ras protein were used in nude mouse xenografts compared to NSCLC H1650 cells that lack the relevant mutation. Pre-treatment with KRbz-ADV completely abrogated engraftment of both H441 and H1725 cells and compared to 100% engraftment and tumor growth in animals that received untreated tumor cells or a control vector. The above studies provide proof that inhibition of Ras expression by anti-Ras agents causes inhibition of tumor growth in animals. Anti-Ras DNAzymes chosen from in vitro assays are further tested in similar mouse xenograft models. Active DNAzymes are subsequently tested in combination with standard chemotherapies.

Indications

Particular degenerative and disease states that are associated with Ras expression modulation include but are not limited to cancer, for example lung cancer, colorectal cancer, bladder cancer, pancreatic cancer, breast cancer, prostate cancer and/or any other diseases or conditions that are related to or will respond to the levels of Ras in a cell or tissue, alone or in combination with other therapies.

The present body of knowledge in Ras research indicates the need for methods to assay Ras activity and for compounds that can regulate Ras expression for research, diagnostic, and therapeutic use.

The use of monoclonal antibodies, chemotherapy, radiation therapy, and analgesics, are all non-limiting examples of methods that can be combined with or used in conjunction with the nucleic acid molecules (e.g. DNAzymes) of the instant invention. Common chemotherapies that can be combined with nucleic acid molecules of the instant invention include various combinations of cytotoxic drugs to kill cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention (e.g. DNAzyme molecules) are hence within the scope of the instant invention.

Diagnostic Uses

The nucleic acid molecules of this invention (e.g., enzymatic nucleic acid molecules) are used as diagnostic tools to examine genetic drift and mutations within diseased cells or to detect the presence of Ras RNA in a cell. The close relationship between enzymatic nucleic acid molecule activity and the structure of the target RNA allows the detection of mutations in any region of the molecule that alters the base-pairing and three-dimensional structure of the target RNA. Using multiple enzymatic nucleic acid molecules described in this invention, one maps nucleotide changes which are important to RNA structure and function in vitro, as well as in cells and tissues. Cleavage of target RNAs with enzymatic nucleic acid molecules are used to inhibit gene expression and define the role (essentially) of specified gene products in the progression of disease. In this manner, other genetic targets are defined as important mediators of the disease. These experiments lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are known in the art, and include detection of the presence of mRNAs associated with Ras-related condition. Such RNA is detected by determining the presence of a cleavage product after treatment with an enzymatic nucleic acid molecule using standard methodology.

In a specific example, enzymatic nucleic acid molecules that cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., Ras) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is described, for example, in George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

### Example 6

### Identification of Potential Target Sites in Human HIV RNA

The sequence of human HIV genes are screened for accessible sites using a computer-folding algorithm. Regions of the RNA that do not form secondary folding structures and contained potential enzymatic nucleic acid molecule and/or antisense binding/cleavage sites are identified. The sequences of these binding/cleavage sites are shown in Tables VI to XI.

### Example 6

### Selection of Enzymatic Nucleic Acid Cleavage Sites in Human HIV RNA

Enzymatic nucleic acid molecule target sites were chosen by analyzing sequences of Human HIV (Genbank accession No: NM_{—}005228) and prioritizing the sites on the basis of folding. Enzymatic nucleic acid molecules were designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core were eliminated from consideration. As noted below, varying binding arm lengths can be chosen to optimize activity. Generally, at least 5 bases on each arm are able to bind to, or otherwise interact with, the target RNA.

### Example 8

### Chemical Synthesis and Purification of Ribozymes and Antisense for Efficient Cleavage and/or Blocking of HIV Activity

Enzymatic nucleic acid molecules and antisense constructs are designed to anneal to various sites in the RNA message. The binding arms of the enzymatic nucleic acid molecules are complementary to the target site sequences described above, while the antisense constructs are fully complementary to the target site sequences described above. The enzymatic nucleic acid molecules and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Enzymatic nucleic acid molecules and antisense constructs are also synthesized from DNA templates using bacteriophage T7 RNA polymerase (Milligan and Uhlenbeck, 1989, Methods Enzymol. 180, 51). Enzymatic nucleic acid molecules and antisense constructs are purified by gel electrophoresis using general methods or are purified by high pressure liquid chromatography (HPLC; See Wincott et al., supra; the totality of which is hereby incorporated herein by reference) and are resuspended in water. The sequences of the chemically synthesized enzymatic nucleic acid molecules used in this study are shown below in Table XI. The sequences of the chemically synthesized antisense constructs used in this study are complementary sequences to the Substrate sequences shown below as in Tables VI to XI.

### Example 8

### Enzymatic Nucleic Acid Molecule Cleavage of HIV RNA Target in vitro

Enzymatic nucleic acid molecules targeted to the human HIV RNA are designed and synthesized as described above. These enzymatic nucleic acid molecules are tested for cleavage activity in vitro, for example, using the following procedure. The target sequences and the nucleotide location within the HIV RNA are given in Tables VI to XI.

Cleavage Reactions: Full-length or partially full-length, internally-labeled target RNA for enzymatic nucleic acid molecule cleavage assay is prepared by in vitro transcription in the presence of [a-³²p] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2× enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

Indications

Particular degenerative and disease states that can be associated with HIV expression modulation include but are not limited to acquired immunodeficiency disease (AIDS) and related diseases and conditions, including but not limited to Kaposi's sarcoma, lymphoma, cervical cancer, squamous cell carcinoma, cardiac myopathy, rheumatic diseases, and opportunistic infection, for example Pneumocystis carinii, Cytomegalovirus, Herpes simplex, Mycobacteria, Cryptococcus, Toxoplasma, Progressive multifocal leucoencepalopathy (Papovavirus), Mycobacteria, Aspergillus, Cryptococcus, Candida, Cryptosporidium, Isospora belli, Microsporidia and any other diseases or conditions that are related to or will respond to the levels of HIV in a cell or tissue, alone or in combination with other therapies

The present body of knowledge in HIV research indicates the need for methods to assay HIV activity and for compounds that can regulate HIV expression for research, diagnostic, and therapeutic use.

The use of antiviral compounds, monoclonal antibodies, chemotherapy, radiation therapy, analgesics, and/or anti-inflammatory compounds, are all non-limiting examples of a methods that can be combined with or used in conjunction with the nucleic acid molecules (e.g. ribozymes and antisense molecules) of the instant invention. Examples of antiviral compounds that can be used in conjunction with the nucleic acid molecules of the invention include but are not limited to AZT (also known as zidovudine or ZDV), ddC (zalcitabine), ddl (dideoxyinosine), d4T (stavudine), and 3TC (lamivudine) Ribavirin, delvaridine (Rescriptor), nevirapine (Viramune), efravirenz (Sustiva), ritonavir (Norvir), saquinivir (Invirase), indinavir (Crixivan), amprenivir (Agenerase), nelfinavir (Viracept), and/or lopinavir (Kaletra). Common chemotherapies that can be combined with nucleic acid molecules of the instant invention include various combinations of cytotoxic drugs to kill cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention (e.g. ribozymes and antisense molecules) are hence within the scope of the instant invention.

Diagnostic uses

The nucleic acid molecules of this invention (e.g., enzymatic nucleic acid molecules) are used as diagnostic tools to examine genetic drift and mutations within diseased cells or to detect the presence of HIV RNA in a cell. The close relationship between enzymatic nucleic acid molecule activity and the structure of the target RNA allows the detection of mutations in any region of the molecule which alters the base-pairing and three-dimensional structure of the target RNA. Using multiple enzymatic nucleic acid molecules described in this invention, one maps nucleotide changes which are important to RNA structure and function in vitro, as well as in cells and tissues. Cleavage of target RNAs with enzymatic nucleic acid molecules are used to inhibit gene expression and define the role (essentially) of specified gene products in the progression of disease. In this manner, other genetic targets are defined as important mediators of the disease. These experiments lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with HIV-related condition. Such RNA is detected by determining the presence of a cleavage product after treatment with an enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HIV) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is more fully described in George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

### Example 10

### Identification of Potential Target Sites in Human HER2 RNA

The sequence of human HER2 genes were screened for accessible sites using a computer-folding algorithm. Regions of the RNA that do not form secondary folding structures and contained potential enzymatic nucleic acid molecule and/or antisense binding/cleavage sites were identified. The sequences of these binding/cleavage sites are shown in Tables IV and V.

### Example 10

### Selection of Enzymatic Nucleic Acid Cleavage Sites in Human HER2 RNA

Enzymatic nucleic acid molecule target sites were chosen by analyzing sequences of Human HER2 (Genbank accession No: X03363) and prioritizing the sites on the basis of folding. Enzymatic nucleic acid molecules were designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core were eliminated from consideration. As noted below, variable binding arm lengths are chosen to optimize activity. Generally, at least 5 bases on each arm are able to bind to, or otherwise interact with, the target RNA.

### Example 12

### Chemical Synthesis and Purification of Ribozymes and Antisense for Efficient Cleavage and/or Blocking of HER2 Expression

DNAzyme molecules are designed to anneal to various sites in the RNA message. The binding arms of the DNAzyme molecules are complementary to the target site sequences described above. The DNAzymes were chemically synthesized. The method of synthesis used followed the procedure for nucleic acid synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%. The sequences of the chemically synthesized DNAzyme molecules used in this study are shown below in Table V.

### Example 13

### DNAzyme Cleavage of HER2 RNA Target in vitro

DNAzymes targeted to the human HER2 RNA are designed and synthesized as described above. These enzymatic nucleic acid molecules can be tested for cleavage activity in vitro, for example, using the following procedure. The target sequences and the nucleotide location within the HER2 RNA are given in Tables IV and V.

Cleavage Reactions:

Ribozymes and substrates were synthesized in 96-well format using 0.2 μmol scale. Substrates were 5′-32P labeled and gel purified using 7.5% polyacrylamide gels, and eluting into water. Assays were done by combining trace substrate with 500 nM Ribozyme or greater, and initiated by adding final concentrations of 40 mM Mg⁺², and 50 mM Tris-Cl pH 8.0. For each ribozyme/substrate combination a control reaction was done to ensure cleavage was not the result of non-specific substrate degradation. A single three hour time point was taken and run on a 15% polyacrylamide gel to asses cleavage activity. Gels were dried and scanned using a Molecular Dynamics Phosphorimager and quantified using Molecular Dynamics ImageQuant software. Percent cleaved was determined by dividing values for cleaved substrate bands by full-length (uncleaved) values plus cleaved values and multiplying by 100 (% cleaved=[C/(U+C)]*100).

### Example 14

### DNAzyme Cleavage of HER2 RNA Target in vivo

Cell Culture Review

The greatest HER2 specific effects have been observed in cancer cell lines that express high levels of HER2 protein (as measured by ELISA). Specifically, in one study that treated five human breast cancer cell lines with the HER2 antibody (anti-erbB2-sFv), the greatest inhibition of cell growth was seen in three cell lines (MDA-MB-361, SKBR-3 and BT-474) that express high levels of HER2 protein. No inhibition of cell growth was observed in two cell lines (MDA-MB-231 and MCF-7) that express low levels of HER2 protein (Wright, M., Grim, J., Deshane, J., Kim, M., Strong, T. V., Siegel, G. P., Curiel, D. T. (1997) An intracellular anti-erbB-2 single-chain antibody is specifically cytotoxic to human breast carcinoma cells overexpressing erbB-2. Gene Therapy 4: 317-322). Another group successfully used SKBR-3 cells to show HER2 antisense oligonucleotide-mediated inhibition of HER2 protein expression and HER2 RNA knockdown (Vaughn, J. P., Iglehart, J. D., Demirdji, S., Davis, P., Babiss, L. E., Caruthers, M. H., Marks, J. R. (1995) Antisense DNA downregulation of the ERBB2 oncogene measured by a flow cytometric assay. Proc Natl Acad Sci USA 92: 8338-8342). Other groups have also demonstrated a decrease in the levels of HER2 protein, HER2 mRNA and/or cell proliferation in cultured cells using anti-HER2 DNAzymes or antisense molecules (Suzuki T., Curcio, L. D., Tsai, J. and Kashani-Sabet M. (1997) Anti-c-erb-B-2 Ribozyme for Breast Cancer. In Methods in Molecular Medicine, Vol. 11, Therapeutic Applications of Ribozmes, Human Press, Inc., Totowa, N.J.; Weichen, K., Zimmer, C. and Dietel, M. (1997) Selection of a high activity c-erbB-2 ribozyme using a fusion gene of c-erbB-2 and the enhanced green fluorescent protein. Cancer Gene Therapy 5: 45-51; Czubayko, F., Downing, S. G., Hsieh, S. S., Goldstein, D. J., Lu P. Y., Trapnell, B. C. and Wellstein, A. (1997) Adenovirus-mediated transduction of ribozymes abrogates HER-2/neu and pleiotrophin expression and inhibits tumor cell proliferation. Gene Ther. 4: 943-949; Colomer, R., Lupu, R., Bacus, S. S. and Gelmann, E. P. (1994) erbB-2 antisense oligonucloetides inhibit the proliferation of breast carcinoma cells with erbB-2 oncogene amplification. British J. Cancer 70: 819-825; Betram et al., 1994). Because cell lines that express higher levels of HER2 have been more sensitive to anti-HER2 agents, we prefer using several medium to high expressing cell lines, including SKBR-3 and T47D, for DNAzyme screens in cell culture.

A variety of endpoints have been used in cell culture models to look at HER2-mediated effects after treatment with anti-HER2 agents. Phenotypic endpoints include inhibition of cell proliferation, apoptosis assays and reduction of HER2 protein expression. Because overexpression of HER2 is directly associated with increased proliferation of breast and ovarian tumor cells, a proliferation endpoint for cell culture assays will preferably be used as the primary screen. There are several methods by which this endpoint can be measured. Following treatment of cells with DNAzymes, cells are allowed to grow (typically 5 days) after which either the cell viability, the incorporation of [³H] thymidine into cellular DNA and/or the cell density can be measured. The assay of cell density is very straightforward and can be done in a 96-well format using commercially available fluorescent nucleic acid stains (such as Syto® 13 or CyQuant®). The assay using CyQuant® is described herein and is currently being employed to screen ˜100 DNAzymes targeting HER2 (details below).

As a secondary, confirmatory endpoint a DNAzyme-mediated decrease in the level of HER2 protein expression can be evaluated using a HER2-specific ELISA.

Validation of Cell Lines and DNAzyme Treatment Conditions

Two human breast cancer cell lines (T47D and SKBR-3) that are known to express medium to high levels of HER2 protein, respectively, are considered for DNAzyme screening. In order to validate these cell lines for HER2-mediated sensitivity, both cell lines are treated with the HER2 specific antibody, Herceptin® (Genentech) and its effect on cell proliferation is determined. Herceptin® is added to cells at concentrations ranging from 0-8 μM in medium containing either no serum (OptiMem), 0.1% or 0.5% FBS and efficacy is determined via cell proliferation. Maximal inhibition of proliferation (˜50%) in both cell lines is typically observed after addition of Herceptin® at 0.5 nM in medium containing 0.1% or no FBS. The fact that both cell lines are sensitive to an anti-HER2 agent (Herceptin®) supports their use in experiments testing anti-HER2 DNAzymes.

Prior to DNAzyrne screening, the choice of the optimal lipid(s) and conditions for DNAzyme delivery is determined empirically for each cell line. Applicant has established a panel of cationic lipids (lipids as described in PCT application WO99/05094) that can be used to deliver DNAzymes to cultured cells and are very useful for cell proliferation assays that are typically 3-5 days in length. (Additional description of useful lipids is provided above, and those skilled in the art are also familiar with a variety of lipids that can be used for delivery of oligonucleotide to cells in culture.) Initially, this panel of lipid delivery vehicles is screened in SKBR-3 and T47D cells using previously established control oligonucleotides. Specific lipids and conditions for optimal delivery are selected for each cell line based on these screens. These conditions are used to deliver HER2 specific DNAzymes to cells for primary (inhibition of cell proliferation) and secondary (decrease in HER2 protein) efficacy endpoints.

Primary Screen: Inhibition of Cell Proliferation

DNAzyme screens are performed using an automated, high throughput 96-well cell proliferation assay. Cell proliferation is measured over a 5-day treatment period using the nucleic acid stain CyQuant® for determining cell density. The growth of cells treated with DNAzyme/lipid complexes is compared to both untreated cells and to cells treated with Scrambled-arm Attenuated core Controls. SACs can no longer bind to the target site due to the scrambled arm sequence and have nucleotide changes in the core that greatly diminish DNAzyme cleavage. These SACs are used to determine non-specific inhibition of cell growth caused by DNAzyme chemistry (i.e. multiple 2′ O-Me modified nucleotides and a 3′ inverted abasic). Lead DNAzymes are chosen from the primary screen based on their ability to inhibit cell proliferation in a specific manner. Dose response assays are carried out on these leads and a subset was advanced into a secondary screen using the level of HER2 protein as an endpoint.

Secondary Screen: Decrease in HER2 Protein and/or RNA

A secondary screen that measures the effect of anti-HER2 DNAzymes on HER2 protein and/or RNA levels is used to affirm preliminary findings. A robust HER2 ELISA for both T47D and SKBR-3 cells has been established and is available for use as an additional endpoint. In addition, a real time RT-PCR assay (TaqMan assay) has been developed to assess HER2 RNA reduction compared to an actin RNA control. Dose response activity of nucleic acid molecules of the instant invention can be used to assess both HER2 protein and RNA reduction endpoints.

DNAzyme Mechanism Assays

A TaqMan® assay for measuring the DNAzyme-mediated decrease in HER2 RNA has also been established. This assay is based on PCR technology and can measure in real time the production of HER2 mRNA relative to a standard cellular MRNA such as GAPDH. This RNA assay is used to establish proof that lead DNAzymes are working through an RNA cleavage mechanism and result in a decrease in the level of HER2 mRNA, thus leading to a decrease in cell surface HER2 protein receptors and a subsequent decrease in tumor cell proliferation.

Animal Models

Evaluating the efficacy of anti-HER2 agents in animal models is an important prerequisite to human clinical trials. As in cell culture models, the most HER2 sensitive mouse tumor xenografts are those derived from human breast carcinoma cells that express high levels of HER2 protein. In a recent study, nude mice bearing BT-474 xenografts were sensitive to the anti-HER2 humanized monoclonal antibody Herceptin®, resulting in an 80% inhibition of tumor growth at a 1 mg kg dose (ip, 2×week for 4-5 weeks). Tumor eradication was observed in 3 of 8 mice treated in this manner (Baselga, J., Norton, L. Albanell, J., Kim, Y. M. and Mendelsohn, J. (1998) Recombinant humanized anti-HER2 antibody (Herceptin) enhances the antitumor activity of paclitaxel and doxorubicin against HER2/neu overexpressing human breast cancer xenografts. Cancer Res. 15: 2825-2831). This same study compared the efficacy of Herceptin® alone or in combination with the commonly used chemotherapeutics, paclitaxel or doxorubicin. Although, all three anti-HER2 agents caused modest inhibition of tumor growth, the greatest antitumor activity was produced by the combination of Herceptin® and paclitaxel (93% inhibition of tumor growth vs 35% with paclitaxel alone). The above studies provide proof that inhibition of HER2 expression by anti-HER2 agents causes inhibition of tumor growth in animals. Lead anti-HER2 DNAzymes chosen from in vitro assays are further tested in mouse xenograft models. DNAzymes are first tested alone and then in combination with standard chemotherapies.

Animal Model Development

Three human breast tumor cell lines (T47D, SKBR-3 and BT-474) were characterized to establish their growth curves in mice. These three cell lines have been implanted into the mammary papillae of both nude and SCID mice and primary tumor volumes are measured 3 times per week. Growth characteristics of these tumor lines using a Matrigel implantation format can also be established. The use of two other breast cell lines that have been engineered to express high levels of HER2 can also be used in the described studies. The tumor cell line(s) and implantation method that supports the most consistent and reliable tumor growth is used in animal studies testing the lead HER2 DNAzyme(s). DNAzymes are administered by daily subcutaneous injection or by continuous subcutaneous infusion from Alzet mini osmotic pumps beginning 3 days after tumor implantation and continuing for the duration of the study. Group sizes of at least 10 animals are employed. Efficacy is determined by statistical comparison of tumor volume of DNAzyme-treated animals to a control group of animals treated with saline alone. Because the growth of these tumors is generally slow (45-60 days), an initial endpoint is the time in days it takes to establish an easily measurable primary tumor (i.e. 50-100 mm³) in the presence or absence of DNAzyme treatment.

Clinical Summary

Overview

Breast cancer is a common cancer in women and also occurs in men to a lesser degree. The incidence of breast cancer in the United States is ˜180,000 cases per year and ˜46,000 die each year of the disease. In addition, 21,000 new cases of ovarian cancer per year lead to ˜13,000 deaths (data from Hung, M.-C., Matin, A., Zhang, Y., Xing, X., Sorgi, F., Huang, L. and Yu, D. (1995) HER-2/neu-targeting gene therapy—a review. Gene 159: 65-71 and the Surveillance, Epidemiology and End Results Program, NCI Surveillance, Epidemiology and End Results Program (SEER) Cancer Statistics Review: http://www.seer.ims.nci.nih.gov/Publications/CSR1973_{—}1996/). Ovarian cancer is a potential secondary indication for anti-HER2 DNAzyme therapy.

A full review of breast cancer is given in the NCI PDQ for Breast Cancer (NCI PDQ/Treatment/Health Professionals/Breast Cancer: http://cancernet.nci.nih.gov/clinpdq/soa/Breast_cancer_Physician.html; NCI PDQ/Treatment/Patients/Breast Cancer: http://cancernet.nci.nih.gov/clinpdq/pif/Breast_cancer_Patient.html). A brief overview is given here. Breast cancer is evaluated or “staged” on the basis of tumor size, and whether it has spread to lymph nodes and/or other parts of the body. In Stage I breast cancer, the cancer is no larger than 2 centimeters and has not spread outside of the breast. In Stage II, the patient's tumor is 2-5 centimeters but cancer may have spread to the axillary lymph nodes. By Stage III, metastasis to the lymph nodes is typical, and tumors are ≧5 centimeters. Additional tissue involvement (skin, chest wall, ribs, muscles etc.) may also be noted. Once cancer has spread to additional organs of the body, it is classed as Stage IV.

Almost all breast cancers (>90%) are detected at Stage I or II, but 31% of these are already lymph node positive. The 5-year survival rate for node negative patients (with standard surgery/radiation/chemotherapy/hormone regimens) is 97%; however, involvement of the lymph nodes reduces the 5-year survival to only 77%. Involvement of other organs (≧Stage III) drastically reduces the overall survival, to 22% at 5 years. Thus, chance of recovery from breast cancer is highly dependent on early detection. Because up to 10% of breast cancers are hereditary, those with a family history are considered to be at high risk for breast cancer and should be monitored very closely.

Therapy

Breast cancer is highly treatable and often curable when detected in the early stages. (For a complete review of breast cancer treatments, see the NCI PDQ for Breast Cancer.) Common therapies include surgery, radiation therapy, chemotherapy and hormonal therapy. Depending upon many factors, including the tumor size, lymph node involvement and location of the lesion, surgical removal varies from lumpectomy (removal of the tumor and some surrounding tissue) to mastectomy (removal of the breast, lymph nodes and some or all of the underlying chest muscle). Even with successful surgical resection, as many as 21% of the patients may ultimately relapse (10-20 years). Thus, once local disease is controlled by surgery, adjuvant radiation treatments, chemotherapies and/or hormonal therapies are typically used to reduce the rate of recurrence and improve survival. The therapy regimen employed depends not only on the stage of the cancer at its time of removal, but other variables such the type of cancer (ductal or lobular), whether lymph nodes were involved and removed, age and general health of the patient and if other organs are involved.

Common chemotherapies include various combinations of cytotoxic drugs to kill the cancer cells. These drugs include paclitaxel (Taxol), docetaxel, cisplatin, methotrexate, cyclophosphamide, doxorubin, fluorouracil etc. Significant toxicities are associated with these cytotoxic therapies. Well-characterized toxicities include nausea and vomiting, myelosuppression, alopecia and mucosity. Serious cardiac problems are also associated with certain of the combinations, e.g. doxorubin and paclitaxel, but are less common.

Testing for estrogen and progesterone receptors helps to determine whether certain anti-hormone therapies might be helpful in inhibiting tumor growth. If either or both receptors are present, therapies to interfere with the action of the hormone ligands, can be given in combination with chemotherapy and are generally continued for several years. These adjuvant therapies are called SERMs, selective estrogen receptor modulators, and they can give beneficial estrogen-like effects on bone and lipid metabolism while antagonizing estrogen in reproductive tissues. Tamoxifen is one such compound. The primary toxic effect associated with the use of tamoxifen is a 2 to 7-fold increase in the rate of endometrial cancer. Blood clots in the legs and lung and the possibility of stroke are additional side effects. However, tamoxifen has been determined to reduce breast cancer incidence by 49% in high-risk patients and an extensive, somewhat controversial, clinical study is underway to expand the prophylactic use of tamoxifen. Another SERM, raloxifene, was also shown to reduce the incidence of breast cancer in a large clinical trial where it was being used to treat osteoporosis. In additional studies, removal of the ovaries and/or drugs to keep the ovaries from working are being tested.

Bone marrow transplantation is being studied in clinical trials for breast cancers that have become resistant to traditional chemotherapies or where >3 lymph nodes are involved. Marrow is removed from the patient prior to high-dose chemotherapy to protect it from being destroyed, and then replaced after the chemotherapy. Another type of “transplant” involves the exogenous treatment of peripheral blood stem cells with drugs to kill cancer cells prior to replacing the treated cells in the bloodstream.

One biological treatment, a humanized monoclonal anti-HER2 antibody, Herceptin® (Genentech) has been approved by the FDA as an additional treatment for HER2 positive tumors. Herceptin® binds with high affinity to the extracellular domain of HER2 and thus blocks its signaling action. Herceptin® can be used alone or in combination with chemotherapeutics (i.e. paclitaxel, docetaxel, cisplatin, etc.) (Pegram, M. D., Lipton, A., Hayes, D. F., Weber, B. L., Baselga, J. M., Tripathy, D., Baly, D., Baughman, S. A., Twaddell, T., Glaspy, J. A. and Slamon, D. J. (1998) Phase II study of receptor-enhanced chemosensitivity using recombinant humanized anti-p185HER2/neu monoclonal antibody plus cisplatin in patients with HER2/neu-overexpressing metastatic breast cancer refractory to chemotherapy treatment. J. Clin. Oncol. 16: 2659-2671). In Phase III studies, Herceptin® significantly improved the response rate to chemotherapy as well as improving the time to progression (Ross, J. S. and Fletcher, J. A. (1998) The HER-2/neu oncogene in breast cancer: Prognostic factor, predictive factor and target for therapy. Oncologist 3: 1998). The most common side effects attributed to Herceptin® are fever and chills, pain, asthenia, nausea, vomiting, increased cough, diarrhea, headache, dyspnea, infection, rhinitis, and insomnia. Herceptin® in combination with chemotherapy (paclitaxel) can lead to cardiotoxicity (Sparano, J. A. (1999) Doxorubicin/taxane combinations: Cardiac toxicity and pharmacokinetics. Semin. Oncol. 26: 14-19), leukopenia, anemia, diarrhea, abdominal pain and infection.

HER2 Protein Levels for Patient Screening and as a Potential Endpoint

Because elevated HER2 levels can be detected in at least 30% of breast cancers, breast cancer patients can be pre-screened for elevated HER2 prior to admission to initial clinical trials testing an anti-HER2 DNAzyme. Initial HER2 levels can be determined (by ELISA) from tumor biopsies or resected tumor samples.

During clinical trials, it may be possible to monitor circulating HER2 protein by ELISA (Ross and Fletcher, 1998). Evaluation of serial blood/serum samples over the course of the anti-HER2 DNAzyme treatment period could be useful in determining early indications of efficacy. In fact, the clinical course of Stage IV breast cancer was correlated with shed HER2 protein fragment following a dose-intensified paclitaxel monotherapy. In all responders, the HER2 serum level decreased below the detection limit (Luftner, D., Schnabel. S. and Possinger, K. (1999) c-erbB-2 in serum of patients receiving fractionated paclitaxel chemotherapy. Int. J Biol. Markers 14: 55-59).

Two cancer-associated antigens, CA27.29 and CA15.3, can also be measured in the serum. Both of these glycoproteins have been used as diagnostic markers for breast cancer. CA27.29 levels are higher than CA15.3 in breast cancer patients; the reverse is true in healthy individuals. Of these two markers, CA27.29 was found to better discriminate primary cancer from healthy subjects. In addition, a statistically significant and direct relationship was shown between CA27.29 and large vs small tumors and node postive vs node negative disease (Gion, M., Mione, R., Leon, A. E. and Dittadi, R. (1999) Comparison of the diagnostic accuracy of CA27.29 and CA15.3 in primary breast cancer. Clin. Chem. 45: 630-637). Moreover, both cancer antigens were found to be suitable for the detection of possible metastases during follow-up (Rodriguez de Paterna, L., Arnaiz, F., Estenoz, J. Ortuno, B. and Lanzos E. (1999) Study of serum tumor markers CEA, CA15.3, CA27.29 as diagnostic parameters in patients with breast carcinoma. Int. J. Biol. Markers 10: 24-29). Thus, blocking breast tumor growth may be reflected in lower CA27.29 and/or CA15.3 levels compared to a control group. FDA submissions for the use of CA27.29 and CA15.3 for monitoring metastatic breast cancer patients have been filed (reviewed in Beveridge, R. A. (1999) Review of clinical studies of CA27.29 in breast cancer management. Int. J. Biol. Markers 14: 36-39). Fully automated methods for measurement of either of these markers are commercially available.

Indications

Particular degenerative and disease states that can be associated with HER2 expression modulation include but are not limited to cancer, for example breast cancer and ovarian cancer and/or any other diseases or conditions that are related to or will respond to the levels of HER2 in a cell or tissue, alone or in combination with other therapies

The present body of knowledge in HER2 research indicates the need for methods to assay HER2 activity and for compounds that can regulate HER2 expression for research, diagnostic, and therapeutic use.

The use of monoclonal antibodies, chemotherapy, radiation therapy, and analgesics, are all non-limiting examples of methods that can be combined with or used in conjunction with the nucleic acid molecules (e.g. DNAzymes) of the instant invention. Common chemotherapies that can be combined with nucleic acid molecules of the instant invention include various combinations of cytotoxic drugs to kill cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention (e.g. DNAzyme molecules) are hence within the scope of the instant invention.

Diagnostic Uses

The nucleic acid molecules of this invention (e.g., enzymatic nucleic acid molecules) can be used as diagnostic tools to examine genetic drift and mutations within diseased cells or to detect the presence of HER2 RNA in a cell. The close relationship between enzymatic nucleic acid molecule activity and the structure of the target RNA allows the detection of mutations in any region of the molecule that alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with HER2-related condition. Such RNA is detected by determining the presence of a cleavage product after treatment with an enzymatic nucleic acid molecule using standard methodology.

In a specific example, enzymatic nucleic acid molecules that cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HER2) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is more fully described in George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

Additional Uses

Potential uses of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to modulate gene expression of target genes in bacterial, microbial, fungal, viral, and eukaryotic systems including plant or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations which is not specifically disclosed herein. Thus, for example, in each instance herein any of the terms “comprising”, “consisting essentially of” and “consisting of” can be replaced with either of the other two terms. The terms and expressions that have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed can be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the claims that follow.

TABLE I

| | | | | | |
|---|---|---|---|---|---|
| Reagent | Equivalents | Amount | Wait Time* DNA | Wait Time* 2′-O-methyl | Wait Time*RNA |

A. 2.5 μmol Synthesis Cycle ABI 394 Instrument

| | | | | | | | | | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| | | | | | | | | | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | Equivalents: DNA/ | Amount: DNA/2′-O- | | | | Wait Time* 2′-O- | | | |
|---|---|---|---|---|---|---|---|---|---|
| Reagent | 2′-O-methyl/Ribo | methyl/Ribo | | Wait Time* DNA | | methyl | | Wait Time* Ribo | |
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

TABLE II
Human K-Ras DNAzyme and Substrate Sequence

| | | | | |
|---|---|---|---|---|
| | | Seq | | Seq |
| Pos | Substrate | ID | DNAzyme | ID |

| | | | | |
|---|---|---|---|---|
| 10 | CCUAGGCG G CGGCCGCG | 1 | CGCGGCCG GGCTAGCTACAACGA CGCCTAGG | 2329 |
| 13 | AGGCGGCG G CCGCGGCG | 2 | CGCCGCGG GGCTAGCTACAACGA CGCCGCCT | 2330 |
| 16 | CGGCGGCC G CGGCGGCG | 3 | CGCCGCCG GGCTAGCTACAACGA GGCCGCCG | 2331 |
| 19 | CGGCCGCG G CGGCGGAG | 4 | CTCCGCCG GGCTAGCTACAACGA CGCGGCCG | 2332 |
| 22 | CCGCGGCG G CGGAGGCA | 5 | TGCCTCCG GGCTAGCTACAACGA CGCCGCGG | 2333 |
| 28 | CGGCGGAG G CAGCAGCG | 6 | CGCTGCTG GGCTAGCTACAACGA CTCCGCCG | 2334 |
| 31 | CGGAGGCA G CAGCGGCG | 7 | CGCCGCTG GGCTAGCTACAACGA TGCCTCCG | 2335 |
| 34 | AGGCAGCA G CGGCGGCG | 8 | CGCCGCCG GGCTAGCTACAACGA TGCTGCCT | 2336 |
| 37 | CAGCAGCG G CGGCGGCA | 9 | TGCCGCCG GGCTAGCTACAACGA CGCTGCTG | 2337 |
| 40 | CAGCGGCG G CGGCAGUG | 10 | CACTGCCG GGCTAGCTACAACGA CGCCGCTG | 2338 |
| 43 | CGGCGGCG G CAGUGGCG | 11 | CGCCACTG GGCTAGCTACAACGA CGCCGCCG | 2339 |
| 46 | CGGCGGCA G UGGCGGCG | 12 | CGCCGCCA GGCTAGCTACAACGA TGCCGCCG | 2340 |
| 49 | CGGCAGUG G CGGCGGCG | 13 | CGCCGCCG GGCTAGCTACAACGA CACTGCCG | 2341 |
| 52 | CAGUGGCG G CGGCGAAG | 14 | CTTCGCCG GGCTAGCTACAACGA CGCCACTG | 2342 |
| 55 | UGGCGGCG G CGAAGGUG | 15 | CACCTTCG GGCTAGCTACAACGA CGCCGCCA | 2343 |
| 61 | CGGCGAAG G UGGCGGCG | 16 | CGCCGCCA GGCTAGCTACAACGA CTTCGCCG | 2344 |
| 64 | CGAAGGUG G CGGCGGCU | 17 | AGCCGCCG GGCTAGCTACAACGA CACCTTCG | 2345 |
| 67 | AGGUGGCG G CGGCUCGG | 18 | CCGAGCCG GGCTAGCTACAACGA CGCCACCT | 2346 |
| 70 | UGGCGGCG G CUCGGCCA | 19 | TGGCCGAG GGCTAGCTACAACGA CGCCGCCA | 2347 |
| 75 | GCGGCUCG G CCAGUACU | 20 | AGTACTGG GGCTAGCTACAACGA CGAGCCGC | 2348 |
| 79 | CUCGGCCA G UACUCCCG | 21 | CGGGAGTA GGCTAGCTACAACGA TGGCCGAG | 2349 |
| 81 | CGGCCAGU A CUCCCGGC | 22 | GCCGGGAG GGCTAGCTACAACGA ACTGGCCG | 2350 |
| 88 | UACUCCCG G CCCCCGCC | 23 | GGCGGGGG GGCTAGCTACAACGA CGGGAGTA | 2351 |
| 94 | CGGCCCCC G CCAUUUCG | 24 | CGAAATGG GGCTAGCTACAACGA GGGGGCCG | 2352 |
| 97 | CCCCCGCC A UUUCGGAC | 25 | GTCCGAAA GGCTAGCTACAACGA GGCGGGGG | 2353 |
| 104 | CAUUUCGG A CUGGGAGC | 26 | GCTCCCAG GGCTAGCTACAACGA CCGAAATG | 2354 |
| 111 | GACUGGGA G CGAGCGCG | 27 | CGCGCTCG GGCTAGCTACAACGA TCCCAGTC | 2355 |
| 115 | GGGAGCGA G CGCGGCGC | 28 | GCGCCGCG GGCTAGCTACAACGA TCGCTCCC | 2356 |
| 117 | GAGCGAGC G CGGCGCAG | 29 | CTGCGCCG GGCTAGCTACAACGA GCTCGCTC | 2357 |
| 120 | CGAGCGCG G CGCACGCA | 30 | TGCCTGCG GGCTAGCTACAACGA CGCGCTCC | 2358 |
| 122 | AGCGCGGC G CAGGCACU | 31 | AGTGCCTG GCCTAGCTACAACGA GCCGCGCT | 2359 |
| 126 | CGGCGCAG G CACUGAAG | 32 | CTTCAGTG GGCTAGCTACAACGA CTGCGCCG | 2360 |
| 128 | GCGCAGGC A CUGAAGGC | 33 | GCCTTCAG GGCTAGCTACAACGA GCCTGCGC | 2361 |
| 135 | CACUGAAG G CGGCGGCG | 34 | CGCCGCCG GGCTAGCTACAACGA CTTCAGTG | 2362 |
| 138 | UGAAGGCG G CGGCGGGG | 35 | CCCCGCCG GGCTAGCTACAACGA CGCCTTCA | 2363 |
| 141 | AGGCGGCG G CGGGGCCA | 36 | TGGCCCCG GGCTAGCTACAACGA CGCCGCCT | 2364 |
| 146 | GCGGCGGG G CCAGAGGC | 37 | GCCTCTGG GGCTAGCTACAACGA CCCGCCGC | 2365 |
| 153 | GGCCAGAG G CUCAGCGG | 38 | CCGCTGAG GGCTAGCTACAACGA CTCGCGCC | 2366 |
| 158 | GAGGCUCA G CGGCUCCC | 39 | GGGAGCCG GGCTAGCTACAACGA TGAGCCTC | 2367 |
| 161 | GCUCAGCG G CUCCCAGG | 40 | CCTGGGAG GGCTAGCTACAACGA CGCTGAGC | 2368 |
| 169 | GCUCCCAG G UGCGGGAG | 41 | CTCCCGCA GGCTAGCTACAACGA CTGGGAGC | 2369 |
| 171 | UCCCAGGU G CGGGAGAG | 42 | CTCTCCCG GGCTAGCTACAACGA ACCTGGGA | 2370 |
| 182 | GGAGAGAG G CCUGCUGA | 43 | TCAGCAGG GGCTAGCTACAACGA CTCTCTCC | 2371 |
| 186 | AGAGGCCU G CUGAAAAU | 44 | ATTTTCAG GGCTAGCTACAACGA AGGCCTCT | 2372 |
| 193 | UGCUGAAA A UGACUGAA | 45 | TTCAGTCA GGCTAGCTACAACGA TTTCAGCA | 2373 |
| 196 | UGAAAAUG A CUGAAUAU | 46 | ATATTCAG GGCTAGCTACAACGA CATTTTCA | 2374 |
| 201 | AUGACUGA A UAUAAACU | 47 | AGTTTATA GGCTAGCTACAACGA TCAGTCAT | 2375 |
| 203 | GACUGAAU A UAAACUUG | 48 | CAAGTTTA GGCTAGCTACAACGA ATTCAGTC | 2376 |
| 207 | GAAUAUAA A CUUGUGGU | 49 | ACCACAAG GGCTAGCTACAACGA TTATATTC | 2377 |
| 211 | AUAAACUU G UGGUAGUU | 50 | AACTACCA GGCTAGCTACAACGA AAGTTTAT | 2378 |
| 214 | AACUUGUG G UAGUUGGA | 51 | TCCAACTA GGCTAGCTACAACGA CACAAGTT | 2379 |
| 217 | UUGUGGUA G UUGGAGCU | 52 | AGCTCCAA GGCTAGCTACAACGA TACCACAA | 2380 |
| 223 | UAGUUGGA G CUUGUGGC | 53 | GCCACAAG GGCTAGCTACAACGA TCCAACTA | 2381 |
| 227 | UGGAGCUU G UGGCGUAG | 54 | CTACGCCA GGCTAGCTACAACGA AAGCTCCA | 2382 |
| 230 | AGCUUGUG G CGUAGGCA | 55 | TGCCTACG GGCTAGCTACAACGA CACAAGCT | 2383 |
| 232 | CUUGUGGC G UAGGCAAG | 56 | CTTGCCTA GGCTAGCTACAACGA GCCACAAG | 2384 |
| 236 | UGGCGUAG G CAAGAGUG | 57 | CACTCTTG GGCTAGCTACAACGA CTACGCCA | 2385 |
| 242 | AGGCAAGA G UGCCUUGA | 58 | TCAAGGCA GGCTAGCTACAACGA TCTTGCCT | 2386 |
| 244 | GCAAGAGU G CCUUGACG | 59 | CGTCAAGG GGCTAGCTACAACGA ACTCTTGC | 2387 |
| 250 | GUGCCUUG A CGAUACAG | 60 | CTGTATCG GGCTAGCTACAACGA CAAGGCAC | 2388 |
| 253 | CCUUGACG A UACAGCUA | 61 | TAGCTGTA GGCTAGCTACAACGA CGTCAAGG | 2389 |
| 255 | UUGACGAU A CAGCUAAU | 62 | ATTAGCTG GGCTAGCTACAACGA ATCGTCAA | 2390 |
| 258 | ACGAUACA G CUAAUUCA | 63 | TGAATTAG GGCTAGCTACAACGA TGTATCGT | 2391 |
| 262 | UACAGCUA A UUCAGAAU | 64 | ATTCTGAA GGCTAGCTACAACGA TAGCTGTA | 2392 |
| 269 | AAUUCAGA A UCAUUUUG | 65 | CAAAATGA GGCTAGCTACAACGA TCTGAATT | 2393 |
| 272 | UCAGAAUC A UUUUGUGG | 66 | CCACAAAA GGCTAGCTACAACGA GATTCTGA | 2394 |
| 277 | AUCAUUUU G UGGACGAA | 67 | TTCGTCCA GGCTAGCTACAACGA AAAATGAT | 2395 |
| 281 | UUUUGUGG A CGAAUAUG | 68 | CATATTCG GGCTAGCTACAACGA CCACAAAA | 2396 |
| 285 | GUGGACGA A UAUGAUCC | 69 | GGATCATA GGCTAGCTACAACGA TCGTCCAC | 2397 |
| 287 | GGACGAAU A UGAUCCAA | 70 | TTGGATCA GGCTAGCTACAACGA ATTCGTCC | 2398 |
| 290 | CGAAUAUG A UCCAUCAA | 71 | TTGTTGGA GGCTAGCTACAACGA CATATTCG | 2399 |
| 295 | AUGAUCCA A CAAUAGAG | 72 | CTCTATTG GGCTAGCTACAACGA TGGATCAT | 2400 |
| 298 | AUCCAACA A UAGAGGAU | 73 | ATCCTCTA GGCTAGCTACAACGA TGTTGGAT | 2401 |
| 305 | AAUAGAGG A UUCCUACA | 74 | TGTAGGAA GGCTAGCTACAACGA CCTCTATT | 2402 |
| 311 | GGAUUCCU A CAGGAAGC | 75 | GCTTCCTG GGCTAGCTACAACGA AGGAATCC | 2403 |
| 318 | UACAGGAA G CAAGUAGU | 76 | ACTACTTG GGCTAGCTACAACGA TTCCTGTA | 2404 |
| 322 | GGAAGCAA G UAGUAAUU | 77 | AATTACTA GGCTAGCTACAACGA TTGCTTCC | 2405 |
| 325 | AGCAAGUA G UAAUUGAU | 78 | ATCAATTA GGCTAGCTACAACGA TACTTGCT | 2406 |
| 328 | AAGUAGUA A UUGAUGGA | 79 | TCCATCAA GGCTAGCTACAACGA TACTACTT | 2407 |
| 332 | AGUAAUUG A UGGAGAAA | 80 | TTTCTCCA GGCTAGCTACAACGA CAATTACT | 2408 |
| 340 | AUGGAGAA A CCUGUCUC | 81 | GAGACAGG GGCTAGCTACAACGA TTCTCCAT | 2409 |
| 344 | AGAAACCU G UCUCUUGG | 82 | CCAAGAGA GGCTAGCTACAACGA AGGTTTCT | 2410 |
| 353 | UCUCUUGG A UAUUCUCG | 83 | CGAGAATA GGCTAGCTACAACGA CCAAGAGA | 2411 |
| 355 | UCUUGGAU A UUCUCGAC | 84 | GTCGAGAA GGCTAGCTACAACGA ATCCAAGA | 2412 |
| 362 | UAUUCUCG A CACAGCAG | 85 | CTGCTGTG GGCTAGCTACAACGA CGAGAATA | 2413 |
| 364 | UUCUCGAC A CAGCAGGU | 86 | ACCTGCTG GGCTAGCTACAACGA GTCGAGAA | 2414 |
| 367 | UCGACACA G CAGGUCAA | 87 | TTGACCTG GGCTAGCTACAACGA TGTGTCGA | 2415 |
| 371 | CACAGCAG G UCAAGAGG | 88 | CCTCTTGA GGCTAGCTACAACGA CTGCTGTG | 2416 |
| 381 | CAAGAGGA G UACAGUGC | 89 | GCACTGTA GGCTAGCTACAACGA TCCTCTTG | 2417 |
| 383 | AGAGGAGU A CAGUGCAA | 90 | TTGCACTG GGCTAGCTACAACGA ACTCCTCT | 2418 |
| 386 | GGAGUACA G UGCAAUGA | 91 | TCATTGCA GGCTAGCTACAACGA TGTACTCC | 2419 |
| 388 | AGUACAGU G CAAUGAGG | 92 | CCTCATTG GGCTAGCTACAACGA ACTGTACT | 2420 |
| 391 | ACAGUGCA A UGAGGGAC | 93 | GTCCCTCA GGCTAGCTACAACGA TGCACTGT | 2421 |
| 398 | AAUGAGGG A CCAGUACA | 94 | TGTACTGG GGCTAGCTACAACGA CCCTCATT | 2422 |
| 402 | AGGGACCA G UACAUGAG | 95 | CTCATGTA GGCTAGCTACAACGA TGGTCCCT | 2423 |
| 404 | GGACCAGU A CAUGAGGA | 96 | TCCTCATG GGCTAGCTACAACGA ACTGGTCC | 2424 |
| 406 | ACCAGUAC A UGAGGACU | 97 | AGTCCTCA GGCTAGCTACAACGA GTACTGGT | 2425 |
| 412 | ACAUGAGG A CUGGGGAG | 98 | CTCCCCAG GGCTAGCTACAACGA CCTCATGT | 2426 |
| 422 | UGGGGAGG G CUUUCUUU | 99 | AAAGAAAG GGCTAGCTACAACGA CCTCCCCA | 2427 |
| 431 | CUUUCUUU G UGUAUUUG | 100 | CAAATACA GGCTAGCTACAACGA AAAGAAAG | 2428 |
| 433 | UUCUUUGU G UAUUUGCC | 101 | GGCAAATA GGCTAGCTACAACGA ACAAAGAA | 2429 |
| 435 | CUUUGUGU A UUUGCCAU | 102 | ATGGCAAA GGCTAGCTACAACGA ACACAAAG | 2430 |
| 439 | GUGUAUUU G CCAUAAAU | 103 | ATTTATGG GGCTAGCTACAACGA AAATACAC | 2431 |
| 442 | UAUUUGCC A UAAAUAAU | 104 | ATTATTTA GGCTAGCTACAACGA GGCAAATA | 2432 |
| 446 | UGCCAUAA A UAAUACUA | 105 | TAGTATTA GGCTAGCTACAACGA TTATGGCA | 2433 |
| 449 | CAUAAAUA A UACUAAAU | 106 | ATTTAGTA GGCTAGCTACAACGA TATTTATG | 2434 |
| 451 | UAAAUAAU A CUAAAUCA | 107 | TGATTTAG GGCTAGCTACAACGA ATTATTTA | 2435 |
| 456 | AAUACUAA A UCAUUUGA | 108 | TCAAATGA GGCTAGCTACAACGA TTAGTATT | 2436 |
| 459 | ACUAAAUC A UUUGAAGA | 109 | TCTTCAAA GGCTAGCTACAACGA GATTTAGT | 2437 |
| 467 | AUUUGAAG A UAUUCACC | 110 | GGTGAATA GGCTAGCTACAACGA CTTCAAAT | 2438 |
| 469 | UUGAAGAU A UUCACCAU | 111 | ATGGTGAA GGCTAGCTACAACGA ATCTTCAA | 2439 |
| 473 | AGAUAUUC A CCAUUAUA | 112 | TATAATGG GGCTAGCTACAACGA GAATATCT | 2440 |
| 476 | UAUUCACC A UUAUAGAG | 113 | CTCTATAA GGCTAGCTACAACGA GGTGAATA | 2441 |
| 479 | UCACCAUU A UAGAGAAC | 114 | GTTCTCTA GGCTAGCTACAACGA AATGGTGA | 2442 |
| 486 | UAUAGAGA A CAAAUUAA | 115 | TTAATTTG GGCTAGCTACAACGA TCTCTATA | 2443 |
| 490 | GAGAACAA A UUAAAAGA | 116 | TCTTTTAA GGCTAGCTACAACGA TTGTTCTC | 2444 |
| 499 | UUAAAAGA G UUAAGGAC | 117 | GTCCTTAA GGCTAGCTACAACGA TCTTTTAA | 2445 |
| 506 | AGUUAAGG A CUCUGAAG | 118 | CTTCAGAG GGCTAGCTACAACGA CCTTAACT | 2446 |
| 515 | CUCUGAAG A UGUACCUA | 119 | TAGGTACA GGCTAGCTACAACGA CTTCAGAG | 2447 |
| 517 | CUGAAGAU G UACCUAUG | 120 | CATAGGTA GGCTAGCTACAACGA ATCTTCAG | 2448 |
| 519 | GAAGAUGU A CCUAUGGU | 121 | ACCATAGG GGCTAGCTACAACGA ACATCTTC | 2449 |
| 523 | AUGUACCU A UGGUCCUA | 122 | TAGGACCA GGCTAGCTACAACGA AGGTACAT | 2450 |
| 526 | UACCUAUG G UCCUAGUA | 123 | TACTAGGA GGCTAGCTACAACGA CATAGGTA | 2451 |
| 532 | UGGUCCUA G UAGGAAAU | 124 | ATTTCCTA GGCTAGCTACAACGA TAGGACCA | 2452 |
| 539 | AGUAGGAA A UAAAUGUG | 125 | CACATTTA GGCTAGCTACAACGA TTCCTACT | 2453 |
| 543 | GGAAAUAA A UGUGAUUU | 126 | AAATCACA GGCTAGCTACAACGA TTATTTCC | 2454 |
| 545 | AAAUAAAU G UGAUUUGC | 127 | GCAAATCA GGCTAGCTACAACGA ATTTATTT | 2455 |
| 548 | UAAAUGUG A UUUGCCUU | 128 | AAGGCAAA GGCTAGCTACAACGA CACATTTA | 2456 |
| 552 | UGUGAUUU G CCUUCUAG | 129 | CTAGAAGG GGCTAGCTACAACGA AAATCACA | 2457 |
| 562 | CUUCUAGA A CAGUAGAC | 130 | GTCTACTG GGCTAGCTACAACGA TCTAGAAG | 2458 |
| 565 | CUAGAACA G UAGACACA | 131 | TGTGTCTA GGCTAGCTACAACGA TGTTCTAG | 2459 |
| 569 | AACAGUAG A CACAAAAC | 132 | GTTTTGTG GGCTAGCTACAACGA CTACTGTT | 2460 |
| 571 | CAGUAGAC A CAAAACAG | 133 | CTGTTTTG GGCTAGCTACAACGA GTCTACTG | 2461 |
| 576 | GACACAAA A CAGGCUCA | 134 | TGAGCCTG GGCTAGCTACAACGA TTTGTGTC | 2462 |
| 580 | CAAAACAG G CUCAGGAC | 135 | GTCCTGAG GGCTAGCTACAACGA CTGTTTTG | 2463 |
| 587 | GGCUCAGG A CUUAGCAA | 136 | TTGCTAAG GGCTAGCTACAACGA CCTGAGCC | 2464 |
| 592 | AGGACUUA G CAAGAAGU | 137 | ACTTCTTG GGCTAGCTACAACGA TAAGTCCT | 2465 |
| 599 | AGCAAGAA G UUAUGGAA | 138 | TTCCATAA GGCTAGCTACAACGA TTCTTGCT | 2466 |
| 602 | AAGAAGUU A UGGAAUUC | 139 | GAATTCCA GGCTAGCTACAACGA AACTTCTT | 2467 |
| 607 | GUUAUGGA A UUCCUUUU | 140 | AAAAGGAA GGCTAGCTACAACGA TCCATAAC | 2468 |
| 616 | UUCCUUUU A UUGAAACA | 141 | TGTTTCAA GGCTAGCTACAACGA AAAAGGAA | 2469 |
| 622 | UUAUUGAA A CAUCAGCA | 142 | TGCTGATG GGCTAGCTACAACGA TTCAATAA | 2470 |
| 624 | AUUGAAAC A UCAGCAAA | 143 | TTTGCTGA GGCTAGCTACAACGA GTTTCAAT | 2471 |
| 628 | AAACAUCA G CAAAGACA | 144 | TGTCTTTG GGCTAGCTACAACGA TGATGTTT | 2472 |
| 634 | CAGCAAAG A CAAGACAG | 145 | CTGTCTTG GGCTAGCTACAACGA CTTTGCTG | 2473 |
| 639 | AAGACAAG A CAGGGUGU | 146 | ACACCCTG GGCTAGCTACAACGA CTTGTCTT | 2474 |
| 644 | AAGACAGG G UGUUGAUG | 147 | CATCAACA GGCTAGCTACAACGA CCTGTCTT | 2475 |
| 646 | GACAGGGU G UUGAUGAU | 148 | ATCATCAA GGCTAGCTACAACGA ACCCTGTC | 2476 |
| 650 | GGGUGUUG A UGAUGCCU | 149 | AGGCATCA GGCTAGCTACAACGA CAACACCC | 2477 |
| 653 | UGUUGAUG A UGCCUUCU | 150 | AGAAGGCA GGCTAGCTACAACGA CATCAACA | 2478 |
| 655 | UUGAUGAU G CCUUCUAU | 151 | ATAGAAGG GGCTAGCTACAACGA ATCATCAA | 2479 |
| 662 | UGCCUUCU A UACAUUAG | 152 | CTAATGTA GGCTAGCTACAACGA AGAAGGCA | 2480 |
| 664 | CCUUCUAU A CAUUAGUU | 153 | AACTAATG GGCTAGCTACAACGA ATAGAAGG | 2481 |
| 666 | UUCUAUAC A UUAGUUCG | 154 | CGAACTAA GGCTAGCTACAACGA GTATAGAA | 2482 |
| 670 | AUACAUUA G UUCGAGAA | 155 | TTCTCGAA GGCTAGCTACAACGA TAATGTAT | 2483 |
| 679 | UUCGAGAA A UUCGAAAA | 156 | TTTTCGAA GGCTAGCTACAACGA TTCTCGAA | 2484 |
| 687 | AUUCGAAA A CAUAAAGA | 157 | TCTTTATG GGCTAGCTACAACGA TTTCGAAT | 2485 |
| 689 | UCGAAAAC A UAAAGAAA | 158 | TTTCTTTA GGCTAGCTACAACGA GTTTTCGA | 2486 |
| 700 | AAGAAAAG A UGAGCAAA | 159 | TTTGCTCA GGCTAGCTACAACGA CTTTTCTT | 2487 |
| 704 | AAAGAUGA G CAAAGAUG | 160 | CATCTTTG GGCTAGCTACAACGA TCATCTTT | 2488 |
| 710 | GAGCAAAG A UGGUAAAA | 161 | TTTTACCA GGCTAGCTACAACGA CTTTGCTC | 2489 |
| 713 | CAAAGAUG G UAAAAAGA | 162 | TCTTTTTA GGCTAGCTACAACGA CATCTTTG | 2490 |
| 732 | AAAAAGAA G UCAAAGAC | 163 | GTCTTTGA GGCTAGCTACAACGA TTCTTTTT | 2491 |
| 739 | AGUCAAAG A CAAAGUGU | 164 | ACACTTTG GGCTAGCTACAACGA CTTTGACT | 2492 |
| 744 | AAGACAAA G UGUGUAAU | 165 | ATTACACA GGCTAGCTACAACGA TTTGTCTT | 2493 |
| 746 | GACAAAGU G UGUAAUUA | 166 | TAATTACA GGCTAGCTACAACGA ACTTTGTC | 2494 |
| 748 | CAAAGUGU G UAAUUAUG | 167 | CATAATTA GGCTAGCTACAACGA ACACTTTG | 2495 |
| 751 | AGUGUGUA A UUAUGUAA | 168 | TTACATAA GGCTAGCTACAACGA TACACACT | 2496 |
| 754 | GUGUAAUU A UGUAAAUA | 169 | TATTTACA GGCTAGCTACAACGA AATTACAC | 2497 |
| 756 | GUAAUUAU G UAAAUACA | 170 | TGTATTTA GGCTAGCTACAACGA ATAATTAC | 2498 |
| 760 | UUAUGUAA A UACAAUUU | 171 | AAATTGTA GGCTAGCTACAACGA TTACATAA | 2499 |
| 762 | AUGUAAAU A CAAUUUGU | 172 | ACAAATTG GGCTAGCTACAACGA ATTTACAT | 2500 |
| 765 | UAAAUACA A UUUGUACU | 173 | AGTACAAA GGCTAGCTACAACGA TGTATTTA | 2501 |
| 769 | UACAAUUU G UACUUUUU | 174 | AAAAAGTA GGCTAGCTACAACGA AAATTGTA | 2502 |
| 771 | CAAUUUGU A CUUUUUUC | 175 | GAAAAAAG GGCTAGCTACAACGA ACAAATTG | 2503 |
| 785 | UUCUUAAG G CAUACUAG | 176 | CTAGTATG GGCTAGCTACAACGA CTTAAGAA | 2504 |
| 787 | CUUAAGGC A UACUAGUA | 177 | TACTAGTA GGCTAGCTACAACGA GCCTTAAG | 2505 |
| 789 | UAAGGCAU A CUAGUACA | 178 | TGTACTAG GGCTAGCTACAACGA ATGCCTTA | 2506 |
| 793 | GCAUACUA G UACAAGUG | 179 | CACTTGTA GGCTAGCTACAACGA TAGTATGC | 2507 |
| 795 | AUACUAGU A CAAGUGGU | 180 | ACCACTTG GGCTAGCTACAACGA ACTAGTAT | 2508 |
| 799 | UAGUACAA G UGGUAAUU | 181 | AATTACCA GGCTAGCTACAACGA TTGTACTA | 2509 |
| 802 | UACAAGUG G UAAUUUUU | 182 | AAAAATTA GGCTAGCTACAACGA CACTTGTA | 2510 |
| 805 | AAGUGGUA A UUUUUGUA | 183 | TACAAAAA GGCTAGCTACAACGA TACCACTT | 2511 |
| 811 | UAAUUUUU G UACAUUAC | 184 | GTAATGTA GGCTAGCTACAACGA AAAAATTA | 2512 |
| 813 | AUUUUUGU A CAUUACAC | 185 | GTGTAATG GGCTAGCTACAACGA ACAAAAAT | 2513 |
| 815 | UUUUGUAC A UUACACUA | 186 | TAGTGTAA GGCTAGCTACAACGA GTACAAAA | 2514 |
| 818 | UGUACAUU A CACUAAAU | 187 | ATTTAGTG GGCTAGCTACAACGA AATGTACA | 2515 |
| 820 | UACAUUAC A CUAAAUUA | 188 | TAATTTAG GGCTAGCTACAACGA GTAATGTA | 2516 |
| 825 | UACACUAA A UUAUUAGC | 189 | GCTAATAA GGCTAGCTACAACGA TTAGTGTA | 2517 |
| 828 | ACUAAAUU A UUAGCAUU | 190 | AATGCTAA GGCTAGCTACAACGA AATTTAGT | 2518 |
| 832 | AAUUAUUA G CAUUUGUU | 191 | AACAAATG GGCTAGCTACAACGA TAATAATT | 2519 |
| 834 | UUAUUAGC A UUUGUUUU | 192 | AAAACAAA GGCTAGCTACAACGA GCTAATAA | 2520 |
| 838 | UAGCAUUU G UUUUAGCA | 193 | TGCTAAAA GGCTAGCTACAACGA AAATGCTA | 2521 |
| 844 | UUGUUUUA G CAUUACCU | 194 | AGGTAATG GGCTAGCTACAACGA TAAAACAA | 2522 |
| 846 | GUUUUAGC A UUACCUAA | 195 | TTAGGTAA GGCTAGCTACAACGA GCTAAAAC | 2523 |
| 849 | UUAGCAUU A CCUAAUUU | 196 | AAATTAGG GGCTAGCTACAACGA AATGCTAA | 2524 |
| 854 | AUUACCUA A UUUUUUUC | 197 | GAAAAAAA GGCTAGCTACAACGA TAGGTAAT | 2525 |
| 865 | UUUUUCCU G CUCCAUGC | 198 | GCATGGAG GGCTAGCTACAACGA AGGAAAAA | 2526 |
| 870 | CCUGCUCC A UGCAGACU | 199 | AGTCTGCA GGCTAGCTACAACGA GGAGCAGG | 2527 |
| 872 | UGCUCCAU G CAGACUGU | 200 | ACAGTCTG GGCTAGCTACAACGA ATGGAGCA | 2528 |
| 876 | CCAUGCAG A CUGUUAGC | 201 | GCTAACAG GGCTAGCTACAACGA CTGCATGG | 2529 |
| 879 | UGCAGACU G UUAGCUUU | 202 | AAAGCTAA GGCTAGCTACAACGA AGTCTGCA | 2530 |
| 883 | GACUGUUA G CUUUUACC | 203 | GGTAAAAG GGCTAGCTACAACGA TAACAGTC | 2531 |
| 889 | UAGCUUUU A CCUUAAAU | 204 | ATTTAAGG GGCTAGCTACAACGA AAAAGCTA | 2532 |
| 896 | UACCUUAA A UGCUUAUU | 205 | AATAAGCA GGCTAGCTACAACGA TTAAGGTA | 2533 |
| 898 | CCUUAAAU G CUUAUUUU | 206 | AAAATAAG GGCTAGCTACAACGA ATTTAAGG | 2534 |
| 902 | AAAUGCUU A UUUUAAAA | 207 | TTTTAAAA GGCTAGCTACAACGA AAGCATTT | 2535 |
| 910 | AUUUUAAA A UGACAGUG | 208 | CACTGTCA GGCTAGCTACAACGA TTTAAAAT | 2536 |
| 913 | UUAAAAUG A CAGUGGAA | 209 | TTCCACTG GGCTAGCTACAACGA CATTTTAA | 2537 |
| 916 | AAAUGACA G UGGAAGUU | 210 | AACTTCCA GGCTAGCTACAACGA TGTCATTT | 2538 |
| 922 | CAGUGGAA G UUUUUUUU | 211 | AAAAAAAA GGCTAGCTACAACGA TTCCACTG | 2539 |
| 939 | UCCUCGAA G UGCCAGUA | 212 | TACTGGCA GGCTAGCTACAACGA TTCGAGGA | 2540 |
| 941 | CUCGAAGU G CCAGUAUU | 213 | AATACTGG GGCTAGCTACAACGA ACTTCGAG | 2541 |
| 945 | AAGUGCCA G UAUUCCCA | 214 | TGGGAATA GGCTAGCTACAACGA TGGCACTT | 2542 |
| 947 | GUGCCAGU A UUCCCAGA | 215 | TCTGGGAA GGCTAGCTACAACGA ACTGGCAC | 2543 |
| 956 | UUCCCAGA G UUUUGGUU | 216 | AACCAAAA GGCTAGCTACAACGA TCTGGGAA | 2544 |
| 962 | GAGUUUUG G UUUUUGAA | 217 | TTCAAAAA GGCTAGCTACAACGA CAAAACTC | 2545 |
| 970 | GUUUUUGA A CUAGCAAU | 218 | ATTGCTAG GGCTAGCTACAACGA TCAAAAAC | 2546 |
| 974 | UUGAACUA G CAAUGCCU | 219 | AGGCATTG GGCTAGCTACAACGA TAGTTCAA | 2547 |
| 977 | AACUAGCA A UGCCUGUG | 220 | CACAGGCA GGCTAGCTACAACGA TGCTAGTT | 2548 |
| 979 | CUAGCAAU G CCUGUGAA | 221 | TTCACAGG GGCTAGCTACAACGA ATTGCTAG | 2549 |
| 983 | CAAUGCCU G UGAAAAAG | 222 | CTTTTTCA GGCTAGCTACAACGA AGGCATTG | 2550 |
| 994 | AAAAAGAA A CUGAAUAC | 223 | GTATTCAG GGCTAGCTACAACGA TTCTTTTT | 2551 |
| 999 | GAAACUGA A UACCUAAG | 224 | CTTAGGTA GGCTAGCTACAACGA TCAGTTTC | 2552 |
| 1001 | AACUGAAU A CCUAAGAU | 225 | ATCTTAGG GGCTAGCTACAACGA ATTCAGTT | 2553 |
| 1008 | UACCUAAG A UUUCUGUC | 226 | GACAGAAA GGCTAGCTACAACGA CTTAGGTA | 2554 |
| 1014 | AGAUUUCU G UCUUGGGG | 227 | CCCCAAGA GGCTAGCTACAACGA AGAAATCT | 2555 |
| 1022 | GUCUUGGG G UUUUUGGU | 228 | ACCAAAAA GGCTAGCTACAACGA CCCAAGAC | 2556 |
| 1029 | GGUUUUUG G UGCAUGCA | 229 | TGCATGCA GGCTAGCTACAACGA CAAAAACC | 2557 |
| 1031 | UUUUUGGU G CAUGCAGU | 230 | ACTGCATG GGCTAGCTACAACGA ACCAAAAA | 2558 |
| 1033 | UUUGGUGC A UGCAGUUG | 231 | CAACTGCA GGCTAGCTACAACGA GCACCAAA | 2559 |
| 1035 | UGGUGCAU G CAGUUGAU | 232 | ATCAACTG GGCTAGCTACAACGA ATGCACCA | 2560 |
| 1038 | UGCAUGCA G UUGAUUAC | 233 | GTAATCAA GGCTAGCTACAACGA TGCATGCA | 2561 |
| 1042 | UGCAGUUG A UUACUUCU | 234 | AGAAGTAA GGCTAGCTACAACGA CAACTGCA | 2562 |
| 1045 | AGUUGAUU A CUUCUUAU | 235 | ATAAGAAG GGCTAGCTACAACGA AATCAACT | 2563 |
| 1052 | UACUUCUU A UUUUUCUU | 236 | AAGAAAAA GGCTAGCTACAACGA AAGAAGTA | 2564 |
| 1061 | UUUUUCUU A CCAAGUGU | 237 | ACACTTGG GGCTAGCTACAACGA AAGAAAAA | 2565 |
| 1066 | CUUACCAA G UGUGAAUG | 238 | CATTCACA GGCTAGCTACAACGA TTGGTAAG | 2566 |
| 1068 | UACCAAGU G UGAAUGUU | 239 | AACATTCA GGCTAGCTACAACGA ACTTGGTA | 2567 |
| 1072 | AAGUGUGA A UGUUGGUG | 240 | CACCAACA GGCTAGCTACAACGA TCACACTT | 2568 |
| 1074 | GUGUGAAU G UUGGUGUG | 241 | CACACCAA GGCTAGCTACAACGA ATTCACAC | 2569 |
| 1078 | GAAUGUUG G UGUGAAAC | 242 | GTTTCACA GGCTAGCTACAACGA CAACATTC | 2570 |
| 1080 | AUGUUGGU G UGAAACAA | 243 | TTGTTTCA GGCTAGCTACAACGA ACCAACAT | 2571 |
| 1085 | GGUGUGAA A CAAAUUAA | 244 | TTAATTTG GGCTAGCTACAACGA TTCACACC | 2572 |
| 1089 | UGAAACAA A UUAAUGAA | 245 | TTCATTAA GGCTAGCTACAACGA TTGTTTCA | 2573 |
| 1093 | ACAAAUUA A UGAAGCUU | 246 | AAGCTTCA GGCTAGCTACAACGA TAATTTGT | 2574 |
| 1098 | UUAAUGAA G CUUUUGAA | 247 | TTCAAAAG GGCTAGCTACAACGA TTCATTAA | 2575 |
| 1106 | GCUUUUGA A UCAUCCCU | 248 | AGGGATGA GGCTAGCTACAACGA TCAAAAGC | 2576 |
| 1109 | UUUGAAUC A UCCCUAUU | 249 | AATAGGGA GGCTAGCTACAACGA GATTCAAA | 2577 |
| 1115 | UCAUCCCU A UUCUGUGU | 250 | ACACAGAA GGCTAGCTACAACGA AGGGATGA | 2578 |
| 1120 | CCUAUUCU G UGUUUUAU | 251 | ATAAAACA GGCTAGCTACAACGA AGAATAGG | 2579 |
| 1122 | UAUUCUGU G UUUUAUCU | 252 | AGATAAAA GGCTAGCTACAACGA ACAGAATA | 2580 |
| 1127 | UGUGUUUU A UCUAGUCA | 253 | TGACTAGA GGCTAGCTACAACGA AAAACACA | 2581 |
| 1132 | UUUAUCUA G UCACAUAA | 254 | TTATGTGA GGCTAGCTACAACGA TAGATAAA | 2582 |
| 1135 | AUCUAGUC A CAUAAAUG | 255 | CATTTATG GGCTAGCTACAACGA GACTAGAT | 2583 |
| 1137 | CUAGUCAC A UAAAUGGA | 256 | TCCATTTA GGCTAGCTACAACGA GTGACTAG | 2584 |
| 1141 | UCACAUAA A UGGAUUAA | 257 | TTAATCCA GGCTAGCTACAACGA TTATGTGA | 2585 |
| 1145 | AUAAAUGG A UUAAUUAC | 258 | GTAATTAA GGCTAGCTACAACGA CCATTTAT | 2586 |
| 1149 | AUGGAUUA A UUACUAAU | 259 | ATTAGTAA GGCTAGCTACAACGA TAATCCAT | 2587 |
| 1152 | GAUUAAUU A CUAAUUUC | 260 | GAAATTAG GGCTAGCTACAACGA AATTAATC | 2588 |
| 1156 | AAUUACUA A UUUCAGUU | 261 | AACTGAAA GGCTAGCTACAACGA TAGTAATT | 2589 |
| 1162 | UAAUUUCA G UUGAGACC | 262 | GGTCTCAA GGCTAGCTACAACGA TGAAATTA | 2590 |
| 1168 | CAGUUGAG A CCUUCUAA | 263 | TTAGAAGG GGCTAGCTACAACGA CTCAACTG | 2591 |
| 1176 | ACCUUCUA A UUGGUUUU | 264 | AAAACCAA GGCTAGCTACAACGA TAGAAGGT | 2592 |
| 1180 | UCUAAUUG G UUUUUACU | 265 | AGTAAAAA GGCTAGCTACAACGA CAATTAGA | 2593 |
| 1186 | UGGUUUUU A CUGAAACA | 266 | TGTTTCAG GGCTAGCTACAACGA AAAAACCA | 2594 |
| 1192 | UUACUGAA A CAUUGAGG | 267 | CCTCAATG GGCTAGCTACAACGA TTCAGTAA | 2595 |
| 1194 | ACUGAAAC A UUGAGGGA | 268 | TCCCTCAA GGCTAGCTACAACGA GTTTCAGT | 2596 |
| 1202 | AUUGAGGG A CACAAAUU | 269 | AATTTGTG GGCTAGCTACAACGA CCCTCAAT | 2597 |
| 1204 | UGAGGGAC A CAAAUUUA | 270 | TAAATTTG GGCTAGCTACAACGA GTCCCTCA | 2598 |
| 1208 | GGACACAA A UUUAUGGG | 271 | CCCATAAA GGCTAGCTACAACGA TTGTGTCC | 2599 |
| 1212 | ACAAAUUU A UGGGCUUC | 272 | GAAGCCCA GGCTAGCTACAACGA AAATTTGT | 2600 |
| 1216 | AUUUAUGG G CUUCCUGA | 273 | TCAGGAAG GGCTAGCTACAACGA CCATAAAT | 2601 |
| 1224 | GCUUCCUG A UGAUGAUU | 274 | AATCATCA GGCTAGCTACAACGA CAGGAAGC | 2602 |
| 1227 | UCCUGAUG A UGAUUCUU | 275 | AAGAATCA GGCTAGCTACAACGA CATCAGGA | 2603 |
| 1230 | UGAUGAUG A UUCUUCUA | 276 | TAGAAGAA GGCTAGCTACAACGA CATCATCA | 2604 |
| 1240 | UCUUCUAG G CAUCAUGU | 277 | ACATGATG GGCTAGCTACAACGA CTAGAAGA | 2605 |
| 1242 | UUCUAGGC A UCAUGUCC | 278 | GGACATGA GGCTAGCTACAACGA GCCTAGAA | 2606 |
| 1245 | UAGGCAUC A UGUCCUAU | 279 | ATAGGACA GGCTAGCTACAACGA GATGCCTA | 2607 |
| 1247 | GGCAUCAU G UCCUAUAG | 280 | CTATAGGA GGCTAGCTACAACGA ATGATGCC | 2608 |
| 1252 | CAUGUCCU A UAGUUUGU | 281 | ACAAACTA GGCTAGCTACAACGA AGGACATG | 2609 |
| 1255 | GUCCUAUA G UUUGUCAU | 282 | ATGACAAA GGCTAGCTACAACGA TATAGGAC | 2610 |
| 1259 | UAUAGUUU G UCAUCCCU | 283 | AGGGATGA GGCTAGCTACAACGA AAACTATA | 2611 |
| 1262 | AGUUUGUC A UCCCUGAU | 284 | ATCAGGGA GGCTAGCTACAACGA GACAAACT | 2612 |
| 1269 | CAUCCCUG A UGAAUGUA | 285 | TACATTCA GGCTAGCTACAACGA CAGGGATG | 2613 |
| 1273 | CCUGAUGA A UGUAAAGU | 286 | ACTTTACA GGCTAGCTACAACGA TCATCAGG | 2614 |
| 1275 | UGAUGAAU G UAAAGUUA | 287 | TAACTTTA GGCTAGCTACAACGA ATTCATCA | 2615 |
| 1280 | AAUGUAAA G UUACACUG | 288 | CAGTGTAA GGCTAGCTACAACGA TTTACATT | 2616 |
| 1283 | GUAAAGUU A CACUGUUC | 289 | GAACAGTG GGCTAGCTACAACGA AACTTTAC | 2617 |
| 1285 | AAAGUUAC A CUGUUCAC | 290 | GTGAACAG GGCTAGCTACAACGA GTAACTTT | 2618 |
| 1288 | GUUACACU G UUCACAAA | 291 | TTTGTGAA GGCTAGCTACAACGA AGTGTAAC | 2619 |
| 1292 | CACUGUUC A CAAAGGUU | 292 | AACCTTTG GGCTAGCTACAACGA GAACAGTG | 2620 |
| 1298 | UCACAAAG G UUUUGUCU | 293 | AGACAAAA GGCTAGCTACAACGA CTTTGTGA | 2621 |
| 1303 | AAGGUUUU G UCUCCUUU | 294 | AAAGGAGA GGCTAGCTACAACGA AAAACCTT | 2622 |
| 1314 | UCCUUUCC A CUGCUAUU | 295 | AATAGCAG GGCTAGCTACAACGA GGAAAGGA | 2623 |
| 1317 | UUUCCACU G CUAUUAGU | 296 | ACTAATAG GGCTAGCTACAACGA AGTGGAAA | 2624 |
| 1320 | CCACUGCU A UUAGUCAU | 297 | ATGACTAA GGCTAGCTACAACGA AGCAGTGG | 2625 |
| 1324 | UGCUAUUA G UCAUGGUC | 298 | GACCATGA GGCTAGCTACAACGA TAATAGCA | 2626 |
| 1327 | UAUUAGUC A UGGUCACU | 299 | AGTGACCA GGCTAGCTACAACGA GACTAATA | 2627 |
| 1330 | UAGUCAUG G UCACUCUC | 300 | GAGAGTGA GGCTAGCTACAACGA CATGACTA | 2628 |
| 1333 | UCAUGGUC A CUCUCCCC | 301 | GGGGAGAG GGCTAGCTACAACGA GACCATGA | 2629 |
| 1345 | UCCCCAAA A UAUUAUAU | 302 | ATATAATA GGCTAGCTACAACGA TTTGGGGA | 2630 |
| 1347 | CCCAAAAU A UUAUAUUU | 303 | AAATATAA GGCTAGCTACAACGA ATTTTGGG | 2631 |
| 1350 | AAAAUAUU A UAUUUUUU | 304 | AAAAAATA GGCTAGCTACAACGA AATATTTT | 2632 |
| 1352 | AAUAUUAU A UUUUUUCU | 305 | AGAAAAAA GGCTAGCTACAACGA ATAATATT | 2633 |
| 1361 | UUUUUUCU A UAAAAAGA | 306 | TCTTTTTA GGCTAGCTACAACGA AGAAAAAA | 2634 |
| 1375 | AGAAAAAA A UGGAAAAA | 307 | TTTTTCCA GGCTAGCTACAACGA TTTTTTCT | 2635 |
| 1385 | GGAAAAAA A UUACAAGG | 308 | CCTTGTAA GGCTAGCTACAACGA TTTTTTCC | 2636 |
| 1388 | AAAAAAUU A CAAGGCAA | 309 | TTGCCTTG GGCTAGCTACAACGA AATTTTTT | 2637 |
| 1393 | AUUACAAG G CAAUGGAA | 310 | TTCCATTG GGCTAGCTACAACGA CTTGTAAT | 2638 |
| 1396 | ACAAGGCA A UGGAAACU | 311 | AGTTTCCA GGCTAGCTACAACGA TGCCTTGT | 2639 |
| 1402 | CAAUGGAA A CUAUUAUA | 312 | TATAATAG GGCTAGCTACAACGA TTCCATTG | 2640 |
| 1405 | UGGAAACU A UUAUAAGG | 313 | CCTTATAA GGCTAGCTACAACGA AGTTTCCA | 2641 |
| 1408 | AAACUAUU A UAAGGCCA | 314 | TGGCCTTA GGCTAGCTACAACGA AATAGTTT | 2642 |
| 1413 | AUUAUAAG G CCAUUUCC | 315 | GGAAATGG GGCTAGCTACAACGA CTTATAAT | 2643 |
| 1416 | AUAAGGCC A UUUCCUUU | 316 | AAAGGAAA GGCTAGCTACAACGA GGCCTTAT | 2644 |
| 1427 | UCCUUUUC A CAUUAGAU | 317 | ATCTAATG GGCTAGCTACAACGA GAAAAGGA | 2645 |
| 1429 | CUUUUCAC A UUAGAUAA | 318 | TTATCTAA GGCTAGCTACAACGA GTGAAAAG | 2646 |
| 1434 | CACAUUAG A UAAAUUAC | 319 | GTAATTTA GGCTAGCTACAACGA CTAATGTG | 2647 |
| 1438 | UUAGAUAA A UUACUAUA | 320 | TATAGTAA GGCTAGCTACAACGA TTATCTAA | 2648 |
| 1441 | GAUAAAUU A CUAUAAAG | 321 | CTTTATAG GGCTAGCTACAACGA AATTTATC | 2649 |
| 1444 | AAAUUACU A UAAAGACU | 322 | AGTCTTTA GGCTAGCTACAACGA AGTAATTT | 2650 |
| 1450 | CUAUAAAG A CUCCUAAU | 323 | ATTAGGAG GGCTAGCTACAACGA CTTTATAG | 2651 |
| 1457 | GACUCCUA A UAGCUUUU | 324 | AAAAGCTA GGCTAGCTACAACGA TAGGAGTC | 2652 |
| 1460 | UCCUAAUA G CUUUUUCC | 325 | GGAAAAAG GGCTAGCTACAACGA TATTAGGA | 2653 |
| 1470 | UUUUUCCU G UUAAGGCA | 326 | TGCCTTAA GGCTAGCTACAACGA AGGAAAAA | 2654 |
| 1476 | CUGUUAAG G CAGACCCA | 327 | TGGGTCTG GGCTAGCTACAACGA CTTAACAG | 2655 |
| 1480 | UAAGGCAG A CCCAGUAU | 328 | ATACTGGG GGCTAGCTACAACGA CTGCCTTA | 2656 |
| 1485 | CAGACCCA G UAUGAAUG | 329 | CATTCATA GGCTAGCTACAACGA TGGGTCTG | 2657 |
| 1487 | GACCCAGU A UGAAUGGG | 330 | CCCATTCA GGCTAGCTACAACGA ACTGGGTC | 2658 |
| 1491 | CAGUAUGA A UGGGAUUA | 331 | TAATCCCA GGCTAGCTACAACGA TCATACTG | 2659 |
| 1496 | UGAAUGGG A UUAUUAUA | 332 | TATAATAA GGCTAGCTACAACGA CCCATTCA | 2660 |
| 1499 | AUGGGAUU A UUAUAGCA | 333 | TGCTATAA GGCTAGCTACAACGA AATCCCAT | 2661 |
| 1502 | GGAUUAUU A UAGCAACC | 334 | GGTTGCTA GGCTAGCTACAACGA AATAATCC | 2662 |
| 1505 | UUAUUAUA G CAACCAUU | 335 | AATGGTTG GGCTAGCTACAACGA TATAATAA | 2663 |
| 1508 | UUAUAGCA A CCAUUUUG | 336 | CAAAATGG GGCTAGCTACAACGA TGCTATAA | 2664 |
| 1511 | UAGCAACC A UUUUGGGG | 337 | CCCCAAAA GGCTAGCTACAACGA GGTTGCTA | 2665 |
| 1519 | AUUUUGGG G CUAUAUUU | 338 | AAATATAG GGCTAGCTACAACGA CCCAAAAT | 2666 |
| 1522 | UUGGGGCU A UAUUUACA | 339 | TGTAAATA GGCTAGCTACAACGA AGCCCCAA | 2667 |
| 1524 | GGGGCUAU A UUUACAUG | 340 | CATGTAAA GGCTAGCTACAACGA ATAGCCCC | 2668 |
| 1528 | CUAUAUUU A CAUGCUAC | 341 | GTAGCATG GGCTAGCTACAACGA AAATATAG | 2669 |
| 1530 | AUAUUUAC A UGCUACUA | 342 | TAGTAGCA GGCTAGCTACAACGA GTAAATAT | 2670 |
| 1532 | AUUUACAU G CUACUAAA | 343 | TTTAGTAG GGCTAGCTACAACGA ATGTAAAT | 2671 |
| 1535 | UACAUGCU A CUAAAUUU | 344 | AAATTTAG GGCTAGCTACAACGA AGCATGTA | 2672 |
| 1540 | GCUACUAA A UUUUUAUA | 345 | TATAAAAA GGCTAGCTACAACGA TTAGTAGC | 2673 |
| 1546 | AAAUUUUU A UAAUAAUU | 346 | AATTATTA GGCTAGCTACAACGA AAAAATTT | 2674 |
| 1549 | UUUUUAUA A UAAUUGAA | 347 | TTCAATTA GGCTAGCTACAACGA TATAAAAA | 2675 |
| 1552 | UUAUAAUA A UUGAAAAG | 348 | CTTTTCAA GGCTAGCTACAACGA TATTATAA | 2676 |
| 1561 | UUGAAAAG A UUUUAACA | 349 | TGTTAAAA GGCTAGCTACAACGA CTTTTCAA | 2677 |
| 1567 | AGAUUUUA A CAAGUAUA | 350 | TATACTTG GGCTAGCTACAACGA TAAAATCT | 2678 |
| 1571 | UUUAACAA G UAUAAAAA | 351 | TTTTTATA GGCTAGCTACAACGA TTGTTAAA | 2679 |
| 1573 | UAACAAGU A UAAAAAAA | 352 | TTTTTTTA GGCTAGCTACAACGA ACTTGTTA | 2680 |
| 1581 | AUAAAAAA A UUCUCAUA | 353 | TATGAGAA GGCTAGCTACAACGA TTTTTTAT | 2681 |
| 1587 | AAAUUCUC A UAGGAAUU | 354 | AATTCCTA GGCTAGCTACAACGA GAGAATTT | 2682 |
| 1593 | UCAUAGGA A UUAAAUGU | 355 | ACATTTAA GGCTAGCTACAACGA TCCTATGA | 2683 |
| 1598 | GGAAUUAA A UGUAGUCU | 356 | AGACTACA GGCTAGCTACAACGA TTAATTCC | 2684 |
| 1600 | AAUUAAAU G UAGUCUCC | 357 | GGAGACTA GGCTAGCTACAACGA ATTTAATT | 2685 |
| 1603 | UAAAUGUA G UCUCCCUG | 358 | CAGGGAGA GGCTAGCTACAACGA TACATTTA | 2686 |
| 1611 | GUCUCCCU G UGUCAGAC | 359 | GTCTGACA GGCTAGCTACAACGA AGGGAGAC | 2687 |
| 1613 | CUCCCUGU G UCAGACUG | 360 | CAGTCTGA GGCTAGCTACAACGA ACAGGGAG | 2688 |
| 1618 | UGUGUCAG A CUGCUCUU | 361 | AAGAGCAG GGCTAGCTACAACGA CTGACACA | 2689 |
| 1621 | GUCAGACU G CUCUUUCA | 362 | TGAAAGAG GGCTAGCTACAACGA AGTCTGAC | 2690 |
| 1629 | GCUCUUUC A UAGUAUAA | 363 | TTATACTA GGCTAGCTACAACGA GAAAGAGC | 2691 |
| 1632 | CUUUCAUA G UAUAACUU | 364 | AAGTTATA GGCTAGCTACAACGA TATGAAAG | 2692 |
| 1634 | UUCAUAGU A UAACUUUA | 365 | TAAAGTTA GGCTAGCTACAACGA ACTATGAA | 2693 |
| 1637 | AUAGUAUA A CUUUAAAU | 366 | ATTTAAAG GGCTAGCTACAACGA TATACTAT | 2694 |
| 1644 | AACUUUAA A UCUUUUCU | 367 | AGAAAAGA GGCTAGCTACAACGA TTAAAGTT | 2695 |
| 1656 | UUUCUUGA A CUUGAGUC | 368 | GACTCAAG GGCTAGCTACAACGA TGAAGAAA | 2696 |
| 1662 | CAAGUUGA G UCUUUGAA | 369 | TTCAAAGA GGCTAGCTACAACGA TCAAGTTG | 2697 |
| 1672 | CUUUGAAG A UAGUUUUA | 370 | TAAAACTA GGCTAGCTACAACGA CTTCAAAG | 2698 |
| 1675 | UGAAGAUA G UUUUAAUU | 371 | AATTAAAA GGCTAGCTACAACGA TATCTTCA | 2699 |
| 1681 | UAGUUUUA A UUCUGCUU | 372 | AAGCAGAA GGCTAGCTACAACGA TAAAACTA | 2700 |
| 1686 | UUAAUUCU G CUUGUGAC | 373 | GTCACAAG GGCTAGCTACAACGA AGAATTAA | 2701 |
| 1690 | UUCUGCUU G UGACAUUA | 374 | TAATGTCA GGCTAGCTACAACGA AAGCAGAA | 2702 |
| 1693 | UGCUUGUG A CAUUAAAA | 375 | TTTTAATG GGCTAGCTACAACGA CACAAGCA | 2703 |
| 1695 | CUUGUGAC A UUAAAAGA | 376 | TCTTTTAA GGCTAGCTACAACGA GTCACAAG | 2704 |
| 1703 | AUUAAAAG A UUAUUUGG | 377 | CCAAATAA GGCTAGCTACAACGA CTTTTAAT | 2705 |
| 1706 | AAAAGAUU A UUUGGGCC | 378 | GGCCCAAA GGCTAGCTACAACGA AATCTTTT | 2706 |
| 1712 | UUAUUUGG G CCAGUUAU | 379 | ATAACTGG GGCTAGCTACAACGA CCAAATAA | 2707 |
| 1716 | UUGGGCCA G UUAUAGCU | 380 | AGCTATAA GGCTAGCTACAACGA TGGCCCAA | 2708 |
| 1719 | GGCCAGUU A UAGCUUAU | 381 | ATAAGCTA GGCTAGCTACAACGA AACTGGCC | 2709 |
| 1722 | CAGUUAUA G CUUAUUAG | 382 | CTAATAAG GGCTAGCTACAACGA TATAACTG | 2710 |
| 1726 | UAUAGCUU A UUAGGUGU | 383 | ACACCTAA GGCTAGCTACAACGA AAGCTATA | 2711 |
| 1731 | CUUAUUAG G UGUUGAAG | 384 | CTTCAACA GGCTAGCTACAACGA CTAATAAG | 2712 |
| 1733 | UAUUAGGU G UUGAAGAG | 385 | CTCTTCAA GGCTAGCTACAACGA ACCTAATA | 2713 |
| 1742 | UUGAAGAG A CCAAGGUU | 386 | AACCTTGG GGCTAGCTACAACGA CTCTTCAA | 2714 |
| 1748 | AGACCAAG G UUGCAAGC | 387 | GCTTGCAA GGCTAGCTACAACGA CTTGGTCT | 2715 |
| 1751 | CCAAGGUU G CAAGCCAG | 388 | CTGGCTTG GGCTAGCTACAACGA AACCTTGG | 2716 |
| 1755 | GGUUGCAA G CCAGGCCC | 389 | GGGCCTGG GGCTAGCTACAACGA TTGCAACC | 2717 |
| 1760 | CAAGCCAG G CCCUGUGU | 390 | ACACAGGG GGCTAGCTACAACGA CTGGCTTG | 2718 |
| 1765 | CAGGCCCU G UGUGAACC | 391 | GGTTCACA GGCTAGCTACAACGA AGGGCCTG | 2719 |
| 1767 | GGCCCUGU G UGAACCUU | 392 | AAGGTTCA GGCTAGCTACAACGA ACAGGGCC | 2720 |
| 1772 | CUGUGUGA A CCUUGAGC | 393 | GCTCAAGG GGCTAGCTACAACGA TCACACAG | 2721 |
| 1778 | AACCUUGA G CUUUCAUA | 394 | TATGAAAG GGCTAGCTACAACGA TCAAGGTT | 2722 |
| 1784 | GAGCUUUC A UAGAGAGU | 395 | ACTCTCTA GGCTAGCTACAACGA GAAAGCTC | 2723 |
| 1791 | CAUAGAGA G UUUCACAG | 396 | CTGTGAAA GGCTAGCTACAACGA TCTCTATG | 2724 |
| 1796 | AGAGUUUC A CAGCAUGG | 397 | CCATGCTG GGCTAGCTACAACGA GAAACTCT | 2725 |
| 1799 | GUUUCACA G CAUGGACU | 398 | AGTCCATG GGCTAGCTACAACGA TGTGAAAC | 2726 |
| 1801 | UUCACAGC A UGGACUGU | 399 | ACAGTCCA GGCTAGCTACAACGA GCTGTGAA | 2727 |
| 1805 | CAGCAUGG A CUGUGUGC | 400 | GCACACAG GGCTAGCTACAACGA CCATGCTG | 2728 |
| 1808 | CAUGGACU G UGUGCCCC | 401 | GGGGCACA GGCTAGCTACAACGA AGTCCATG | 2729 |
| 1810 | UGGACUGU G UGCCCCAC | 402 | GTGGGGCA GGCTAGCTACAACGA ACAGTCCA | 2730 |
| 1812 | GACUGUGU G CCCCACGG | 403 | CCGTGGGG GGCTAGCTACAACGA ACACAGTC | 2731 |
| 1817 | UGUGCCCC A CGGUCAUC | 404 | GATGACCG GGCTAGCTACAACGA GGGGCACA | 2732 |
| 1820 | GCCCCACG G UCAUCCGA | 405 | TCGGATGA GGCTAGCTACAACGA CGTGGGGC | 2733 |
| 1823 | CCACGGUC A UCCGAGUG | 406 | CACTCGGA GGCTAGCTACAACGA GACCGTGG | 2734 |
| 1829 | UCAUCCGA G UGGUUGUA | 407 | TACAACCA GGCTAGCTACAACGA TCGGATGA | 2735 |
| 1832 | UCCGAGUG G UUGUACGA | 408 | TCGTACAA GGCTAGCTACAACGA CACTCGGA | 2736 |
| 1835 | GAGUGGUU G UACGAUGC | 409 | GCATCGTA GGCTAGCTACAACGA AACCACTC | 2737 |
| 1837 | GUGGUUGU A CGAUGCAU | 410 | ATGCATCG GGCTAGCTACAACGA ACAACCAC | 2738 |
| 1840 | GUUGUACG A UGCAUUGG | 411 | CCAATGCA GGCTAGCTACAACGA CGTACAAC | 2739 |
| 1842 | UGUACGAU G CAUUGGUU | 412 | AACCAATG GGCTAGCTACAACGA ATCGTACA | 2740 |
| 1844 | UACGAUGC A UUGGUUAG | 413 | CTAACCAA GGCTAGCTACAACGA GCATCGTA | 2741 |
| 1848 | AUGCAUUG G UUAGUCAA | 414 | TTGACTAA GGCTAGCTACAACGA CAATGCAT | 2742 |
| 1852 | AUUGGUUA G UCAAAAAU | 415 | ATTTTTGA GGCTAGCTACAACGA TAACCAAT | 2743 |
| 1859 | AGUCAAAA A UGGGGAGG | 416 | CCTCCCCA GGCTAGCTACAACGA TTTTGACT | 2744 |
| 1869 | GGGGAGGG A CUAGGGCA | 417 | TGCCCTAG GGCTAGCTACAACGA CCCTCCCC | 2745 |
| 1875 | GGACUAGG G CAGUUUGG | 418 | CCAAACTG GGCTAGCTACAACGA CCTAGTCC | 2746 |
| 1878 | CUAGGGCA G UUUGGAUA | 419 | TATCCAAA GGCTAGCTACAACGA TGCCCTAG | 2747 |
| 1884 | CAGUUUGG A UAGCUCAA | 420 | TTGAGCTA GGCTAGCTACAACGA CCAAACTG | 2748 |
| 1887 | UUUGGAUA G CUCAACAA | 421 | TTGTTGAG GGCTAGCTACAACGA TATCCAAA | 2749 |
| 1892 | AUAGCUCA A CAAGAUAC | 422 | GTATCTTG GGCTAGCTACAACGA TGAGCTAT | 2750 |
| 1897 | UCAACAAG A UACAAUCU | 423 | AGATTGTA GGCTAGCTACAACGA CTTGTTGA | 2751 |
| 1899 | AACAAGAU A CAAUCUCA | 424 | TGAGATTG GGCTAGCTACAACGA ATCTTGTT | 2752 |
| 1902 | AAGAUACA A UCUCACUC | 425 | GAGTGAGA GGCTAGCTACAACGA TGTATCTT | 2753 |
| 1907 | ACAAUCUC A CUCUGUGG | 426 | CCACAGAG GGCTAGCTACAACGA GAGATTGT | 2754 |
| 1912 | CUCACUCU G UGGUGGUC | 427 | GACCACCA GGCTAGCTACAACGA AGAGTGAG | 2755 |
| 1915 | ACUCUGUG G UGGUCCUG | 428 | CAGGACCA GGCTAGCTACAACGA CACAGAGT | 2756 |
| 1918 | CUGUGGUG G UCCUGCUG | 429 | CAGCAGGA GGCTAGCTACAACGA CACCACAG | 2757 |
| 1923 | GUGGUCCU G CUGACAAA | 430 | TTTGTCAG GGCTAGCTACAACGA AGGACCAC | 2758 |
| 1927 | UCCUGCUG A CAAAUCAA | 431 | TTGATTTG GGCTAGCTACAACGA CAGCAGGA | 2759 |
| 1931 | GCUGACAA A UCAAGAGC | 432 | GCTCTTGA GGCTAGCTACAACGA TTGTCAGC | 2760 |
| 1938 | AAUCAAGA G CAUUGCUU | 433 | AAGCAATG GGCTAGCTACAACGA TCTTGATT | 2761 |
| 1940 | UCAAGAGC A UUGCUUUU | 434 | AAAAGCAA GGCTAGCTACAACGA GCTCTTGA | 2762 |
| 1943 | AGAGCAUU G CUUUUGUU | 435 | AACAAAAG GGCTAGCTACAACGA AATGCTCT | 2763 |
| 1949 | UUGCUUUU G UUUCUUAA | 436 | TTAAGAAA GGCTAGCTACAACGA AAAAGCAA | 2764 |
| 1962 | UUAAGAAA A CAAACUCU | 437 | AGAGTTTG GGCTAGCTACAACGA TTTCTTAA | 2765 |
| 1966 | GAAAACAA A CUCUUUUU | 438 | AAAAAGAG GGCTAGCTACAACGA TTGTTTTC | 2766 |
| 1980 | UUUUAAAA A UUACUUUU | 439 | AAAAGTAA GGCTAGCTACAACGA TTTTAAAA | 2767 |
| 1983 | UAAAAAUU A CUUUUAAA | 440 | TTTAAAAG GGCTAGCTACAACGA AATTTTTA | 2768 |
| 1991 | ACUUUUAA A UAUUAACU | 441 | AGTTAATA GGCTAGCTACAACGA TTAAAAGT | 2769 |
| 1993 | UUUUAAAU A UUAACUCA | 442 | TGAGTTAA GGCTAGCTACAACGA ATTTAAAA | 2770 |
| 1997 | AAAUAUUA A CUCAAAAG | 443 | CTTTTGAG GGCTAGCTACAACGA TAATATTT | 2771 |
| 2005 | ACUCAAAA G UUGAGAUU | 444 | AATCTCAA GGCTAGCTACAACGA TTTTGAGT | 2772 |
| 2011 | AAGUUGAG A UUUUGGGG | 445 | CCCCAAAA GGCTAGCTACAACGA CTCAACTT | 2773 |
| 2019 | AUUUUGGG G UGGUGGUG | 446 | CACCACCA GGCTAGCTACAACGA CCCAAAAT | 2774 |
| 2022 | UUGGGGUG G UGGUGUGC | 447 | GCACACCA GGCTAGCTACAACGA CACCCCAA | 2775 |
| 2025 | GGGUGGUG G UGUGCCAA | 448 | TTGGCACA GGCTAGCTACAACGA CACCACCC | 2776 |
| 2027 | GUGGUGGU G UGCCAAGA | 449 | TCTTGGCA GGCTAGCTACAACGA ACCACCAC | 2777 |
| 2029 | GGUGGUGU G CCAAGACA | 450 | TGTCTTGG GGCTAGCTACAACGA ACACCACC | 2778 |
| 2035 | GUGCCAAG A CAUUAAUU | 451 | AATTAATG GGCTAGCTACAACGA CTTGGCAC | 2779 |
| 2037 | GCCAAGAC A UUAAUUUU | 452 | AAAATTAA GGCTAGCTACAACGA GTCTTGGC | 2780 |
| 2041 | AGACAUUA A UUUUUUUU | 453 | AAAAAAAA GGCTAGCTACAACGA TAATGTCT | 2781 |
| 2054 | UUUUUUAA A CAAUGAAG | 454 | CTTCATTG GGCTAGCTACAACGA TTAAAAAA | 2782 |
| 2057 | UUUAAACA A UGAAGUGA | 455 | TCACTTCA GGCTAGCTACAACGA TGTTTAAA | 2783 |
| 2062 | ACAAUGAA G UGAAAAAG | 456 | CTTTTTCA GGCTAGCTACAACGA TTCATTGT | 2784 |
| 2070 | GUGAAAAA G UUUUACAA | 457 | TTGTAAAA GGCTAGCTACAACGA TTTTTCAC | 2785 |
| 2075 | AAAGUUUU A CAAUCUCU | 458 | AGAGATTG GGCTAGCTACAACGA AAAACTTT | 2786 |
| 2078 | GUUUUACA A UCUCUAGG | 459 | CCTAGAGA GGCTAGCTACAACGA TGTAAAAC | 2787 |
| 2086 | AUCUCUAG G UUUGGCUA | 460 | TAGCCAAA GGCTAGCTACAACGA CTAGAGAT | 2788 |
| 2091 | UAGGUUUG G CUAGUUCU | 461 | AGAACTAG GGCTAGCTACAACGA CAAACCTA | 2789 |
| 2095 | UUUGGCUA G UUCUCUUA | 462 | TAAGAGAA GGCTAGCTACAACGA TAGCCAAA | 2790 |
| 2104 | UUCUCUUA A CACUGGUU | 463 | AACCAGTG GGCTAGCTACAACGA TAAGAGAA | 2791 |
| 2106 | CUCUUAAC A CUGGUUAA | 464 | TTAACCAG GGCTAGCTACAACGA GTTAAGAG | 2792 |
| 2110 | UAACACUG G UUAAAUUA | 465 | TAATTTAA GGCTAGCTACAACGA CAGTGTTA | 2793 |
| 2115 | CUGGUUAA A UUAACAUU | 466 | AATGTTAA GGCTAGCTACAACGA TTAACCAG | 2794 |
| 2119 | UUAAAUUA A CAUUGCAU | 467 | ATGCAATG GGCTAGCTACAACGA TAATTTAA | 2795 |
| 2121 | AAAUUAAC A UUGCAUAA | 468 | TTATGCAA GGCTAGCTACAACGA GTTAATTT | 2796 |
| 2124 | UUAACAUU G CAUAAACA | 469 | TGTTTATG GGCTAGCTACAACGA AATGTTAA | 2797 |
| 2126 | AACAUUGC A UAAACACU | 470 | AGTGTTTA GGCTAGCTACAACGA GCAATGTT | 2798 |
| 2130 | UUGCAUAA A CACUUUUC | 471 | GAAAAGTG GGCTAGCTACAACGA TTATGCAA | 2799 |
| 2132 | GCAUAAAC A CUUUUCAA | 472 | TTGAAAAG GGCTAGCTACAACGA GTTTATGC | 2800 |
| 2141 | CUUUUCAA G UCUGAUCC | 473 | GGATCAGA GGCTAGCTACAACGA TTGAAAAG | 2801 |
| 2146 | CAAGUCUG A UCCAUAUU | 474 | AATATGGA GGCTAGCTACAACGA CAGACTTG | 2802 |
| 2150 | UCUGAUCC A UAUUUAAU | 475 | ATTAAATA GGCTAGCTACAACGA GGATCAGA | 2803 |
| 2152 | UGAUCCAU A UUUAAUAA | 476 | TTATTAAA GGCTAGCTACAACGA ATGGATCA | 2804 |
| 2157 | CAUAUUUA A UAAUGCUU | 477 | AAGCATTA GGCTAGCTACAACGA TAAATATG | 2805 |
| 2160 | AUUUAAUA A UGCUUUAA | 478 | TTAAAGCA GGCTAGCTACAACGA TATTAAAT | 2806 |
| 2162 | UUAAUAAU G CUUUAAAA | 479 | TTTTAAAG GGCTAGCTACAACGA ATTATTAA | 2807 |
| 2170 | GCUUUAAA A UAAAAAUA | 480 | TATTTTTA GGCTAGCTACAACGA TTTAAAGC | 2808 |
| 2176 | AAAUAAAA A UAAAAACA | 481 | TGTTTTTA GGCTAGCTACAACGA TTTTATTT | 2809 |
| 2182 | AAAUAAAA A CAAUCCUU | 482 | AAGGATTG GGCTAGCTACAACGA TTTTATTT | 2810 |
| 2185 | UAAAAACA A UCCUUUUG | 483 | CAAAAGGA GGCTAGCTACAACGA TGTTTTTA | 2811 |
| 2194 | UCCUUUUG A UAAAUUUA | 484 | TAAATTTA GGCTAGCTACAACGA CAAAAGGA | 2812 |
| 2198 | UUUGAUAA A UUUAAAAU | 485 | ATTTTAAA GGCTAGCTACAACGA TTATCAAA | 2813 |
| 2205 | AAUUUAAA A UGUUACUU | 486 | AAGTAACA GGCTAGCTACAACGA TTTAAATT | 2814 |
| 2207 | UUUAAAAU G UUACUUAU | 487 | ATAAGTAA GGCTAGCTACAACGA ATTTTAAA | 2815 |
| 2210 | AAAAUGUU A CUUAUUUU | 488 | AAAATAAG GGCTAGCTACAACGA AACATTTT | 2816 |
| 2214 | UGUUACUU A UUUUAAAA | 489 | TTTTAAAA GGCTAGCTACAACGA AAGTAACA | 2817 |
| 2222 | AUUUUAAA A UAAAUGAA | 490 | TTCATTTA GGCTAGCTACAACGA TTTAAAAT | 2818 |
| 2226 | UAAAAUAA A UGAAGUGA | 491 | TCACTTCA GGCTAGCTACAACGA TTATTTTA | 2819 |
| 2231 | UAAAUGAA G UGAGAUGG | 492 | CCATCTCA GGCTAGCTACAACGA TTCATTTA | 2820 |
| 2236 | GAAGUGAG A UGGCAUGG | 493 | CCATGCCA GGCTAGCTACAACGA CTCACTTC | 2821 |
| 2239 | GUGAGAUG G CAUGGUGA | 494 | TCACCATG GGCTAGCTACAACGA CATCTCAC | 2822 |
| 2241 | GAGAUGGC A UGGUGAGG | 495 | CCTCACCA GGCTAGCTACAACGA GCCATCTC | 2823 |
| 2244 | AUGGCAUG G UGAGGUGA | 496 | TCACCTCA GGCTAGCTACAACGA CATGCCAT | 2824 |
| 2249 | AUGGUGAG G UGAAAGUA | 497 | TACTTTCA GGCTAGCTACAACGA CTCACCAT | 2825 |
| 2255 | AGGUGAAA G UAUCACUG | 498 | CAGTGATA GGCTAGCTACAACGA TTTCACCT | 2826 |
| 2257 | GUGAAAGU A UCACUGGA | 499 | TCCAGTGA GGCTAGCTACAACGA ACTTTCAC | 2827 |
| 2260 | AAAGUAUC A CUGGACUA | 500 | TAGTCCAG GGCTAGCTACAACGA GATACTTT | 2828 |
| 2265 | AUCACUGG A CUAGGUUG | 501 | CAACCTAG GGCTAGCTACAACGA CCAGTGAT | 2829 |
| 2270 | UGGACUAG G UUGUUGGU | 502 | ACCAACAA GGCTAGCTACAACGA CTAGTCCA | 2830 |
| 2273 | ACUAGGUU G UUGGUGAC | 503 | GTCACCAA GGCTAGCTACAACGA AACCTAGT | 2831 |
| 2277 | GGUUGUUG G UGACUUAG | 504 | CTAAGTCA GGCTAGCTACAACGA CAACAACC | 2832 |
| 2280 | UGUUGGUG A CUUAGGUU | 505 | AACCTAAG GGCTAGCTACAACGA CACCAACA | 2833 |
| 2286 | UGACUUAG G UUCUAGAU | 506 | ATCTAGAA GGCTAGCTACAACGA CTAAGTCA | 2834 |
| 2293 | GGUUCUAG A UAGGUGUC | 507 | GACACCTA GGCTAGCTACAACGA CTAGAACC | 2835 |
| 2297 | CUAGAUAG G UGUCUUUU | 508 | AAAAGACA GGCTAGCTACAACGA CTATCTAG | 2836 |
| 2299 | AGAUAGGU G UCUUUUAG | 509 | CTAAAAGA GGCTAGCTACAACGA ACCTATCT | 2837 |
| 2309 | CUUUUAGG A CUCUGAUU | 510 | AATCAGAG GGCTAGCTACAACGA CCTAAAAG | 2838 |
| 2315 | GGACUCUG A UUUUGAGG | 511 | CCTCAAAA GGCTAGCTACAACGA CAGAGTCC | 2839 |
| 2324 | UUUUGAGG A CAUCACUU | 512 | AAGTGATG GGCTAGCTACAACGA CCTCAAAA | 2840 |
| 2326 | UUGAGGAC A UCACUUAC | 513 | GTAAGTGA GGCTAGCTACAACGA GTCCTCAA | 2841 |
| 2329 | AGGACAUC A CUUACUAU | 514 | ATAGTAAG GGCTAGCTACAACGA GATGTCCT | 2842 |
| 2333 | CAUCACUU A CUAUCCAU | 515 | ATGGATAG GGCTAGCTACAACGA AAGTGATG | 2843 |
| 2336 | CACUUACU A UCCAUUUC | 516 | GAAATGGA GGCTAGCTACAACGA AGTAAGTG | 2844 |
| 2340 | UACUAUCC A UUUCUUCA | 517 | TGAAGAAA GGCTAGCTACAACGA GGATAGTA | 2845 |
| 2348 | AUUUCUUC A UGUUAAAA | 518 | TTTTAACA GGCTAGCTACAACGA GAAGAAAT | 2846 |
| 2350 | UUCUUCAU G UUAAAAGA | 519 | TCTTTTAA GGCTAGCTACAACGA ATGAAGAA | 2847 |
| 2360 | UAAAAGAA G UCAUCUCA | 520 | TGAGATGA GGCTAGCTACAACGA TTCTTTTA | 2848 |
| 2363 | AAGAAGUC A UCUCAAAC | 521 | GTTTGAGA GGCTAGCTACAACGA GACTTCTT | 2849 |
| 2370 | CAUCUCAA A CUCUUAGU | 522 | ACTAAGAG GGCTAGCTACAACGA TTGAGATG | 2850 |
| 2377 | AACUCUUA G UUUUUUUU | 523 | AAAAAAAA GGCTAGCTACAACGA TAAGAGTT | 2851 |
| 2390 | UUUUUUUU A CACUAUGU | 524 | ACATAGTG GGCTAGCTACAACGA AAAAAAAA | 2852 |
| 2392 | UUUUUUAC A CUAUGUGA | 525 | TCACATAG GGCTAGCTACAACGA GTAAAAAA | 2853 |
| 2395 | UUUACACU A UGUGAUUU | 526 | AAATCACA GGCTAGCTACAACGA AGTGTAAA | 2854 |
| 2397 | UACACUAU G UGAUUUAU | 527 | ATAAATCA GGCTAGCTACAACGA ATAGTGTA | 2855 |
| 2400 | ACUAUGUG A UUUAUAUU | 528 | AATATAAA GGCTAGCTACAACGA CACATAGT | 2856 |
| 2404 | UGUGAUUU A UAUUCCAU | 529 | ATGGAATA GGCTAGCTACAACGA AAATCACA | 2857 |
| 2406 | UGAUUUAU A UUCCAUUU | 530 | AAATGGAA GGCTAGCTACAACGA ATAAATCA | 2858 |
| 2411 | UAUAUUCC A UUUACAUA | 531 | TATGTAAA GGCTAGCTACAACGA GGAATATA | 2859 |
| 2415 | UUCCAUUU A CAUAAGGA | 532 | TCCTTATG GGCTAGCTACAACGA AAATGGAA | 2860 |
| 2417 | CCAUUUAC A UAAGGAUA | 533 | TATCCTTA GGCTAGCTACAACGA GTAAATGG | 2861 |
| 2423 | ACAUAAGG A UACACUUA | 534 | TAAGTGTA GGCTAGCTACAACGA CCTTATGT | 2862 |
| 2425 | AUAAGGAU A CACUUAUU | 535 | AATAAGTG GGCTAGCTACAACGA ATCCTTAT | 2863 |
| 2427 | AAGGAUAC A CUUAUUUG | 536 | CAAATAAG GGCTAGCTACAACGA GTATCCTT | 2864 |
| 2431 | AUACACUU A UUUGUCAA | 537 | TTGACAAA GGCTAGCTACAACGA AAGTGTAT | 2865 |
| 2435 | ACUUAUUU G UCAAGCUC | 538 | GAGCTTGA GGCTAGCTACAACGA AAATAAGT | 2866 |
| 2440 | UUUGUCAA G CUCAGCAC | 539 | GTGCTGAG GGCTAGCTACAACGA TTGACAAA | 2867 |
| 2445 | CAAGCUCA G CACAAUCU | 540 | AGATTGTG GGCTAGCTACAACGA TGAGCTTG | 2868 |
| 2447 | AGCUCAGC A CAAUCUGU | 541 | ACAGATTG GGCTAGCTACAACGA GCTGAGCT | 2869 |
| 2450 | UCAGCACA A UCUGUAAA | 542 | TTTACAGA GGCTAGCTACAACGA TGTGCTGA | 2870 |
| 2454 | CACAAUCU G UAAAUUUU | 543 | AAAATTTA GGCTAGCTACAACGA AGATTGTG | 2871 |
| 2458 | AUCUGUAA A UUUUUAAC | 544 | GTTAAAAA GGCTAGCTACAACGA TTACAGAT | 2872 |
| 2465 | AAUUUUUA A CCUAUGUU | 545 | AACATAGG GGCTAGCTACAACGA TAAAAATT | 2873 |
| 2469 | UUUAACCU A UGUUACAC | 546 | GTGTAACA GGCTAGCTACAACGA AGGTTAAA | 2874 |
| 2471 | UAACCUAU G UUACACCA | 547 | TGGTGTAA GGCTAGCTACAACGA ATAGGTTA | 2875 |
| 2474 | CCUAUGUU A CACCAUCU | 548 | AGATGGTG GGCTAGCTACAACGA AACATAGG | 2876 |
| 2476 | UAUGUUAC A CCAUCUUC | 549 | GAAGATGG GGCTAGCTACAACGA GTAACATA | 2877 |
| 2479 | GUUACACC A UCUUCAGU | 550 | ACTGAAGA GGCTAGCTACAACGA GGTGTAAC | 2878 |
| 2486 | CAUCUUCA G UGCCAGUC | 551 | GACTGGCA GGCTAGCTACAACGA TGAAGATG | 2879 |
| 2488 | UCUUCAGU G CCAGUCUU | 552 | AAGACTGG GGCTAGCTACAACGA ACTGAAGA | 2880 |
| 2492 | CAGUGCCA G UCUUGGGC | 553 | GCCCAAGA GGCTAGCTACAACGA TGGCACTG | 2881 |
| 2499 | AGUCUUGG G CAAAAUUG | 554 | CAATTTTG GGCTAGCTACAACGA CCAAGACT | 2882 |
| 2504 | UGGGCAAA A UUGUGCAA | 555 | TTGCACAA GGCTAGCTACAACGA TTTGCCCA | 2883 |
| 2507 | GCAAAAUU G UGCAAGAG | 556 | CTCTTGCA GGCTAGCTACAACGA AATTTTGC | 2884 |
| 2509 | AAAAUUGU G CAAGAGGU | 557 | ACCTCTTG GGCTAGCTACAACGA ACAATTTT | 2885 |
| 2516 | UGCAAGAG G UGAAGUUU | 558 | AAACTTCA GGCTAGCTACAACGA CTCTTGCA | 2886 |
| 2521 | GAGGUGAA G UUUAUAUU | 559 | AATATAAA GGCTAGCTACAACGA TTCACCTC | 2887 |
| 2525 | UGAAGUUU A UAUUUGAA | 560 | TTCAAATA GGCTAGCTACAACGA AAACTTCA | 2888 |
| 2527 | AAGUUUAU A UUUGAAUA | 561 | TATTCAAA GGCTAGCTACAACGA ATAAACTT | 2889 |
| 2533 | AUAUUUGA A UAUCCAUU | 562 | AATGGATA GGCTAGCTACAACGA TCAAATAT | 2890 |
| 2535 | AUUUGAAU A UCCAUUCU | 563 | AGAATGGA GGCTAGCTACAACGA ATTCAAAT | 2891 |
| 2539 | GAAUAUCC A UUCUCGUU | 564 | AACGAGAA GGCTAGCTACAACGA GGATATTC | 2892 |
| 2545 | CCAUUCUC G UUUUAGGA | 565 | TCCTAAAA GGCTAGCTACAACGA GAGAATGG | 2893 |
| 2553 | GUUUUAGG A CUCUUCUU | 566 | AAGAAGAG GGCTAGCTACAACGA CCTAAAAC | 2894 |
| 2564 | CUUCUUCC A UAUUAGUG | 567 | CACTAATA GGCTAGCTACAACGA GGAAGAAG | 2895 |
| 2566 | UCUUCCAU A UUAGUGUC | 568 | GACACTAA GGCTAGCTACAACGA ATGGAAGA | 2896 |
| 2570 | CCAUAUUA G UGUCAUCU | 569 | AGATGACA GGCTAGCTACAACGA TAATATGG | 2897 |
| 2572 | AUAUUAGU G UCAUCUUG | 570 | CAAGATGA GGCTAGCTACAACGA ACTAATAT | 2898 |
| 2575 | UUAGUGUC A UCUUGCCU | 571 | AGGCAAGA GGCTAGCTACAACGA GACACTAA | 2899 |
| 2580 | GUCAUCUU G CCUCCCUA | 572 | TAGGGAGG GGCTAGCTACAACGA AAGATGAC | 2900 |
| 2588 | GCCUCCCU A CCUUCCAC | 573 | GTGGAAGG GGCTAGCTACAACGA AGGGAGGC | 2901 |
| 2595 | UACCUUCC A CAUGCCCC | 574 | GGGGCATG GGCTAGCTACAACGA GGAAGGTA | 2902 |
| 2597 | CCUUCCAC A UGCCCCAU | 575 | ATGGGGCA GGCTAGCTACAACGA GTGGAAGG | 2903 |
| 2599 | UUCCACAU G CCCCAUGA | 576 | TCATGGGG GGCTAGCTACAACGA ATGTGGAA | 2904 |
| 2604 | CAUGCCCC A UGACUUGA | 577 | TCAAGTCA GGCTAGCTACAACGA GGGGCATG | 2905 |
| 2607 | GCCCCAUG A CUUGAUGC | 578 | GCATCAAG GGCTAGCTACAACGA CATGGGGC | 2906 |
| 2612 | AUGACUUG A UGCAGUUU | 579 | AAACTGCA GGCTAGCTACAACGA CAAGTCAT | 2907 |
| 2614 | GACUUGAU G CAGUUUUA | 580 | TAAAACTG GGCTAGCTACAACGA ATCAAGTC | 2908 |
| 2617 | UUGAUGCA G UUUUAAUA | 581 | TATTAAAA GGCTAGCTACAACGA TGCATCAA | 2909 |
| 2623 | CAGUUUUA A UACUUGUA | 582 | TACAAGTA GGCTAGCTACAACGA TAAAACTG | 2910 |
| 2625 | GUUUUAAU A CUUGUAAU | 583 | ATTACAAG GGCTAGCTACAACGA ATTAAAAC | 2911 |
| 2629 | UAAUACUU G UAAUUCCC | 584 | GGGAATTA GGCTAGCTACAACGA AAGTATTA | 2912 |
| 2632 | UACUUGUA A UUCCCCUA | 585 | TAGGGGAA GGCTAGCTACAACGA TACAAGTA | 2913 |
| 2641 | UUCCCCUA A CCAUAAGA | 586 | TCTTATGG GGCTAGCTACAACGA TAGGGGAA | 2914 |
| 2644 | CCCUAACC A UAAGAUUU | 587 | AAATCTTA GGCTAGCTACAACGA GGTTAGGG | 2915 |
| 2649 | ACCAUAAG A UUUACUGC | 588 | GCAGTAAA GGCTAGCTACAACGA CTTATGGT | 2916 |
| 2653 | UAAGAUUU A CUGCUGCU | 589 | AGCAGCAG GGCTAGCTACAACGA AAATCTTA | 2917 |
| 2656 | GAUUUACU G CUGCUGUG | 590 | CACAGCAG GGCTAGCTACAACGA AGTAAATC | 2918 |
| 2659 | UUACUGCU G CUGUGGAU | 591 | ATCCACAG GGCTAGCTACAACGA AGCAGTAA | 2919 |
| 2662 | CUGCUGCU G UGGAUAUC | 592 | GATATCCA GGCTAGCTACAACGA AGCAGCAG | 2920 |
| 2666 | UGCUGUGG A UAUCUCCA | 593 | TGGAGATA GGCTAGCTACAACGA CCACAGCA | 2921 |
| 2668 | CUGUGGAU A UCUCCAUG | 594 | CATGGAGA GGCTAGCTACAACGA ATCCACAG | 2922 |
| 2674 | AUAUCUCC A UGAAGUUU | 595 | AAACTTCA GGCTAGCTACAACGA GGAGATAT | 2923 |
| 2679 | UCCAUGAA G UUUUCCCA | 596 | TGGGAAAA GGCTAGCTACAACGA TTCATGGA | 2924 |
| 2687 | GUUUUCCC A CUGAGUCA | 597 | TGACTCAG GGCTAGCTACAACGA GGGAAAAC | 2925 |
| 2692 | CCCACUGA G UCACAUCA | 598 | TGATGTGA GGCTAGCTACAACGA TCAGTGGG | 2926 |
| 2695 | ACUGAGUC A CAUCAGAA | 599 | TTCTGATG GGCTAGCTACAACGA GACTCAGT | 2927 |
| 2697 | UGAGUCAC A UCAGAAAU | 600 | ATTTCTGA GGCTAGCTACAACGA GTGACTCA | 2928 |
| 2704 | CAUCAGAA A UGCCCUAC | 601 | GTAGGGCA GGCTAGCTACAACGA TTCTGATG | 2929 |
| 2706 | UCAGAAAU G CCCUACAU | 602 | ATGTAGGG GGCTAGCTACAACGA ATTTCTGA | 2930 |
| 2711 | AAUGCCCU A CAUCUUAU | 603 | ATAAGATG GGCTAGCTACAACGA AGGGCATT | 2931 |
| 2713 | UGCCCUAC A UCUUAUUU | 604 | AAATAAGA GGCTAGCTACAACGA GTAGGGCA | 2932 |
| 2718 | UACAUCUU A UUUUCCUC | 605 | GAGGAAAA GGCTAGCTACAACGA AAGATGTA | 2933 |
| 2730 | UCCUCAGG G CUCAAGAG | 606 | CTCTTGAG GGCTAGCTACAACGA CCTGAGGA | 2934 |
| 2740 | UCAAGAGA A UCUGACAG | 607 | CTGTCAGA GGCTAGCTACAACGA TCTCTTGA | 2935 |
| 2745 | AGAAUCUG A CAGAUACC | 608 | GGTATCTG GGCTAGCTACAACGA CAGATTCT | 2936 |
| 2749 | UCUGACAG A UACCAUAA | 609 | TTATGGTA GGCTAGCTACAACGA CTGTCAGA | 2937 |
| 2751 | UGACAGAU A CCAUAAAG | 610 | CTTTATGG GGCTAGCTACAACGA ATCTGTCA | 2938 |
| 2754 | CAGAUACC A UAAAGGGA | 611 | TCCCTTTA GGCTAGCTACAACGA GGTATCTG | 2939 |
| 2762 | AUAAAGGG A UUUGACCU | 612 | AGGTCAAA GGCTAGCTACAACGA CCCTTTAT | 2940 |
| 2767 | GGGAUUUG A CCUAAUCA | 613 | TGATTAGG GGCTAGCTACAACGA CAAATCCC | 2941 |
| 2772 | UUGACCUA A UCACUAAU | 614 | ATTAGTGA GGCTAGCTACAACGA TAGGTCAA | 2942 |
| 2775 | ACCUAAUC A CUAAUUUU | 615 | AAAATTAG GGCTAGCTACAACGA GATTAGGT | 2943 |
| 2779 | AAUCACUA A UUUUCAGG | 616 | CCTGAAAA GGCTAGCTACAACGA TAGTGATT | 2944 |
| 2787 | AUUUUCAG G UGGUGGCU | 617 | AGCCACCA GGCTAGCTACAACGA CTGAAAAT | 2945 |
| 2790 | UUCAGGUG G UGGCUGAU | 618 | ATCAGCCA GGCTAGCTACAACGA CACCTGAA | 2946 |
| 2793 | AGGUGGUG G CUGAUGCU | 619 | AGCATCAG GGCTAGCTACAACGA CACCACCT | 2947 |
| 2797 | GGUGGCUG A UGCUUUGA | 620 | TCAAAGCA GGCTAGCTACAACGA CAGCCACC | 2948 |
| 2799 | UGGCUGAU G CUUUGAAC | 621 | GTTCAAAG GGCTAGCTACAACGA ATCAGCCA | 2949 |
| 2806 | UGCUUUGA A CAUCUCUU | 622 | AAGAGATG GGCTAGCTACAACGA TCAAAGCA | 2950 |
| 2808 | CUUUGAAC A UCUCUUUG | 623 | CAAAGAGA GGCTAGCTACAACGA GTTCAAAG | 2951 |
| 2816 | AUCUCUUU G CUGCCCAA | 624 | TTGGGCAG GGCTAGCTACAACGA AAAGAGAT | 2952 |
| 2819 | UCUUUGCU G CCCAAUCC | 625 | GGATTGGG GGCTAGCTACAACGA AGCAAAGA | 2953 |
| 2824 | GCUGCCCA A UCCAUUAG | 626 | CTAATGGA GGCTAGCTACAACGA TGGGCAGC | 2954 |
| 2828 | CCCAAUCC A UUAGCGAC | 627 | GTCGCTAA GGCTAGCTACAACGA GGATTGGG | 2955 |
| 2832 | AUCCAUUA G CGACAGUA | 628 | TACTGTCG GGCTAGCTACAACGA TAATGGAT | 2956 |
| 2835 | CAUUAGCG A CAGUAGGA | 629 | TCCTACTG GGCTAGCTACAACGA CGCTAATG | 2957 |
| 2838 | UAGCGACA G UAGGAUUU | 630 | AAATCCTA GGCTAGCTACAACGA TGTCGCTA | 2958 |
| 2843 | ACAGUAGG A UUUUUCAA | 631 | TTGAAAAA GGCTAGCTACAACGA CCTACTGT | 2959 |
| 2851 | AUUUUUCA A CCCUGGUA | 632 | TACCAGGG GGCTAGCTACAACGA TGAAAAAT | 2960 |
| 2857 | CAACCCUG G UAUGAAUA | 633 | TATTCATA GGCTAGCTACAACGA CAGGGTTG | 2961 |
| 2859 | ACCCUGGU A UGAAUAGA | 634 | TCTATTCA GGCTAGCTACAACGA ACCAGGGT | 2962 |
| 2863 | UGGUAUGA A UAGACAGA | 635 | TCTGTCTA GGCTAGCTACAACGA TCATACCA | 2963 |
| 2867 | AUGAAUAG A CAGAACCC | 636 | GGGTTCTG GGCTAGCTACAACGA CTATTCAT | 2964 |
| 2872 | UAGACAGA A CCCUAUCC | 637 | GGATAGGG GGCTAGCTACAACGA TCTGTCTA | 2965 |
| 2877 | AGAACCCU A UCCAGUGG | 638 | CCACTGGA GGCTAGCTACAACGA AGGGTTCT | 2966 |
| 2882 | CCUAUCCA G UGGAAGGA | 639 | TCCTTCCA GGCTAGCTACAACGA TGGATAGG | 2967 |
| 2893 | GAAGGAGA A UUUAAUAA | 640 | TTATTAAA GGCTAGCTACAACGA TCTCCTTC | 2968 |
| 2898 | AGAAUUUA A UAAAGAUA | 641 | TATCTTTA GGCTAGCTACAACGA TAAATTCT | 2969 |
| 2904 | UAAUAAAG A UAGUGCAG | 642 | CTGCACTA GGCTAGCTACAACGA CTTTATTA | 2970 |
| 2907 | UAAAGAUA G UGCAGAAA | 643 | TTTCTGCA GGCTAGCTACAACGA TATCTTTA | 2971 |
| 2909 | AAGAUAGU G CAGAAAGA | 644 | TCTTTCTG GGCTAGCTACAACGA ACTATCTT | 2972 |
| 2918 | CAGAAAGA A UUCCUUAG | 645 | CTAAGGAA GGCTAGCTACAACGA TCTTTCTG | 2973 |
| 2927 | UUCCUUAG G UAAUCUAU | 646 | ATAGATTA GGCTAGCTACAACGA CTAAGGAA | 2974 |
| 2930 | CUUAGGUA A UCUAUAAC | 647 | GTTATAGA GGCTAGCTACAACGA TACCTAAG | 2975 |
| 2934 | GGUAAUCU A UAACUAGG | 648 | CCTAGTTA GGCTAGCTACAACGA AGATTACC | 2976 |
| 2937 | AAUCUAUA A CUAGGACU | 649 | AGTCCTAG GGCTAGCTACAACGA TATAGATT | 2977 |
| 2943 | UAACUAGG A CUACUCCU | 650 | AGGAGTAG GGCTAGCTACAACGA CCTAGTTA | 2978 |
| 2946 | CUAGGACU A CUCCUGGU | 651 | ACCAGGAG GGCTAGCTACAACGA AGTCCTAG | 2979 |
| 2953 | UACUCCUG G UAACAGUA | 652 | TACTGTTA GGCTAGCTACAACGA CAGGAGTA | 2980 |
| 2956 | UCCUGGUA A CAGUAAUA | 653 | TATTACTG GGCTAGCTACAACGA TACCAGGA | 2981 |
| 2959 | UGGUAACA G UAAUACAU | 654 | ATGTATTA GGCTAGCTACAACGA TGTTACCA | 2982 |
| 2962 | UAACAGUA A UACAUUCC | 655 | GGAATGTA GGCTAGCTACAACGA TACTGTTA | 2983 |
| 2964 | ACAGUAAU A CAUUCCAU | 656 | ATGGAATG GGCTAGCTACAACGA ATTACTGT | 2984 |
| 2966 | AGUAAUAC A UUCCAUUG | 657 | CAATGGAA GGCTAGCTACAACGA GTATTACT | 2985 |
| 2971 | UACAUUCC A UUGUUUUA | 658 | TAAAACAA GGCTAGCTACAACGA GGAATGTA | 2986 |
| 2974 | AUUCCAUU G UUUUAGUA | 659 | TACTAAAA GGCTAGCTACAACGA AATGGAAT | 2987 |
| 2980 | UUGUUUUA G UAACCAGA | 660 | TCTGGTTA GGCTAGCTACAACGA TAAAACAA | 2988 |
| 2983 | UUUUAGUA A CCAGAAAU | 661 | ATTTCTGG GGCTAGCTACAACGA TACTAAAA | 2989 |
| 2990 | AACCAGAA A UCUUCAUG | 662 | CATGAAGA GGCTAGCTACAACGA TTCTGGTT | 2990 |
| 2996 | AAAUCUUC A UGCAAUGA | 663 | TCATTGCA GGCTAGCTACAACGA GAAGATTT | 2991 |
| 2998 | AUCUUCAU G CAAUGAAA | 664 | TTTCATTG GGCTAGCTACAACGA ATGAAGAT | 2992 |
| 3001 | UUCAUGCA A UGAAAAAU | 665 | ATTTTTCA GGCTAGCTACAACGA TGCATGAA | 2993 |
| 3008 | AAUGAAAA A UACUUUAA | 666 | TTAAAGTA GGCTAGCTACAACGA TTTTCATT | 2994 |
| 3010 | UGAAAAAU A CUUUAAUU | 667 | AATTAAAG GGCTAGCTACAACGA ATTTTTCA | 2995 |
| 3016 | AUACUUUA A UUCAUGAA | 668 | TTCATGAA GGCTAGCTACAACGA TAAAGTAT | 2996 |
| 3020 | UUUAAUUC A UGAAGCUU | 669 | AAGCTTCA GGCTAGCTACAACGA GAATTAAA | 2997 |
| 3025 | UUCAUGAA G CUUACUUU | 670 | AAAGTAAG GGCTAGCTACAACGA TTCATGAA | 2998 |
| 3029 | UGAAGCUU A CUUUUUUU | 671 | AAAAAAAG GGCTAGCTACAACGA AAGCTTCA | 2999 |
| 3044 | UUUUUUUG G UGUCAGAG | 672 | CTCTGACA GGCTAGCTACAACGA CAAAAAAA | 3000 |
| 3046 | UUUUUGGU G UCAGAGUC | 673 | GACTCTGA GGCTAGCTACAACGA ACCAAAAA | 3001 |
| 3052 | GUGUCAGA G UCUCGCUC | 674 | GAGCGAGA GGCTAGCTACAACGA TCTGACAC | 3002 |
| 3057 | AGAGUCUC G CUCUUGUC | 675 | GACAAGAG GGCTAGCTACAACGA GAGACTCT | 3003 |
| 3063 | UCGCUCUU G UCACCCAG | 676 | CTGGGTGA GGCTAGCTACAACGA AAGAGCGA | 3004 |
| 3066 | CUCUUGUC A CCCAGGCU | 677 | AGCCTGGG GGCTAGCTACAACGA GACAAGAG | 3005 |
| 3072 | UCACCCAG G CUGGAAUG | 678 | CATTCCAG GGCTAGCTACAACGA CTGGGTGA | 3006 |
| 3078 | AGGCUGGA A UGCAGUGG | 679 | CCACTGCA GGCTAGCTACAACGA TCCAGCCT | 3007 |
| 3080 | GCUGGAAU G CAGUGGCG | 680 | CGCCACTG GGCTAGCTACAACGA ATTCCAGC | 3008 |
| 3083 | GGAAUGCA G UGGCGCCA | 681 | TGGCGCCA GGCTAGCTACAACGA TGCATTCC | 3009 |
| 3086 | AUGCAGUG G CGCCAUCU | 682 | AGATGGCG GGCTAGCTACAACGA CACTGCAT | 3010 |
| 3088 | GCAGUGGC G CCAUCUCA | 683 | TGAGATGG GGCTAGCTACAACGA GCCACTGC | 3011 |
| 3091 | GUGGCGCC A UCUCAGCU | 684 | AGCTGAGA GGCTAGCTACAACGA GGCGCCAC | 3012 |
| 3097 | CCAUCUCA G CUCACUGC | 685 | GCAGTGAG GGCTAGCTACAACGA TGAGATGG | 3013 |
| 3101 | CUCAGCUC A CUGCAACC | 686 | GGTTGCAG GGCTAGCTACAACGA GAGCTGAG | 3014 |
| 3104 | AGCUCACU G CAACCUUC | 687 | GAAGGTTG GGCTAGCTACAACGA AGTGAGCT | 3015 |
| 3107 | UCACUCCA A CCUUCCAU | 688 | ATGGAAGG GGCTAGCTACAACGA TGCAGTGA | 3016 |
| 3114 | AACCUUCC A UCUUCCCA | 689 | TGGGAAGA GGCTAGCTACAACGA GGAAGGTT | 3017 |
| 3124 | CUUCCCAG G UUCAAGCG | 690 | CGCTTGAA GGCTAGCTACAACGA CTGGGAAG | 3018 |
| 3130 | AGGUUCAA G CGAUUCUC | 691 | GAGAATCG GGCTAGCTACAACGA TTGAACCT | 3019 |
| 3133 | UUCAAGCG A UUCUCGUG | 692 | CACGAGAA GGCTAGCTACAACGA CGCTTGAA | 3020 |
| 3139 | CGAUUCUC G UGCCUCGG | 693 | CCGAGGCA GGCTAGCTACAACGA GAGAATCG | 3021 |
| 3141 | AUUCUCGU G CCUCGGCC | 694 | GGCCGAGG GGCTAGCTACAACGA ACGAGAAT | 3022 |
| 3147 | GUGCCUCG G CCUCCUGA | 695 | TCAGGAGG GGCTAGCTACAACGA CGAGGCAC | 3023 |
| 3156 | CCUCCUGA G UAGCUGGG | 696 | CCCAGCTA GGCTAGCTACAACGA TCAGGAGG | 3024 |
| 3159 | CCUGAGUA G CUGGGAUU | 697 | AATCCCAG GGCTAGCTACAACGA TACTCAGG | 3025 |
| 3165 | UAGCUGGG A UUACAGGC | 698 | GCCTGTAA GGCTAGCTACAACGA CCCAGCTA | 3026 |
| 3168 | CUGGGAUU A CAGGCGUG | 699 | CACGCCTG GGCTAGCTACAACGA AATCCCAG | 3027 |
| 3172 | GAUUACAG G CGUGUGCA | 700 | TGCACACG GGCTAGCTACAACGA CTGTAATC | 3028 |
| 3174 | UUACAGGC G UGUGCACU | 701 | AGTGCACA GGCTAGCTACAACGA GCCTGTAA | 3029 |
| 3176 | ACAGGCGU G UGCACUAC | 702 | GTAGTGCA GGCTAGCTACAACGA ACGCCTGT | 3030 |
| 3178 | AGGCGUGU G CACUACAC | 703 | GTGTAGTG GGCTAGCTACAACGA ACACGCCT | 3031 |
| 3180 | GCGUGUGC A CUACACUC | 704 | GAGTGTAG GGCTAGCTACAACGA GCACACGC | 3032 |
| 3183 | UGUGCACU A CACUCAAC | 705 | GTTGAGTG GGCTAGCTACAACGA AGTGCACA | 3033 |
| 3185 | UGCACUAC A CUCAACUA | 706 | TAGTTGAG GGCTAGCTACAACGA GTAGTGCA | 3034 |
| 3190 | UACACUCA A CUAAUUUU | 707 | AAAATTAG GGCTAGCTACAACGA TGAGTGTA | 3035 |
| 3194 | CUCAACUA A UUUUUGUA | 708 | TACAAAAA GGCTAGCTACAACGA TAGTTGAG | 3036 |
| 3200 | UAAUUUUU G UAUUUUUA | 709 | TAAAAATA GGCTAGCTACAACGA AAAAATTA | 3037 |
| 3202 | AUUUUUGU A UUUUUAGG | 710 | CCTAAAAA GGCTAGCTACAACGA ACAAAAAT | 3038 |
| 3215 | UAGGAGAG A CGGGGUUU | 711 | AAACCCCG GGCTAGCTACAACGA CTCTCCTA | 3039 |
| 3220 | GAGACGGG G UUUCACCU | 712 | AGGTGAAA GGCTAGCTACAACGA CCCGTCTC | 3040 |
| 3225 | GGGGUUUC A CCUGUUGG | 713 | CCAACAGG GGCTAGCTACAACGA GAAACCCC | 3041 |
| 3229 | UUUCACCU G UUGGCCAG | 714 | CTGGCCAA GGCTAGCTACAACGA AGGTGAAA | 3042 |
| 3233 | ACCUGUUG G CCAGGCUG | 715 | CAGCCTGG GGCTAGCTACAACGA CAACAGGT | 3043 |
| 3238 | UUGGCCAG G CUGGUCUC | 716 | GAGACCAG GGCTAGCTACAACGA CTGGCCAA | 3044 |
| 3242 | CCAGGCUG G UCUCGAAC | 717 | GTTCGAGA GGCTAGCTACAACGA CAGCCTGG | 3045 |
| 3249 | GGUCUCGA A CUCCUGAC | 718 | GTCAGGAG GGCTAGCTACAACGA TCGAGACC | 3046 |
| 3256 | AACUCCUG A CCUCAAGU | 719 | ACTTGAGG GGCTAGCTACAACGA CAGGAGTT | 3047 |
| 3263 | GACCUCAA G UGAUUCAC | 720 | GTGAATCA GGCTAGCTACAACGA TTGAGGTC | 3048 |
| 3266 | CUCAAGUG A UUCACCCA | 721 | TGGGTGAA GGCTAGCTACAACGA CACTTGAG | 3049 |
| 3270 | AGUGAUUC A CCCACCUU | 722 | AAGGTGGG GGCTAGCTACAACGA GAATCACT | 3050 |
| 3274 | AUUCACCC A CCUUGGCC | 723 | GGCCAAGG GGCTAGCTACAACGA GGGTGAAT | 3051 |
| 3280 | CCACCUUG G CCUCAUAA | 724 | TTATGAGG GGCTAGCTACAACGA CAAGGTGG | 3052 |
| 3285 | UUGGCCUC A UAAACCUG | 725 | CAGGTTTA GGCTAGCTACAACGA GAGGCCAA | 3053 |
| 3289 | CCUCAUAA A CCUGUUUU | 726 | AAAACAGG GGCTAGCTACAACGA TTATGAGG | 3054 |
| 3293 | AUAAACCU G UUUUGCAG | 727 | CTGCAAAA GGCTAGCTACAACGA AGGTTTAT | 3055 |
| 3298 | CCUGUUUU G CAGAACUC | 728 | GAGTTCTG GGCTAGCTACAACGA AAAACAGG | 3056 |
| 3303 | UUUGCAGA A CUCAUUUA | 729 | TAAATGAG GGCTAGCTACAACGA TCTGCAAA | 3057 |
| 3307 | CAGAACUC A UUUAUUCA | 730 | TGAATAAA GGCTAGCTACAACGA GAGTTCTG | 3058 |
| 3311 | ACUCAUUU A UUCAGCAA | 731 | TTGCTGAA GGCTAGCTACAACGA AAATGAGT | 3059 |
| 3316 | UUUAUUCA G CAAAUAUU | 732 | AATATTTG GGCTAGCTACAACGA TGAATAAA | 3060 |
| 3320 | UUCAGCAA A UAUUUAUU | 733 | AATAAATA GGCTAGCTACAACGA TTGCTGAA | 3061 |
| 3322 | CAGCAAAU A UUUAUUGA | 734 | TCAATAAA GGCTAGCTACAACGA ATTTGCTG | 3062 |
| 3326 | AAAUAUUU A UUGAGUGC | 735 | GCACTCAA GGCTAGCTACAACGA AAATATTT | 3063 |
| 3331 | UUUAUUGA G UGCCUACC | 736 | GGTAGGCA GGCTAGCTACAACGA TCAATAAA | 3064 |
| 3333 | UAUUGAGU G CCUACCAG | 737 | CTGGTAGG GGCTAGCTACAACGA ACTCAATA | 3065 |
| 3337 | GAGUGCCU A CCAGAUGC | 738 | GCATCTGG GGCTAGCTACAACGA AGGCACTC | 3066 |
| 3342 | CCUACCAG A UGCCAGUC | 739 | GACTGGCA GGCTAGCTACAACGA CTGGTAGG | 3067 |
| 3344 | UACCAGAU G CCAGUCAC | 740 | GTGACTGG GGCTAGCTACAACGA ATCTGGTA | 3068 |
| 3348 | AGAUGCCA G UCACCGCA | 741 | TGCGGTGA GGCTAGCTACAACGA TGGCATCT | 3069 |
| 3351 | UGCCAGUC A CCGCACAA | 742 | TTGTGCGG GGCTAGCTACAACGA GACTGGCA | 3070 |
| 3354 | CAGUCACC G CACAAGGC | 743 | GCCTTGTG GGCTAGCTACAACGA GGTGACTG | 3071 |
| 3356 | GUCACCGC A CAAGGCAC | 744 | GTGCCTTG GGCTAGCTACAACGA GCGGTGAC | 3072 |
| 3361 | CGCACAAG G CACUGGGU | 745 | ACCCAGTG GGCTAGCTACAACGA CTTGTGCG | 3073 |
| 3363 | CACAAGGC A CUGGGUAU | 746 | ATACCCAG GGCTAGCTACAACGA GCCTTGTG | 3074 |
| 3368 | GGCACUGG G UAUAUGGU | 747 | ACCATATA GGCTAGCTACAACGA CCAGTGCC | 3075 |
| 3370 | CACUGGGU A UAUGGUAU | 748 | ATACCATA GGCTAGCTACAACGA ACCCAGTG | 3076 |
| 3372 | CUGGGUAU A UGGUAUCC | 749 | GGATACCA GGCTAGCTACAACGA ATACCCAG | 3077 |
| 3375 | GGUAUAUG G UAUCCCCA | 750 | TGGGGATA GGCTAGCTACAACGA CATATACC | 3078 |
| 3377 | UAUAUGGU A UCCCCAAA | 751 | TTTGGGGA GGCTAGCTACAACGA ACCATATA | 3079 |
| 3385 | AUCCCCAA A CAAGAGAC | 752 | GTCTCTTG GGCTAGCTACAACGA TTGGGGAT | 3080 |
| 3392 | AACAAGAG A CAUAAUCC | 753 | GGATTATG GGCTAGCTACAACGA CTCTTGTT | 3081 |
| 3394 | CAAGAGAC A UAAUCCCG | 754 | CGGGATTA GGCTAGCTACAACGA GTCTCTTG | 3082 |
| 3397 | GAGACAUA A UCCCGGUC | 755 | GACCGGGA GGCTAGCTACAACGA TATGTCTC | 3083 |
| 3403 | UAAUCCCG G UCCUUAGG | 756 | CCTAAGGA GGCTAGCTACAACGA CGGGATTA | 3084 |
| 3411 | GUCCUUAG G UACUGCUA | 757 | TAGCAGTA GGCTAGCTACAACGA CTAAGGAC | 3085 |
| 3413 | CCUUAGGU A CUGCUAGU | 758 | ACTAGCAG GGCTAGCTACAACGA ACCTAAGG | 3086 |
| 3416 | UAGGUACU G CUAGUGUG | 759 | CACACTAG GGCTAGCTACAACGA AGTACCTA | 3087 |
| 3420 | UACUGCUA G UGUGGUCU | 760 | AGACCACA GGCTAGCTACAACGA TAGCAGTA | 3088 |
| 3422 | CUGCUAGU G UGGUCUGU | 761 | ACAGACCA GGCTAGCTACAACGA ACTAGCAG | 3089 |
| 3425 | CUAGUGUG G UCUGUAAU | 762 | ATTACAGA GGCTAGCTACAACGA CACACTAG | 3090 |
| 3429 | UGUGGUCU G UAAUAUCU | 763 | AGATATTA GGCTAGCTACAACGA AGACCACA | 3091 |
| 3432 | GGUCUGUA A UAUCUUAC | 764 | GTAAGATA GGCTAGCTACAACGA TACAGACC | 3092 |
| 3434 | UCUGUAAU A UCUUACUA | 765 | TAGTAAGA GGCTAGCTACAACGA ATTACAGA | 3093 |
| 3439 | AAUAUCUU A CUAAGGCC | 766 | GGCCTTAG GGCTAGCTACAACGA AAGATATT | 3094 |
| 3445 | UUACUAAG G CCUUUGGU | 767 | ACCAAAGG GGCTAGCTACAACGA CTTAGTAA | 3095 |
| 3452 | GGCCUUUG G UAUACGAC | 768 | GTCGTATA GGCTAGCTACAACGA CAAAGGCC | 3096 |
| 3454 | CCUUUGGU A UACGACCC | 769 | GGGTCGTA GGCTAGCTACAACGA ACCAAAGG | 3097 |
| 3456 | UUUGGUAU A CGACCCAG | 770 | CTGGGTCG GGCTAGCTACAACGA ATACCAAA | 3098 |
| 3459 | GGUAUACG A CCCAGAGA | 771 | TCTCTGGG GGCTAGCTACAACGA CGTATACC | 3099 |
| 3467 | ACCCAGAG A UAACACGA | 772 | TCGTGTTA GGCTAGCTACAACGA CTCTGGGT | 3100 |
| 3470 | CAGAGAUA A CACGAUGC | 773 | GCATCGTG GGCTAGCTACAACGA TATCTCTG | 3101 |
| 3472 | GAGAUAAC A CGAUGCGU | 774 | ACGCATCG GGCTAGCTACAACGA GTTATCTC | 3102 |
| 3475 | AUAACACG A UGCGUAUU | 775 | AATACGCA GGCTAGCTACAACGA CGTGTTAT | 3103 |
| 3477 | AACACGAU G CGUAUUUU | 776 | AAAATACG GGCTAGCTACAACGA ATCGTGTT | 3104 |
| 3479 | CACGAUGC G UAUUUUAG | 777 | CTAAAATA GGCTAGCTACAACGA GCATCGTG | 3105 |
| 3481 | CGAUGCGU A UUUUAGUU | 778 | AACTAAAA GGCTAGCTACAACGA ACGCATCG | 3106 |
| 3487 | GUAUUUUA G UUUUGCAA | 779 | TTGCAAAA GGCTAGCTACAACGA TAAAATAC | 3107 |
| 3492 | UUAGUUUU G CAAAGAAG | 780 | CTTCTTTG GGCTAGCTACAACGA AAAACTAA | 3108 |
| 3503 | AAGAAGGG G UUUGGUCU | 781 | AGACCAAA GGCTAGCTACAACGA CCCTTCTT | 3109 |
| 3508 | GGGGUUUG G UCUCUGUG | 782 | CACAGAGA GGCTAGCTACAACGA CAAACCCC | 3110 |
| 3514 | UGGUCUCU G UGCCAGCU | 783 | AGCTGGCA GGCTAGCTACAACGA AGAGACCA | 3111 |
| 3516 | GUCUCUGU G CCAGCUCU | 784 | AGAGCTGG GGCTAGCTACAACGA ACAGAGAC | 3112 |
| 3520 | CUGUGCCA G CUCUAUAA | 785 | TTATAGAG GGCTAGCTACAACGA TGGCACAG | 3113 |
| 3525 | CCAGCUCU A UAAUUGUU | 786 | AACAATTA GGCTAGCTACAACGA AGAGCTGG | 3114 |
| 3528 | GCUCUAUA A UUGUUUUG | 787 | CAAAACAA GGCTAGCTACAACGA TATAGAGC | 3115 |
| 3531 | CUAUAAUU G UUUUGCUA | 788 | TAGCAAAA GGCTAGCTACAACGA AATTATAG | 3116 |
| 3536 | AUUGUUUU G CUACGAUU | 789 | AATCGTAG GGCTAGCTACAACGA AAAACAAT | 3117 |
| 3539 | GUUUUGCU A CGAUUCCA | 790 | TGGAATCG GGCTAGCTACAACGA AGCAAAAC | 3118 |
| 3542 | UUGCUACG A UUCCACUG | 791 | CAGTGGAA GGCTAGCTACAACGA CGTAGCAA | 3119 |
| 3547 | ACGAUUCC A CUGAAACU | 792 | AGTTTCAG GGCTAGCTACAACGA GGAATCGT | 3120 |
| 3553 | CCACUGAA A CUCUUCGA | 793 | TCGAAGAG GGCTAGCTACAACGA TTCAGTGG | 3121 |
| 3561 | ACUCUUCG A UCAAGCUA | 794 | TAGCTTGA GGCTAGCTACAACGA CGAAGAGT | 3122 |
| 3566 | UCGAUCAA G CUACUUUA | 795 | TAAAGTAG GGCTAGCTACAACGA TTGATCGA | 3123 |
| 3569 | AUCAAGCU A CUUUAUGU | 796 | ACATAAAG GGCTAGCTACAACGA AGCTTGAT | 3124 |
| 3574 | GCUACUUU A UGUAAAUC | 797 | GATTTACA GGCTAGCTACAACGA AAAGTAGC | 3125 |
| 3576 | UACUUUAU G UAAAUCAC | 798 | GTGATTTA GGCTAGCTACAACGA ATAAAGTA | 3126 |
| 3580 | UUAUGUAA A UCACUUCA | 799 | TGAAGTGA GGCTAGCTACAACGA TTACATAA | 3127 |
| 3583 | UGUAAAUC A CUUCAUUG | 800 | CAATGAAG GGCTAGCTACAACGA GATTTACA | 3128 |
| 3588 | AUCACUUC A UUGUUUUA | 801 | TAAAACAA GGCTAGCTACAACGA GAAGTGAT | 3129 |
| 3591 | ACUUCAUU G UUUUAAAG | 802 | CTTTAAAA GGCTAGCTACAACGA AATGAAGT | 3130 |
| 3602 | UUAAAGGA A UAAACUUG | 803 | CAAGTTTA GGCTAGCTACAACGA TCCTTTAA | 3131 |
| 3606 | AGGAAUAA A CUUGAUUA | 804 | TAATCAAG GGCTAGCTACAACGA TTATTCCT | 3132 |
| 3611 | UAAACUUG A UUAUAUUG | 805 | CAATATAA GGCTAGCTACAACGA CAAGTTTA | 3133 |
| 3614 | ACUUGAUU A UAUUGUUU | 806 | AAACAATA GGCTAGCTACAACGA AATCAAGT | 3134 |
| 3616 | UUGAUUAU A UUGUUUUU | 807 | AAAAACAA GGCTAGCTACAACGA ATAATCAA | 3135 |
| 3619 | AUUAUAUU G UUUUUUUA | 808 | TAAAAAAA GGCTAGCTACAACGA AATATAAT | 3136 |
| 3627 | GUUUUUUU A UUUGGCAU | 809 | ATGCCAAA GGCTAGCTACAACGA AAAAAAAC | 3137 |
| 3632 | UUUAUUUG G CAUAACUG | 810 | CAGTTATG GGCTAGCTACAACGA CAAATAAA | 3138 |
| 3634 | UAUUUGGC A UAACUGUG | 811 | CACAGTTA GGCTAGCTACAACGA GCCAAATA | 3139 |
| 3637 | UUGGCAUA A CUGUGAUU | 812 | AATCACAG GGCTAGCTACAACGA TATGCCAA | 3140 |
| 3640 | GCAUAACU G UGAUUCUU | 813 | AAGAATCA GGCTAGCTACAACGA AGTTATGC | 3141 |
| 3643 | UAACUGUG A UUCUUUUA | 814 | TAAAAGAA GGCTAGCTACAACGA CACAGTTA | 3142 |
| 3654 | CUUUUAGG A CAAUUACU | 815 | AGTAATTG GGCTAGCTACAACGA CCTAAAAG | 3143 |
| 3657 | UUAGGACA A UUACUGUA | 816 | TACAGTAA GGCTAGCTACAACGA TGTCCTAA | 3144 |
| 3660 | GGACAAUU A CUGUACAC | 817 | GTGTACAG GGCTAGCTACAACGA AATTGTCC | 3145 |
| 3663 | CAAUUACU G UACACAUU | 818 | AATGTGTA GGCTAGCTACAACGA AGTAATTG | 3146 |
| 3665 | AUUACUGU A CACAUUAA | 819 | TTAATGTG GGCTAGCTACAACGA ACAGTAAT | 3147 |
| 3667 | UACUGUAC A CAUUAAGG | 820 | CCTTAATG GGCTAGCTACAACGA GTACAGTA | 3148 |
| 3669 | CUGUACAC A UUAAGGUG | 821 | CACCTTAA GGCTAGCTACAACGA GTGTACAG | 3149 |
| 3675 | ACAUUAAG G UGUAUGUC | 822 | GACATACA GGCTAGCTACAACGA CTTAATGT | 3150 |
| 3677 | AUUAAGGU G UAUGUCAG | 823 | CTGACATA GGCTAGCTACAACGA ACCTTAAT | 3151 |
| 3679 | UAAGGUGU A UGUCAGAU | 824 | ATCTGACA GGCTAGCTACAACGA ACACCTTA | 3152 |
| 3681 | AGGUGUAU G UCAGAUAU | 825 | ATATCTGA GGCTAGCTACAACGA ATACACCT | 3153 |
| 3686 | UAUGUCAG A UAUUCAUA | 826 | TATGAATA GGCTAGCTACAACGA CTGACATA | 3154 |
| 3688 | UGUCAGAU A UUCAUAUU | 827 | AATATGAA GGCTAGCTACAACGA ATCTGACA | 3155 |
| 3692 | AGAUAUUC A UAUUGACC | 828 | GGTCAATA GGCTAGCTACAACGA GAATATCT | 3156 |
| 3694 | AUAUUCAU A UUGACCCA | 829 | TGGGTCAA GGCTAGCTACAACGA ATGAATAT | 3157 |
| 3698 | UCAUAUUG A CCCAAAUG | 830 | CATTTGGG GGCTAGCTACAACGA CAATATGA | 3158 |
| 3704 | UGACCCAA A UGUGUAAU | 831 | ATTACACA GGCTAGCTACAACGA TTGGGTCA | 3159 |
| 3706 | ACCCAAAU G UGUAAUAU | 832 | ATATTACA GGCTAGCTACAACGA ATTTGGGT | 3160 |
| 3708 | CCAAAUGU G UAAUAUUC | 833 | GAATATTA GGCTAGCTACAACGA ACATTTGG | 3161 |
| 3711 | AAUGUGUA A UAUUCCAG | 834 | CTGGAATA GGCTAGCTACAACGA TACACATT | 3162 |
| 3713 | UGUGUAAU A UUCCAGUU | 835 | AACTGGAA GGCTAGCTACAACGA ATTACACA | 3163 |
| 3719 | AUAUUCCA G UUUUCUCU | 836 | AGAGAAAA GGCTAGCTACAACGA TGGAATAT | 3164 |
| 3728 | UUUUCUCU G CAUAAGUA | 837 | TACTTATG GGCTAGCTACAACGA AGAGAAAA | 3165 |
| 3730 | UUCUCUGC A UAAGUAAU | 838 | ATTACTTA GGCTAGCTACAACGA GCAGAGAA | 3166 |
| 3734 | CUGCAUAA G UAAUUAAA | 839 | TTTAATTA GGCTAGCTACAACGA TTATGCAG | 3167 |
| 3737 | CAUAAGUA A UUAAAAUA | 840 | TATTTTAA GGCTAGCTACAACGA TACTTATG | 3168 |
| 3743 | UAAUUAAA A UAUACUUA | 841 | TAAGTATA GGCTAGCTACAACGA TTTAATTA | 3169 |
| 3745 | AUUAAAAU A UACUUAAA | 842 | TTTAAGTA GGCTAGCTACAACGA ATTTTAAT | 3170 |
| 3747 | UAAAAUAU A CUUAAAAA | 843 | TTTTTAAG GGCTAGCTACAACGA ATATTTTA | 3171 |
| 3755 | ACUUAAAA A UUAAUAGU | 844 | ACTATTAA GGCTAGCTACAACGA TTTTAAGT | 3172 |
| 3759 | AAAAAUUA A UAGUUUUA | 845 | TAAAACTA GGCTAGCTACAACGA TAATTTTT | 3173 |
| 3762 | AAUUAAUA G UUUUAUCU | 846 | AGATAAAA GGCTAGCTACAACGA TATTAATT | 3174 |
| 3767 | AUAGUUUU A UCUGGGUA | 847 | TACCCAGA GGCTAGCTACAACGA AAAACTAT | 3175 |
| 3773 | UUAUCUGG G UACAAAUA | 848 | TATTTGTA GGCTAGCTACAACGA CCAGATAA | 3176 |
| 3775 | AUCUGGGU A CAAAUAAA | 849 | TTTATTTG GGCTAGCTACAACGA ACCCAGAT | 3177 |
| 3779 | GGGUACAA A UAAACAGU | 850 | ACTGTTTA GGCTAGCTACAACGA TTGTACCC | 3178 |
| 3783 | ACAAAUAA A CAGUGCCU | 851 | AGGCACTG GGCTAGCTACAACGA TTATTTGT | 3179 |
| 3786 | AAUAAACA G UGCCUGAA | 852 | TTCAGGCA GGCTAGCTACAACGA TGTTTATT | 3180 |
| 3788 | UAAACAGU G CCUGAACU | 853 | AGTTCAGG GGCTAGCTACAACGA ACTGTTTA | 3181 |
| 3794 | GUGCCUGA A CUAGUUCA | 854 | TGAACTAG GGCTAGCTACAACGA TCAGGCAC | 3182 |
| 3798 | CUGAACUA G UUCACAGA | 855 | TCTGTGAA GGCTAGCTACAACGA TAGTTCAG | 3183 |
| 3802 | ACUAGUUC A CAGACAAG | 856 | CTTGTCTG GGCTAGCTACAACGA GAACTAGT | 3184 |
| 3806 | GUUCACAG A CAAGGGAA | 857 | TTCCCTTG GGCTAGCTACAACGA CTGTGAAC | 3185 |
| 3815 | CAAGGGAA A CUUCUAUG | 858 | CATAGAAG GGCTAGCTACAACGA TTCCCTTG | 3186 |
| 3821 | AAACUUCU A UGUAAAAA | 859 | TTTTTACA GGCTAGCTACAACGA AGAAGTTT | 3187 |
| 3823 | ACUUCUAU G UAAAAAUC | 860 | GATTTTTA GGCTAGCTACAACGA ATAGAAGT | 3188 |
| 3829 | AUGUAAAA A UCACUAUG | 861 | CATAGTGA GGCTAGCTACAACGA TTTTACAT | 3189 |
| 3832 | UAAAAAUC A CUAUGAUU | 862 | AATCATAG GGCTAGCTACAACGA GATTTTTA | 3190 |
| 3835 | AAAUCACU A UGAUUUCU | 863 | AGAAATCA GGCTAGCTACAACGA AGTGATTT | 3191 |
| 3838 | UCACUAUG A UUUCUGAA | 864 | TTCAGAAA GGCTAGCTACAACGA CATAGTGA | 3192 |
| 3846 | AUUUCUGA A UUGCUAUG | 865 | CATAGCAA GGCTAGCTACAACGA TCAGAAAT | 3193 |
| 3849 | UCUGAAUU G CUAUGUGA | 866 | TCACATAG GGCTAGCTACAACGA AATTCAGA | 3194 |
| 3852 | GAAUUGCU A UGUGAAAC | 867 | GTTTCACA GGCTAGCTACAACGA AGCAATTC | 3195 |
| 3854 | AUUGCUAU G UGAAACUA | 868 | TAGTTTCA GGCTAGCTACAACGA ATAGCAAT | 3196 |
| 3859 | UAUGUGAA A CUACAGAU | 869 | ATCTGTAG GGCTAGCTACAACGA TTCACATA | 3197 |
| 3862 | GUGAAACU A CAGAUCUU | 870 | AAGATCTG GGCTAGCTACAACGA AGTTTCAC | 3198 |
| 3866 | AACUACAG A UCUUUGGA | 871 | TCCAAAGA GGCTAGCTACAACGA CTGTAGTT | 3199 |
| 3875 | UCUUUGGA A CACUGUUU | 872 | AAACAGTG GGCTAGCTACAACGA TCCAAAGA | 3200 |
| 3877 | UUUGGAAC A CUGUUUAG | 873 | CTAAACAG GGCTAGCTACAACGA GTTCCAAA | 3201 |
| 3880 | GGAACACU G UUUAGGUA | 874 | TACCTAAA GGCTAGCTACAACGA AGTGTTCC | 3202 |
| 3886 | CUGUUUAG G UAGGGUGU | 875 | ACACCCTA GGCTAGCTACAACGA CTAAACAG | 3203 |
| 3891 | UAGGUAGG G UGUUAAGA | 876 | TCTTAACA GGCTAGCTACAACGA CCTACCTA | 3204 |
| 3893 | GGUAGGGU G UUAAGACU | 877 | AGTCTTAA GGCTAGCTACAACGA ACCCTACC | 3205 |
| 3899 | GUGUUAAG A CUUGACAC | 878 | GTGTCAAG GGCTAGCTACAACGA CTTAACAC | 3206 |
| 3904 | AAGACUUG A CACAGUAC | 879 | GTACTGTG GGCTAGCTACAACGA CAAGTCTT | 3207 |
| 3906 | GACUUGAC A CAGUACCU | 880 | AGGTACTG GGCTAGCTACAACGA GTCAAGTC | 3208 |
| 3909 | UUGACACA G UACCUCGU | 881 | ACGAGGTA GGCTAGCTACAACGA TGTGTCAA | 3209 |
| 3911 | GACACAGU A CCUCGUUU | 882 | AAACGAGG GGCTAGCTACAACGA ACTGTGTC | 3210 |
| 3916 | AGUACCUC G UUUCUACA | 883 | TGTAGAAA GGCTAGCTACAACGA GAGGTACT | 3211 |
| 3922 | UCGUUUCU A CACAGAGA | 884 | TCTCTGTG GGCTAGCTACAACGA AGAAACGA | 3212 |
| 3924 | GUUUCUAC A CAGAGAAA | 885 | TTTCTCTG GGCTAGCTACAACGA GTAGAAAC | 3213 |
| 3936 | AGAAAGAA A UGGCCAUA | 886 | TATGGCCA GGCTAGCTACAACGA TTCTTTCT | 3214 |
| 3939 | AAGAAAUG G CCAUACUU | 887 | AAGTATGG GGCTAGCTACAACGA CATTTCTT | 3215 |
| 3942 | AAAUGGCC A UACUUCAG | 888 | CTGAAGTA GGCTAGCTACAACGA GGCCATTT | 3216 |
| 3944 | AUGGCCAU A CUUCAGGA | 889 | TCCTGAAG GGCTAGCTACAACGA ATGGCCAT | 3217 |
| 3953 | CUUCAGGA A CUGCAGUG | 890 | CACTGCAG GGCTAGCTACAACGA TCCTGAAG | 3218 |
| 3956 | CAGGAACU G CAGUGCUU | 891 | AAGCACTG GGCTAGCTACAACGA AGTTCCTG | 3219 |
| 3959 | GAACUGCA G UGCUUAUG | 892 | CATAAGCA GGCTAGCTACAACGA TGCAGTTC | 3220 |
| 3961 | ACUGCAGU G CUUAUGAG | 893 | CTCATAAG GGCTAGCTACAACGA ACTGCAGT | 3221 |
| 3965 | CAGUGCUU A UGAGGGGA | 894 | TCCCCTCA GGCTAGCTACAACGA AAGCACTG | 3222 |
| 3973 | AUGAGGGG A UAUUUAGG | 895 | CCTAAATA GGCTAGCTACAACGA CCCCTCAT | 3223 |
| 3975 | GAGGGGAU A UUUAGGCC | 896 | GGCCTAAA GGCTAGCTACAACGA ATCCCCTC | 3224 |
| 3981 | AUAUUUAG G CCUCUUGA | 897 | TCAAGAGG GGCTAGCTACAACGA CTAAATAT | 3225 |
| 3990 | CCUCUUGA A UUUUUGAU | 898 | ATCAAAAA GGCTAGCTACAACGA TCAAGAGG | 3226 |
| 3997 | AAUUUUUG A UGUAGAUG | 899 | CATCTACA GGCTAGCTACAACGA CAAAAATT | 3227 |
| 3999 | UUUUUGAU G UAGAUGGG | 900 | CCCATCTA GGCTAGCTACAACGA ATCAAAAA | 3228 |
| 4003 | UGAUGUAG A UGGGCAUU | 901 | AATGCCCA GGCTAGCTACAACGA CTACATCA | 3229 |
| 4007 | GUAGAUGG G CAUUUUUU | 902 | AAAAAATG GGCTAGCTACAACGA CCATCTAC | 3230 |
| 4009 | AGAUGGGC A UUUUUUUA | 903 | TAAAAAAA GGCTAGCTACAACGA GCCCATCT | 3231 |
| 4020 | UUUUUAAG G UAGUGGUU | 904 | AACCACTA GGCTAGCTACAACGA CTTAAAAA | 3232 |
| 4023 | UUAAGGUA G UGGUUAAU | 905 | ATTAACCA GGCTAGCTACAACGA TACCTTAA | 3233 |
| 4026 | AGGUAGUG G UUAAUUAC | 906 | GTAATTAA GGCTAGCTACAACGA CACTACCT | 3234 |
| 4030 | AGUGGUUA A UUACCUUU | 907 | AAAGGTAA GGCTAGCTACAACGA TAACCACT | 3235 |
| 4033 | GGUUAAUU A CCUUUAUG | 908 | CATAAAGG GGCTAGCTACAACGA AATTAACC | 3236 |
| 4039 | UUACCUUU A UGUGAACU | 909 | AGTTCACA GGCTAGCTACAACGA AAAGGTAA | 3237 |
| 4041 | ACCUUUAU G UGAACUUU | 910 | AAAGTTCA GGCTAGCTACAACGA ATAAAGGT | 3238 |
| 4045 | UUAUGUGA A CUUUGAAU | 911 | ATTCAAAG GGCTAGCTACAACGA TCACATAA | 3239 |
| 4052 | AACUUUGA A UGGUUUAA | 912 | TTAAACCA GGCTAGCTACAACGA TCAAAGTT | 3240 |
| 4055 | UUUGAAUG G UUUAACAA | 913 | TTGTTAAA GGCTAGCTACAACGA CATTCAAA | 3241 |
| 4060 | AUGGUUUA A CAAAAGAU | 914 | ATCTTTTG GGCTAGCTACAACGA TAAACCAT | 3242 |
| 4067 | AACAAAAG A UUUGUUUU | 915 | AAAACAAA GGCTAGCTACAACGA CTTTTGTT | 3243 |
| 4071 | AAAGAUUU G UUUUUGUA | 916 | TACAAAAA GGCTAGCTACAACGA AAATCTTT | 3244 |
| 4077 | UUGUUUUU G UAGAGAUU | 917 | AATCTCTA GGCTAGCTACAACGA AAAAACAA | 3245 |
| 4083 | UUGUAGAG A UUUUAAAG | 918 | CTTTAAAA GGCTAGCTACAACGA CTCTACAA | 3246 |
| 4099 | GGGGGAGA A UUCUAGAA | 919 | TTCTAGAA GGCTAGCTACAACGA TCTCCCCC | 3247 |
| 4108 | UUCUAGAA A UAAAUGUU | 920 | AACATTTA GGCTAGCTACAACGA TTCTAGAA | 3248 |
| 4112 | AGAAAUAA A UGUUACCU | 921 | AGGTAACA GGCTAGCTACAACGA TTATTTCT | 3249 |
| 4114 | AAAUAAAU G UUACCUAA | 922 | TTAGGTAA GGCTAGCTACAACGA ATTTATTT | 3250 |
| 4117 | UAAAUGUU A CCUAAUUA | 923 | TAATTAGG GGCTAGCTACAACGA AACATTTA | 3251 |
| 4122 | GUUACCUA A UUAUUACA | 924 | TGTAATAA GGCTAGCTACAACGA TAGGTAAC | 3252 |
| 4125 | ACCUAAUU A UUACAGCC | 925 | GGCTGTAA GGCTAGCTACAACGA AATTAGGT | 3253 |
| 4128 | UAAUUAUU A CAGCCUUA | 926 | TAAGGCTG GGCTAGCTACAACGA AATAATTA | 3254 |
| 4131 | UUAUUACA G CCUUAAAG | 927 | CTTTAAGG GGCTAGCTACAACGA TGTAATAA | 3255 |
| 4140 | CCUUAAAG A CAAAAAUC | 928 | GATTTTTG GGCTAGCTACAACGA CTTTAAGG | 3256 |
| 4146 | AGACAAAA A UCCUUGUU | 929 | AACAAGGA GGCTAGCTACAACGA TTTTGTCT | 3257 |
| 4152 | AAAUCCUU G UUGAAGUU | 930 | AACTTCAA GGCTAGCTACAACGA AAGGATTT | 3258 |
| 4158 | UUGUUGAA G UUUUUUUA | 931 | TAAAAAAA GGCTAGCTACAACGA TTCAACAA | 3259 |
| 4174 | AAAAAAAG A CUAAAUUA | 932 | TAATTTAG GGCTAGCTACAACGA CTTTTTTT | 3260 |
| 4179 | AAGACUAA A UUACAUAG | 933 | CTATGTAA GGCTAGCTACAACGA TTAGTCTT | 3261 |
| 4182 | ACUAAAUU A CAUAGACU | 934 | AGTCTATG GGCTAGCTACAACGA AATTTAGT | 3262 |
| 4184 | UAAAUUAC A UAGACUUA | 935 | TAAGTCTA GGCTAGCTACAACGA GTAATTTA | 3263 |
| 4188 | UUACAUAG A CUUAGGCA | 936 | TGCCTAAG GGCTAGCTACAACGA CTATGTAA | 3264 |
| 4194 | AGACUUAG G CAUUAACA | 937 | TGTTAATG GGCTAGCTACAACGA CTAAGTCT | 3265 |
| 4196 | ACUUAGGC A UUAACAUG | 938 | CATGTTAA GGCTAGCTACAACGA GCCTAAGT | 3266 |
| 4200 | AGGCAUUA A CAUGUUUG | 939 | CAAACATG GGCTAGCTACAACGA TAATGCCT | 3267 |
| 4202 | GCAUUAAC A UGUUUGUG | 940 | CACAAACA GGCTAGCTACAACGA GTTAATGC | 3268 |
| 4204 | AUUAACAU G UUUGUGGA | 941 | TCCACAAA GGCTAGCTACAACGA ATGTTAAT | 3269 |
| 4208 | ACAUGUUU G UGGAAGAA | 942 | TTCTTCCA GGCTAGCTACAACGA AAACATGT | 3270 |
| 4216 | GUGGAAGA A UAUAGCAG | 943 | CTGCTATA GGCTAGCTACAACGA TCTTCCAC | 3271 |
| 4218 | GGAAGAAU A UAGCAGAC | 944 | GTCTGCTA GGCTAGCTACAACGA ATTCTTCC | 3272 |
| 4221 | AGAAUAUA G CAGACGUA | 945 | TACGTCTG GGCTAGCTACAACGA TATATTCT | 3273 |
| 4225 | UAUAGCAG A CGUAUAUU | 946 | AATATACG GGCTAGCTACAACGA CTGCTATA | 3274 |
| 4227 | UAGCAGAC G UAUAUUGU | 947 | ACAATATA GGCTAGCTACAACGA GTCTGCTA | 3275 |
| 4229 | GCAGACGU A UAUUGUAU | 948 | ATACAATA GGCTAGCTACAACGA ACGTCTGC | 3276 |
| 4231 | AGACGUAU A UUGUAUCA | 949 | TGATACAA GGCTAGCTACAACGA ATACGTCT | 3277 |
| 4234 | CGUAUAUU G UAUCAUUU | 950 | AAATGATA GGCTAGCTACAACGA AATATACG | 3278 |
| 4236 | UAUAUUGU A UCAUUUGA | 951 | TCAAATGA GGCTAGCTACAACGA ACAATATA | 3279 |
| 4239 | AUUGUAUC A UUUGAGUG | 952 | CACTCAAA GGCTAGCTACAACGA GATACAAT | 3280 |
| 4245 | UCAUUUGA G UGAAUGUU | 953 | AACATTCA GGCTAGCTACAACGA TCAAATGA | 3281 |
| 4249 | UUGAGUGA A UGUUCCCA | 954 | TGGGAACA GGCTAGCTACAACGA TCACTCAA | 3282 |
| 4251 | GAGUGAAU G UUCCCAAG | 955 | CTTGGGAA GGCTAGCTACAACGA ATTCACTC | 3283 |
| 4259 | GUUCCCAA G UAGGCAUU | 956 | AATGCCTA GGCTAGCTACAACGA TTGGGAAC | 3284 |
| 4263 | CCAAGUAG G CAUUCUAG | 957 | CTAGAATG GGCTAGCTACAACGA CTACTTGG | 3285 |
| 4265 | AAGUAGGC A UUCUAGGC | 958 | GCCTAGAA GGCTAGCTACAACGA GCCTACTT | 3286 |
| 4272 | CAUUCUAG G CUCUAUUU | 959 | AAATAGAG GGCTAGCTACAACGA CTAGAATG | 3287 |
| 4277 | UAGGCUCU A UUUAACUG | 960 | CAGTTAAA GGCTAGCTACAACGA AGAGCCTA | 3288 |
| 4282 | UCUAUUUA A CUGAGUCA | 961 | TGACTCAG GGCTAGCTACAACGA TAAATAGA | 3289 |
| 4287 | UUAACUGA G UCACACUG | 962 | CAGTGTGA GGCTAGCTACAACGA TCAGTTAA | 3290 |
| 4290 | ACUGAGUC A CACUGCAU | 963 | ATGCAGTG GGCTAGCTACAACGA GACTCAGT | 3291 |
| 4292 | UGAGUCAC A CUGCAUAG | 964 | CTATGCAG GGCTAGCTACAACGA GTGACTCA | 3292 |
| 4295 | GUCACACU G CAUAGGAA | 965 | TTCCTATG GGCTAGCTACAACGA AGTGTGAC | 3293 |
| 4297 | CACACUGC A UAGGAAUU | 966 | AATTCCTA GGCTAGCTACAACGA GCAGTGTG | 3294 |
| 4303 | GCAUAGGA A UUUAGAAC | 967 | GTTCTAAA GGCTAGCTACAACGA TCCTATGC | 3295 |
| 4310 | AAUUUAGA A CCUAACUU | 968 | AAGTTAGG GGCTAGCTACAACGA TCTAAATT | 3296 |
| 4315 | AGAACCUA A CUUUUAUA | 969 | TATAAAAG GGCTAGCTACAACGA TAGGTTCT | 3297 |
| 4321 | UAACUUUU A UAGGUUAU | 970 | ATAACCTA GGCTAGCTACAACGA AAAAGTTA | 3298 |
| 4325 | UUUUAUAG G UUAUCAAA | 971 | TTTGATAA GGCTAGCTACAACGA CTATAAAA | 3299 |
| 4328 | UAUAGGUU A UCAAAACU | 972 | AGTTTTGA GGCTAGCTACAACGA AACCTATA | 3300 |
| 4334 | UUAUCAAA A CUGUUGUC | 973 | GACAACAG GGCTAGCTACAACGA TTTGATAA | 3301 |
| 4337 | UCAAAACU G UUGUCACC | 974 | GGTGACAA GGCTAGCTACAACGA AGTTTTGA | 3302 |
| 4340 | AAACUGUU G UCACCAUU | 975 | AATGGTGA GGCTAGCTACAACGA AACAGTTT | 3303 |
| 4343 | CUGUUGUC A CCAUUGCA | 976 | TGCAATGG GGCTAGCTACAACGA GACAACAG | 3304 |
| 4346 | UUGUCACC A UUGCACAA | 977 | TTGTGCAA GGCTAGCTACAACGA GGTGACAA | 3305 |
| 4349 | UCACCAUU G CACAAUUU | 978 | AAATTGTG GGCTAGCTACAACGA AATGGTGA | 3306 |
| 4351 | ACCAUUGC A CAAUUUUG | 979 | CAAAATTG GGCTAGCTACAACGA GCAATGGT | 3307 |
| 4354 | AUUGCACA A UUUUGUCC | 980 | GGACAAAA GGCTAGCTACAACGA TGTGCAAT | 3308 |
| 4359 | ACAAUUUU G UCCUAAUA | 981 | TATTAGGA GGCTAGCTACAACGA AAAATTGT | 3309 |
| 4365 | UUGUCCUA A UAUAUACA | 982 | TGTATATA GGCTAGCTACAACGA TAGGACAA | 3310 |
| 4367 | GUCCUAAU A UAUACAUA | 983 | TATGTATA GGCTAGCTACAACGA ATTAGGAC | 3311 |
| 4369 | CCUAAUAU A UACAUAGA | 984 | TCTATGTA GGCTAGCTACAACGA ATATTAGG | 3312 |
| 4371 | UAAUAUAU A CAUAGAAA | 985 | TTTCTATG GGCTAGCTACAACGA ATATATTA | 3313 |
| 4373 | AUAUAUAC A UAGAAACU | 986 | AGTTTCTA GGCTAGCTACAACGA GTATATAT | 3314 |
| 4379 | ACAUAGAA A CUUUGUGG | 987 | CCACAAAG GGCTAGCTACAACGA TTCTATGT | 3315 |
| 4384 | GAAACUUU G UGGGGCAU | 988 | ATGCCCCA GGCTAGCTACAACGA AAAGTTTC | 3316 |
| 4389 | UUUGUGGG G CAUGUUAA | 989 | TTAACATG GGCTAGCTACAACGA CCCACAAA | 3317 |
| 4391 | UGUGGGGC A UGUUAAGU | 990 | ACTTAACA GGCTAGCTACAACGA GCCCCACA | 3318 |
| 4393 | UGGGGCAU G UUAAGUUA | 991 | TAACTTAA GGCTAGCTACAACGA ATGCCCCA | 3319 |
| 4398 | CAUGUUAA G UUACAGUU | 992 | AACTGTAA GGCTAGCTACAACGA TTAACATG | 3320 |
| 4401 | GUUAAGUU A CAGUUUGC | 993 | GCAAACTG GGCTAGCTACAACGA AACTTAAC | 3321 |
| 4404 | AAGUUACA G UUUGCACA | 994 | TGTGCAAA GGCTAGCTACAACGA TGTAACTT | 3322 |
| 4408 | UACAGUUU G CACAAGUU | 995 | AACTTGTG GGCTAGCTACAACGA AAACTGTA | 3323 |
| 4410 | CAGUUUGC A CAAGUUCA | 996 | TGAACTTG GGCTAGCTACAACGA GCAAACTG | 3324 |
| 4414 | UUGCACAA G UUCAUCUC | 997 | GAGATGAA GGCTAGCTACAACGA TTGTGCAA | 3325 |
| 4418 | ACAAGUUC A UCUCAUUU | 998 | AAATGAGA GGCTAGCTACAACGA GAACTTGT | 3326 |
| 4423 | UUCAUCUC A UUUGUAUU | 999 | AATACAAA GGCTAGCTACAACGA GAGATGAA | 3327 |
| 4427 | UCUCAUUU G UAUUCCAU | 1000 | ATGGAATA GGCTAGCTACAACGA AAATGAGA | 3328 |
| 4429 | UCAUUUGU A UUCCAUUG | 1001 | CAATGGAA GGCTAGCTACAACGA ACAAATGA | 3329 |
| 4434 | UGUAUUCC A UUGAUUUU | 1002 | AAAATCAA GGCTAGCTACAACGA GGAATACA | 3330 |
| 4438 | UUCCAUUG A UUUUUUUU | 1003 | AAAAAAAA GGCTAGCTACAACGA CAATGGAA | 3331 |
| 4457 | UCUUCUAA A CAUUUUUU | 1004 | AAAAAATG GGCTAGCTACAACGA TTAGAAGA | 3332 |
| 4459 | UUCUAAAC A UUUUUUCU | 1005 | AGAAAAAA GGCTAGCTACAACGA GTTTAGAA | 3333 |
| 4473 | UCUUCAAA A CAGUAUAU | 1006 | ATATACTG GGCTAGCTACAACGA TTTGAAGA | 3334 |
| 4476 | UCAAAACA G UAUAUAUA | 1007 | TATATATA GGCTAGCTACAACGA TGTTTTGA | 3335 |
| 4478 | AAAACAGU A UAUAUAAC | 1008 | GTTATATA GGCTAGCTACAACGA ACTGTTTT | 3336 |
| 4480 | AACAGUAU A UAUAACUU | 1009 | AAGTTATA GGCTAGCTACAACGA ATACTGTT | 3337 |
| 4482 | CAGUAUAU A UAACUUUU | 1010 | AAAAGTTA GGCTAGCTACAACGA ATATACTG | 3338 |
| 4485 | UAUAUAUA A CUUUUUUU | 1011 | AAAAAAAG GGCTAGCTACAACGA TATATATA | 3339 |
| 4499 | UUUAGGGG A UUUUUUUU | 1012 | AAAAAAAA GGCTAGCTACAACGA CCCCTAAA | 3340 |
| 4510 | UUUUUUAG A CAGCAAAA | 1013 | TTTTGCTG GGCTAGCTACAACGA CTAAAAAA | 3341 |
| 4513 | UUUAGACA G CAAAAAAC | 1014 | GTTTTTTG GGCTAGCTACAACGA TGTCTAAA | 3342 |
| 4520 | AGCAAAAA A CUAUCUGA | 1015 | TCAGATAG GGCTAGCTACAACGA TTTTTGCT | 3343 |
| 4523 | AAAAAACU A UCUGAAGA | 1016 | TCTTCAGA GGCTAGCTACAACGA AGTTTTTT | 3344 |
| 4531 | AUCUGAAG A UUUCCAUU | 1017 | AATGGAAA GGCTAGCTACAACGA CTTCAGAT | 3345 |
| 4537 | AGAUUUCC A UUUGUCAA | 1018 | TTGACAAA GGCTAGCTACAACGA GGAAATCT | 3346 |
| 4541 | UUCCAUUU G UCAAAAAG | 1019 | CTTTTTGA GGCTAGCTACAACGA AAATGGAA | 3347 |
| 4549 | GUCAAAAA G UAAUGAUU | 1020 | AATCATTA GGCTAGCTACAACGA TTTTTGAC | 3348 |
| 4552 | AAAAAGUA A UGAUUUCU | 1021 | AGAAATCA GGCTAGCTACAACGA TACTTTTT | 3349 |
| 4555 | AAGUAAUG A UUUCUUGA | 1022 | TCAAGAAA GGCTAGCTACAACGA CATTACTT | 3350 |
| 4563 | AUUUCUUG A UAAUUGUG | 1023 | CACAATTA GGCTAGCTACAACGA CAAGAAAT | 3351 |
| 4566 | UCUUGAUA A UUGUGUAG | 1024 | CTACACAA GGCTAGCTACAACGA TATCAAGA | 3352 |
| 4569 | UGAUAAUU G UGUAGUGA | 1025 | TCACTACA GGCTAGCTACAACGA AATTATCA | 3353 |
| 4571 | AUAAUUGU G UAGUGAAU | 1026 | ATTCACTA GGCTAGCTACAACGA ACAATTAT | 3354 |
| 4574 | AUUGUGUA G UGAAUGUU | 1027 | AACATTCA GGCTAGCTACAACGA TACACAAT | 3355 |
| 4578 | UGUAGUGA A UGUUUUUU | 1028 | AAAAAACA GGCTAGCTACAACGA TCACTACA | 3356 |
| 4580 | UAGUGAAU G UUUUUUAG | 1029 | CTAAAAAA GGCTAGCTACAACGA ATTCACTA | 3357 |
| 4590 | UUUUUAGA A CCCAGCAG | 1030 | CTGCTGGG GGCTAGCTACAACGA TCTAAAAA | 3358 |
| 4595 | AGAACCCA G CAGUUACC | 1031 | GGTAACTG GGCTAGCTACAACGA TGGGTTCT | 3359 |
| 4598 | ACCCAGCA G UUACCUUG | 1032 | CAAGGTAA GGCTAGCTACAACGA TGCTGGGT | 3360 |
| 4601 | CAGCAGUU A CCUUGAAA | 1033 | TTTCAAGG GGCTAGCTACAACGA AACTGCTG | 3361 |
| 4610 | CCUUGAAA G CUGAAUUU | 1034 | AAATTCAG GGCTAGCTACAACGA TTTCAAGG | 3362 |
| 4615 | AAAGCUGA A UUUAUAUU | 1035 | AATATAAA GGCTAGCTACAACGA TCAGCTTT | 3363 |
| 4619 | CUGAAUUU A UAUUUAGU | 1036 | ACTAAATA GGCTAGCTACAACGA AAATTCAG | 3364 |
| 4621 | GAAUUUAU A UUUAGUAA | 1037 | TTACTAAA GGCTAGCTACAACGA ATAAATTC | 3365 |
| 4626 | UAUAUUUA G UAACUUCU | 1038 | AGAAGTTA GGCTAGCTACAACGA TAAATATA | 3366 |
| 4629 | AUUUAGUA A CUUCUGUG | 1039 | CACAGAAG GGCTAGCTACAACGA TACTAAAT | 3367 |
| 4635 | UAACUUCU G UGUUAAUA | 1040 | TATTAACA GGCTAGCTACAACGA AGAAGTTA | 3368 |
| 4637 | ACUUCUGU G UUAAUACU | 1041 | AGTATTAA GGCTAGCTACAACGA ACAGAAGT | 3369 |
| 4641 | CUGUGUUA A UACUGGAU | 1042 | ATCCAGTA GGCTAGCTACAACGA TAACACAG | 3370 |
| 4643 | GUGUUAAU A CUGGAUAG | 1043 | CTATCCAG GGCTAGCTACAACGA ATTAACAC | 3371 |
| 4648 | AAUACUGG A UAGCAUGA | 1044 | TCATGCTA GGCTAGCTACAACGA CCAGTATT | 3372 |
| 4651 | ACUGGAUA G CAUGAAUU | 1045 | AATTCATG GGCTAGCTACAACGA TATCCAGT | 3373 |
| 4653 | UGGAUAGC A UGAAUUCU | 1046 | AGAATTCA GGCTAGCTACAACGA GCTATCCA | 3374 |
| 4657 | UAGCAUGA A UUCUGCAU | 1047 | ATGCAGAA GGCTAGCTACAACGA TCATGCTA | 3375 |
| 4662 | UGAAUUCU G CAUUGAGA | 1048 | TCTCAATG GGCTAGCTACAACGA AGAATTCA | 3376 |
| 4664 | AAUUCUGC A UUGAGAAA | 1049 | TTTCTCAA GGCTAGCTACAACGA GCAGAATT | 3377 |
| 4672 | AUUGAGAA A CUGAAUAG | 1050 | CTATTCAG GGCTAGCTACAACGA TTCTCAAT | 3378 |
| 4677 | GAAACUGA A UAGCUGUC | 1051 | GACAGCTA GGCTAGCTACAACGA TCAGTTTC | 3379 |
| 4680 | ACUGAAUA G CUGUCAUA | 1052 | TATGACAG GGCTAGCTACAACGA TATTCAGT | 3380 |
| 4683 | GAAUAGCU G UCAUAAAA | 1053 | TTTTATGA GGCTAGCTACAACGA AGCTATTC | 3381 |
| 4686 | UAGCUGUC A UAAAAUGC | 1054 | GCATTTTA GGCTAGCTACAACGA GACAGCTA | 3382 |
| 4691 | GUCAUAAA A UGCUUUCU | 1055 | AGAAAGCA GGCTAGCTACAACGA TTTATGAC | 3383 |
| 4693 | CAUAAAAU G CUUUCUUU | 1056 | AAAGAAAG GGCTAGCTACAACGA ATTTTATG | 3384 |
| 4713 | AAAGAAAG A UACUCACA | 1057 | TGTGAGTA GGCTAGCTACAACGA CTTTCTTT | 3385 |
| 4715 | AGAAAGAU A CUCACAUG | 1058 | CATGTGAG GGCTAGCTACAACGA ATCTTTCT | 3386 |
| 4719 | AGAUACUC A CAUGAGUU | 1059 | AACTCATG GGCTAGCTACAACGA GAGTATCT | 3387 |
| 4721 | AUACUCAC A UGAGUUCU | 1060 | AGAACTCA GGCTAGCTACAACGA GTGAGTAT | 3388 |
| 4725 | UCACAUGA G UUCUUGAA | 1061 | TTCAAGAA GGCTAGCTACAACGA TCATGTGA | 3389 |
| 4736 | CUUGAAGA A UAGUCAUA | 1062 | TATGACTA GGCTAGCTACAACGA TCTTCAAG | 3390 |
| 4739 | GAAGAAUA G UCAUAACU | 1063 | AGTTATGA GGCTAGCTACAACGA TATTCTTC | 3391 |
| 4742 | GAAUAGUC A UAACUAGA | 1064 | TCTAGTTA GGCTAGCTACAACGA GACTATTC | 3392 |
| 4745 | UAGUCAUA A CUAGAUUA | 1065 | TAATCTAG GGCTAGCTACAACGA TATGACTA | 3393 |
| 4750 | AUAACUAG A UUAAGAUC | 1066 | GATCTTAA GGCTAGCTACAACGA CTAGTTAT | 3394 |
| 4756 | AGAUUAAG A UCUGUGUU | 1067 | AACACAGA GGCTAGCTACAACGA CTTAATCT | 3395 |
| 4760 | UAAGAUCU G UGUUUUAG | 1068 | CTAAAACA GGCTAGCTACAACGA AGATCTTA | 3396 |
| 4762 | AGAUCUGU G UUUUAGUU | 1069 | AACTAAAA GGCTAGCTACAACGA ACAGATCT | 3397 |
| 4768 | GUGUUUUA G UUUAAUAG | 1070 | CTATTAAA GGCTAGCTACAACGA TAAAACAC | 3398 |
| 4773 | UUAGUUUA A UAGUUUGA | 1071 | TCAAACTA GGCTAGCTACAACGA TAAACTAA | 3399 |
| 4776 | GUUUAAUA G UUUGAAGU | 1072 | ACTTCAAA GGCTAGCTACAACGA TATTAAAC | 3400 |
| 4783 | AGUUUGAA G UGCCUGUU | 1073 | AACAGGCA GGCTAGCTACAACGA TTCAAACT | 3401 |
| 4785 | UUUGAAGU G CCUGUUUG | 1074 | CAAACAGG GGCTAGCTACAACGA ACTTCAAA | 3402 |
| 4789 | AAGUGCCU G UUUGGGAU | 1075 | ATCCCAAA GGCTAGCTACAACGA AGGCACTT | 3403 |
| 4796 | UGUUUGGG A UAAUGAUA | 1076 | TATCATTA GGCTAGCTACAACGA CCCAAACA | 3404 |
| 4799 | UUGGGAUA A UGAUAGGU | 1077 | ACCTATCA GGCTAGCTACAACGA TATCCCAA | 3405 |
| 4802 | GGAUAAUG A UAGGUAAU | 1078 | ATTACCTA GGCTAGCTACAACGA CATTATCC | 3406 |
| 4806 | AAUGAUAG G UAAUUUAG | 1079 | CTAAATTA GGCTAGCTACAACGA CTATCATT | 3407 |
| 4809 | GAUAGGUA A UUUAGAUG | 1080 | CATCTAAA GGCTAGCTACAACGA TACCTATC | 3408 |
| 4815 | UAAUUUAG A UGAAUUUA | 1081 | TAAATTCA GGCTAGCTACAACGA CTAAATTA | 3409 |
| 4819 | UUAGAUGA A UUUAGGGG | 1082 | CCCCTAAA GGCTAGCTACAACGA TCATCTAA | 3410 |
| 4836 | AAAAAAAA G UUAUCUGC | 1083 | GCAGATAA GGCTAGCTACAACGA TTTTTTTT | 3411 |
| 4839 | AAAAAGUU A UCUGCAGU | 1084 | ACTGCAGA GGCTAGCTACAACGA AACTTTTT | 3412 |
| 4843 | AGUUAUCU G CAGUUAUG | 1085 | CATAACTG GGCTAGCTACAACGA AGATAACT | 3413 |
| 4846 | UAUCUGCA G UUAUGUUG | 1086 | CAACATAA GGCTAGCTACAACGA TGCAGATA | 3414 |
| 4849 | CUGCAGUU A UGUUGAGG | 1087 | CCTCAACA GGCTAGCTACAACGA AACTGCAG | 3415 |
| 4851 | GCAGUUAU G UUGAGGGC | 1088 | GCCCTCAA GGCTAGCTACAACGA ATAACTGC | 3416 |
| 4858 | UGUUGAGG G CCCAUCUC | 1089 | GAGATGGG GGCTAGCTACAACGA CCTCAACA | 3417 |
| 4862 | GAGGGCCC A UCUCUCCC | 1090 | GGGAGAGA GGCTAGCTACAACGA GGGCCCTC | 3418 |
| 4874 | CUCCCCCC A CACCCCCA | 1091 | TGGGGGTG GGCTAGCTACAACGA GGGGGGAG | 3419 |
| 4876 | CCCCCCAC A CCCCCACA | 1092 | TGTGGGGG GGCTAGCTACAACGA GTGGGGGG | 3420 |
| 4882 | ACACCCCC A CAGAGCUA | 1093 | TAGCTCTG GGCTAGCTACAACGA GGGGGTGT | 3421 |
| 4887 | CCCACAGA G CUAACUGG | 1094 | CCAGTTAG GGCTAGCTACAACGA TCTGTGGG | 3422 |
| 4891 | CAGAGCUA A CUGGGUUA | 1095 | TAACCCAG GGCTAGCTACAACGA TAGCTCTG | 3423 |
| 4896 | CUAACUGG G UUACAGUG | 1096 | CACTGTAA GGCTAGCTACAACGA CCAGTTAG | 3424 |
| 4899 | ACUGGGUU A CAGUGUUU | 1097 | AAACACTG GGCTAGCTACAACGA AACCCAGT | 3425 |
| 4902 | GGGUUACA G UGUUUUAU | 1098 | ATAAAACA GGCTAGCTACAACGA TGTAACCC | 3426 |
| 4904 | GUUACAGU G UUUUAUCC | 1099 | GGATAAAA GGCTAGCTACAACGA ACTGTAAC | 3427 |
| 4909 | AGUGUUUU A UCCGAAAG | 1100 | CTTTCGGA GGCTAGCTACAACGA AAAACACT | 3428 |
| 4917 | AUCCGAAA G UUUCCAAU | 1101 | ATTGGAAA GGCTAGCTACAACGA TTTCGGAT | 3429 |
| 4924 | AGUUUCCA A UUCCACUG | 1102 | CAGTGGAA GGCTAGCTACAACGA TGGAAACT | 3430 |
| 4929 | CCAAUUCC A CUGUCUUG | 1103 | CAAGACAG GGCTAGCTACAACGA GGAATTGG | 3431 |
| 4932 | AUUCCACU G UCUUGUGU | 1104 | ACACAAGA GGCTAGCTACAACGA AGTGGAAT | 3432 |
| 4937 | ACUGUCUU G UGUUUUCA | 1105 | TGAAAACA GGCTAGCTACAACGA AAGACAGT | 3433 |
| 4939 | UGUCUUGU G UUUUCAUG | 1106 | CATGAAAA GGCTAGCTACAACGA ACAAGACA | 3434 |
| 4945 | GUGUUUUC A UGUUGAAA | 1107 | TTTCAACA GGCTAGCTACAACGA GAAAACAC | 3435 |
| 4947 | GUUUUCAU G UUGAAAAU | 1108 | ATTTTCAA GGCTAGCTACAACGA ATGAAAAC | 3436 |
| 4954 | UGUUGAAA A UACUUUUG | 1109 | CAAAAGTA GGCTAGCTACAACGA TTTCAACA | 3437 |
| 4956 | UUGAAAAU A CUUUUGCA | 1110 | TGCAAAAG GGCTAGCTACAACGA ATTTTCAA | 3438 |
| 4962 | AUACUUUU G CAUUUUUC | 1111 | GAAAAATG GGCTAGCTACAACGA AAAAGTAT | 3439 |
| 4964 | ACUUUUGC A UUUUUCCU | 1112 | AGGAAAAA GGCTAGCTACAACGA GCAAAAGT | 3440 |
| 4977 | UCCUUUGA G UGCCAAUU | 1113 | AATTGGCA GGCTAGCTACAACGA TCAAAGGA | 3441 |
| 4979 | CUUUGAGU G CCAAUUUC | 1114 | GAAATTGG GGCTAGCTACAACGA ACTCAAAG | 3442 |
| 4983 | GAGUGCCA A UUUCUUAC | 1115 | GTAAGAAA GGCTAGCTACAACGA TGGCACTC | 3443 |
| 4990 | AAUUUCUU A CUAGUACU | 1116 | AGTACTAG GGCTAGCTACAACGA AAGAAATT | 3444 |
| 4994 | UCUUACUA G UACUAUUU | 1117 | AAATAGTA GGCTAGCTACAACGA TAGTAAGA | 3445 |
| 4996 | UUACUAGU A CUAUUUCU | 1118 | AGAAATAG GGCTAGCTACAACGA ACTAGTAA | 3446 |
| 4999 | CUAGUACU A UUUCUUAA | 1119 | TTAAGAAA GGCTAGCTACAACGA AGTACTAG | 3447 |
| 5007 | AUUUCUUA A UGUAACAU | 1120 | ATGTTACA GGCTAGCTACAACGA TAAGAAAT | 3448 |
| 5009 | UUCUUAAU G UAACAUGU | 1121 | ACATGTTA GGCTAGCTACAACGA ATTAAGAA | 3449 |
| 5012 | UUAAUGUA A CAUGUUUA | 1122 | TAAACATG GGCTAGCTACAACGA TACATTAA | 3450 |
| 5014 | AAUGUAAC A UGUUUACC | 1123 | GGTAAACA GGCTAGCTACAACGA GTTACATT | 3451 |
| 5016 | UGUAACAU G UUUACCUG | 1124 | CAGGTAAA GGCTAGCTACAACGA ATGTTACA | 3452 |
| 5020 | ACAUGUUU A CCUGGCCU | 1125 | AGGCCAGG GGCTAGCTACAACGA AAACATGT | 3453 |
| 5025 | UUUACCUG G CCUGUCUU | 1126 | AAGACAGG GGCTAGCTACAACGA CAGGTAAA | 3454 |
| 5029 | CCUGGCCU G UCUUUUAA | 1127 | TTAAAAGA GGCTAGCTACAACGA AGGCCAGG | 3455 |
| 5037 | GUCUUUUA A CUAUUUUU | 1128 | AAAAATAG GGCTAGCTACAACGA TAAAAGAC | 3456 |
| 5040 | UUUUAACU A UUUUUGUA | 1129 | TACAAAAA GGCTAGCTACAACGA AGTTAAAA | 3457 |
| 5046 | CUAUUUUU G UAUAGUCU | 1130 | ACACTATA GGCTAGCTACAACGA AAAAATAG | 3458 |
| 5048 | AUUUUUGU A UAGUGUAA | 1131 | TTACACTA GGCTAGCTACAACGA ACAAAAAT | 3459 |
| 5051 | UUUGUAUA G UCUAAACU | 1132 | AGTTTACA GGCTAGCTACAACGA TATACAAA | 3460 |
| 5053 | UGUAUAGU G UAAACUGA | 1133 | TCAGTTTA GGCTAGCTACAACGA ACTATACA | 3461 |
| 5057 | UAGUGUAA A CUGAAACA | 1134 | TGTTTCAG GGCTAGCTACAACGA TTACACTA | 3462 |
| 5063 | AAACUGAA A CAUGCACA | 1135 | TGTGCATG GGCTAGCTACAACGA TTCAGTTT | 3463 |
| 5065 | ACUGAAAC A UGCACAUU | 1136 | AATGTGCA GGCTAGCTACAACGA GTTTCAGT | 3464 |
| 5067 | UGAAACAU G CACAUUUU | 1137 | AAAATGTG GGCTAGCTACAACGA ATGTTTCA | 3465 |
| 5069 | AAACAUGC A CAUUUUGU | 1138 | ACAAAATG GGCTAGCTACAACGA GCATGTTT | 3466 |
| 5071 | ACAUGCAC A UUUUGUAC | 1139 | GTACAAAA GGCTAGCTACAACGA GTGCATGT | 3467 |
| 5076 | CACAUUUU G UACAUUGU | 1140 | ACAATGTA GGCTAGCTACAACGA AAAATGTG | 3468 |
| 5078 | CAUUUUGU A CAUUGUGC | 1141 | GCACAATG GGCTAGCTACAACGA ACAAAATG | 3469 |
| 5080 | UUUUGUAC A UUGUGCUU | 1142 | AAGCACAA GGCTAGCTACAACGA GTACAAAA | 3470 |
| 5083 | UGUACAUU G UGCUUUCU | 1143 | AGAAAGCA GGCTAGCTACAACGA AATGTACA | 3471 |
| 5085 | UACAUUGU G CUUUCUUU | 1144 | AAAGAAAG GGCTAGCTACAACGA ACAATGTA | 3472 |
| 5095 | UUUCUUUU G UGGGUCAU | 1145 | ATGACCCA GGCTAGCTACAACGA AAAAGAAA | 3473 |
| 5099 | UUUUGUGG G UCAUAUGC | 1146 | GCATATGA GGCTAGCTACAACGA CCACAAAA | 3474 |
| 5102 | UGUGGGUC A UAUGCAGU | 1147 | ACTGCATA GGCTAGCTACAACGA GACCCACA | 3475 |
| 5104 | UGGGUCAU A UGCAGUGU | 1148 | ACACTGCA GGCTAGCTACAACGA ATGACCCA | 3476 |
| 5106 | GGUCAUAU G CAGUGUGA | 1149 | TCACACTG GGCTAGCTACAACGA ATATGACC | 3477 |
| 5109 | CAUAUGCA G UGUGAUCC | 1150 | GGATCACA GGCTAGCTACAACGA TGCATATG | 3478 |
| 5111 | UAUGCAGU G UGAUCCAG | 1151 | CTGGATCA GGCTAGCTACAACGA ACTGCATA | 3479 |
| 5114 | GCAGUGUG A UCCAGUUG | 1152 | CAACTGGA GGCTAGCTACAACGA CACACTGC | 3480 |
| 5119 | GUGAUCCA G UUGUUUUC | 1153 | GAAAACAA GGCTAGCTACAACGA TGGATCAC | 3481 |
| 5122 | AUCCAGUU G UUUUCCAU | 1154 | ATGGAAAA GGCTAGCTACAACGA AACTGGAT | 3482 |
| 5129 | UGUUUUCC A UCAUUUGG | 1155 | CCAAATGA GGCTAGCTACAACGA GGAAAACA | 3483 |
| 5132 | UUUCCAUC A UUUGGUUG | 1156 | CAACCAAA GGCTAGCTACAACGA GATGGAAA | 3484 |
| 5137 | AUCAUUUG G UUGCGCUG | 1157 | CAGCGCAA GGCTAGCTACAACGA CAAATGAT | 3485 |
| 5140 | AUUUGGUU G CGCUGACC | 1158 | GGTCAGCG GGCTAGCTACAACGA AACCAAAT | 3486 |
| 5142 | UUGGUUGC G CUGACCUA | 1159 | TAGGTCAG GGCTAGCTACAACGA GCAACCAA | 3487 |
| 5146 | UUGCGCUG A CCUAGGAA | 1160 | TTCCTAGG GGCTAGCTACAACGA CAGCGCAA | 3488 |
| 5154 | ACCUAGGA A UGUUGGUC | 1161 | GACCAACA GGCTAGCTACAACGA TCCTAGGT | 3489 |
| 5156 | CUAGGAAU G UUGGUCAU | 1162 | ATGACCAA GGCTAGCTACAACGA ATTCCTAG | 3490 |
| 5160 | GAAUGUUG G UCAUAUCA | 1163 | TGATATGA GGCTAGCTACAACGA CAACATTC | 3491 |
| 5163 | UGUUGGUC A UAUCAAAC | 1164 | GTTTGATA GGCTAGCTACAACGA GACCAACA | 3492 |
| 5165 | UUGGUCAU A UCAAACAU | 1165 | ATGTTTGA GGCTAGCTACAACGA ATGACCAA | 3493 |
| 5170 | CAUAUCAA A CAUUAAAA | 1166 | TTTTAATG GGCTAGCTACAACGA TTGATATG | 3494 |
| 5172 | UAUCAAAC A UUAAAAAU | 1167 | ATTTTTAA GGCTAGCTACAACGA GTTTGATA | 3495 |
| 5179 | CAUUAAAA A UGACCACU | 1168 | AGTGGTCA GGCTAGCTACAACGA TTTTAATG | 3496 |
| 5182 | UAAAAAUG A CCACUCUU | 1169 | AAGAGTGG GGCTAGCTACAACGA CATTTTTA | 3497 |
| 5185 | AAAUGACC A CUCUUUUA | 1170 | TAAAAGAG GGCTAGCTACAACGA GGTCATTT | 3498 |
| 5194 | CUCUUUUA A UGAAAUUA | 1171 | TAATTTCA GGCTAGCTACAACGA TAAAAGAG | 3499 |
| 5199 | UUAAUGAA A UUAACUUU | 1172 | AAAGTTAA GGCTAGCTACAACGA TTCATTAA | 3500 |
| 5203 | UGAAAUUA A CUUUUAAA | 1173 | TTTAAAAG GGCTAGCTACAACGA TAATTTCA | 3501 |
| 5211 | ACUUUUAA A UGUUUAUA | 1174 | TATAAACA GGCTAGCTACAACGA TTAAAAGT | 3502 |
| 5213 | UUUUAAAU G UUUAUAGG | 1175 | CCTATAAA GGCTAGCTACAACGA ATTTAAAA | 3503 |
| 5217 | AAAUGUUU A UAGGAGUA | 1176 | TACTCCTA GGCTAGCTACAACGA AAACATTT | 3504 |
| 5223 | UUAUAGGA G UAUGUGCU | 1177 | AGCACATA GGCTAGCTACAACGA TCCTATAA | 3505 |
| 5225 | AUAGGAGU A UGUGCUGU | 1178 | ACAGCACA GGCTAGCTACAACGA ACTCCTAT | 3506 |
| 5227 | AGGAGUAU G UGCUGUGA | 1179 | TCACAGCA GGCTAGCTACAACGA ATACTCCT | 3507 |
| 5229 | GAGUAUGU G CUGUGAAG | 1180 | CTTCACAG GGCTAGCTACAACGA ACATACTC | 3508 |
| 5232 | UAUGUGCU G UGAAGUGA | 1181 | TCACTTCA GGCTAGCTACAACGA AGCACATA | 3509 |
| 5237 | GCUGUGAA G UGAUCUAA | 1182 | TTAGATCA GGCTAGCTACAACGA TTCACAGC | 3510 |
| 5240 | GUGAAGUG A UCUAAAAU | 1183 | ATTTTAGA GGCTAGCTACAACGA CACTTCAC | 3511 |
| 5247 | GAUCUAAA A UUUGUAAU | 1184 | ATTACAAA GGCTAGCTACAACGA TTTAGATC | 3512 |
| 5251 | UAAAAUUU G UAAUAUUU | 1185 | AAATATTA GGCTAGCTACAACGA AAATTTTA | 3513 |
| 5254 | AAUUUGUA A UAUUUUUG | 1186 | CAAAAATA GGCTAGCTACAACGA TACAAATT | 3514 |
| 5256 | UUUGUAAU A UUUUUGUC | 1187 | GACAAAAA GGCTAGCTACAACGA ATTACAAA | 3515 |
| 5262 | AUAUUUUU G UCAUGAAC | 1188 | GTTCATGA GGCTAGCTACAACGA AAAAATAT | 3516 |
| 5265 | UUUUUGUC A UGAACUGU | 1189 | ACAGTTCA GGCTAGCTACAACGA GACAAAAA | 3517 |
| 5269 | UGUCAUGA A CUGUACUA | 1190 | TAGTACAG GGCTAGCTACAACGA TCATGACA | 3518 |
| 5272 | CAUGAACU G UACUACUC | 1191 | GAGTAGTA GGCTAGCTACAACGA AGTTCATG | 3519 |
| 5274 | UGAACUGU A CUACUCCU | 1192 | AGGAGTAG GGCTAGCTACAACGA ACAGTTCA | 3520 |
| 5277 | ACUGUACU A CUCCUAAU | 1193 | ATTAGGAG GGCTAGCTACAACGA AGTACAGT | 3521 |
| 5284 | UACUCCUA A UUAUUGUA | 1194 | TACAATAA GGCTAGCTACAACGA TAGGAGTA | 3522 |
| 5287 | UCCUAAUU A UUGUAAUG | 1195 | CATTACAA GGCTAGCTACAACGA AATTAGGA | 3523 |
| 5290 | UAAUUAUU G UAAUGUAA | 1196 | TTACATTA GGCTAGCTACAACGA AATAATTA | 3524 |
| 5293 | UUAUUGUA A UGUAAUAA | 1197 | TTATTACA GGCTAGCTACAACGA TACAATAA | 3525 |
| 5295 | AUUGUAAU G UAAUAAAA | 1198 | TTTTATTA GGCTAGCTACAACGA ATTACAAT | 3526 |
| 5298 | GUAAUGUA A UAAAAAUA | 1199 | TATTTTTA GGCTAGCTACAACGA TACATTAC | 3527 |
| 5304 | UAAUAAAA A UAGUUACA | 1200 | TGTAACTA GGCTAGCTACAACGA TTTTATTA | 3528 |
| 5307 | UAAAAAUA G UUACAGUG | 1201 | CACTGTAA GGCTAGCTACAACGA TATTTTTA | 3529 |
| 5310 | AAAUAGUU A CAGUGACU | 1202 | AGTCACTG GGCTAGCTACAACGA AACTATTT | 3530 |
| 5313 | UAGUUACA G UGACUAUG | 1203 | CATAGTCA GGCTAGCTACAACGA TGTAACTA | 3531 |
| 5316 | UUACAGUG A CUAUGAGU | 1204 | ACTCATAG GGCTAGCTACAACGA CACTGTAA | 3532 |
| 5319 | CAGUGACU A UGAGUGUG | 1205 | CACACTCA GGCTAGCTACAACGA AGTCACTG | 3533 |
| 5323 | GACUAUGA G UGUGUAUU | 1206 | AATACACA GGCTAGCTACAACGA TCATAGTC | 3534 |
| 5325 | CUAUGAGU G UGUAUUUA | 1207 | TAAATACA GGCTAGCTACAACGA ACTCATAG | 3535 |
| 5327 | AUGAGUGU G UAUUUAUU | 1208 | AATAAATA GGCTAGCTACAACGA ACACTCAT | 3536 |
| 5329 | GAGUGUGU A UUUAUUCA | 1209 | TGAATAAA GGCTAGCTACAACGA ACACACTC | 3537 |
| 5333 | GUGUAUUU A UUCAUGCA | 1210 | TGCATGAA GGCTAGCTACAACGA AAATACAC | 3538 |
| 5337 | AUUUAUUC A UGCAAAUU | 1211 | AATTTGCA GGCTAGCTACAACGA GAATAAAT | 3539 |
| 5339 | UUAUUCAU G CAAAUUUG | 1212 | CAAATTTG GGCTAGCTACAACGA ATGAATAA | 3540 |
| 5343 | UCAUGCAA A UUUGAACU | 1213 | AGTTCAAA GGCTAGCTACAACGA TTGCATGA | 3541 |
| 5349 | AAAUUUGA A CUGUUUCC | 1214 | GCAAACAG GGCTAGCTACAACGA TCAAATTT | 3542 |
| 5352 | UUUGAACU G UUUGCCCC | 1215 | GGGGCAAA GGCTAGCTACAACGA AGTTCAAA | 3543 |
| 5356 | AACUGUUU G CCCCGAAA | 1216 | TTTCGGGG GGCTAGCTACAACGA AAACAGTT | 3544 |
| 5364 | GCCCCGAA A UGGAUAUG | 1217 | CATATCCA GGCTAGCTACAACGA TTCGGGGC | 3545 |
| 5368 | CGAAAUGG A UAUGGAUA | 1218 | TATCCATA GGCTAGCTACAACGA CCATTTCG | 3546 |
| 5370 | AAAUGGAU A UGGAUACU | 1219 | AGTATCCA GGCTAGCTACAACGA ATCCATTT | 3547 |
| 5374 | GGAUAUGG A UACUUUAU | 1220 | ATAAAGTA GGCTAGCTACAACGA CCATATCC | 3548 |
| 5376 | AUAUGGAU A CUUUAUAA | 1221 | TTATAAAG GGCTAGCTACAACGA ATCCATAT | 3549 |
| 5381 | GAUACUUU A UAAGCCAU | 1222 | ATGGCTTA GGCTAGCTACAACGA AAAGTATC | 3550 |
| 5385 | CUUUAUAA G CCAUAGAC | 1223 | GTCTATGG GGCTAGCTACAACGA TTATAAAG | 3551 |
| 5388 | UAUAAGCC A UAGACACU | 1224 | AGTGTCTA GGCTAGCTACAACGA GGCTTATA | 3552 |
| 5392 | AGCCAUAG A CACUAUAG | 1225 | CTATAGTG GGCTAGCTACAACGA CTATGGCT | 3553 |
| 5394 | CCAUAGAC A CUAUAGUA | 1226 | TACTATAG GGCTAGCTACAACGA GTCTATGG | 3554 |
| 5397 | UAGACACU A UAGUAUAC | 1227 | GTATACTA GGCTAGCTACAACGA AGTGTCTA | 3555 |
| 5400 | ACACUAUA G UAUACCAG | 1228 | CTGGTATA GGCTAGCTACAACGA TATAGTGT | 3556 |
| 5402 | ACUAUAGU A UACCAGUG | 1229 | CACTGGTA GGCTAGCTACAACGA ACTATAGT | 3557 |
| 5404 | UAUAGUAU A CCAGUGAA | 1230 | TTCACTGG GGCTAGCTACAACGA ATACTATA | 3558 |
| 5408 | GUAUACCA G UGAAUCUU | 1231 | AAGATTCA GGCTAGCTACAACGA TGGTATAC | 3559 |
| 5412 | ACCAGUGA A UCUUUUAU | 1232 | ATAAAAGA GGCTAGCTACAACGA TCACTGGT | 3560 |
| 5419 | AAUCUUUU A UGCAGCUU | 1233 | AAGCTGCA GGCTAGCTACAACGA AAAAGATT | 3561 |
| 5421 | UCUUUUAU G CAGCUUGU | 1234 | ACAAGCTG GGCTAGCTACAACGA ATAAAAGA | 3562 |
| 5424 | UUUAUGCA G CUUGUUAG | 1235 | CTAACAAG GGCTAGCTACAACGA TGCATAAA | 3563 |
| 5428 | UGCAGCUU G UUAGAAGU | 1236 | ACTTCTAA GGCTAGCTACAACGA AAGCTGCA | 3564 |
| 5435 | UGUUAGAA G UAUCCUUU | 1237 | AAAGGATA GGCTAGCTACAACGA TTCTAACA | 3565 |
| 5437 | UUAGAAGU A UCCUUUUA | 1238 | TAAAAGGA GGCTAGCTACAACGA ACTTCTAA | 3566 |
| 5445 | AUCCUUUU A UUUUCUAA | 1239 | TTAGAAAA GGCTAGCTACAACGA AAAAGGAT | 3567 |
| 5457 | UCUAAAAG G UGCUGUGG | 1240 | CCACAGCA GGCTAGCTACAACGA CTTTTAGA | 3568 |
| 5459 | UAAAAGGU G CUGUGGAU | 1241 | ATCCACAG GGCTAGCTACAACGA ACCTTTTA | 3569 |
| 5462 | AAGGUGCU G UGGAUAUU | 1242 | AATATCCA GGCTAGCTACAACGA AGCACCTT | 3570 |
| 5466 | UGCUGUGG A UAUUAUGU | 1243 | ACATAATA GGCTAGCTACAACGA CCACAGCA | 3571 |
| 5468 | CUGUGGAU A UUAUGUAA | 1244 | TTACATAA GGCTAGCTACAACGA ATCCACAG | 3572 |
| 5471 | UGGAUAUU A UGUAAAGG | 1245 | CCTTTACA GGCTAGCTACAACGA AATATCCA | 3573 |
| 5473 | GAUAUUAU G UAAAGGCG | 1246 | CGCCTTTA GGCTAGCTACAACGA ATAATATC | 3574 |
| 5479 | AUGUAAAG G CGUGUUUG | 1247 | CAAACACG GGCTAGCTACAACGA CTTTACAT | 3575 |
| 5481 | GUAAAGGC G UGUUUGCU | 1248 | AGCAAACA GGCTAGCTACAACGA GCCTTTAC | 3576 |
| 5483 | AAAGGCGU G UUUGCUUA | 1249 | TAAGCAAA GGCTAGCTACAACGA ACGCCTTT | 3577 |
| 5487 | GCGUGUUU G CUUAAACA | 1250 | TGTTTAAG GGCTAGCTACAACGA AAACACGC | 3578 |
| 5493 | UUGCUUAA A CAAUUUUC | 1251 | GAAAATTG GGCTAGCTACAACGA TTAAGCAA | 3579 |
| 5496 | CUUAAACA A UUUUCCAU | 1252 | ATGGAAAA GGCTAGCTACAACGA TGTTTAAG | 3580 |
| 5503 | AAUUUUCC A UAUUUAGA | 1253 | TCTAAATA GGCTAGCTACAACGA GGAAAATT | 3581 |
| 5505 | UUUUCCAU A UUUAGAAG | 1254 | CTTCTAAA GGCTAGCTACAACGA ATGGAAAA | 3582 |
| 5513 | AUUUAGAA G UAGAUGCA | 1255 | TGCATCTA GGCTAGCTACAACGA TTCTAAAT | 3583 |
| 5517 | AGAAGUAG A UGCAAAAC | 1256 | GTTTTGCA GGCTAGCTACAACGA CTACTTCT | 3584 |
| 5519 | AAGUAGAU G CAAAACAA | 1257 | TTGTTTTG GGCTAGCTACAACGA ATCTACTT | 3585 |
| 5524 | GAUGCAAA A CAAAUCUG | 1258 | CAGATTTG GGCTAGCTACAACGA TTTGCATC | 3586 |
| 5528 | CAAAACAA A UCUGCCUU | 1259 | AAGGCAGA GGCTAGCTACAACGA TTGTTTTG | 3587 |
| 5532 | ACAAAUCU G CCUUUAUG | 1260 | CATAAAGG GGCTAGCTACAACGA AGATTTGT | 3588 |
| 5538 | CUGCCUUU A UGACAAAA | 1261 | TTTTGTCA GGCTAGCTACAACGA AAAGGCAG | 3589 |
| 5541 | CCUUUAUG A CAAAAAAA | 1262 | TTTTTTTG GGCTAGCTACAACGA CATAAAGG | 3590 |
| 5549 | ACAAAAAA A UAGGAUAA | 1263 | TTATCCTA GGCTAGCTACAACGA TTTTTTGT | 3591 |
| 5554 | AAAAUAGG A UAACAUUA | 1264 | TAATGTTA GGCTAGCTACAACGA CCTATTTT | 3592 |
| 5557 | AUAGGAUA A CAUUAUUU | 1265 | AAATAATG GGCTAGCTACAACGA TATCCTAT | 3593 |
| 5559 | AGGAUAAC A UUAUUUAU | 1266 | ATAAATAA GGCTAGCTACAACGA GTTATCCT | 3594 |
| 5562 | AUAACAUU A UUUAUUUA | 1267 | TAAATAAA GGCTAGCTACAACGA AATGTTAT | 3595 |
| 5566 | CAUUAUUU A UUUAUUUC | 1268 | GAAATAAA GGCTAGCTACAACGA AAATAATG | 3596 |
| 5570 | AUUUAUUU A UUUCCUUU | 1269 | AAAGGAAA GGCTAGCTACAACGA AAATAAAT | 3597 |
| 5580 | UUCCUUUU A UCAAUAAG | 1270 | CTTATTGA GGCTAGCTACAACGA AAAAGGAA | 3598 |
| 5584 | UUUUAUCA A UAAGGUAA | 1271 | TTACCTTA GGCTAGCTACAACGA TGATAAAA | 3599 |
| 5589 | UCAAUAAG G UAAUUGAU | 1272 | ATCAATTA GGCTAGCTACAACGA CTTATTGA | 3600 |
| 5592 | AUAAGGUA A UUGAUACA | 1273 | TGTATCAA GGCTAGCTACAACGA TACCTTAT | 3601 |
| 5596 | GGUAAUUG A UACACAAC | 1274 | GTTGTGTA GGCTAGCTACAACGA CAATTACC | 3602 |
| 5598 | UAAUUGAU A CACAACAG | 1275 | CTGTTGTG GGCTAGCTACAACGA ATCAATTA | 3603 |
| 5600 | AUUGAUAC A CAACAGGU | 1276 | ACCTGTTG GGCTAGCTACAACGA GTATCAAT | 3604 |
| 5603 | GAUACACA A CAGGUGAC | 1277 | GTCACCTG GGCTAGCTACAACGA TGTGTATC | 3605 |
| 5607 | CACAACAG G UGACUUGG | 1278 | CCAAGTCA GGCTAGCTACAACGA CTGTTGTG | 3606 |
| 5610 | AACAGGUG A CUUGGUUU | 1279 | AAACCAAG GGCTAGCTACAACGA CACCTGTT | 3607 |
| 5615 | GUGACUUG G UUUUAGGC | 1280 | GCCTAAAA GGCTAGCTACAACGA CAAGTCAC | 3608 |
| 5622 | GGUUUUAG G CCCAAAGG | 1281 | CCTTTGGG GGCTAGCTACAACGA CTAAAACC | 3609 |
| 5630 | GCCCAAAG G UAGCAGCA | 1282 | TGCTGCTA GGCTAGCTACAACGA CTTTGGGC | 3610 |
| 5633 | CAAAGGUA G CAGCAGCA | 1283 | TGCTGCTG GGCTAGCTACAACGA TACCTTTG | 3611 |
| 5636 | AGGUAGCA G CAGCAACA | 1284 | TGTTGCTG GGCTAGCTACAACGA TGCTACCT | 3612 |
| 5639 | UAGCAGCA G CAACAUUA | 1285 | TAATGTTG GGCTAGCTACAACGA TGCTGCTA | 3613 |
| 5642 | CAGCAGCA A CAUUAAUA | 1286 | TATTAATG GGCTAGCTACAACGA TGCTGCTG | 3614 |
| 5644 | GCAGCAAC A UUAAUAAU | 1287 | ATTATTAA GGCTAGCTACAACGA GTTGCTGC | 3615 |
| 5648 | CAACAUUA A UAAUGGAA | 1288 | TTCCATTA GGCTAGCTACAACGA TAATGTTG | 3616 |
| 5651 | CAUUAAUA A UGGAAAUA | 1289 | TATTTCCA GGCTAGCTACAACGA TATTAATG | 3617 |
| 5657 | UAAUGGAA A UAAUUGAA | 1290 | TTCAATTA GGCTAGCTACAACGA TTCCATTA | 3618 |
| 5660 | UGGAAAUA A UUGAAUAG | 1291 | CTATTCAA GGCTAGCTACAACGA TATTTCCA | 3619 |
| 5665 | AUAAUUGA A UAGUUAGU | 1292 | ACTAACTA GGCTAGCTACAACGA TCAATTAT | 3620 |
| 5668 | AUUGAAUA G UUAGUUAU | 1293 | ATAACTAA GGCTAGCTACAACGA TATTCAAT | 3621 |
| 5672 | AAUAGUUA G UUAUGUAU | 1294 | ATACATAA GGCTAGCTACAACGA TAACTATT | 3622 |
| 5675 | AGUUAGUU A UGUAUGUU | 1295 | AACATACA GGCTAGCTACAACGA AACTAACT | 3623 |
| 5677 | UUAGUUAU G UAUGUUAA | 1296 | TTAACATA GGCTAGCTACAACGA ATAACTAA | 3624 |
| 5679 | AGUUAUGU A UGUUAAUG | 1297 | CATTAACA GGCTAGCTACAACGA ACATAACT | 3625 |
| 5681 | UUAUGUAU G UUAAUGCC | 1298 | GGCATTAA GGCTAGCTACAACGA ATACATAA | 3626 |
| 5685 | GUAUGUUA A UGCCAGUC | 1299 | GACTGGCA GGCTAGCTACAACGA TAACATAC | 3627 |
| 5687 | AUGUUAAU G CCAGUCAC | 1300 | GTGACTGG GGCTAGCTACAACGA ATTAACAT | 3628 |
| 5691 | UAAUGCCA G UCACCAGC | 1301 | GCTGGTGA GGCTAGCTACAACGA TGGCATTA | 3629 |
| 5694 | UGCCAGUC A CCAGCAGG | 1302 | CCTGCTGG GGCTAGCTACAACGA GACTGGCA | 3630 |
| 5698 | AGUCACCA G CAGGCUAU | 1303 | ATAGCCTG GGCTAGCTACAACGA TGGTGACT | 3631 |
| 5702 | ACCAGCAG G CUAUUUCA | 1304 | TGAAATAG GGCTAGCTACAACGA CTGCTGGT | 3632 |
| 5705 | AGCAGGCU A UUUCAAGG | 1305 | CCTTGAAA GGCTAGCTACAACGA AGCCTGCT | 3633 |
| 5713 | AUUUCAAG G UCAGAAGU | 1306 | ACTTCTGA GGCTAGCTACAACGA CTTGAAAT | 3634 |
| 5720 | GGUCAGAA G UAAUGACU | 1307 | AGTCATTA GGCTAGCTACAACGA TTCTGACC | 3635 |
| 5723 | CAGAAGUA A UGACUCCA | 1308 | TGGAGTCA GGCTAGCTACAACGA TACTTCTG | 3636 |
| 5726 | AAGUAAUG A CUCCAUAC | 1309 | GTATGCAG GGCTAGCTACAACGA CATTACTT | 3637 |
| 5731 | AUGACUCC A UACAUAUU | 1310 | AATATGTA GGCTAGCTACAACGA GGAGTCAT | 3638 |
| 5733 | GACUCCAU A CAUAUUAU | 1311 | ATAATATG GGCTAGCTACAACGA ATGGAGTC | 3639 |
| 5735 | CUCCAUAC A UAUUAUUU | 1312 | AAATAATA GGCTAGCTACAACGA GTATGGAG | 3640 |
| 5737 | CCAUACAU A UUAUUUAU | 1313 | ATAAATAA GGCTAGCTACAACGA ATGTATGG | 3641 |
| 5740 | UACAUAUU A UUUAUUUC | 1314 | GAAATAAA GGCTAGCTACAACGA AATATGTA | 3642 |
| 5744 | UAUUAUUU A UUUCUAUA | 1315 | TATAGAAA GGCTAGCTACAACGA AAATAATA | 3643 |
| 5750 | UUAUUUCU A UAACUACA | 1316 | TGTAGTTA GGCTAGCTACAACGA AGAAATAA | 3644 |
| 5753 | UUUCUAUA A CUACAUUU | 1317 | AAATGTAG GGCTAGCTACAACGA TATAGAAA | 3645 |
| 5756 | CUAUAACU A CAUUUAAA | 1318 | TTTAAATG GGCTAGCTACAACGA AGTTATAG | 3646 |
| 5758 | AUAACUAC A UUUAAAUC | 1319 | GATTTAAA GGCTAGCTACAACGA GTAGTTAT | 3647 |
| 5764 | ACAUUUAA A UCAUUACC | 1320 | GGTAATGA GGCTAGCTACAACGA TTAAATGT | 3648 |
| 5767 | UUUAAAUC A UUACCAGG | 1321 | CCTGGTAA GGCTAGCTACAACGA GATTTAAA | 3649 |

Input Sequence = NM_004985.
Cut Site = R/Y
Arm Length = 8.
Core Sequence = GGCTAGCTACAACGA
NM_004985 (Homo sapiens v-Ki-ras2 Kirsten rat sarcoma 2 viral oncogene homolog (KRas2), mRNA; 5775 nt)

TABLE III
Human H-Ras DNAzyme and Target molecules

| | | | | |
|---|---|---|---|---|
| | | Seq | | Seq |
| Pos | Substrate | ID | DNAzyme | ID |

| | | | | |
|---|---|---|---|---|
| 9 | GGAUCCCA G CCUUUCCC | 1322 | GGGAAAGG GGCTAGCTACAACGA TGGGATCC | 3650 |
| 20 | UUUCCCCA G CCCGUAGC | 1323 | GCTACGGG GGCTAGCTACAACGA TGGGGAAA | 3651 |
| 24 | CCCAGCCC G UAGCCCCG | 1324 | CGGGGCTA GGCTAGCTACAACGA GGGCTGGG | 3652 |
| 27 | AGCCCGUA G CCCCGGGA | 1325 | TCCCGGGG GGCTAGCTACAACGA TACGGGCT | 3653 |
| 35 | GCCCCGGG A CCUCCGCG | 1326 | CGCGGAGG GGCTAGCTACAACGA CCCGGGGC | 3654 |
| 41 | GGACCUCC G CGGUGGGC | 1327 | GCCCACCG GGCTAGCTACAACGA GGAGGTCC | 3655 |
| 44 | CCUCCGCG G UGGGCGGC | 1328 | GCCGCCCA GGCTAGCTACAACGA CGCGGAGG | 3656 |
| 48 | CGCGGUGG G CGGCGCCG | 1329 | CGGCGCCG GGCTAGCTACAACGA CCACCGCG | 3657 |
| 51 | GGUGGGCG G CGCCGCGC | 1330 | GCGCGGCG GGCTAGCTACAACGA CGCCCACC | 3658 |
| 53 | UGGGCGGC G CCGCGCUG | 1331 | CAGCGCGG GGCTAGCTACAACGA GCCGCCCA | 3659 |
| 56 | GCGGCGCC G CGCUGCCG | 1332 | CGGCAGCG GGCTAGCTACAACGA GGCGCCGC | 3660 |
| 58 | GGCGCCGC G CUGCCGGC | 1333 | GCCGGCAG GGCTAGCTACAACGA GCGGCGCC | 3661 |
| 61 | GCCGCGCU G CCGGCGCA | 1334 | TGCGCCGG GGCTAGCTACAACGA AGCGCGGC | 3662 |
| 65 | CGCUGCCG G CGCAGGGA | 1335 | TCCCTGCG GGCTAGCTACAACGA CGGCAGCG | 3663 |
| 67 | CUGCCGGC G CAGGGAGG | 1336 | CCTCCCTG GGCTAGCTACAACGA GCCGGCAG | 3664 |
| 76 | CAGGGAGG G CCUCUGGU | 1337 | ACCAGAGG GGCTAGCTACAACGA CCTCCCTG | 3665 |
| 83 | GGCCUCUG G UGCACCGG | 1338 | CCGGTGCA GGCTAGCTACAACGA CAGAGGCC | 3666 |
| 85 | CCUCUGGU G CACCGGCA | 1339 | TGCCGGTG GGCTAGCTACAACGA ACCAGAGG | 3667 |
| 87 | UCUGGUGC A CCGGCACC | 1340 | GGTGCCGG GGCTAGCTACAACGA GCACCAGA | 3668 |
| 91 | GUGCACCG G CACCGCUG | 1341 | CAGCGGTG GGCTAGCTACAACGA CGGTGCAC | 3669 |
| 93 | GCACCGGC A CCGCUGAG | 1342 | CTCAGCGG GGCTAGCTACAACGA GCCGGTGC | 3670 |
| 96 | CCGGCACC G CUGAGUCG | 1343 | CGACTCAG GGCTAGCTACAACGA GGTGCCGG | 3671 |
| 101 | ACCGCUGA G UCGGGUUC | 1344 | GAACCCGA GGCTAGCTACAACGA TCAGCGGT | 3672 |
| 106 | UGAGUCGG G UUCUCUCG | 1345 | CGAGAGAA GGCTAGCTACAACGA CCGACTCA | 3673 |
| 114 | GUUCUCUC G CCGGCCUG | 1346 | CAGGCCGG GGCTAGCTACAACGA GAGAGAAC | 3674 |
| 118 | UCUCGCCG G CCUGUUCC | 1347 | GGAACAGG GGCTAGCTACAACGA CGGCGAGA | 3675 |
| 122 | GCCGGCCU G UUCCCGGG | 1348 | CCCGGGAA GGCTAGCTACAACGA AGGCCGGC | 3676 |
| 134 | CCGGGAGA G CCCGGGGC | 1349 | GCCCCGGG GGCTAGCTACAACGA TCTCCCGG | 3677 |
| 141 | AGCCCGGG G CCCUGCUC | 1350 | GAGCAGGG GGCTAGCTACAACGA CCCGGGCT | 3678 |
| 146 | GGGGCCCU G CUCGGAGA | 1351 | TCTCCGAG GGCTAGCTACAACGA AGGGCCCC | 3679 |
| 154 | GCUCGGAG A UGCCGCCC | 1352 | GGGCGGCA GGCTAGCTACAACGA CTCCGAGC | 3680 |
| 156 | UCGGAGAU G CCGCCCCC | 1353 | CGGGGCGG GGCTAGCTACAACGA ATCTCCGA | 3681 |
| 159 | GAGAUGCC G CCCCGGGC | 1354 | GCCCGCGG GGCTAGCTACAACGA GGCATCTC | 3682 |
| 166 | CGCCCCGG G CCCCCAGA | 1355 | TCTGCGGG GGCTAGCTACAACGA CCGGGGCG | 3683 |
| 174 | GCCCCCAG A CACCGGCU | 1356 | AGCCGGTG GGCTAGCTACAACGA CTGGGGCC | 3684 |
| 176 | CCCCAGAC A CCGGCUCC | 1357 | GGAGCCGG GGCTAGCTACAACGA GTCTGGGG | 3685 |
| 180 | AGACACCG G CUCCCUGG | 1358 | CCAGGGAC GGCTAGCTACAACGA CGCTGTCT | 3686 |
| 188 | GCUCCCUG G CCUUCCUC | 1359 | GAGGAAGG GGCTAGCTACAACGA CAGGGAGC | 3687 |
| 199 | UUCCUCGA G CAACCCCG | 1360 | CGGGGTTG GGCTAGCTACAACGA TCGAGGAA | 3688 |
| 202 | CUCGACCA A CCCCGAGC | 1361 | GCTCGGGG GGCTAGCTACAACGA TGCTCGAC | 3689 |
| 209 | AACCCCGA G CUCGGCUC | 1362 | GAGCCGAG GGCTAGCTACAACGA TCGGGCTT | 3690 |
| 214 | CGACCUCG G CUCCGGUC | 1363 | GACCGGAG GGCTAGCTACAACGA CGAGCTCG | 3691 |
| 220 | CGGCUCCG G UCUCCAGC | 1364 | GCTGGAGA GGCTAGCTACAACGA CGGAGCCG | 3692 |
| 227 | GGUCUCCA G CCAAGCCC | 1365 | GGGCTTGG GGCTAGCTACAACGA TGGACACC | 3693 |
| 232 | CCAGCCAA G CCCAACCC | 1366 | GGGTTGGG GGCTAGCTACAACGA TTGGCTGG | 3694 |
| 237 | CAAGCCCA A CCCCGAGA | 1367 | TCTCGGGG GGCTAGCTACAACGA TGGGCTTG | 3695 |
| 247 | CCCGAGAG G CCGCGGCC | 1368 | GGCCGCGG GGCTAGCTACAACGA CTCTCGGG | 3696 |
| 250 | GAGAGGCC G CGGCCCUA | 1369 | TAGGGCCG GGCTAGCTACAACGA GGCCTCTC | 3697 |
| 253 | AGGCCGCG G CCCUACUG | 1370 | CAGTAGGG GGCTAGCTACAACGA CGCGGCCT | 3698 |
| 258 | GCGGCCCU A CUGGCUCC | 1371 | GGAGCCAG GGCTAGCTACAACGA AGGGCCGC | 3699 |
| 262 | CCCUACUG G CUCCGCCU | 1372 | AGGCGGAG GGCTAGCTACAACGA CAGTAGGG | 3700 |
| 267 | CUGGCUCC G CCUCCCCC | 1373 | GCGGGACG GGCTAGCTACAACGA GGAGCCAG | 3701 |
| 274 | CGCCUCCC G CGUUGCUC | 1374 | GAGCAACG GGCTAGCTACAACGA GGGAGGCG | 3702 |
| 276 | CCUCCCGC G UUGCUCCC | 1375 | GGGAGCAA GGCTAGCTACAACGA GCGGGAGG | 3703 |
| 279 | CCCGCGUU G CUCCCGGA | 1376 | TCCGGGAG GGCTAGCTACAACGA AACGCGGG | 3704 |
| 289 | UCCCGGAA G CCCCGCCC | 1377 | GGGCGGGG GGCTAGCTACAACGA TTCCGGGA | 3705 |
| 294 | GAACCCCC G CCCGACCG | 1378 | CGGTCGGG GGCTAGCTACAACGA GGGGCTTC | 3706 |
| 299 | CCCGCCCC A CCGCGGCU | 1379 | AGCCGCGG GGCTAGCTACAACGA CGGGCGGG | 3707 |
| 302 | GCCCGACC G CGGCUCCU | 1380 | AGGAGCCG GGCTAGCTACAACGA GGTCGGGC | 3708 |
| 305 | CGACCGCG G CUCCUGAC | 1381 | GTCAGGAC GGCTAGCTACAACGA CGCGGTCG | 3709 |
| 312 | GGCUCCUG A CAGACGGG | 1382 | CCCGTCTG GGCTAGCTACAACGA CAGGACCC | 3710 |
| 316 | CCUGACAG A CGGGCCGC | 1383 | GCGGCCCG GGCTAGCTACAACGA CTGTCAGG | 3711 |
| 320 | ACAGACGG G CCGCUCAG | 1384 | CTGAGCGG GGCTAGCTACAACGA CCGTCTGT | 3712 |
| 323 | CACGGGCC G CUCAGCCA | 1385 | TGGCTGAG GGCTAGCTACAACGA GGCCCGTC | 3713 |
| 328 | GCCCCUCA G CCAACCGG | 1386 | CCGGTTGG GGCTAGCTACAACGA TGAGCGGC | 3714 |
| +G | | | | |
| 332 | CUCAGCCA A CCGGGGUG | 1387 | CACCCCGG GGCTAGCTACAACGA TGGCTGAG | 3715 |
| 338 | CAACCGGG G UGGGGCGG | 1388 | CCGCCCCA GGCTAGCTACAACGA CCCGGTTG | 3716 |
| 343 | GGGCUGGG G CGGGGCCC | 1389 | GGGCCCCG GGCTAGCTACAACGA CCCACCCC | 3717 |
| 348 | GGGGCGGG G CCCGAUGG | 1390 | CCATCGGG GGCTAGCTACAACGA CCCGCCCC | 3718 |
| 353 | GGGGCCCG A UGGCGCGC | 1391 | GCGCGCCA GGCTAGCTACAACGA CGGGCCCC | 3719 |
| 356 | GCCCGAUG G CGCGCAGC | 1392 | GCTGCGCG GGCTAGCTACAACGA CATCCGGC | 3720 |
| 358 | CCGAUGGC G CGCAGCCA | 1393 | TGGCTGCG GGCTAGCTACAACGA GCCATCGG | 3721 |
| 360 | GAUGGCGC G CAGCCAAU | 1394 | ATTGGCTG GGCTAGCTACAACGA GCGCCATC | 3722 |
| 363 | GGCGCGCA G CCAAUGGU | 1395 | ACCATTGG GGCTAGCTACAACGA TGCGCGCC | 3723 |
| 367 | CGCAGCCA A UGGUAGGC | 1396 | GCCTACCA GGCTAGCTACAACGA TGGCTGCG | 3724 |
| 370 | AGCCAAUG G UAGGCCGC | 1397 | GCGGCCTA GGCTAGCTACAACGA CATTGGCT | 3725 |
| 374 | AAUGGUAG G CCGCGCCU | 1398 | AGGCGCGG GGCTAGCTACAACGA CTACCATT | 3726 |
| 377 | GGUAGGCC G CGCCUGGC | 1399 | GCCAGGCG GGCTAGCTACAACGA GGCCTACC | 3727 |
| 379 | UAGGCCGC G CCUGGCAG | 1400 | CTGCCAGG GGCTAGCTACAACGA GCGCCCTA | 3728 |
| 384 | CGCGCCUG G CAGACGGA | 1401 | TCCGTCTG GGCTAGCTACAACGA CAGGCGCG | 3729 |
| 388 | CCUGGCAG A CGGACGGG | 1402 | CCCGTCCG GGCTAGCTACAACGA CTGCCAGG | 3730 |
| 392 | GCAGACGG A CGGGCGCG | 1403 | CGCGCCCG GGCTAGCTACAACGA CCGTCTGC | 3731 |
| 396 | ACGGACGG G CGCGGGGC | 1404 | GCCCCGCG GGCTAGCTACAACGA CCGTCCGT | 3732 |
| 398 | GGACGGGC G CGGGGCGG | 1405 | CCGCCCCG GGCTAGCTACAACGA GCCCGTCC | 3733 |
| 403 | GGCGCGGG G CGGGGCGU | 1406 | ACGCCCCG GGCTAGCTACAACGA CCCGCGCC | 3734 |
| 408 | GGGGCGGG G CGUGCGCA | 1407 | TGCGCACG GGCTAGCTACAACGA CCCGCCCC | 3735 |
| 410 | GGCGGGGC G UGCGCAGG | 1408 | CCTGCGCA GGCTAGCTACAACGA GCCCCGCC | 3736 |
| 412 | CGGGGCGU G CGCAGGCC | 1409 | GGCCTGCG GGCTAGCTACAACGA ACGCCCCG | 3737 |
| 414 | GGGCGUGC G CAGGCCCG | 1410 | CGGGCCTG GGCTAGCTACAACGA GCACGCCC | 3738 |
| 418 | GUGCGCAG G CCCGCCCG | 1411 | CGGGCGGG GGCTAGCTACAACGA CTGCGCAC | 3739 |
| 422 | GCAGGCCC G CCCGAGUC | 1412 | CACTCGGG GGCTAGCTACAACGA GGGCCTGC | 3740 |
| 428 | CCGCCCGA G UCUCCGCC | 1413 | GGCGGAGA GGCTAGCTACAACGA TCGGGCGG | 3741 |
| 434 | GAGUCUCC G CCGCCCGU | 1414 | ACGGGCGG GGCTAGCTACAACGA GGAGACTC | 3742 |
| 437 | UCUCCGCC G CCCGUGCC | 1415 | GGCACGGG GGCTAGCTACAACGA GGCGGAGA | 3743 |
| 441 | CGCCGCCC G UGCCCUGC | 1416 | GCAGGGCA GGCTAGCTACAACGA GGGCGGCG | 3744 |
| 443 | CCGCCCGU G CCCUGCGC | 1417 | GCGCAGGG GGCTAGCTACAACGA ACGGGCGG | 3745 |
| 448 | CGUGCCCU G CGCCCGCA | 1418 | TGCGGGCG GGCTAGCTACAACGA AGGGCACG | 3746 |
| 450 | UGCCCUGC G CCCGCAAC | 1419 | GTTGCGGG GGCTAGCTACAACGA GCAGGGCA | 3747 |
| 454 | CUGCGCCC G CAACCCGA | 1420 | TCGGGTTG GGCTAGCTACAACGA GGGCGCAG | 3748 |
| 457 | CGCCCGCA A CCCGAGCC | 1421 | GGCTCGGG GGCTAGCTACAACGA TGCGGGCG | 3749 |
| 463 | CAACCCGA G CCGCACCC | 1422 | GGGTGCGG GGCTAGCTACAACGA TCGGGTTG | 3750 |
| 466 | CCCGAGCC G CACCCGCC | 1423 | GGCGGGTG GGCTAGCTACAACGA GGCTCGGG | 3751 |
| 468 | CGAGCCGC A CCCGCCGC | 1424 | GCGGCGGG GGCTAGCTACAACGA GCGGCTCG | 3752 |
| 472 | CCGCACCC G CCGCGGAC | 1425 | GTCCGCGG GGCTAGCTACAACGA GGGTGCGG | 3753 |
| 475 | CACCCGCC G CGGACGGA | 1426 | TCCGTCCG GGCTAGCTACAACGA GGCGGGTG | 3754 |
| 479 | CGCCGCGG A CGGAGCCC | 1427 | GGGCTCCG GGCTAGCTACAACGA CCGCGGCG | 3755 |
| 484 | CGGACGGA G CCCAUGCG | 1428 | CGCATGGG GGCTAGCTACAACGA TCCGTCCG | 3756 |
| 488 | CGGAGCCC A UGCGCGGG | 1429 | CCCGCGCA GGCTAGCTACAACGA GGGCTCCG | 3757 |
| 490 | GAGCCCAU G CGCGGGGC | 1430 | GCCCCGCG GGCTAGCTACAACGA ATGGGCTC | 3758 |
| 492 | GCCCAUGC G CGGGGCGA | 1431 | TCGCCCCG GGCTAGCTACAACGA GCATGGGC | 3759 |
| 497 | UGCGCGGG G CGAACCGC | 1432 | GCGGTTCG GGCTAGCTACAACGA CCCGCGCA | 3760 |
| 501 | CGGGGCGA A CCGCGCGC | 1433 | GCGCGCGG GGCTAGCTACAACGA TCGCCCCG | 3761 |
| 504 | GGCGAACC G CGCGCCCC | 1434 | GGGGCGCG GGCTAGCTACAACGA GGTTCGCC | 3762 |
| 506 | CGAACCGC G CGCCCCCG | 1435 | CGGGGGCG GGCTAGCTACAACGA GCGGTTCG | 3763 |
| 508 | AACCGCGC G CCCCCGCC | 1436 | GGCGGGGG GGCTAGCTACAACGA GCGCGGTT | 3764 |
| 514 | GCGCCCCC G CCCCCGCC | 1437 | GGCGGGGG GGCTAGCTACAACGA GGGGGCGC | 3765 |
| 520 | CCGCCCCC G CCCCGCCC | 1438 | GGGCGGGG GGCTAGCTACAACGA GGGGGCGG | 3766 |
| 525 | CCCGCCCC G CCCCGGCC | 1439 | GGCCGGGG GGCTAGCTACAACGA GGGGCGGG | 3767 |
| 531 | CCGCCCCG G CCUCGGCC | 1440 | GGCCGAGG GGCTAGCTACAACGA CGGGGCGG | 3768 |
| 537 | CGGCCUCG G CCCCGGCC | 1441 | GGCCGGGG GGCTAGCTACAACGA CGAGGCCG | 3769 |
| 543 | CGGCCCCG G CCCUGGCC | 1442 | GGCCAGGG GGCTAGCTACAACGA CGGGGCCG | 3770 |
| 549 | CGGCCCUC G CCCCGGGG | 1443 | CCCCGGGG GGCTAGCTACAACGA CAGGGCCG | 3771 |
| 558 | CCCCGGGG G CAGUCGCG | 1444 | CGCGACTG GGCTAGCTACAACGA CCCCGGGG | 3772 |
| 561 | CGGGGGCA G UCGCGCCU | 1445 | AGGCGCGA GGCTAGCTACAACGA TGCCCCCG | 3773 |
| 564 | GGGCAGUC G CGCCUGUG | 1446 | CACAGGCG GGCTAGCTACAACGA GACTGCCC | 3774 |
| 566 | GCAGUCGC G CCUGUGAA | 1447 | TTCACAGG GGCTAGCTACAACGA GGCACTGC | 3775 |
| 570 | UCGCGCCU G UGAACGGU | 1448 | ACCGTTCA GGCTAGCTACAACGA AGGCGCGA | 3776 |
| 574 | GCCUGUGA A CGGUGAGU | 1449 | ACTCACCG GGCTAGCTACAACGA TCACAGGC | 3777 |
| 577 | UGUGAACC G UGAGUGCG | 1450 | CGCACTCA GGCTAGCTACAACGA CGTTCACA | 3778 |
| 581 | AACGGUGA G UGCGGGCA | 1451 | TGCCCGCA GGCTAGCTACAACGA TCACCGTT | 3779 |
| 583 | CGGUGAGU G CGGGCAGG | 1452 | CCTGCCCG GGCTAGCTACAACGA ACTCACCG | 3780 |
| 587 | GAGUGCGG G CAGGGAUC | 1453 | GATCCCTG GGCTAGCTACAACGA CCGCACTC | 3781 |
| 593 | GGGCAGGG A UCGGCCGG | 1454 | CCGGCCGA GGCTAGCTACAACGA CCCTGCCC | 3782 |
| 597 | AGGGAUCG G CCGGGCCG | 1455 | CGGCCCGG GGCTAGCTACAACGA CGATCCCT | 3783 |
| 602 | UCGGCCGG G CCGCGCGC | 1456 | GCGCGCGG GGCTAGCTACAACGA CCGGCCGA | 3784 |
| 605 | GCCGGGCC G CGCGCCCU | 1457 | AGGGCGCG GGCTAGCTACAACGA GGCCCGGC | 3785 |
| 607 | CGGGCCGC G CGCCCUCC | 1458 | GGAGGGCG GGCTAGCTACAACGA GCGGCCCG | 3786 |
| 609 | GGCCGCGC G CCCUCCUC | 1459 | GAGGAGGG GGCTAGCTACAACGA GCGCGGCC | 3787 |
| 618 | CCCUCCUC G CCCCCAGG | 1460 | CCTGGGGG GGCTAGCTACAACGA GAGGAGGG | 3788 |
| 626 | GCCCCCAG G CGGCAGCA | 1461 | TGCTGCCG GGCTAGCTACAACGA CTGGGGGC | 3789 |
| 629 | CCCAGGCG G CAGCAAUA | 1462 | TATTGCTG GGCTAGCTACAACGA CGCCTGGG | 3790 |
| 632 | AGGCGGCA G CAAUACGC | 1463 | GCGTATTG GGCTAGCTACAACGA TGCCGCCT | 3791 |
| 635 | CGGCAGCA A UACGCGCG | 1464 | CGCGCGTA GGCTAGCTACAACGA TGCTGCCG | 3792 |
| 637 | GCAGCAAU A CGCGCGGC | 1465 | GCCGCGCG GGCTAGCTACAACGA ATTGCTGC | 3793 |
| 639 | AGCAAUAC G CGCGGCGC | 1466 | GCGCCGCG GGCTAGCTACAACGA GTATTGCT | 3794 |
| 641 | CAAUACGC G CGGCGCGG | 1467 | CCGCGCCG GGCTAGCTACAACGA GCGTATTG | 3795 |
| 644 | UACGCGCG G CGCGGGCC | 1468 | GGCCCGCG GGCTAGCTACAACGA CGCGCGTA | 3796 |
| 646 | CGCGCGGC G CGGGCCGG | 1469 | CCGGCCCG GGCTAGCTACAACGA GCCGCGCG | 3797 |
| 650 | CGGCGCGG G CCGGGGGC | 1470 | GCCCCCGG GGCTAGCTACAACGA CCGCGCCG | 3798 |
| 657 | GGCCGGGG G CGCGGGGC | 1471 | GCCCCGCG GGCTAGCTACAACGA CCCCGGCC | 3799 |
| 659 | CCGGGGGC G CGGGGCCG | 1472 | CGGCCCCG GGCTAGCTACAACGA GCCCCCGG | 3800 |
| 664 | GGCGCGGG G CCGGCGGG | 1473 | CCCGCCGG GGCTAGCTACAACGA CCCGCGCC | 3801 |
| 668 | CGGGGCCG G CGGGCGUA | 1474 | TACGCCCG GGCTAGCTACAACGA CGGCCCCG | 3802 |
| 672 | GCCGGCGG G CGUAAGCG | 1475 | CGCTTACG GGCTAGCTACAACGA CCGCCGGC | 3803 |
| 674 | CGGCGGGC G UAAGCGGC | 1476 | GCCGCTTA GGCTAGCTACAACGA GCCCGCCG | 3804 |
| 678 | GGGCGUAA G CGGCGGCG | 1477 | CGCCGCCG GGCTAGCTACAACGA TTACGCCC | 3805 |
| 681 | CGUAAGCG G CGGCGGCG | 1478 | CGCCGCCG GGCTAGCTACAACGA CGCTTACG | 3806 |
| 684 | AAGCGGCG G CGGCGGCG | 1479 | CGCCGCCG GGCTAGCTACAACGA CGCCGCTT | 3807 |
| 687 | CGGCGGCG G CGGCGGCG | 1480 | CGCCGCCG GGCTAGCTACAACGA CGCCGCCG | 3808 |
| 690 | CGGCGGCG G CGGCGGGU | 1481 | ACCCGCCG GGCTAGCTACAACGA CGCCGCCG | 3809 |
| 693 | CGGCGGCG G CGGGUGGG | 1482 | CCCACCCG GGCTAGCTACAACGA CGCCGCCG | 3810 |
| 697 | GGCGGCGG G UGGGUGGG | 1483 | CCCACCCA GGCTAGCTACAACGA CCGCCGCC | 3811 |
| 701 | GCGGGUGG G UGGGGCCG | 1484 | CGGCCCCA GGCTAGCTACAACGA CCACCCGC | 3812 |
| 706 | UGGGUGGG G CCGGGCGG | 1485 | CCGCCCGG GGCTAGCTACAACGA CCCACCCA | 3813 |
| 711 | GGGGCCGG G CGGGGCCC | 1486 | GGGCCCCG GGCTAGCTACAACGA CCGGCCCC | 3814 |
| 716 | CGGGCGGG G CCCGCGGG | 1487 | CCCGCGGG GGCTAGCTACAACGA CCCGCCCG | 3815 |
| 720 | CGGGGCCC G CGGGCACA | 1488 | TGTGCCCG GGCTAGCTACAACGA GGGCCCCG | 3816 |
| 724 | GCCCGCGG G CACAGGUG | 1489 | CACCTGTG GGCTAGCTACAACGA CCGCGGGC | 3817 |
| 726 | CCGCGGGC A CAGGUGAG | 1490 | CTCACCTG GGCTAGCTACAACGA GCCCGCGG | 3818 |
| 730 | GGGCACAG G UGAGCGGG | 1491 | CCCGCTCA GGCTAGCTACAACGA CTGTGCCC | 3819 |
| 734 | ACAGGUGA G CGGGCGUC | 1492 | GACGCCCG GGCTAGCTACAACGA TCACCTGT | 3820 |
| 738 | GUGAGCGG G CGUCGGGG | 1493 | CCCCGACG GGCTAGCTACAACGA CCGCTCAC | 3821 |
| 740 | GAGCGGGC G UCGGGGGC | 1494 | GCCCCCGA GGCTAGCTACAACGA GCCCGCTC | 3822 |
| 747 | CGUCGGGG G CUGCGGCG | 1495 | CGCCGCAG GGCTAGCTACAACGA CCCCGACG | 3823 |
| 750 | CGGGGGCU G CGGCGGGC | 1496 | GCCCGCCG GGCTAGCTACAACGA AGCCCCCG | 3824 |
| 753 | GGGCUGCG G CGGGCGGG | 1497 | CCCGCCCG GGCTAGCTACAACGA CGCAGCCC | 3825 |
| 757 | UGCGGCGG G CGGGGGCC | 1498 | GGCCCCCG GGCTAGCTACAACGA CCGCCGCA | 3826 |
| 763 | GGGCGGGG G CCCCUUCC | 1499 | GGAAGGGG GGCTAGCTACAACGA CCCCGCCC | 3827 |
| 780 | UCCCUGGG G CCUGCGGG | 1500 | CCCGCAGG GGCTAGCTACAACGA CCCAGGGA | 3828 |
| 784 | UGGGGCCU G CGGGAAUC | 1501 | GATTCCCG GGCTAGCTACAACGA AGGCCCCA | 3829 |
| 790 | CUGCGGGA A UCCGGGCC | 1502 | GGCCCGGA GGCTAGCTACAACGA TCCCGCAG | 3830 |
| 796 | GAAUCCGG G CCCCACCC | 1503 | GGGTGGGG GGCTAGCTACAACGA CCGGATTC | 3831 |
| 801 | CGGGCCCC A CCCGUGGC | 1504 | GCCACGGG GGCTAGCTACAACGA GGGGCCCG | 3832 |
| 805 | CCCCACCC G UGGCCUCG | 1505 | CGAGGCCA GGCTAGCTACAACGA GGGTGGGG | 3833 |
| 808 | CACCCGUG G CCUCGCGC | 1506 | GCGCGAGG GGCTAGCTACAACGA CACGGGTG | 3834 |
| 813 | GUGGCCUC G CGCUGGGC | 1507 | GCCCAGCG GGCTAGCTACAACGA GAGGCCAC | 3835 |
| 815 | GGCCUCGC G CUGGGCAC | 1508 | GTGCCCAG GGCTAGCTACAACGA GCGAGGCC | 3836 |
| 820 | CGCGCUGG G CACGGUCC | 1509 | GGACCGTG GGCTAGCTACAACGA CCAGCGCG | 3837 |
| 822 | CGCUGGGC A CGGUCCCC | 1510 | GGGGACCG GGCTAGCTACAACGA GCCCAGCG | 3838 |
| 825 | UGGGCACG G UCCCCACG | 1511 | CGTGGGGA GGCTAGCTACAACGA CGTGCCCA | 3839 |
| 831 | CGGUCCCC A CGCCGGCG | 1512 | CGCCGGCG GGCTAGCTACAACGA GGGGACCG | 3840 |
| 833 | GUCCCCAC G CCGGCGUA | 1513 | TACGCCGG GGCTAGCTACAACGA GTGGGGAC | 3841 |
| 837 | CCACGCCG G CGUACCCG | 1514 | CGGGTACG GGCTAGCTACAACGA CGGCGTGG | 3842 |
| 839 | ACGCCGGC G UACCCGGG | 1515 | CCCGGGTA GGCTAGCTACAACGA GCCGGCGT | 3843 |
| 841 | GCCGGCGU A CCCGGGAG | 1516 | CTCCCGGG GGCTAGCTACAACGA ACGCCGGC | 3844 |
| 849 | ACCCGGGA G CCUCGGGC | 1517 | GCCCGAGG GGCTAGCTACAACGA TCCCGGGT | 3845 |
| 856 | AGCCUCGG G CCCGGCGC | 1518 | GCGCCGGG GGCTAGCTACAACGA CCGAGGCT | 3846 |
| 861 | CGGGCCCG G CGCCCUCA | 1519 | TGAGGGCG GGCTAGCTACAACGA CGGGCCCG | 3847 |
| 863 | GGCCCGGC G CCCUCACA | 1520 | TGTGAGGG GGCTAGCTACAACGA GCCGGGCC | 3848 |
| 869 | GCGCCCUC A CACCCGGG | 1521 | CCCGGGTG GGCTAGCTACAACGA GAGGGCGC | 3849 |
| 871 | GCCCUCAC A CCCGGGGG | 1522 | CCCCCGGG GGCTAGCTACAACGA GTGAGGGC | 3850 |
| 879 | ACCCGGGG G CGUCUGGG | 1523 | CCCAGACG GGCTAGCTACAACGA CCCCGGGT | 3851 |
| 881 | CCGGGGGC G UCUGGGAG | 1524 | CTCCCAGA GGCTAGCTACAACGA GCCCCCGG | 3852 |
| 893 | GGGAGGAG G CGGCCGCG | 1525 | CGCGGCCG GGCTAGCTACAACGA CTCCTCCC | 3853 |
| 896 | AGGAGGCG G CCGCGGCC | 1526 | GGCCGCGG GGCTAGCTACAACGA CGCCTCCT | 3854 |
| 899 | AGGCGGCC G CGGCCACG | 1527 | CGTGGCCG GGCTAGCTACAACGA GGCCGCCT | 3855 |
| 902 | CGGCCGCG G CCACGGCA | 1528 | TGCCGTGG GGCTAGCTACAACGA CGCGGCCG | 3856 |
| 905 | CCGCGGCC A CGGCACGC | 1529 | GCGTGCCG GGCTAGCTACAACGA GGCCGCGG | 3857 |
| 908 | CGGCCACG G CACGCCCG | 1530 | CGGGCGTG GGCTAGCTACAACGA CGTGGCCG | 3858 |
| 910 | GCCACGGC A CGCCCGGG | 1531 | CCCGGGCG GGCTAGCTACAACGA GCCGTGGC | 3859 |
| 912 | CACGGCAC G CCCGGGCA | 1532 | TGCCCGGG GGCTAGCTACAACGA GTGCCGTG | 3860 |
| 918 | ACGCCCGG G CACCCCCG | 1533 | CGGGGGTG GGCTAGCTACAACGA CCGGGCGT | 3861 |
| 920 | GCCCGGGC A CCCCCGAU | 1534 | ATCGGGGG GGCTAGCTACAACGA GCCCGGGC | 3862 |
| 927 | CACCCCCG A UUCAGCAU | 1535 | ATGCTGAA GGCTAGCTACAACGA CGGGGGTG | 3863 |
| 932 | CCGAUUCA G CAUCACAG | 1536 | CTGTGATG GGCTAGCTACAACGA TGAATCGG | 3864 |
| 934 | GAUUCAGC A UCACAGGU | 1537 | ACCTGTGA GGCTAGCTACAACGA GCTGAATC | 3865 |
| 937 | UCAGCAUC A CAGGUCGC | 1538 | GCGACCTG GGCTAGCTACAACGA GATGCTGA | 3866 |
| 941 | CAUCACAG G UCGCGGAC | 1539 | GTCCGCGA GGCTAGCTACAACGA CTGTGATG | 3867 |
| 944 | CACAGGUC G CGGACCAG | 1540 | CTGGTCCG GGCTAGCTACAACGA GACCTGTG | 3868 |
| 948 | GGUCGCGG A CCAGGCCG | 1541 | CGGCCTGG GGCTAGCTACAACGA CCGCGACC | 3869 |
| 953 | CGGACCAG G CCGGGGGC | 1542 | GCCCCCGG GGCTAGCTACAACGA CTGGTCCG | 3870 |
| 960 | GGCCGGGG G CCUCAGCC | 1543 | GGCTGAGG GGCTAGCTACAACGA CCCCGGCC | 3871 |
| 966 | GGGCCUCA G CCCCAGUG | 1544 | CACTGGGG GGCTAGCTACAACGA TGAGGCCC | 3872 |
| 972 | CAGCCCCA G UGCCUUUU | 1545 | AAAAGGCA GGCTAGCTACAACGA TGGGGCTG | 3873 |
| 974 | GCCCCAGU G CCUUUUCC | 1546 | GGAAAAGG GGCTAGCTACAACGA ACTGGGGC | 3874 |
| 991 | CUCUCCGG G UCUCCCGC | 1547 | GCGGGAGA GGCTAGCTACAACGA CCGGAGAG | 3875 |
| 998 | GGUCUCCC G CGCCGCUU | 1548 | AAGCGGCG GGCTAGCTACAACGA GGGAGACC | 3876 |
| 1000 | UCUCCCGC G CCGCUUCU | 1549 | ACAAGCGG GGCTAGCTACAACCA GCGGGAGA | 3877 |
| 1003 | CCCGCCCC G CUUCUCGG | 1550 | CCGAGAAG GGCTAGCTACAACGA GGCGCGGG | 3878 |
| 1011 | GCUUCUCG G CCCCUUCC | 1551 | GGAAGGCG GGCTAGCTACAACGA CGAGAAGC | 3879 |
| 1021 | CCCUUCCU G UCGCUCAG | 1552 | CTCAGCGA GGCTAGCTACAACGA AGGAAGGG | 3880 |
| 1024 | UUCCUGUC G CUCAGUCC | 1553 | GGACTGAG GGCTAGCTACAACGA GACAGGAA | 3881 |
| 1029 | GUCGCUCA G UCCCUGCU | 1554 | AGCAGGGA GGCTAGCTACAACGA TGAGCGAC | 3882 |
| 1035 | CAGUCCCU G CUUCCCAG | 1555 | CTGGGAAG GGCTAGCTACAACGA AGGCACTG | 3883 |
| 1046 | UCCCAGGA G CUCCUCUG | 1556 | CAGAGGAG GGCTAGCTACAACGA TCCTGGGA | 3884 |
| 1054 | GCUCCUCU G UCUUCUCC | 1557 | GCAGAAGA GGCTAGCTACAACGA AGAGGAGC | 3885 |
| 1064 | CUUCUCCA G CUUUCUGU | 1558 | ACAGAAAG GGCTAGCTACAACGA TGGAGAAG | 3886 |
| 1071 | AGCUUUCU G UGGCUGAA | 1559 | TTCAGCCA GGCTAGCTACAACGA AGAAAGCT | 3887 |
| 1074 | UUUCUGUG G CUGAAAGA | 1560 | TCTTTCAG GGCTAGCTACAACGA CACAGAAA | 3888 |
| 1082 | GCUGAAAG A UGCCCCCG | 1561 | CGGGGGCA GGCTAGCTACAACGA CTTTCAGC | 3889 |
| 1084 | UGAAAGAU G CCCCCGGU | 1562 | ACCGGGGG GGCTAGCTACAACGA ATCTTTCA | 3890 |
| 1091 | UGCCCCCG G UUCCCCGC | 1563 | GCGGGGAA GGCTAGCTACAACGA CGGGGGCA | 3891 |
| 1098 | GGUUCCCC G CCGGGGGU | 1564 | ACCCCCGG GGCTAGCTACAACGA GGGGAACC | 3892 |
| 1105 | CGCCGGGG G UGCGGGGC | 1565 | GCCCCGCA GGCTAGCTACAACGA CCCCGGCG | 3893 |
| 1107 | CCGGGGGU G CGGGGCGC | 1566 | GCGCCCCG GGCTAGCTACAACGA ACCCCCGG | 3894 |
| 1112 | GGUGCGGG G CGCUGCCC | 1567 | GGGCAGCG GGCTAGCTACAACGA CCCGCACC | 3895 |
| 1114 | UGCGGGGC G CUGCCCGG | 1568 | CCGGGCAG GGCTAGCTACAACGA GCCCCGCA | 3896 |
| 1117 | GGGGCGCU G CCCGGGUC | 1569 | GACCCGGG GGCTAGCTACAACGA AGCGCCCC | 3897 |
| 1123 | CUGCCCGG G UCUGCCCU | 1570 | AGGGCAGA GGCTAGCTACAACGA CCGGGCAG | 3898 |
| 1127 | CCGGGUCU G CCCUCCCC | 1571 | GGGGAGGG GGCTAGCTACAACGA AGACCCGG | 3899 |
| 1139 | UCCCCUCG G CGGCGCCU | 1572 | AGGCGCCG GGCTAGCTACAACGA CGAGGGGA | 3900 |
| 1142 | CCUCGGCG G CGCCUAGU | 1573 | ACTAGGCG GGCTAGCTACAACGA CGCCGAGG | 3901 |
| 1144 | UCGGCGGC G CCUAGUAC | 1574 | GTACTAGG GGCTAGCTACAACGA GCCGCCGA | 3902 |
| 1149 | GGCGCCUA G UACGCAGU | 1575 | ACTGCGTA GGCTAGCTACAACGA TAGGCGCC | 3903 |
| 1151 | CGCCUAGU A CGCAGUAG | 1576 | CTACTGCG GGCTAGCTACAACGA ACTAGGCG | 3904 |
| 1153 | CCUAGUAC G CAGUAGGC | 1577 | GCCTACTG GGCTAGCTACAACGA GTACTAGG | 3905 |
| 1156 | AGUACGCA G UAGGCGCU | 1578 | AGCGCCTA GGCTAGCTACAACGA TGCGTACT | 3906 |
| 1160 | CGCAGUAG G CGCUCAGC | 1579 | GCTGAGCG GGCTAGCTACAACGA CTACTGCG | 3907 |
| 1162 | CAGUAGGC G CUCAGCAA | 1580 | TTGCTGAG GGCTAGCTACAACGA GCCTACTG | 3908 |
| 1167 | GGCGCUCA G CAAAUACU | 1581 | AGTATTTG GGCTAGCTACAACGA TGAGCGCC | 3909 |
| 1171 | CUCAGCAA A UACUUCUC | 1582 | GACAAGTA GGCTACCTACAACGA TTGCTGAG | 3910 |
| 1173 | CAGCAAAU A CUUGUCGG | 1583 | CCGACAAG GGCTAGCTACAACGA ATTTGCTG | 3911 |
| 1177 | AAAUACUU G UCGGAGGC | 1584 | GCCTCCGA GGCTAGCTACAACGA AAGTATTT | 3912 |
| 1184 | UCUCGGAG G CACCAGCG | 1585 | CGCTGGTG GGCTAGCTACAACGA CTCCGACA | 3913 |
| 1186 | UCGGAGGC A CCAGCGCC | 1586 | GGCGCTGG GGCTAGCTACAACGA GCCTCCGA | 3914 |
| 1190 | AGGCACCA G CGCCGCGG | 1587 | CCGCGGCG GGCTAGCTACAACGA TGGTGCCT | 3915 |
| 1192 | GCACCAGC G CCGCGGGG | 1588 | CCCCGCGG GGCTAGCTACAACGA GCTGGTGC | 3916 |
| 1195 | CCAGCGCC G CGGGGCCU | 1589 | AGGCCCCG GGCTAGCTACAACGA GGCGCTGG | 3917 |
| 1200 | GCCGCGGG G CCUGCAGG | 1590 | CCTGCAGG GGCTAGCTACAACGA CCCGCGGC | 3918 |
| 1204 | CGGGGCCU G CAGGCUGG | 1591 | CCAGCCTG GGCTAGCTACAACGA AGGCCCCG | 3919 |
| 1208 | GCCUGCAG G CUGGCACU | 1592 | AGTGCCAG GGCTAGCTACAACGA CTGCAGGC | 3920 |
| 1212 | GCAGGCUG G CACUAGCC | 1593 | GGCTAGTG GGCTAGCTACAACGA CAGCCTGC | 3921 |
| 1214 | AGGCUGGC A CUAGCCUG | 1594 | CAGGCTAG GGCTAGCTACAACGA GCCAGCCT | 3922 |
| 1218 | UGGCACUA G CCUGCCCG | 1595 | CGGGCAGG GGCTAGCTACAACGA TAGTGCCA | 3923 |
| 1222 | ACUAGCCU G CCCGGGCA | 1596 | TGCCCGGG GGCTAGCTACAACGA AGGCTAGT | 3924 |
| 1228 | CUGCCCGG G CACGCCGU | 1597 | ACGGCGTG GGCTAGCTACAACGA CCGGGCAG | 3925 |
| 1230 | GCCCGGGC A CGCCGUGG | 1598 | CCACGGCG GGCTAGCTACAACGA GCCCGGGC | 3926 |
| 1232 | CCGGGCAC G CCGUGGCG | 1599 | CGCCACGG GGCTAGCTACAACGA GTGCCCGG | 3927 |
| 1235 | GGCACGCC G UGGCGCGC | 1600 | GCGCGCCA GGCTAGCTACAACGA GGCGTGCC | 3928 |
| 1238 | ACGCCGUG G CGCGCUCC | 1601 | GGAGCGCG GGCTAGCTACAACGA CACGGCGT | 3929 |
| 1240 | GCCGUGGC G CGCUCCGC | 1602 | GCGGAGCG GGCTAGCTACAACGA GCCACGGC | 3930 |
| 1242 | CGUGGCGC G CUCCGCCG | 1603 | CGGCGGAG GGCTAGCTACAACGA GCGCCACG | 3931 |
| 1247 | CGCGCUCC G CCGUGGCC | 1604 | GGCCACGG GGCTAGCTACAACGA GGAGCGCG | 3932 |
| 1250 | GCUCCGCC G UGGCCAGA | 1605 | TCTGGCCA GGCTAGCTACAACGA GGCGGAGC | 3933 |
| 1253 | CCGCCGUG G CCAGACCU | 1606 | AGGTCTGG GGCTAGCTACAACGA CACGGCGG | 3934 |
| 1258 | GUGGCCAG A CCUGUUCU | 1607 | AGAACAGG GGCTAGCTACAACGA CTGGCCAC | 3935 |
| 1262 | CCAGACCU G UUCUGGAG | 1608 | CTCCAGAA GGCTAGCTACAACGA AGGTCTGG | 3936 |
| 1272 | UCUGGAGG A CGGUAACC | 1609 | GGTTACCG GGCTAGCTACAACGA CCTCCAGA | 3937 |
| 1275 | GGAGGACG G UAACCUCA | 1610 | TGAGGTTA GGCTAGCTACAACGA CGTCCTCC | 3938 |
| 1278 | GGACGGUA A CCUCAGCC | 1611 | GGCTGAGG GGCTAGCTACAACGA TACCGTCC | 3939 |
| 1284 | UAACCUCA G CCCUCGGG | 1612 | CCCGAGGG GGCTAGCTACAACGA TGAGGTTA | 3940 |
| 1292 | GCCCUCGG G CGCCUCCC | 1613 | GGGAGGCG GGCTAGCTACAACGA CCGAGGGC | 3941 |
| 1294 | CCUCGGGC G CCUCCCUU | 1614 | AAGGGAGG GGCTAGCTACAACGA GCCCGAGG | 3942 |
| 1305 | UCCCUUUA G CCUUUCUG | 1615 | CAGAAAGG GGCTAGCTACAACGA TAAAGGGA | 3943 |
| 1313 | GCCUUUCU G CCGACCCA | 1616 | TGGGTCGG GGCTAGCTACAACGA AGAAAGGC | 3944 |
| 1317 | UUCUGCCG A CCCAGCAG | 1617 | CTGCTGGG GGCTAGCTACAACGA CGGCAGAA | 3945 |
| 1322 | CCGACCCA G CAGCUUCU | 1618 | AGAAGCTG GGCTAGCTACAACGA TGGGTCGG | 3946 |
| 1325 | ACCCAGCA G CUUCUAAU | 1619 | ATTAGAAG GGCTAGCTACAACGA TGCTGGGT | 3947 |
| 1332 | AGCUUCUA A UUUGGGUG | 1620 | CACCCAAA GGCTAGCTACAACGA TAGAAGCT | 3948 |
| 1338 | UAAUUUGG G UGCGUGGU | 1621 | ACCACGCA GGCTAGCTACAACGA CCAAATTA | 3949 |
| 1340 | AUUUGGGU G CGUGGUUG | 1622 | CAACCACG GGCTAGCTACAACGA ACCCAAAT | 3950 |
| 1342 | UUGGGUGC G UGGUUGAG | 1623 | CTCAACCA GGCTAGCTACAACGA GCACCCAA | 3951 |
| 1345 | GGUGCGUG G UUGAGAGC | 1624 | GCTCTCAA GGCTAGCTACAACGA CACGCACC | 3952 |
| 1352 | GGUUGAGA G CGCUCAGC | 1625 | GCTGAGCG GGCTAGCTACAACGA TCTCAACC | 3953 |
| 1354 | UUGAGAGC G CUCAGCUG | 1626 | CAGCTGAG GGCTAGCTACAACGA GCTCTCAA | 3954 |
| 1359 | AGCGCUCA G CUGUCAGC | 1627 | GCTGACAG GGCTAGCTACAACGA TGAGCGCT | 3955 |
| 1362 | GCUCAGCU G UCAGCCCU | 1628 | AGGGCTGA GGCTAGCTACAACGA AGCTGAGC | 3956 |
| 1366 | AGCUGUCA G CCCUGCCU | 1629 | AGGCAGGG GGCTAGCTACAACGA TGACAGCT | 3957 |
| 1371 | UCAGCCCU G CCUUUGAG | 1630 | CTCAAAGG GGCTAGCTACAACGA AGGGCTGA | 3958 |
| 1381 | CUUUGAGG G CUGGGUCC | 1631 | GGACCCAG GGCTAGCTACAACGA CCTCAAAG | 3959 |
| 1386 | AGGGCUGG G UCCCUUUU | 1632 | AAAAGGGA GGCTAGCTACAACGA CCAGCCCT | 3960 |
| 1398 | CUUUUCCC A UCACUGGG | 1633 | CCCAGTGA GGCTAGCTACAACGA GGGAAAAG | 3961 |
| 1401 | UUCCCAUC A CUGGGUCA | 1634 | TGACCCAG GGCTAGCTACAACGA GATGGGAA | 3962 |
| 1406 | AUCACUGG G UCAUUAAG | 1635 | CTTAATGA GGCTAGCTACAACGA CCAGTGAT | 3963 |
| 1409 | ACUGGGUC A UUAAGAGC | 1636 | GCTCTTAA GGCTAGCTACAACGA GACCCAGT | 3964 |
| 1416 | CAUUAAGA G CAAGUGGG | 1637 | CCCACTTG GGCTAGCTACAACCA TCTTAATG | 3965 |
| 1420 | AAGAGCAA G UGGGGGCG | 1638 | CGCCCCCA GGCTAGCTACAACGA TTGCTCTT | 3966 |
| 1426 | AAGUGGGG G CGAGGCGA | 1639 | TCGCCTCG GGCTAGCTACAACGA CCCCACTT | 3967 |
| 1431 | GGGGCGAG G CGACAGCC | 1640 | GGCTGTCG GGCTAGCTACAACGA CTCGCCCC | 3968 |
| 1434 | GCGAGGCG A CAGCCCUC | 1641 | GAGGGCTG GGCTAGCTACAACGA CGCCTCGC | 3969 |
| 1437 | AGGCGACA G CCCUCCCG | 1642 | CGGGAGGG GGCTAGCTACAACGA TGTCGCCT | 3970 |
| 1445 | GCCCUCCC G CACGCUGG | 1643 | CCAGCGTG GGCTAGCTACAACGA GGGAGGGC | 3971 |
| 1447 | CCUCCCGC A CGCUGGGU | 1644 | ACCCAGCG GGCTAGCTACAACGA GCGGGAGG | 3972 |
| 1449 | UCCCGCAC G CUGGGUUG | 1645 | CAACCCAG GGCTAGCTACAACGA GTGCGGGA | 3973 |
| 1454 | CACGCUGG G UUGCAGCU | 1646 | AGCTGCAA GGCTAGCTACAACGA CCAGCGTG | 3974 |
| 1457 | GCUGGGUU G CAGCUGCA | 1647 | TGCAGCTG GGCTAGCTACAACGA AACCCAGC | 3975 |
| 1460 | GGGUUGCA G CUGCACAG | 1648 | CTGTGCAG GGCTAGCTACAACGA TGCAACCC | 3976 |
| 1463 | UUGCAGCU G CACAGGUA | 1649 | TACCTGTG GGCTAGCTACAACGA AGCTGCAA | 3977 |
| 1465 | GCAGCUGC A CAGGUAGG | 1650 | CCTACCTG GGCTAGCTACAACGA GCAGCTGC | 3978 |
| 1469 | CUGCACAG G UAGGCACG | 1651 | CGTGCCTA GGCTAGCTACAACGA CTGTGCAG | 3979 |
| 1473 | ACAGGUAG G CACGCUGC | 1652 | CCAGCGTG GGCTAGCTACAACGA CTACCTGT | 3980 |
| 1475 | AGGUAGGC A CGCUGCAG | 1653 | CTGCAGCG GGCTAGCTACAACGA GCCTACCT | 3981 |
| 1477 | GUAGGCAC G CUGCAGUC | 1654 | GACTGCAG GGCTAGCTACAACGA GTGCCTAC | 3982 |
| 1480 | GGCACGCU G CAGUCCUU | 1655 | AAGGACTG GGCTAGCTACAACGA AGCGTGCC | 3983 |
| 1483 | ACGCUGCA G UCCUUGCU | 1656 | AGCAAGGA GGCTAGCTACAACGA TGCAGCGT | 3984 |
| 1489 | CAGUCCUU G CUGCCUGG | 1657 | CCAGGCAG GGCTAGCTACAACGA AAGGACTG | 3985 |
| 1492 | UCCUUGCU G CCUGGCGU | 1658 | ACGCCAGG GGCTAGCTACAACGA AGCAAGGA | 3986 |
| 1497 | GCUGCCUG G CGUUGGGG | 1659 | CCCCAACG GGCTAGCTACAACGA CAGGCAGC | 3987 |
| 1499 | UGCCUGGC G UUGGGGCC | 1660 | GGCCCCAA GGCTAGCTACAACGA GCCAGGCA | 3988 |
| 1505 | GCGUUGGG G CCCAGGGA | 1661 | TCCCTGGG GGCTAGCTACAACGA CCCAACGC | 3989 |
| 1513 | GCCCAGGG A CCGCUGUG | 1662 | CACAGCGG GGCTAGCTACAACGA CCCTGGGC | 3990 |
| 1516 | CAGGGACC G CUGUGGGU | 1663 | ACCCACAG GGCTAGCTACAACGA GGTCCCTG | 3991 |
| 1519 | GGACCGCU G UGGGUUUG | 1664 | CAAACCCA GGCTAGCTACAACGA AGCGGTCC | 3992 |
| 1523 | CGCUGUGG G UUUGCCCU | 1665 | AGGGCAAA GGCTAGCTACAACGA CCACAGCG | 3993 |
| 1527 | GUGGGUUU G CCCUUCAG | 1666 | CTGAAGGG GGCTAGCTACAACGA AAACCCAC | 3994 |
| 1536 | CCCUUCAG A UGGCCCUG | 1667 | CAGGGCCA GGCTAGCTACAACGA CTGAAGGG | 3995 |
| 1539 | UUCAGAUG G CCCUGCCA | 1668 | TGGCAGGG GGCTAGCTACAACGA CATCTGAA | 3996 |
| 1544 | AUGGCCCU G CCAGCAGC | 1669 | GCTGCTGG GGCTAGCTACAACGA AGGGCCAT | 3997 |
| 1548 | CCCUGCCA G CAGCUGCC | 1670 | GGCAGCTG GGCTAGCTACAACGA TGGCAGGG | 3998 |
| 1551 | UGCCAGCA G CUGCCCUG | 1671 | CAGGGCAG GGCTAGCTACAACGA TGCTGGCA | 3999 |
| 1554 | CAGCAGCU G CCCUGUGG | 1672 | CCACAGGG GGCTAGCTACAACGA AGCTGCTG | 4000 |
| 1559 | GCUGCCCU G UGGGGCCU | 1673 | AGGCCCCA GGCTAGCTACAACGA AGGGCAGC | 4001 |
| 1564 | CCUGUGGG G CCUGGGGC | 1674 | GCCCCAGG GGCTAGCTACAACGA CCCACAGG | 4002 |
| 1571 | GGCCUGGG G CUGGGCCU | 1675 | AGGCCCAG GGCTAGCTACAACGA CCCAGGCC | 4003 |
| 1576 | GGGGCUGG G CCUGGGCC | 1676 | GGCCCAGG GGCTAGCTACAACGA CCAGCCCC | 4004 |
| 1582 | GGGCCUGG G CCUGGCUG | 1677 | CAGCCAGG GGCTAGCTACAACGA CCAGGCCC | 4005 |
| 1587 | UGGGCCUG G CUGACCAG | 1678 | CTGCTCAG GGCTAGCTACAACGA CAGGCCCA | 4006 |
| 1592 | CUGGCUGA G CAGGGCCC | 1679 | GGGCCCTG GGCTAGCTACAACGA TCAGCCAG | 4007 |
| 1597 | UGAGCAGG G CCCUCCUU | 1680 | AAGGAGGG GGCTAGCTACAACGA CCTGCTCA | 4008 |
| 1607 | CCUCCUUG G CAGGUGGG | 1681 | CCCACCTG GGCTACCTACAACGA CAAGGAGG | 4009 |
| 1611 | CUUCGCAG G UGGGGCAG | 1682 | CTGCCCCA GGCTAGCTACAACGA CTGCCAAG | 4010 |
| 1616 | CAGGUGGG G CAGGAGAC | 1683 | GTCTCCTG GGCTAGCTACAACGA CCCACCTG | 4011 |
| 1623 | GGCAGGAG A CCCUGUAG | 1684 | CTACAGGG GGCTAGCTACAACGA CTCCTGCC | 4012 |
| 1628 | GAGACCCU G UAGGAGGA | 1685 | TCCTCCTA GGCTAGCTACAACGA AGGGTCTC | 4013 |
| 1636 | GUAGGAGG A CCCCGGGC | 1686 | GCCCGGGG GGCTAGCTACAACGA CCTCCTAC | 4014 |
| 1643 | GACCCCGG G CCGCAGGC | 1687 | GCCTGCGG GGCTAGCTACAACGA CCGGGGTC | 4015 |
| 1646 | CCCGGGCC G CAGGCCCC | 1688 | GGGGCCTG GGCTAGCTACAACGA GGCCCGGG | 4016 |
| 1650 | GGCCGCAG G CCCCUGAG | 1689 | CTCAGGGG GGCTAGCTACAACGA CTGCGGCC | 4017 |
| 1661 | CCUGAGCA G CGAUGACG | 1690 | CGTCATCG GGCTAGCTACAACGA TCCTCAGG | 4018 |
| 1664 | GAGGAGCG A UGACGGAA | 1691 | TTCCGTCA GGCTAGCTACAACGA CGCTCCTC | 4019 |
| 1667 | GAGCGAUG A CGGAAUAU | 1692 | ATATTCCG GGCTAGCTACAACGA CATCGCTC | 4020 |
| 1672 | AUGACGGA A UAUAAGCU | 1693 | AGCTTATA GGCTAGCTACAACGA TCCGTCAT | 4021 |
| 1674 | GACGGAAU A UAAGCUGG | 1694 | CCAGCTTA GGCTAGCTACAACGA ATTCCGTC | 4022 |
| 1678 | GAAUAUAA G CUGGUGGU | 1695 | ACCACCAG GGCTAGCTACAACGA TTATATTC | 4023 |
| 1682 | AUAAGCUG G UGGUGGUG | 1696 | CACCACCA GGCTAGCTACAACGA CAGCTTAT | 4024 |
| 1685 | AGCUGGUG G UGGUGGGC | 1697 | GCCCACCA GGCTAGCTACAACGA CACCAGCT | 4025 |
| 1688 | UGGUGGUG G UGGGCGCC | 1698 | GGCGCCCA GGCTAGCTACAACGA CACCACCA | 4026 |
| 1692 | GGUGGUGG G CGCCGGCG | 1699 | CGCCGGCG GGCTAGCTACAACGA CCACCACC | 4027 |
| 1694 | UGGUGGGC G CCGGCGGU | 1700 | ACCGCCGG GGCTAGCTACAACGA GCCCACCA | 4028 |
| 1698 | GGGCGCCG G CGGUGUGG | 1701 | CCACACCG GGCTAGCTACAACCA CGGCGCCC | 4029 |
| 1701 | CGCCGGCG G UGUGGGCA | 1702 | TGCCCACA GGCTAGCTACAACGA CGCCGGCG | 4030 |
| 1703 | CCGGCGGU G UGGGCAAG | 1703 | CTTGCCCA GGCTAGCTACAACGA ACCGCCGG | 4031 |
| 1707 | CGGUGUGG G CAAGAGUG | 1704 | CACTCTTG GGCTAGCTACAACGA CCACACCG | 4032 |
| 1713 | GGGCAAGA G UGCGCUGA | 1705 | TCAGCGCA GGCTAGCTACAACGA TCTTGCCC | 4033 |
| 1715 | GCAAGAGU G CGCUGACC | 1706 | GGTCAGCG GGCTAGCTACAACGA ACTCTTGC | 4034 |
| 1717 | AAGAGUGC G CUGACCAU | 1707 | ATGGTCAG GGCTAGCTACAACGA GCACTCTT | 4035 |
| 1721 | GUGCGCUG A CCAUCCAG | 1708 | CTGGATGG GGCTAGCTACAACGA CAGCGCAC | 4036 |
| 1724 | CGCUGACC A UCCAGCUG | 1709 | CAGCTGGA GGCTAGCTACAACGA GGTCAGCG | 4037 |
| 1729 | ACCAUCCA G CUGAUCCA | 1710 | TGGATCAG GGCTAGCTACAACGA TGGATGGT | 4038 |
| 1733 | UCCAGCUG A UCCAGAAC | 1711 | GTTCTGGA GGCTAGCTACAACGA CAGCTGGA | 4039 |
| 1740 | GAUCCAGA A CCAUUUUG | 1712 | CAAAATGG GGCTAGCTACAACGA TCTGGATC | 4040 |
| 1743 | CCAGAACC A UUUUGUGG | 1713 | CCACAAAA GGCTAGCTACAACGA GGTTCTGG | 4041 |
| 1748 | ACCAUUUU G UGGACGAA | 1714 | TTCGTCCA GGCTAGCTACAACGA AAAATGGT | 4042 |
| 1752 | UUUUGUGG A CGAAUACG | 1715 | CGTATTCG GGCTAGCTACAACGA CCACAAAA | 4043 |
| 1756 | GUGGACGA A UACGACCC | 1716 | GGGTCGTA GGCTAGCTACAACGA TCGTCCAC | 4044 |
| 1758 | GGACGAAU A CGACCCCA | 1717 | TGGGGTCG GGCTAGCTACAACGA ATTCGTCC | 4045 |
| 1761 | CGAAUACG A CCCCACUA | 1718 | TAGTGGGG GGCTAGCTACAACGA CGTATTCG | 4046 |
| 1766 | ACGACCCC A CUAUAGAG | 1719 | CTCTATAG GGCTAGCTACAACGA GGGGTCGT | 4047 |
| 1769 | ACCCCACU A UAGAGGAU | 1720 | ATCCTCTA GGCTAGCTACAACGA AGTGGGGT | 4048 |
| 1776 | UAUAGAGG A UUCCUACC | 1721 | GGTAGGAA GGCTAGCTACAACGA CCTCTATA | 4049 |
| 1782 | GGAUUCCU A CCGGAAGC | 1722 | GCTTCCGG GGCTAGCTACAACGA AGGAATCC | 4050 |
| 1789 | UACCGGAA G CAGGUGGU | 1723 | ACCACCTG GGCTAGCTACAACGA TTCCGGTA | 4051 |
| 1793 | GGAAGCAG G UGGUCAUU | 1724 | AATGACCA GGCTAGCTACAACGA CTGCTTCC | 4052 |
| 1796 | AGCAGGUG G UCAUUGAU | 1725 | ATCAATGA GGCTAGCTACAACGA CACCTGCT | 4053 |
| 1799 | AGGUGGUC A UUGAUGGG | 1726 | CCCATCAA GGCTAGCTACAACGA GACCACCT | 4054 |
| 1803 | GGUCAUUG A UGGGGAGA | 1727 | TCTCCCCA GGCTAGCTACAACGA CAATGACC | 4055 |
| 1811 | AUGGGGAG A CGUGCCUG | 1728 | CAGGCACG GGCTAGCTACAACGA CTCCCCAT | 4056 |
| 1813 | GGGGAGAC G UGCCUGUU | 1729 | AACAGGCA GGCTAGCTACAACGA GTCTCCCC | 4057 |
| 1815 | GGAGACGU G CCUGUUGG | 1730 | CCAACAGG GGCTAGCTACAACGA ACGTCTCC | 4058 |
| 1819 | ACGUGCCU G UUGGACAU | 1731 | ATGTCCAA GGCTAGCTACAACGA AGGCACGT | 4059 |
| 1824 | CCUGUUGG A CAUCCUGG | 1732 | CCAGGATG GGCTAGCTACAACGA CCAACAGG | 4060 |
| 1826 | UGUUGGAC A UCCUGGAU | 1733 | ATCCAGGA GGCTAGCTACAACGA GTCCAACA | 4061 |
| 1833 | CAUCCUGG A UACCGCCG | 1734 | CGGCGGTA GGCTAGCTACAACGA CCAGGATG | 4062 |
| 1835 | UCCUGGAU A CCGCCGGC | 1735 | GCCGGCGG GGCTAGCTACAACGA ATCCAGGA | 4063 |
| 1838 | UGGAUACC G CCGGCCAG | 1736 | CTGGCCGG GGCTAGCTACAACGA GGTATCCA | 4064 |
| 1842 | UACCGCCG G CCAGGAGG | 1737 | CCTCCTGG GGCTAGCTACAACGA CGGCGGTA | 4065 |
| 1852 | CAGGAGGA G UACAGCGC | 1738 | GCGCTGTA GGCTAGCTACAACGA TCCTCCTG | 4066 |
| 1854 | GGAGGAGU A CAGCGCCA | 1739 | TGGCGCTG GGCTAGCTACAACGA ACTCCTCC | 4067 |
| 1857 | GGAGUACA G CGCCAUGC | 1740 | GCATGGCG GGCTAGCTACAACGA TGTACTCC | 4068 |
| 1859 | AGUACAGC G CCAUGCGG | 1741 | CCGCATGG GGCTAGCTACAACGA GCTGTACT | 4069 |
| 1862 | ACAGCGCC A UGCGGGAC | 1742 | GTCCCGCA GGCTAGCTACAACGA GGCGCTGT | 4070 |
| 1864 | AGCGCCAU G CGGGACCA | 1743 | TGGTCCCG GGCTAGCTACAACGA ATGGCGCT | 4071 |
| 1869 | CAUGCGGG A CCAGUACA | 1744 | TGTACTGG GGCTAGCTACAACGA CCCGCATG | 4072 |
| 1873 | CGGGACCA G UACAUGCG | 1745 | CGCATGTA GGCTAGCTACAACGA TGGTCCCG | 4073 |
| 1875 | GGACCAGU A CAUGCGCA | 1746 | TGCGCATG GGCTAGCTACAACGA ACTGGTCC | 4074 |
| 1877 | ACCAGUAC A UGCGCACC | 1747 | GGTGCGCA GGCTAGCTACAACGA GTACTGGT | 4075 |
| 1879 | CAGUACAU G CGCACCGG | 1748 | CCGGTGCG GGCTAGCTACAACGA ATGTACTG | 4076 |
| 1881 | GUACAUGC G CACCGGGG | 1749 | CCCCGGTG GGCTAGCTACAACGA GCATGTAC | 4077 |
| 1883 | ACAUGCGC A CCGGGGAG | 1750 | CTCCCCGG GGCTAGCTACAACGA GCGCATGT | 4078 |
| 1893 | CGGGGAGG G CUUCCUGU | 1751 | ACAGGAAG GGCTAGCTACAACGA CCTCCCCG | 4079 |
| 1900 | GGCUUCCU G UGUGUGUU | 1752 | AACACACA GGCTAGCTACAACGA AGGAAGCC | 4080 |
| 1902 | CUUCCUGU G UGUGUUUG | 1753 | CAAACACA GGCTAGCTACAACGA ACAGGAAG | 4081 |
| 1904 | UCCUGUGU G UGUUUGCC | 1754 | GGCAAACA GGCTAGCTACAACGA ACACAGGA | 4082 |
| 1906 | CUGUGUGU G UUUGCCAU | 1755 | ATGGCAAA GGCTAGCTACAACGA ACACACAG | 4083 |
| 1910 | GUGUGUUU G CCAUCAAC | 1756 | GTTGATGG GGCTAGCTACAACGA AAACACAC | 4084 |
| 1913 | UGUUUGCC A UCAACAAC | 1757 | GTTGTTGA GGCTAGCTACAACGA GGCAAACA | 4085 |
| 1917 | UGCCAUCA A CAACACCA | 1758 | TGGTGTTG GGCTAGCTACAACGA TGATGGCA | 4086 |
| 1920 | CAUCAACA A CACCAAGU | 1759 | ACTTGGTG GGCTAGCTACAACGA TGTTGATG | 4087 |
| 1922 | UCAACAAC A CCAAGUCU | 1760 | AGACTTGG GGCTAGCTACAACGA GTTGTTGA | 4088 |
| 1927 | AACACCAA G UCUUUUGA | 1761 | TCAAAAGA GGCTAGCTACAACGA TTGGTGTT | 4089 |
| 1938 | UUUUGAGG A CAUCCACC | 1762 | GGTGGATG GGCTAGCTACAACGA CCTCAAAA | 4090 |
| 1940 | UUGAGGAC A UCCACCAG | 1763 | CTGGTGGA GGCTAGCTACAACGA GTCCTCAA | 4091 |
| 1944 | GGACAUCC A CCAGUACA | 1764 | TGTACTGG GGCTAGCTACAACGA GGATGTCC | 4092 |
| 1948 | AUCCACCA G UACAGGGA | 1765 | TCCCTGTA GGCTAGCTACAACGA TGGTGGAT | 4093 |
| 1950 | CCACCAGU A CAGGGAGC | 1766 | GCTCCCTG GGCTAGCTACAACGA ACTGGTGG | 4094 |
| 1957 | UACAGGGA G CAGAUCAA | 1767 | TTGATCTG GGCTAGCTACAACGA TCCCTGTA | 4095 |
| 1961 | GGGAGCAG A UCAAACGG | 1768 | CCGTTTGA GGCTAGCTACAACGA CTGCTCCC | 4096 |
| 1966 | CAGAUCAA A CGGGUGAA | 1769 | TTCACCCG GGCTAGCTACAACGA TTGATCTG | 4097 |
| 1970 | UCAAACGG G UGAAGGAC | 1770 | GTCCTTCA GGCTAGCTACAACGA CCGTTTGA | 4098 |
| 1977 | GGUGAAGG A CUCGGAUG | 1771 | CATCCGAG GGCTAGCTACAACGA CCTTCACC | 4099 |
| 1983 | GGACUCGG A UGACGUGC | 1772 | GCACGTCA GGCTAGCTACAACGA CCGAGTCC | 4100 |
| 1986 | CUCGGAUG A CGUGCCCA | 1773 | TGGGCACG GGCTAGCTACAACGA CATCCGAG | 4101 |
| 1988 | CGGAUGAC G UGCCCAUG | 1774 | CATGGGCA GGCTAGCTACAACGA GTCATCCG | 4102 |
| 1990 | GAUGACGU G CCCAUGGU | 1775 | ACCATGGG GGCTAGCTACAACGA ACGTCATC | 4103 |
| 1994 | ACGUGCCC A UGGUGCUG | 1776 | CAGCACCA GGCTAGCTACAACGA GGGCACGT | 4104 |
| 1997 | UGCCCAUG G UGCUGGUG | 1777 | CACCAGCA GGCTAGCTACAACGA CATGGGCA | 4105 |
| 1999 | CCCAUGGU G CUGGUGGG | 1778 | CCCACCAG GGCTAGCTACAACGA ACCATGGG | 4106 |
| 2003 | UGGUGCUG G UGGGGAAC | 1779 | GTTCCCCA GGCTAGCTACAACGA CAGCACCA | 4107 |
| 2010 | GGUGGGGA A CAAGUGUG | 1780 | CACACTTG GGCTAGCTACAACGA TCCCCACC | 4108 |
| 2014 | GGGAACAA G UGUGACCU | 1781 | AGGTCACA GGCTAGCTACAACGA TTGTTCCC | 4109 |
| 2016 | GAACAAGU G UGACCUGG | 1782 | CCAGGTCA GGCTAGCTACAACGA ACTTGTTC | 4110 |
| 2019 | CAAGUGUG A CCUGGCUG | 1783 | CAGCCAGG GGCTAGCTACAACGA CACACTTG | 4111 |
| 2024 | GUGACCUG G CUGCACGC | 1784 | GCGTGCAG GGCTAGCTACAACGA CAGGTCAC | 4112 |
| 2027 | ACCUGGCU G CACGCACU | 1785 | AGTGCGTG GGCTAGCTACAACGA AGCCAGGT | 4113 |
| 2029 | CUGGCUGC A CGCACUGU | 1786 | ACAGTGCG GGCTAGCTACAACGA GCAGCCAG | 4114 |
| 2031 | GGCUGCAC G CACUGUGG | 1787 | CCACAGTG GGCTAGCTACAACGA GTGCAGCC | 4115 |
| 2033 | CUGCACGC A CUGUGGAA | 1788 | TTCCACAG GGCTAGCTACAACGA GCGTGCAG | 4116 |
| 2036 | CACGCACU G UGGAAUCU | 1789 | AGATTCCA GGCTAGCTACAACGA AGTGCGTG | 4117 |
| 2041 | ACUGUGGA A UCUCGGCA | 1790 | TGCCGAGA GGCTAGCTACAACGA TCCACAGT | 4118 |
| 2047 | GAAUCUCG G CAGGCUCA | 1791 | TGAGCCTG GGCTAGCTACAACGA CGAGATTC | 4119 |
| 2051 | CUCGGCAG G CUCAGGAC | 1792 | GTCCTGAG GGCTAGCTACAACGA CTGCCGAG | 4120 |
| 2058 | GGCUCAGG A CCUCGCCC | 1793 | GGGCGAGG GGCTAGCTACAACGA CCTGAGCC | 4121 |
| 2063 | AGGACCUC G CCCGAAGC | 1794 | GCTTCGGG GGCTAGCTACAACGA GAGGTCCT | 4122 |
| 2070 | CGCCCGAA G CUACGGCA | 1795 | TGCCGTAG GGCTAGCTACAACGA TTCGGGCG | 4123 |
| 2073 | CCGAAGCU A CGGCAUCC | 1796 | GGATGCCG GGCTAGCTACAACGA AGCTTCGG | 4124 |
| 2076 | AAGCUACG G CAUCCCCU | 1797 | AGGGGATG GGCTAGCTACAACGA CGTAGCTT | 4125 |
| 2078 | GCUACGGC A UCCCCUAC | 1798 | GTAGGGGA GGCTAGCTACAACGA GCCGTAGC | 4126 |
| 2085 | CAUCCCCU A CAUCGAGA | 1799 | TCTCGATG GGCTAGCTACAACGA AGGGGATG | 4127 |
| 2087 | UCCCCUAC A UCGAGACC | 1800 | GGTCTCGA GGCTAGCTACAACGA GTAGGGGA | 4128 |
| 2093 | ACAUCGAG A CCUCGGCC | 1801 | GGCCGAGG GGCTAGCTACAACGA CTCGATGT | 4129 |
| 2099 | AGACCUCG G CCAAGACC | 1802 | GGTCTTGG GGCTAGCTACAACGA CGAGGTCT | 4130 |
| 2105 | CGGCCAAG A CCCGGCAG | 1803 | CTGCCGGG GGCTAGCTACAACGA CTTGGCCG | 4131 |
| 2110 | AAGACCCG G CAGGGAGU | 1804 | ACTCCCTG GGCTAGCTACAACGA CGGGTCTT | 4132 |
| 2117 | GGCAGGGA G UGGAGGAU | 1805 | ATCCTCCA GGCTAGCTACAACGA TCCCTGCC | 4133 |
| 2124 | AGUGGAGG A UGCCUUCU | 1806 | AGAAGGCA GGCTAGCTACAACGA CCTCCACT | 4134 |
| 2126 | UGGAGGAU G CCUUCUAC | 1807 | GTAGAAGG GGCTAGCTACAACGA ATCCTCCA | 4135 |
| 2133 | UGCCUUCU A CACGUUGG | 1808 | CCAACGTG GGCTAGCTACAACGA AGAAGGCA | 4136 |
| 2135 | CCUUCUAC A CGUUGGUG | 1809 | CACCAACG GGCTAGCTACAACGA GTAGAAGG | 4137 |
| 2137 | UUCUACAC G UUGGUGCG | 1810 | CGCACCAA GGCTAGCTACAACGA GTGTAGAA | 4138 |
| 2141 | ACACGUUG G UGCGUGAG | 1811 | CTCACGCA GGCTAGCTACAACGA CAACGTGT | 4139 |
| 2143 | ACGUUGGU G CGUGAGAU | 1812 | ATCTCACG GGCTAGCTACAACGA ACCAACGT | 4140 |
| 2145 | GUUGGUGC G UGAGAUCC | 1813 | GGATCTCA GGCTAGCTACAACGA GCACCAAC | 4141 |
| 2150 | UGCGUGAG A UCCGGCAG | 1814 | CTGCCGGA GGCTAGCTACAACGA CTCACGCA | 4142 |
| 2155 | GAGAUCCG G CAGCACAA | 1815 | TTGTGCTG GGCTAGCTACAACGA CGGATCTC | 4143 |
| 2158 | AUCCGGCA G CACAAGCU | 1816 | AGCTTGTG GGCTAGCTACAACGA TGCCGGAT | 4144 |
| 2160 | CCGGCAGC A CAAGCUGC | 1817 | GCAGCTTG GGCTAGCTACAACGA GCTGCCGG | 4145 |
| 2164 | CAGCACAA G CUGCGGAA | 1818 | TTCCGCAG GGCTAGCTACAACGA TTGTGCTG | 4146 |
| 2167 | CACAAGCU G CGGAAGCU | 1819 | AGCTTCCG GGCTAGCTACAACGA AGCTTGTG | 4147 |
| 2173 | CUGCGGAA G CUGAACCC | 1820 | GGGTTCAG GGCTAGCTACAACGA TTCCGCAG | 4148 |
| 2178 | GAAGCUGA A CCCUCCUG | 1821 | CAGGAGGG GGCTAGCTACAACGA TCAGCTTC | 4149 |
| 2187 | CCCUCCUG A UGAGAGUG | 1822 | CACTCTCA GGCTAGCTACAACGA CAGGAGGG | 4150 |
| 2193 | UGAUGAGA G UGGCCCCG | 1823 | CGGGGCCA GGCTAGCTACAACGA TCTCATCA | 4151 |
| 2196 | UGAGAGUG G CCCCGGCU | 1824 | AGCCGGGG GGCTAGCTACAACGA CACTCTCA | 4152 |
| 2202 | UGGCCCCG G CUGCAUGA | 1825 | TCATGCAG GGCTAGCTACAACGA CGGGGCCA | 4153 |
| 2205 | CCCCGGCU G CAUGAGCU | 1826 | AGCTCATG GGCTAGCTACAACGA AGCCGGGG | 4154 |
| 2207 | CCGGCUGC A UGAGCUGC | 1827 | GCAGCTCA GGCTAGCTACAACGA GCAGCCGG | 4155 |
| 2211 | CUGCAUGA G CUGCAAGU | 1828 | ACTTGCAG GGCTAGCTACAACGA TCATGCAG | 4156 |
| 2214 | CAUGAGCU G CAAGUGUG | 1829 | CACACTTG GGCTAGCTACAACGA AGCTCATG | 4157 |
| 2218 | AGCUGCAA G UGUGUGCU | 1830 | AGCACACA GGCTAGCTACAACGA TTGCAGCT | 4158 |
| 2220 | CUGCAAGU G UGUGCUCU | 1831 | AGAGCACA GGCTAGCTACAACGA ACTTGCAG | 4159 |
| 2222 | GCAAGUGU G UGCUCUCC | 1832 | GGAGAGCA GGCTAGCTACAACGA ACACTTGC | 4160 |
| 2224 | AAGUGUGU G CUCUCCUG | 1833 | CAGGAGAG GGCTAGCTACAACGA ACACACTT | 4161 |
| 2233 | CUCUCCUG A CGCAGGUG | 1834 | CACCTGCG GGCTAGCTACAACGA CAGGAGAG | 4162 |
| 2235 | CUCCUGAC G CAGGUGAG | 1835 | CTCACCTG GGCTAGCTACAACGA GTCAGGAG | 4163 |
| 2239 | UGACGCAG G UGAGGGGG | 1836 | CCCCCTCA GGCTAGCTACAACGA CTGCGTCA | 4164 |
| 2248 | UGAGGGGG A CUCCCAGG | 1837 | CCTGGGAG GGCTAGCTACAACGA CCCCCTCA | 4165 |
| 2257 | CUCCCAGG G CGGCCGCC | 1838 | GGCGGCCG GGCTAGCTACAACGA CCTGGGAG | 4166 |
| 2260 | CCAGGGCG G CCGCCACG | 1839 | CGTGGCGG GGCTAGCTACAACGA CGCCCTGG | 4167 |
| 2263 | GGGCGGCC G CCACGCCC | 1840 | GGGCGTGG GGCTAGCTACAACGA GGCCGCCC | 4168 |
| 2266 | CGGCCGCC A CGCCCACC | 1841 | GGTGGGCG GGCTAGCTACAACGA GGCGGCCG | 4169 |
| 2268 | GCCGCCAC G CCCACCGG | 1842 | CCGGTGGG GGCTAGCTACAACGA GTGGCGGC | 4170 |
| 2272 | CCACGCCC A CCGGAUGA | 1843 | TCATCCGG GGCTAGCTACAACGA GGGCGTGG | 4171 |
| 2277 | CCCACCGG A UGACCCCG | 1844 | CGGGGTCA GGCTAGCTACAACGA CCGGTGGG | 4172 |
| 2280 | ACCGGAUG A CCCCGGCU | 1845 | AGCCGGGG GGCTAGCTACAACGA CATCCGGT | 4173 |
| 2286 | UGACCCCG G CUCCCCGC | 1846 | GCGGGGAG GGCTAGCTACAACGA CGGGGTCA | 4174 |
| 2293 | GGCUCCCC G CCCCUGCC | 1847 | GGCAGGGG GGCTAGCTACAACGA GGGGAGCC | 4175 |
| 2299 | CCGCCCCU G CCGGUCUC | 1848 | GAGACCGG GGCTAGCTACAACGA AGGGGCGG | 4176 |
| 2303 | CCCUGCCG G UCUCCUGG | 1849 | CCAGGAGA GGCTAGCTACAACGA CGGCAGGG | 4177 |
| 2311 | GUCUCCUG G CCUGCGGU | 1850 | ACCGCAGG GGCTAGCTACAACGA CAGGAGAC | 4178 |
| 2315 | CCUGGCCU G CGGUCAGC | 1851 | GCTGACCG GGCTAGCTACAACGA AGGCCAGG | 4179 |
| 2318 | GGCCUGCG G UCAGCAGC | 1852 | GCTGCTGA GGCTAGCTACAACGA CGCAGGCC | 4180 |
| 2322 | UGCGGUCA G CAGCCUCC | 1853 | GGAGGCTG GGCTAGCTACAACGA TGACCGCA | 4181 |
| 2325 | GGUCAGCA G CCUCCCUU | 1854 | AAGGGAGG GGCTAGCTACAACGA TGCTGACC | 4182 |
| 2334 | CCUCCCUU G UGCCCCGC | 1855 | GCGGGGCA GGCTAGCTACAACGA AAGGGAGG | 4183 |
| 2336 | UCCCUUGU G CCCCGCCC | 1856 | GGGCGGGG GGCTAGCTACAACGA ACAAGGGA | 4184 |
| 2341 | UGUGCCCC G CCCAGCAC | 1857 | GTGCTGGG GGCTAGCTACAACGA GGGGCACA | 4185 |
| 2346 | CCCGCCCA G CACAAGCU | 1858 | AGCTTGTG GGCTAGCTACAACGA TGGGCGGG | 4186 |
| 2348 | CGCCCAGC A CAAGCUCA | 1859 | TGAGCTTG GGCTAGCTACAACGA GCTGGGCG | 4187 |
| 2352 | CAGCACAA G CUCAGGAC | 1860 | GTCCTGAG GGCTAGCTACAACGA TTGTGCTG | 4188 |
| 2359 | AGCUCAGG A CAUGGAGG | 1861 | CCTCCATG GGCTAGCTACAACGA CCTGAGCT | 4189 |
| 2361 | CUCAGGAC A UGGAGGUG | 1862 | CACCTCCA GGCTAGCTACAACGA GTCCTGAG | 4190 |
| 2367 | ACAUGGAG G UGCCGGAU | 1863 | ATCCGGCA GGCTAGCTACAACGA CTCCATGT | 4191 |
| 2369 | AUGGAGGU G CCGGAUGC | 1864 | GCATCCGG GGCTAGCTACAACGA ACCTCCAT | 4192 |
| 2374 | GGUGCCGG A UGCAGGAA | 1865 | TTCCTGCA GGCTAGCTACAACGA CCGGCACC | 4193 |
| 2376 | UGCCGGAU G CAGGAAGG | 1866 | CCTTCCTG GGCTAGCTACAACGA ATCCGGCA | 4194 |
| 2387 | GGAAGGAG G UGCAGACG | 1867 | CGTCTGCA GGCTAGCTACAACGA CTCCTTCC | 4195 |
| 2389 | AAGGAGGU G CAGACGGA | 1868 | TCCGTCTG GGCTAGCTACAACGA ACCTCCTT | 4196 |
| 2393 | AGGUGCAG A CGGAAGGA | 1869 | TCCTTCCG GGCTAGCTACAACGA CTGCACCT | 4197 |
| 2415 | AAGGAAGG A CGGAAGCA | 1870 | TGCTTCCG GGCTAGCTACAACGA CCTTCCTT | 4198 |
| 2421 | GGACGGAA G CAAGGAAG | 1871 | CTTCCTTG GGCTAGCTACAACGA TTCCGTCC | 4199 |
| 2439 | AAGGAAGG G CUGCUGGA | 1872 | TCCAGCAG GGCTAGCTACAACGA CCTTCCTT | 4200 |
| 2442 | GAAGGGCU G CUGGAGCC | 1873 | GGCTCCAG GGCTAGCTACAACGA AGCCCTTC | 4201 |
| 2448 | CUGCUGGA G CCCAGUCA | 1874 | TGACTGGG GGCTAGCTACAACGA TCCAGCAG | 4202 |
| 2453 | GGAGCCCA G UCACCCCG | 1875 | CGGGGTGA GGCTAGCTACAACGA TGGGCTCC | 4203 |
| 2456 | GCCCAGUC A CCCCGGGA | 1876 | TCCCGGGG GGCTAGCTACAACGA GACTGGGC | 4204 |
| 2464 | ACCCCGGG A CCGUGGGC | 1877 | GCCCACGG GGCTAGCTACAACGA CCCGGGGT | 4205 |
| 2467 | CCGGGACC G UGGGCCGA | 1878 | TCGGCCCA GGCTAGCTACAACGA GGTCCCGG | 4206 |
| 2471 | GACCGUGG G CCGAGGUG | 1879 | CACCTCGG GGCTAGCTACAACGA CCACGGTC | 4207 |
| 2477 | GGGCCGAG G UGACUGCA | 1880 | TGCAGTCA GGCTAGCTACAACGA CTCGGCCC | 4208 |
| 2480 | CCGAGGUG A CUGCAGAC | 1881 | GTCTGCAG GGCTAGCTACAACGA CACCTCGG | 4209 |
| 2483 | AGGUGACU G CAGACCCU | 1882 | AGGGTCTG GGCTAGCTACAACGA AGTCACCT | 4210 |
| 2487 | GACUGCAG A CCCUCCCA | 1883 | TGGGAGGG GGCTAGCTACAACGA CTGCAGTC | 4211 |
| 2501 | CCAGGGAG G CUGUGCAC | 1884 | GTGCACAG GGCTAGCTACAACGA CTCCCTGG | 4212 |
| 2504 | GGGAGGCU G UGCACAGA | 1885 | TCTGTGCA GGCTAGCTACAACGA AGCCTCCC | 4213 |
| 2506 | GAGGCUGU G CACAGACU | 1886 | AGTCTGTG GGCTAGCTACAACGA ACAGCCTC | 4214 |
| 2508 | GGCUGUGC A CAGACUGU | 1887 | ACAGTCTG GGCTAGCTACAACGA GCACAGCC | 4215 |
| 2512 | GUGCACAG A CUGUCUUG | 1888 | CAAGACAG GGCTAGCTACAACGA CTGTGCAC | 4216 |
| 2515 | CACAGACU G UCUUGAAC | 1889 | GTTCAAGA GGCTAGCTACAACGA AGTCTGTG | 4217 |
| 2522 | UGUCUUGA A CAUCCCAA | 1890 | TTGGGATG GGCTAGCTACAACGA TCAAGACA | 4218 |
| 2524 | UCUUGAAC A UCCCAAAU | 1891 | ATTTGGGA GGCTAGCTACAACGA GTTCAAGA | 4219 |
| 2531 | CAUCCCAA A UGCCACCG | 1892 | CGGTGGCA GGCTAGCTACAACGA TTGGGATG | 4220 |
| 2533 | UCCCAAAU G CCACCGGA | 1893 | TCCGGTGG GGCTAGCTACAACGA ATTTGGGA | 4221 |
| 2536 | CAAAUGCC A CCGGAACC | 1894 | GGTTCCGG GGCTAGCTACAACGA GGCATTTG | 4222 |
| 2542 | CCACCGGA A CCCCAGCC | 1895 | GGCTGGGG GGCTAGCTACAACGA TCCGGTGG | 4223 |
| 2548 | GAACCCCA G CCCUUAGC | 1896 | GCTAAGGG GGCTAGCTACAACGA TGGGGTTC | 4224 |
| 2555 | AGCCCUUA G CUCCCCUC | 1897 | GAGGGGAG GGCTAGCTACAACGA TAAGGGCT | 4225 |
| 2568 | CCUCCCAG G CCUCUGUG | 1898 | CACAGAGG GGCTAGCTACAACGA CTGGGAGG | 4226 |
| 2574 | AGGCCUCU G UGGGCCCU | 1899 | AGGGCCCA GGCTAGCTACAACGA AGAGGCCT | 4227 |
| 2578 | CUCUGUGG G CCCUUGUC | 1900 | GACAAGGG GGCTAGCTACAACGA CCACAGAG | 4228 |
| 2584 | GGGCCCUU G UCGGGCAC | 1901 | GTGCCCGA GGCTAGCTACAACGA AAGGGCCC | 4229 |
| 2589 | CUUGUCGG G CACAGAUG | 1902 | CATCTGTG GGCTAGCTACAACGA CCGACAAG | 4230 |
| 2591 | UGUCGGGC A CAGAUGGG | 1903 | CCCATCTG GGCTAGCTACAACGA GCCCGACA | 4231 |
| 2595 | GGGCACAG A UGGGAUCA | 1904 | TGATCCCA GGCTAGCTACAACGA CTGTGCCC | 4232 |
| 2600 | CAGAUGGG A UCACAGUA | 1905 | TACTGTGA GGCTAGCTACAACGA CCCATCTG | 4233 |
| 2603 | AUGGGAUC A CAGUAAAU | 1906 | ATTTACTG GGCTAGCTACAACGA GATCCCAT | 4234 |
| 2606 | GGAUCACA G UAAAUUAU | 1907 | ATAATTTA GGCTAGCTACAACGA TGTGATCC | 4235 |
| 2610 | CACAGUAA A UUAUUGGA | 1908 | TCCAATAA GGCTAGCTACAACGA TTACTGTG | 4236 |
| 2613 | AGUAAAUU A UUGGAUGG | 1909 | CCATCCAA GGCTAGCTACAACGA AATTTACT | 4237 |
| 2618 | AUUAUUGG A UGGUCUUG | 1910 | CAAGACCA GGCTAGCTACAACGA CCAATAAT | 4238 |
| 2621 | AUUGGAUG G UCUUGAUC | 1911 | GATCAAGA GGCTAGCTACAACGA CATCCAAT | 4239 |
| 2627 | UGGUCUUG A UCUUGGUU | 1912 | AACCAAGA GGCTAGCTACAACGA CAAGACCA | 4240 |
| 2633 | UGAUCUUG G UUUUCGGC | 1913 | GCCGAAAA GGCTAGCTACAACGA CAAGATCA | 4241 |
| 2640 | GGUUUUCG G CUGAGGGU | 1914 | ACCCTCAG GGCTAGCTACAACGA CGAAAACC | 4242 |
| 2647 | GGCUGAGG G UGGGACAC | 1915 | GTGTCCCA GGCTAGCTACAACGA CCTCAGCC | 4243 |
| 2652 | AGGGUGGG A CACGGUGC | 1916 | GCACCGTG GGCTAGCTACAACGA CCCACCCT | 4244 |
| 2654 | GGUGGGAC A CGGUGCGC | 1917 | GCGCACCG GGCTAGCTACAACGA GTCCCACC | 4245 |
| 2657 | GGGACACG G UGCGCGUG | 1918 | CACGCGCA GGCTAGCTACAACGA CGTGTCCC | 4246 |
| 2659 | GACACGGU G CGCGUGUG | 1919 | CACACGCG GGCTAGCTACAACGA ACCGTGTC | 4247 |
| 2661 | CACGGUGC G CGUGUGGC | 1920 | GCCACACG GGCTAGCTACAACGA GCACCGTG | 4248 |
| 2663 | CGGUGCGC G UGUGGCCU | 1921 | AGGCCACA GGCTAGCTACAACGA GCGCACCG | 4249 |
| 2665 | GUGCGCGU G UGGCCUGG | 1922 | CCAGGCCA GGCTAGCTACAACGA ACGCGCAC | 4250 |
| 2668 | CGCGUGUG G CCUGGCAU | 1923 | ATGCCAGG GGCTAGCTACAACGA CACACGCG | 4251 |
| 2673 | GUGGCCUG G CAUGAGGU | 1924 | ACCTCATG GGCTAGCTACAACGA CAGGCCAC | 4252 |
| 2675 | GGCCUGGC A UGAGGUAU | 1925 | ATACCTCA GGCTAGCTACAACGA GCCAGGCC | 4253 |
| 2680 | GGCAUGAG G UAUGUCGG | 1926 | CCGACATA GGCTAGCTACAACGA CTCATGCC | 4254 |
| 2682 | CAUGAGGU A UGUCGGAA | 1927 | TTCCGACA GGCTAGCTACAACGA ACCTCATG | 4255 |
| 2684 | UGAGGUAU G UCGGAACC | 1928 | GGTTCCGA GGCTAGCTACAACGA ATACCTCA | 4256 |
| 2690 | AUGUCGGA A CCUCAGGC | 1929 | GCCTGAGG GGCTAGCTACAACGA TCCGACAT | 4257 |
| 2697 | AACCUCAG G CCUGUCCA | 1930 | TGGACAGG GGCTAGCTACAACGA CTGAGGTT | 4258 |
| 2701 | UCAGGCCU G UCCAGCCC | 1931 | GGGCTGGA GGCTAGCTACAACGA AGGCCTGA | 4259 |
| 2706 | CCUGUCCA G CCCUGGGC | 1932 | GCCCAGGG GGCTAGCTACAACGA TGGACAGG | 4260 |
| 2713 | AGCCCUGG G CUCUCCAU | 1933 | ATGGAGAG GGCTAGCTACAACGA CCAGGGCT | 4261 |
| 2720 | GGCUCUCC A UAGCCUUU | 1934 | AAAGGCTA GGCTAGCTACAACGA GGAGAGCC | 4262 |
| 2723 | UCUCCAUA G CCUUUGGG | 1935 | CCCAAAGG GGCTAGCTACAACGA TATGGAGA | 4263 |
| 2740 | AGGGGGAG G UUGGGAGA | 1936 | TCTCCCAA GGCTAGCTACAACGA CTCCCCCT | 4264 |
| 2750 | UGGGAGAG G CCGGUCAG | 1937 | CTGACCGG GGCTAGCTACAACGA CTCTCCCA | 4265 |
| 2754 | AGAGGCCG G UCAGGGGU | 1938 | ACCCCTGA GGCTAGCTACAACGA CGGCCTCT | 4266 |
| 2761 | GGUCAGGG G UCUGGGCU | 1939 | AGCCCAGA GGCTAGCTACAACGA CCCTGACC | 4267 |
| 2767 | GGGUCUGG G CUGUGGUG | 1940 | CACCACAG GGCTAGCTACAACGA CCAGACCC | 4268 |
| 2770 | UCUGGGCU G UGGUGCUC | 1941 | GAGCACCA GGCTAGCTACAACGA AGCCCAGA | 4269 |
| 2773 | GGGCUGUG G UGCUCUCU | 1942 | AGAGAGCA GGCTAGCTACAACGA CACAGCCC | 4270 |
| 2775 | GCUGUGGU G CUCUCUCC | 1943 | GGAGAGAG GGCTAGCTACAACGA ACCACAGC | 4271 |
| 2788 | CUCCUCCC G CCUGCCCC | 1944 | GGGGCAGG GGCTAGCTACAACGA GGGAGGAG | 4272 |
| 2792 | UCCCGCCU G CCCCAGUG | 1945 | CACTGGGG GGCTAGCTACAACGA AGGCGGGA | 4273 |
| 2798 | CUGCCCCA G UGUCCACG | 1946 | CGTGGACA GGCTAGCTACAACGA TGGGGCAG | 4274 |
| 2800 | GCCCCAGU G UCCACGGC | 1947 | GCCGTGGA GGCTAGCTACAACGA ACTGGGGC | 4275 |
| 2804 | CAGUGUCC A CGGCUUCU | 1948 | AGAAGCCG GGCTAGCTACAACGA GGACACTG | 4276 |
| 2807 | UGUCCACG G CUUCUGGC | 1949 | GCCAGAAG GGCTAGCTACAACGA CGTGGACA | 4277 |
| 2814 | GGCUUCUG G CAGAGAGC | 1950 | GCTCTCTG GGCTAGCTACAACGA CAGAAGCC | 4278 |
| 2821 | GGCAGAGA G CUCUGGAC | 1951 | GTCCAGAG GGCTAGCTACAACGA TCTCTGCC | 4279 |
| 2828 | AGCUCUGG A CAAGCAGG | 1952 | CCTGCTTG GGCTAGCTACAACGA CCAGAGCT | 4280 |
| 2832 | CUGGACAA G CAGGCAGA | 1953 | TCTGCCTG GGCTAGCTACAACGA TTGTCCAG | 4281 |
| 2836 | ACAAGCAG G CAGAUCAU | 1954 | ATGATCTG GGCTAGCTACAACGA CTGCTTGT | 4282 |
| 2840 | GCAGGCAG A UCAUAAGG | 1955 | CCTTATGA GGCTAGCTACAACGA CTGCCTGC | 4283 |
| 2843 | GGCAGAUC A UAAGGACA | 1956 | TGTCCTTA GGCTAGCTACAACGA GATCTGCC | 4284 |
| 2849 | UCAUAAGG A CAGAGAGC | 1957 | GCTCTCTG GGCTAGCTACAACGA CCTTATGA | 4285 |
| 2856 | GACAGAGA G CUUACUGU | 1958 | ACAGTAAG GGCTAGCTACAACGA TCTCTGTC | 4286 |
| 2860 | GAGAGCUU A CUGUGCUU | 1959 | AAGCACAG GGCTAGCTACAACGA AAGCTCTC | 4287 |
| 2863 | AGCUUACU G UGCUUCUA | 1960 | TAGAAGCA GGCTAGCTACAACGA AGTAAGCT | 4288 |
| 2865 | CUUACUGU G CUUCUACC | 1961 | GGTAGAAG GGCTAGCTACAACGA ACAGTAAG | 4289 |
| 2871 | GUGCUUCU A CCAACUAG | 1962 | CTAGTTGG GGCTAGCTACAACGA AGAAGCAC | 4290 |
| 2875 | UUCUACCA A CUAGGAGG | 1963 | CCTCCTAG GGCTAGCTACAACGA TGGTAGAA | 4291 |
| 2884 | CUAGGAGG G CGUCCUGG | 1964 | CCAGGACG GGCTAGCTACAACGA CCTCCTAG | 4292 |
| 2886 | AGGAGGGC G UCCUGGUC | 1965 | GACCAGGA GGCTAGCTACAACGA GCCCTCCT | 4293 |
| 2892 | GCGUCCUG G UCCUCCAG | 1966 | CTGGAGGA GGCTAGCTACAACGA CAGGACGC | 4294 |
| 2907 | AGAGGGAG G UGGUUUCA | 1967 | TGAAACCA GGCTAGCTACAACGA CTCCCTCT | 4295 |
| 2910 | GGGAGGUG G UUUCAGGG | 1968 | CCCTGAAA GGCTAGCTACAACGA CACCTCCC | 4296 |
| 2919 | UUUCAGGG G UUGGGGAU | 1969 | ATCCCCAA GGCTAGCTACAACGA CCCTGAAA | 4297 |
| 2926 | GGUUGGGG A UCUGUGCC | 1970 | GGCACAGA GGCTAGCTACAACGA CCCCAACC | 4298 |
| 2930 | GGGGAUCU G UGCCGGUG | 1971 | CACCGGCA GGCTAGCTACAACGA AGATCCCC | 4299 |
| 2932 | GGAUCUGU G CCGGUGGC | 1972 | GCCACCGG GGCTAGCTACAACGA ACAGATCC | 4300 |
| 2936 | CUGUGCCG G UGGCUCUG | 1973 | CAGAGCCA GGCTAGCTACAACGA CGGCACAG | 4301 |
| 2939 | UGCCGGUG G CUCUGGUC | 1974 | GACCAGAG GGCTAGCTACAACGA CACCGGCA | 4302 |
| 2945 | UGGCUCUG G UCUCUGCU | 1975 | AGCAGAGA GGCTAGCTACAACGA CAGAGCCA | 4303 |
| 2951 | UGGUCUCU G CUGGGAGC | 1976 | GCTCCCAG GGCTAGCTACAACGA AGAGACCA | 4304 |
| 2958 | UGCUGGGA G CCUUCUUG | 1977 | CAAGAAGG GGCTAGCTACAACGA TCCCAGCA | 4305 |
| 2967 | CCUUCUUG G CGGUGAGA | 1978 | TCTCACCG GGCTAGCTACAACGA CAAGAAGG | 4306 |
| 2970 | UCUUGGCG G UGAGAGGC | 1979 | GCCTCTCA GGCTAGCTACAACGA CGCCAAGA | 4307 |
| 2977 | GGUGAGAG G CAUCACCU | 1980 | AGGTGATG GGCTAGCTACAACGA CTCTCACC | 4308 |
| 2979 | UGAGAGGC A UCACCUUU | 1981 | AAAGGTGA GGCTAGCTACAACGA GCCTCTCA | 4309 |
| 2982 | GAGGCAUC A CCUUUCCU | 1982 | AGGAAAGG GGCTAGCTACAACGA GATGCCTC | 4310 |
| 2992 | CUUUCCUG A CUUGCUCC | 1983 | GGAGCAAG GGCTAGCTACAACGA CAGGAAAG | 4311 |
| 2996 | CCUCACUU G CUCCCAGC | 1984 | GCTGGGAG GGCTAGCTACAACGA AAGTCAGG | 4312 |
| 3003 | UGCUCCCA G CGUGAAAU | 1985 | ATTTCACG GGCTAGCTACAACGA TGGGAGCA | 4313 |
| 3005 | CUCCCAGC G UGAAAUGC | 1986 | GCATTTCA GGCTAGCTACAACGA GCTGGGAG | 4314 |
| 3010 | AGCGUGAA A UGCACCUG | 1987 | CAGGTGCA GGCTAGCTACAACGA TTCACGCT | 4315 |
| 3012 | CGUGAAAU G CACCUGCC | 1988 | GGCAGGTG GGCTAGCTACAACGA ATTTCACG | 4316 |
| 3014 | UGAAAUGC A CCUGCCAA | 1989 | TTGGCAGG GGCTAGCTACAACGA GCATTTCA | 4317 |
| 3018 | AUGCACCU G CCAAGAAU | 1990 | ATTCTTGG GGCTAGCTACAACGA AGGTGCAT | 4318 |
| 3025 | UGCCAAGA A UGGCAGAC | 1991 | GTCTGCCA GGCTAGCTACAACGA TCTTGGCA | 4319 |
| 3028 | CAAGAAUG G CAGACAUA | 1992 | TATGTCTG GGCTAGCTACAACGA CATTCTTG | 4320 |
| 3032 | AAUGGCAG A CAUAGGGA | 1993 | TCCCTATG GGCTAGCTACAACGA CTGCCATT | 4321 |
| 3034 | UGGCAGAC A UAGGGACC | 1994 | GGTCCCTA GGCTAGCTACAACGA GTCTGCCA | 4322 |
| 3040 | ACAUAGGG A CCCCGCCU | 1995 | AGGCGGGG GGCTAGCTACAACGA CCCTATGT | 4323 |
| 3045 | GGGACCCC G CCUCCUGG | 1996 | CCAGGAGG GGCTAGCTACAACGA GGGGTCCC | 4324 |
| 3054 | CCUCCUGG G CCUUCACA | 1997 | TGTGAAGG GGCTAGCTACAACGA CCAGGAGG | 4325 |
| 3060 | GGGCCUUC A CAUGCCCA | 1998 | TGGGCATG GGCTAGCTACAACGA GAAGGCCC | 4326 |
| 3062 | GCCUUCAC A UGCCCAGU | 1999 | ACTGGGCA GGCTAGCTACAACGA GTGAAGGC | 4327 |
| 3064 | CUUCACAU G CCCAGUUU | 2000 | AAACTGGG GGCTAGCTACAACGA ATGTGAAG | 4328 |
| 3069 | CAUGCCCA G UUUUCUUC | 2001 | GAAGAAAA GGCTAGCTACAACGA TGGGCATG | 4329 |
| 3079 | UUUCUUCG G CUCUGUGG | 2002 | CCACAGAG GGCTAGCTACAACGA CGAAGAAA | 4330 |
| 3084 | UCGGCUCU G UGGCCUGA | 2003 | TCAGGCCA GGCTAGCTACAACGA AGAGCCGA | 4331 |
| 3087 | GCUCUGUG G CCUGAAGC | 2004 | GCTTCAGG GGCTAGCTACAACGA CACAGAGC | 4332 |
| 3094 | GGCCUGAA G CGGUCUGU | 2005 | ACAGACCG GGCTAGCTACAACGA TTCAGGCC | 4333 |
| 3097 | CUGAAGCG G UCUGUGGA | 2006 | TCCACAGA GGCTAGCTACAACGA CGCTTCAG | 4334 |
| 3101 | AGCGGUCU G UGGACCUU | 2007 | AAGGTCCA GGCTAGCTACAACGA AGACCGCT | 4335 |
| 3105 | GUCUGUGG A CCUUGGAA | 2008 | TTCCAAGG GGCTAGCTACAACGA CCACAGAC | 4336 |
| 3114 | CCUUGGAA G UAGGGCUC | 2009 | GAGCCCTA GGCTAGCTACAACGA TTCCAAGG | 4337 |
| 3119 | GAAGUAGG G CUCCAGCA | 2010 | TGCTGGAG GGCTAGCTACAACGA CCTACTTC | 4338 |
| 3125 | GGGCUCCA G CACCGACU | 2011 | AGTCGGTG GGCTAGCTACAACGA TGGAGCCC | 4339 |
| 3127 | GCUCCAGC A CCGACUGG | 2012 | CCAGTCGG GGCTAGCTACAACGA GCTGGAGC | 4340 |
| 3131 | CAGCACCG A CUGGCCUC | 2013 | GAGGCCAG GGCTAGCTACAACGA CGGTGCTG | 4341 |
| 3135 | ACCGACUG G CCUCAGGC | 2014 | GCCTGAGG GGCTAGCTACAACGA CAGTCGGT | 4342 |
| 3142 | GGCCUCAG G CCUCUGCC | 2015 | GGCAGAGG GGCTAGCTACAACGA CTGAGGCC | 4343 |
| 3148 | AGGCCUCU G CCUCAUUG | 2016 | CAATGAGG GGCTAGCTACAACGA AGAGGCCT | 4344 |
| 3153 | UCUGCCUC A UUGGUGGU | 2017 | ACCACCAA GGCTAGCTACAACGA GAGGCAGA | 4345 |
| 3157 | CCUCAUUG G UGGUCGGG | 2018 | CCCGACCA GGCTAGCTACAACGA CAATGAGG | 4346 |
| 3160 | CAUUGGUG G UCGGGUAG | 2019 | CTACCCGA GGCTAGCTACAACGA CACCAATG | 4347 |
| 3165 | GUGGUCGG G UAGCGGCC | 2020 | GGCCGCTA GGCTAGCTACAACGA CCGACCAC | 4348 |
| 3168 | GUCGGGUA G CGGCCAGU | 2021 | ACTGGCCG GGCTAGCTACAACGA TACCCGAC | 4349 |
| 3171 | GGGUAGCG G CCAGUAGG | 2022 | CCTACTGG GGCTAGCTACAACGA CGCTACCC | 4350 |
| 3175 | AGCGGCCA G UAGGGCGU | 2023 | ACGCCCTA GGCTAGCTACAACGA TGGCCGCT | 4351 |
| 3180 | CCAGUAGG G CGUGGGAG | 2024 | CTCCCACG GGCTAGCTACAACGA CCTACTGG | 4352 |
| 3182 | AGUAGGGC G UGGGAGCC | 2025 | GGCTCCCA GGCTAGCTACAACGA GCCCTACT | 4353 |
| 3188 | GCGUGGGA G CCUGGCCA | 2026 | TGGCCAGG GGCTAGCTACAACGA TCCCACGC | 4354 |
| 3193 | GGAGCCUG G CCAUCCCU | 2027 | AGGGATGG GGCTAGCTACAACGA CAGGCTCC | 4355 |
| 3196 | GCCUGGCC A UCCCUGCC | 2028 | GGCAGGGA GGCTAGCTACAACGA GGCCAGGC | 4356 |
| 3202 | CCAUCCCU G CCUCCUGG | 2029 | CCAGGAGG GGCTAGCTACAACGA AGGGATGG | 4357 |
| 3212 | CUCCUGGA G UGGACGAG | 2030 | CTCGTCCA GGCTAGCTACAACGA TCCAGGAG | 4358 |
| 3216 | UGGAGUGG A CGAGGUUG | 2031 | CAACCTCG GGCTAGCTACAACGA CCACTCCA | 4359 |
| 3221 | UGGACGAG G UUGGCAGC | 2032 | GCTGCCAA GGCTAGCTACAACGA CTCGTCCA | 4360 |
| 3225 | CGAGGUUG G CAGCUGGU | 2033 | ACCAGCTG GGCTAGCTACAACGA CAACCTCG | 4361 |
| 3228 | GGUUGGCA G CUGGUCCG | 2034 | CGGACCAG GGCTAGCTACAACGA TGCCAACC | 4362 |
| 3232 | GGCAGCUG G UCCGUCUG | 2035 | CAGACGGA GGCTAGCTACAACGA CAGCTGCC | 4363 |
| 3236 | GCUGGUCC G UCUGCUCC | 2036 | GGAGCAGA GGCTAGCTACAACGA GGACCAGC | 4364 |
| 3240 | GUCCGUCU G CUCCUGCC | 2037 | GGCAGGAG GGCTAGCTACAACGA AGACGGAC | 4365 |
| 3246 | CUGCUCCU G CCCCACUC | 2038 | GAGTGGGG GGCTAGCTACAACGA AGGAGCAG | 4366 |
| 3251 | CCUGCCCC A CUCUCCCC | 2039 | GGGGAGAG GGCTAGCTACAACGA GGGGCAGG | 4367 |
| 3261 | UCUCCCCC G CCCCUGCC | 2040 | GGCAGGGG GGCTAGCTACAACGA GGGGGAGA | 4368 |
| 3267 | CCGCCCCU G CCCUCACC | 2041 | GGTGAGGG GGCTAGCTACAACGA AGGGGCGG | 4369 |
| 3273 | CUGCCCUC A CCCUACCC | 2042 | GGGTAGGG GGCTAGCTACAACGA GAGGGCAG | 4370 |
| 3278 | CUCACCCU A CCCUUGCC | 2043 | GGCAAGGG GGCTAGCTACAACGA AGGGTGAG | 4371 |
| 3284 | CUACCCUU G CCCCACGC | 2044 | GCGTGGGG GGCTAGCTACAACGA AAGGGTAG | 4372 |
| 3289 | CUUGCCCC A CGCCUGCC | 2045 | GGCAGGCG GGCTAGCTACAACGA GGGGCAAG | 4373 |
| 3291 | UGCCCCAC G CCUGCCUC | 2046 | GAGGCAGG GGCTAGCTACAACGA GTGGGGCA | 4374 |
| 3295 | CCACGCCU G CCUCAUGG | 2047 | CCATGAGG GGCTAGCTACAACGA AGGCGTGG | 4375 |
| 3300 | CCUGCCUC A UGGCUGGU | 2048 | ACCAGCCA GGCTAGCTACAACGA GAGGCAGG | 4376 |
| 3303 | GCCUCAUG G CUGGUUGC | 2049 | GCAACCAG GGCTAGCTACAACGA CATGAGGC | 4377 |
| 3307 | CAUGGCUG G UUGCUCUU | 2050 | AAGAGCAA GGCTAGCTACAACGA CAGCCATG | 4378 |
| 3310 | GGCUGGUU G CUCUUGGA | 2051 | TCCAAGAG GGCTAGCTACAACGA AACCAGCC | 4379 |
| 3319 | CUCUUGGA G CCUGGUAG | 2052 | CTACCAGG GGCTAGCTACAACGA TCCAAGAG | 4380 |
| 3324 | GGAGCCUG G UAGUGUCA | 2053 | TGACACTA GGCTAGCTACAACGA CAGGCTCC | 4381 |
| 3327 | GCCUGGUA G UGUCACUG | 2054 | CAGTGACA GGCTAGCTACAACGA TACCAGGC | 4382 |
| 3329 | CUGGUAGU G UCACUGGC | 2055 | GCCAGTGA GGCTAGCTACAACGA ACTACCAG | 4383 |
| 3332 | GUAGUGUC A CUGGCUCA | 2056 | TGAGCCAG GGCTAGCTACAACGA GACACTAC | 4384 |
| 3336 | UGUCACUG G CUCAGCCU | 2057 | AGGCTGAG GGCTAGCTACAACGA CAGTGACA | 4385 |
| 3341 | CUGGCUCA G CCUUGCUG | 2058 | CAGCAAGG GGCTAGCTACAACGA TGAGCCAG | 4386 |
| 3346 | UCAGCCUU G CUGGGUAU | 2059 | ATACCCAG GGCTAGCTACAACGA AAGGCTGA | 4387 |
| 3351 | CUUGCUGG G UAUACACA | 2060 | TGTGTATA GGCTAGCTACAACGA CCAGCAAG | 4388 |
| 3353 | UGCUGGGU A UACACAGG | 2061 | CCTGTGTA GGCTAGCTACAACGA ACCCAGCA | 4389 |
| 3355 | CUGGGUAU A CACAGGCU | 2062 | AGCCTGTG GGCTAGCTACAACGA ATACCCAG | 4390 |
| 3357 | GGGUAUAC A CAGGCUCU | 2063 | AGAGCCTG GGCTAGCTACAACGA GTATACCC | 4391 |
| 3361 | AUACACAG G CUCUGCCA | 2064 | TGGCAGAG GGCTAGCTACAACGA CTGTGTAT | 4392 |
| 3366 | CAGGCUCU G CCACCCAC | 2065 | GTGGGTGG GGCTAGCTACAACGA AGAGCCTG | 4393 |
| 3369 | GCUCUGCC A CCCACUCU | 2066 | AGAGTGGG GGCTAGCTACAACGA GGCAGAGC | 4394 |
| 3373 | UGCCACCC A CUCUGCUC | 2067 | GACCAGAG GGCTAGCTACAACGA GGGTGGCA | 4395 |
| 3378 | CCCACUCU G CUCCAAGG | 2068 | CCTTGGAG GGCTAGCTACAACGA AGAGTGGG | 4396 |
| 3388 | UCCAAGGG G CUUGCCCU | 2069 | AGGGCAAG GGCTAGCTACAACGA CCCTTGGA | 4397 |
| 3392 | AGGGGCUU G CCCUGCCU | 2070 | AGGCAGGG GGCTAGCTACAACGA AAGCCCCT | 4398 |
| 3397 | CUUGCCCU G CCUUGGGC | 2071 | GCCCAAGG GGCTAGCTACAACGA AGGGCAAG | 4399 |
| 3404 | UGCCUUGG G CCAAGUUC | 2072 | GAACTTGG GGCTAGCTACAACGA CCAAGGCA | 4400 |
| 3409 | UGGGCCAA G UUCUAGGU | 2073 | ACCTAGAA GGCTAGCTACAACGA TTGGCCCA | 4401 |
| 3416 | AGUUCUAG G UCUGGCCA | 2074 | TGGCCAGA GGCTAGCTACAACGA CTAGAACT | 4402 |
| 3421 | UAGGUCUG G CCACAGCC | 2075 | GGCTGTGG GGCTAGCTACAACGA CAGACCTA | 4403 |
| 3424 | GUCUGGCC A CAGCCACA | 2076 | TGTGGCTG GGCTAGCTACAACGA GGCCAGAC | 4404 |
| 3427 | UGGCCACA G CCACAGAC | 2077 | GTCTGTGG GGCTAGCTACAACGA TGTGGCCA | 4405 |
| 3430 | CCACAGCC A CAGACAGC | 2078 | GCTGTCTG GGCTAGCTACAACGA GGCTGTGG | 4406 |
| 3434 | AGCCACAG A CAGCUCAG | 2079 | CTGAGCTG GGCTAGCTACAACGA CTGTGGCT | 4407 |
| 3437 | CACAGACA G CUCAGUCC | 2080 | GGACTGAG GGCTAGCTACAACGA TGTCTGTG | 4408 |
| 3442 | ACAGCUCA G UCCCCUGU | 2081 | ACAGGGGA GGCTAGCTACAACGA TGAGCTGT | 4409 |
| 3449 | AGUCCCCU G UGUGGUCA | 2082 | TGACCACA GGCTAGCTACAACGA AGGGGACT | 4410 |
| 3451 | UCCCCUGU G UGGUCAUC | 2083 | GATGACCA GGCTAGCTACAACGA ACAGGGGA | 4411 |
| 3454 | CCUGUGUG G UCAUCCUG | 2084 | CAGGATGA GGCTAGCTACAACGA CACACAGG | 4412 |
| 3457 | GUGUGGUC A UCCUGGCU | 2085 | AGCCAGGA GGCTAGCTACAACGA GACCACAC | 4413 |
| 3463 | UCAUCCUG G CUUCUGCU | 2086 | AGCAGAAG GGCTAGCTACAACGA CAGGATGA | 4414 |
| 3469 | UGGCUUCU G CUGGGGGC | 2087 | GCCCCCAG GGCTAGCTACAACGA AGAAGCCA | 4415 |
| 3476 | UGCUGGGG G CCCACAGC | 2088 | GCTGTGGG GGCTAGCTACAACGA CCCCAGCA | 4416 |
| 3480 | GGGGGCCC A CAGCGCCC | 2089 | GGGCGCTG GGCTAGCTACAACGA GGGCCCCC | 4417 |
| 3483 | GGCCCACA G CGCCCCUG | 2090 | CAGGGGCG GGCTAGCTACAACGA TGTGGGCC | 4418 |
| 3485 | CCCACAGC G CCCCUGGU | 2091 | ACCAGGGG GGCTAGCTACAACGA GCTGTGGG | 4419 |
| 3492 | CGCCCCUG G UGCCCCUC | 2092 | GAGGGGCA GGCTAGCTACAACGA CAGGGGCG | 4420 |
| 3494 | CCCCUGGU G CCCCUCCC | 2093 | GGGAGGGG GGCTAGCTACAACGA ACCAGGGG | 4421 |
| 3511 | CUCCCAGG G CCCGGGUU | 2094 | AACCCGGG GGCTAGCTACAACGA CCTGGGAG | 4422 |
| 3517 | GGGCCCGG G UUGAGGCU | 2095 | AGCCTCAA GGCTAGCTACAACGA CCGGGCCC | 4423 |
| 3523 | GGGUUGAG G CUGGGCCA | 2096 | TGGCCCAG GGCTAGCTACAACGA CTCAACCC | 4424 |
| 3528 | GAGGCUGG G CCAGGCCC | 2097 | GGGCCTGG GGCTAGCTACAACGA CCAGCCTC | 4425 |
| 3533 | UGGGCCAG G CCCUCUGG | 2098 | CCAGAGGG GGCTAGCTACAACGA CTGGCCCA | 4426 |
| 3543 | CCUCUGGG A CGGGGACU | 2099 | AGTCCCCG GGCTAGCTACAACGA CCCAGAGG | 4427 |
| 3549 | GGACGGGG A CUUGUGCC | 2100 | GGCACAAG GGCTAGCTACAACGA CCCCGTCC | 4428 |
| 3553 | GGGGACUU G UGCCCUGU | 2101 | ACAGGGCA GGCTAGCTACAACGA AAGTCCCC | 4429 |
| 3555 | GGACUUGU G CCCUGUCA | 2102 | TGACAGGG GGCTAGCTACAACGA ACAAGTCC | 4430 |
| 3560 | UGUGCCCU G UCAGGGUU | 2103 | AACCCTGA GGCTAGCTACAACGA AGGGCACA | 4431 |
| 3566 | CUGUCAGG G UUCCCUAU | 2104 | ATAGGGAA GGCTAGCTACAACGA CCTGACAG | 4432 |
| 3573 | GGUUCCCU A UCCCUGAG | 2105 | CTCAGGGA GGCTAGCTACAACGA AGGGAACC | 4433 |
| 3582 | UCCCUGAG G UUGGGGGA | 2106 | TCCCCCAA GGCTAGCTACAACGA CTCAGGGA | 4434 |
| 3593 | GGGGGAGA G CUAGCAGG | 2107 | CCTGCTAG GGCTAGCTACAACGA TCTCCCCC | 4435 |
| 3597 | GAGAGCUA G CAGGGCAU | 2108 | ATGCCCTG GGCTAGCTACAACGA TAGCTCTC | 4436 |
| 3602 | CUAGCAGG G CAUGCCGC | 2109 | GCGGCATG GGCTAGCTACAACGA CCTGCTAG | 4437 |
| 3604 | AGCAGGGC A UGCCGCUG | 2110 | CAGCGGCA GGCTAGCTACAACGA GCCCTGCT | 4438 |
| 3606 | CAGGGCAU G CCGCUGGC | 2111 | GCCAGCGG GGCTAGCTACAACGA ATGCCCTG | 4439 |
| 3609 | GGCAUGCC G CUGGCUGG | 2112 | CCAGCCAG GGCTAGCTACAACGA GGCATGCC | 4440 |
| 3613 | UGCCGCUG G CUGGCCAG | 2113 | CTGGCCAG GGCTAGCTACAACGA CAGCGGCA | 4441 |
| 3617 | GCUGGCUG G CCAGGGCU | 2114 | AGCCCTGG GGCTAGCTACAACGA CAGCCAGC | 4442 |
| 3623 | UGGCCAGG G CUGCAGGG | 2115 | CCCTGCAG GGCTAGCTACAACGA CCTGGCCA | 4443 |
| 3626 | CCAGGGCU G CAGGGACA | 2116 | TCTCCCTG GGCTAGCTACAACGA AGCCCTGG | 4444 |
| 3632 | CUGCAGGG A CACUCCCC | 2117 | GGGGAGTG GGCTAGCTACAACCA CCCTGCAG | 4445 |
| 3634 | GCAGGGAC A CUCCCCCU | 2118 | AGGGGGAG GGCTAGCTACAACGA GTCCCTGC | 4446 |
| 3646 | CCCCUUUU G UCCAGGGA | 2119 | TCCCTGGA GGCTAGCTACAACGA AAAAGGGG | 4447 |
| 3655 | UCCAGGGA A UACCACAC | 2120 | GTGTGGTA GGCTAGCTACAACGA TCCCTGGA | 4448 |
| 3657 | CAGGGAAU A CCACACUC | 2121 | GAGTGTGG GGCTAGCTACAACGA ATTCCCTG | 4449 |
| 3660 | GGAAUACC A CACUCGCC | 2122 | GGCGAGTG GGCTAGCTACAACGA GGTATTCC | 4450 |
| 3662 | AAUACCAC A CUCGCCCU | 2123 | AGGGCGAG GGCTAGCTACAACGA GTGGTATT | 4451 |
| 3666 | CCACACUC G CCCUUCUC | 2124 | GAGAAGGG GGCTAGCTACAACGA GAGTGTGG | 4452 |
| 3679 | UCUGUCGA G CGAACACC | 2125 | GGTGTTCG GGCTAGCTACAACGA TGGAGAGA | 4453 |
| 3683 | UCCAGCGA A CACCACAC | 2126 | GTGTGGTG GGCTAGCTACAACGA TCGCTGGA | 4454 |
| 3685 | CAGCGAAC A CCACACUC | 2127 | GAGTGTGG GGCTAGCTACAACGA GTTCGCTG | 4455 |
| 3688 | CGAACACC A CACUCGCC | 2128 | GGCGAGTG GGCTAGCTACAACGA GGTGTTCG | 4456 |
| 3690 | AACACCAC A CUCGCCCU | 2129 | AGGGCGAG GGCTAGCTACAACGA GTGGTGTT | 4457 |
| 3694 | CCACACUC G CCCUUCUC | 2124 | GAGAAGGG GGCTAGCTACAACGA GAGTGTGG | 4452 |
| 3711 | UCCAGGGG A CGCCACAC | 2130 | GTGTGGCG GGCTAGCTACAACGA CCCCTGGA | 4458 |
| 3713 | CAGGGGAC G CCACACUC | 2131 | GAGTGTGG GGCTAGCTACAACGA GTCCCCTG | 4459 |
| 3716 | GGGACGCC A CACUCCCC | 2132 | GGGGAGTG GGCTAGCTACAACGA GGCGTCCC | 4460 |
| 3718 | GACGCCAC A CUCCCCCU | 2133 | AGGGGGAG GGCTAGCTACAACGA GTGGCGTC | 4461 |
| 3730 | CCCCUUCU G UCCAGGGG | 2134 | CCCCTGGA GGCTAGCTACAACGA AGAAGGGG | 4462 |
| 3739 | UCCAGGGG A CGCCACAC | 2130 | GTGTGGCG GGCTAGCTACAACGA CCCCTGGA | 4458 |
| 3741 | CAGGGGAC G CCACACUC | 2131 | GAGTGTGG GGCTAGCTACAACGA GTCCCCTG | 4459 |
| 3744 | GGGACGCC A CACUCCCC | 2132 | GGGGAGTG GGCTAGCTACAACGA GGCGTCCC | 4460 |
| 3746 | GACGCCAC A CUCCCCCU | 2133 | AGGGGGAG GGCTAGCTACAACGA GTGGCGTC | 4461 |
| 3767 | UCCAGGGG A CGCCACAC | 2130 | GTGTGGCG GGCTAGCTACAACGA CCCCTGGA | 4458 |
| 3769 | CAGGGGAC G CCACACUC | 2131 | GAGTGTGG GGCTAGCTACAACGA GTCCCCTG | 4459 |
| 3772 | GGGACGCC A CACUCGCC | 2135 | GGCGAGTG GGCTAGCTACAACGA GGCGTCCC | 4463 |
| 3774 | GACGCCAC A CUCGCCCU | 2136 | AGGGCGAG GGCTAGCTACAACGA GTGGCGTC | 4464 |
| 3778 | CCACACUC G CCCUUCUC | 2124 | GAGAAGGG GGCTAGCTACAACGA GAGTGTGG | 4452 |
| 3795 | UCCAGGGG A CGCCACAC | 2130 | GTGTGGCG GGCTAGCTACAACGA CCCCTGGA | 4458 |
| 3797 | CAGGGGAC G CCACACUC | 2131 | GAGTGTGG GGCTAGCTACAACGA GTCCCCTG | 4459 |
| 3800 | GGGACGCC A CACUCCCC | 2135 | GGCGAGTG GGCTAGCTACAACGA GGCGTCCC | 4463 |
| 3802 | GACGCCAC A CUCGCCCU | 2136 | AGGGCGAG GGCTAGCTACAACGA GTGGCGTC | 4464 |
| 3806 | CCACACUC G CCCUUCUC | 2124 | GAGAAGGG GGCTAGCTACAACGA GAGTGTGG | 4452 |
| 3823 | UCCAGGGG A CGCCACAC | 2130 | CTGTGGCG GGCTAGCTACAACCA CCCCTGGA | 4458 |
| 3825 | CAGGGGAC G CCACACUC | 2131 | GAGTGTGG GGCTAGCTACAACGA GTCCCCTG | 4459 |
| 3828 | GGGACGCC A CACUCGCC | 2135 | GGCGAGTG GGCTAGCTACAACGA GGCGTCCC | 4463 |
| 3830 | GACGCCAC A CUCGCCCU | 2136 | AGGGCGAG GGCTAGCTACAACGA GTGGCGTC | 4464 |
| 3834 | CCACACUC G CCCUUCUG | 2137 | CAGAAGGG GGCTAGCTACAACGA GAGTGTGG | 4465 |
| 3842 | GCCCUUCU G UCCAGGGG | 2138 | CCCCTGGA GGCTAGCTACAACGA AGAAGGGC | 4466 |
| 3851 | UCCAGGGG A CGCCACAC | 2130 | GTGTGGCG GGCTAGCTACAACGA CCCCTGGA | 4458 |
| 3853 | CAGGGGAC G CCACACUC | 2131 | GAGTGTGG GGCTAGCTACAACGA GTCCCCTG | 4459 |
| 3856 | GGGACGCC A CACUCGCC | 2135 | GGCGAGTG GGCTAGCTACAACCA GGCGTCCC | 4463 |
| 3858 | GACGCCAC A CUCGCCCU | 2136 | AGGGCGAG GGCTAGCTACAACGA GTGGCGTC | 4464 |
| 3862 | CCACACUC G CCCUUCUC | 2124 | GAGAAGGG GGCTAGCTACAACGA GAGTGTGG | 4452 |
| 3879 | UCCAGGGG A CGCCACAC | 2130 | GTGTGGCG GGCTAGCTACAACGA CCCCTGGA | 4458 |
| 3881 | CAGGGGAC G CCACACUC | 2131 | GAGTGTGG GGCTAGCTACAACGA GTCCCCTG | 4459 |
| 3884 | GGGACGCC A CACUCGCC | 2135 | GGCGAGTG GGCTAGCTACAACGA GGCGTCCC | 4463 |
| 3886 | GACGCCAC A CUCGCCCU | 2136 | AGGGCGAG GGCTAGCTACAACGA GTGGCGTC | 4464 |
| 3890 | CCACACUC G CCCUUCUC | 2124 | GAGAAGGG GGCTAGCTACAACGA GAGTGTGG | 4452 |
| 3907 | UCCAGGGG A CGCCACAC | 2130 | GTGTGGCG GGCTAGCTACAACCA CCCCTGGA | 4458 |
| 3909 | CAGGGGAC G CCACACUC | 2131 | GAGTGTGG GGCTAGCTACAACGA GTCCCCTG | 4459 |
| 3912 | GGGACGCC A CACUCCCC | 2132 | GGGGAGTG GGCTAGCTACAACGA GGCGTCCC | 4460 |
| 3914 | GACGCCAC A CUCCCCCU | 2133 | AGGGGGAG GGCTAGCTACAACGA GTGGCGTC | 4461 |
| 3926 | CCCCUUCU G UCCAGGGG | 2134 | CCCCTGGA GGCTAGCTACAACGA AGAAGGGG | 4462 |
| 3935 | UCCAGGGG A CGCCACAC | 2130 | GTGTGGCG GGCTAGCTACAACGA CCCCTGGA | 4458 |
| 3937 | CAGGGGAC G CCACACUC | 2131 | GAGTGTGG GGCTAGCTACAACGA GTCCCCTG | 4459 |
| 3940 | GGGACGCC A CACUCCCC | 2132 | GGGGAGTG GGCTAGCTACAACGA GGCGTCCC | 4460 |
| 3942 | GACGCCAC A CUCCCCCU | 2133 | AGGGGGAG GGCTAGCTACAACGA GTGGCGTC | 4461 |
| 3963 | UCCAGGGG A CGCCACAC | 2130 | GTGTGGCG GGCTAGCTACAACGA CCCCTGGA | 4458 |
| 3965 | CAGGGGAC G CCACACUC | 2131 | GAGTGTCG GGCTAGCTACAACGA GTCCCCTG | 4459 |
| 3968 | GGGACGCC A CACUCCCC | 2132 | GGGGAGTG GGCTAGCTACAACGA GGCGTCCC | 4460 |
| 3970 | GACGCCAC A CUCCCCCU | 2133 | AGGGGGAG GGCTAGCTACAACGA GTGGCGTC | 4461 |
| 3991 | UCCAGGGG A CGCCACAC | 2130 | GTGTGGCG GGCTAGCTACAACGA CCCCTGGA | 4458 |
| 3993 | CAGGGGAC G CCACACUC | 2131 | GAGTGTGG GGCTAGCTACAACGA GTCCCCTG | 4459 |
| 3996 | GGGACGCC A CACUCGCC | 2135 | GGCGAGTC GGCTAGCTACAACGA GGCGTCCC | 4463 |
| 3998 | GACGCCAC A CUCGCCCU | 2136 | AGGGCGAG GGCTAGCTACAACGA GTGGCGTC | 4464 |
| 4002 | CCACACUC G CCCUUCUC | 2124 | GAGAAGGG GGCTAGCTACAACGA GAGTGTGG | 4452 |
| 4019 | UCCAGGGG A CGCCACAC | 2130 | GTGTGGCG GGCTAGCTACAACGA CCCCTGGA | 4458 |
| 4021 | CAGGGGAC G CCACACUC | 2131 | GAGTGTGG GGCTAGCTACAACGA GTCCCCTG | 4459 |
| 4024 | GGGACGCC A CACUCCCC | 2132 | GGGGAGTG GGCTAGCTACAACGA GGCGTCCC | 4460 |
| 4026 | GACGCCAC A CUCCCCCU | 2133 | AGGGGGAG GGCTAGCTACAACGA GTGGCGTC | 4461 |
| 4038 | CCCCUUCU G UCCAGGGG | 2134 | CCCCTGGA GGCTAGCTACAACGA AGAAGGGG | 4462 |
| 4047 | UCCAGGGG A CGCCACAC | 2130 | GTGTGGCG GGCTAGCTACAACGA CCCCTGGA | 4458 |
| 4049 | CAGGGGAC G CCACACUC | 2131 | GAGTGTGG GGCTAGCTACAACGA GTCCCCTG | 4459 |
| 4052 | GGGACGCC A CACUCGCC | 2135 | GGCGAGTG GGCTAGCTACAACGA GGCGTCCC | 4463 |
| 4054 | GACGCCAC A CUCGCCCU | 2136 | AGGGCGAG GGCTAGCTACAACGA GTGGCGTC | 4464 |
| 4058 | CCACACUC G CCCUUCUC | 2124 | GAGAAGGG GGCTAGCTACAACGA GAGTGTGG | 4452 |
| 4075 | UCCAGGGG A CGCCACAC | 2130 | GTGTGGCG GGCTAGCTACAACGA CCCCTGGA | 4458 |
| 4077 | CAGGGGAC G CCACACUC | 2131 | GAGTGTGG GGCTAGCTACAACGA GTCCCCTG | 4459 |
| 4080 | GGGACGCC A CACUCGCC | 2135 | GGCGAGTG GGCTAGCTACAACGA GGCGTCCC | 4463 |
| 4082 | GACGCCAC A CUCGCCCU | 2136 | AGGGCGAG GGCTAGCTACAACGA GTGGCGTC | 4464 |
| 4086 | CCACACUC G CCCUUCUC | 2124 | GAGAAGGG GGCTAGCTACAACGA GAGTGTGG | 4452 |
| 4103 | UCCAGGGG A CGCCACAC | 2130 | GTGTGGCG GGCTAGCTACAACGA CCCCTGGA | 4458 |
| 4105 | CAGGGGAC G CCACACUC | 2131 | GAGTGTGG GGCTAGCTACAACGA GTCCCCTG | 4459 |
| 4108 | GGGACGCC A CACUCCCC | 2132 | GGGGAGTG GGCTAGCTACAACGA GGCGTCCC | 4460 |
| 4110 | GACGCCAC A CUCCCCCU | 2133 | AGGGGGAG GGCTAGCTACAACGA GTGGCGTC | 4461 |
| 4131 | UCCAGGGG A CGCCACAC | 2130 | GTGTGGCG GGCTAGCTACAACGA CCCCTGGA | 4458 |
| 4133 | CAGGGGAC G CCACACUC | 2131 | GAGTGTGG GGCTAGCTACAACGA GTCCCCTG | 4459 |
| 4136 | GGGACGCC A CACUCCCC | 2132 | GGGGAGTG GGCTAGCTACAACGA GGCGTCCC | 4460 |
| 4138 | GACGCCAC A CUCCCCCU | 2133 | AGGGGGAG GGCTAGCTACAACGA GTGGCGTC | 4461 |
| 4159 | UCCAGGGG A CGCCACAC | 2130 | GTGTGGCG GGCTAGCTACAACGA CCCCTGGA | 4458 |
| 4161 | CAGGGGAC G CCACACUC | 2131 | GAGTGTGG GGCTAGCTACAACGA GTCCCCTG | 4459 |
| 4164 | GGGACGCC A CACUCCCC | 2132 | GGGGAGTG GGCTAGCTACAACGA GGCGTCCC | 4460 |
| 4166 | GACGCCAC A CUCCCCCU | 2133 | AGGGGGAG GGCTAGCTACAACGA GTGGCGTC | 4461 |
| 4178 | CCCCUUCU G UCCAGGGG | 2134 | CCCCTGGA GGCTAGCTACAACGA AGAAGGGG | 4462 |
| 4187 | UCCAGGGG A CGCCACAC | 2130 | GTGTGGCG GGCTAGCTACAACGA CCCCTGGA | 4458 |
| 4189 | CAGGGGAC G CCACACUC | 2131 | GAGTGTGG GGCTAGCTACAACGA GTCCCCTG | 4459 |
| 4192 | GGGACGCC A CACUCGCC | 2135 | GGCGAGTG GGCTAGCTACAACGA GGCGTCCC | 4463 |
| 4194 | GACGCCAC A CUCGCCCU | 2136 | AGGGCGAG GGCTAGCTACAACGA GTGGCGTC | 4464 |
| 4198 | CCACACUC G CCCUUCUC | 2124 | GAGAAGGG GGCTAGCTACAACGA GAGTGTGG | 4452 |
| 4215 | UCCAGGGG A CGCCACAC | 2130 | GTGTGGCG GGCTAGCTACAACGA CCCCTGGA | 4458 |
| 4217 | CAGGGGAC G CCACACUC | 2131 | GAGTGTGG GGCTAGCTACAACGA GTCCCCTG | 4459 |
| 4220 | GGGACGCC A CACUCCCC | 2132 | GGGGAGTG GGCTAGCTACAACGA GGCGTCCC | 4460 |
| 4222 | GACGCCAC A CUCCCCCU | 2133 | AGGGGGAG GGCTAGCTACAACGA GTGGCGTC | 4461 |
| 4243 | UCCAGGGG A CGCCACAC | 2130 | GTGTGGCG GGCTAGCTACAACGA CCCCTGGA | 4458 |
| 4245 | CAGGGGAC G CCACACUC | 2131 | GAGTGTGG GGCTAGCTACAACGA GTCCCCTG | 4459 |
| 4248 | GGGACGCC A CACUCCCC | 2132 | GGGGAGTG GGCTAGCTACAACGA GGCGTCCC | 4460 |
| 4250 | GACGCCAC A CUCCCCCU | 2133 | AGGGGGAG GGCTAGCTACAACGA GTGGCGTC | 4461 |
| 4271 | UCCAGGGG A CGCCACAC | 2130 | GTGTGGCG GGCTAGCTACAACGA CCCCTGGA | 4458 |
| 4273 | CAGGGGAC G CCACACUC | 2131 | GAGTGTGG GGCTAGCTACAACGA GTCCCCTG | 4459 |
| 4276 | GGGACGCC A CACUCCCC | 2132 | GGGGAGTG GGCTAGCTACAACGA GGCGTCCC | 4460 |
| 4278 | GACGCCAC A CUCCCCCU | 2133 | AGGGGGAG GGCTAGCTACAACGA GTGGCGTC | 4461 |
| 4290 | CCCCUUCU G UCCAGGGG | 2134 | CCCCTGGA GGCTAGCTACAACGA AGAAGGGG | 4462 |
| 4299 | UCCAGGGG A CGCCACAC | 2130 | GTGTGGCG GGCTAGCTACAACGA CCCCTGGA | 4458 |
| 4301 | CAGGGGAC G CCACACUC | 2131 | GAGTGTGG GGCTAGCTACAACGA GTCCCCTG | 4459 |
| 4304 | GGGACGCC A CACUCGCC | 2135 | GGCGAGTG GGCTAGCTACAACGA GGCGTCCC | 4463 |
| 4306 | GACGCCAC A CUCGCCCU | 2136 | AGGGCGAG GGCTAGCTACAACGA GTGGCGTC | 4464 |
| 4310 | CCACACUC G CCCUUCUC | 2124 | GAGAAGGG GGCTAGCTACAACGA GAGTGTGG | 4452 |
| 4327 | UCCAGGGG A CGCCACAC | 2130 | GTGTGGCG GGCTAGCTACAACGA CCCCTGGA | 4458 |
| 4329 | CAGGGGAC G CCACACUC | 2131 | GAGTGTGG GGCTAGCTACAACGA GTCCCCTG | 4459 |
| 4332 | GGGACGCC A CACUCGCC | 2135 | GGCGAGTG GGCTAGCTACAACGA GGCGTCCC | 4463 |
| 4334 | GACGCCAC A CUCGCCCU | 2136 | AGGGCGAG GGCTAGCTACAACGA GTGGCGTC | 4464 |
| 4338 | CCACACUC G CCCUUCUC | 2124 | GAGAAGGG GGCTAGCTACAACGA GAGTGTGG | 4452 |
| 4355 | UCCAGGGG A CGCCACAC | 2130 | GTGTGGCG GGCTAGCTACAACGA CCCCTGGA | 4458 |
| 4357 | CAGGGGAC G CCACACUC | 2131 | GAGTGTGG GGCTAGCTACAACGA GTCCCCTG | 4459 |
| 4360 | GGGACGCC A CACUCGCC | 2135 | GGCGAGTG GGCTAGCTACAACGA GGCGTCCC | 4463 |
| 4362 | GACGCCAC A CUCGCCCU | 2136 | AGGGCGAG GGCTAGCTACAACGA GTGGCGTC | 4464 |
| 4366 | CCACACUC G CCCUUCUC | 2124 | GAGAAGGG GGCTAGCTACAACGA GAGTGTGG | 4452 |
| 4383 | UCCAGGGG A CGCCACAC | 2130 | GTGTGGCG GGCTAGCTACAACGA CCCCTGGA | 4458 |
| 4385 | CAGGGGAC G CCACACUU | 2139 | AAGTGTGG GGCTAGCTACAACGA GTCCCCTG | 4467 |
| 4388 | GGGACGCC A CACUUGCC | 2140 | GGCAAGTG GGCTAGCTACAACGA GGCGTCCC | 4468 |
| 4390 | GACGCCAC A CUUGCCCU | 2141 | AGGGCAAG GGCTAGCTACAACGA GTGGCGTC | 4469 |
| 4394 | CCACACUU G CCCUUCUG | 2142 | CAGAAGGG GGCTAGCTACAACGA AAGTGTGG | 4470 |
| 4402 | GCCCUUCU G UCCAGGGA | 2143 | TCCCTGGA GGCTAGCTACAACGA AGAAGGGC | 4471 |
| 4411 | UCCAGGGA A UGCCACAC | 2144 | GTGTGGCA GGCTAGCTACAACGA TCCCTGGA | 4472 |
| 4413 | CAGGGAAU G CCACACUC | 2145 | GAGTGTGG GGCTAGCTACAACGA ATTCCCTG | 4449 |
| 4416 | GGAAUGCC A CACUCCCC | 2146 | GGGGAGTG GGCTAGCTACAACGA GGCATTCC | 4473 |
| 4418 | AAUGCCAC A CUCCCCCU | 2147 | AGGGGGAG GGCTAGCTACAACGA GTGGCATT | 4474 |
| 4435 | UCUCCCCA G CAGCCUCC | 2148 | GGAGGCTG GGCTAGCTACAACGA TGGGGAGA | 4475 |
| 4438 | CCCCAGCA G CCUCCGAG | 2149 | CTCGGAGG GGCTAGCTACAACGA TGCTGGGG | 4476 |
| 4446 | GCCUCCGA G UGACCAGC | 2150 | GCTGGTCA GGCTAGCTACAACGA TCGGAGGC | 4477 |
| 4449 | UCCGAGUG A CCAGCUUC | 2151 | GAAGCTGG GGCTAGCTACAACGA CACTCGGA | 4478 |
| 4453 | AGUGACCA G CUUCCCCA | 2152 | TGGGGAAG GGCTAGCTACAACGA TGGTCACT | 4479 |
| 4461 | GCUUCCCC A UCGAUAGA | 2153 | TCTATCGA GGCTAGCTACAACGA GGGGAAGC | 4480 |
| 4465 | CCCCAUCG A UAGACUUC | 2154 | GAAGTCTA GGCTAGCTACAACGA CGATGGGG | 4481 |
| 4469 | AUCGAUAG A CUUCCCGA | 2155 | TCGGGAAG GGCTAGCTACAACGA CTATCGAT | 4482 |
| 4479 | UUCCCGAG G CCAGGAGC | 2156 | GCTCCTGG GGCTAGCTACAACGA CTCGGGAA | 4483 |
| 4486 | GGCCAGGA G CCCUCUAG | 2157 | CTAGAGGG GGCTAGCTACAACGA TCCTGGCC | 4484 |
| 4496 | CCUCUAGG G CUGCCGGG | 2158 | CCCGGCAG GGCTAGCTACAACGA CCTAGAGG | 4485 |
| 4499 | CUAGGGCU G CCGGGUGC | 2159 | GCACCCGG GGCTAGCTACAACGA AGCCCTAG | 4486 |
| 4504 | GCUGCCGG G UGCCACCC | 2160 | GGGTGGCA GGCTAGCTACAACGA CCGGCAGC | 4487 |
| 4506 | UGCCGGGU G CCACCCUG | 2161 | CAGGGTGG GGCTAGCTACAACGA ACCCGGCA | 4488 |
| 4509 | CGGGUGCC A CCCUGGCU | 2162 | AGCCAGGG GGCTAGCTACAACGA GGCACCCG | 4489 |
| 4515 | CCACCCUG G CUCCUUCC | 2163 | GGAAGGAG GGCTAGCTACAACGA CAGGGTGG | 4490 |
| 4524 | CUCCUUCC A CACCGUGC | 2164 | GCACGGTG GGCTAGCTACAACGA GGAAGGAG | 4491 |
| 4526 | CCUUCCAC A CCGUGCUG | 2165 | CAGCACGG GGCTAGCTACAACGA GTGGAAGG | 4492 |
| 4529 | UCCACACC G UGCUGGUC | 2166 | GACCAGCA GGCTAGCTACAACGA GGTGTGGA | 4493 |
| 4531 | CACACCGU G CUGGUCAC | 2167 | GTGACCAG GGCTAGCTACAACGA ACGGTGTG | 4494 |
| 4535 | CCGUGCUG G UCACUGCC | 2168 | GGCAGTGA GGCTAGCTACAACGA CAGCACGG | 4495 |
| 4538 | UGCUGGUC A CUGCCUGC | 2169 | GCAGGCAG GGCTAGCTACAACGA GACCAGCA | 4496 |
| 4541 | UGGUCACU G CCUGCUGG | 2170 | CCAGCAGG GGCTAGCTACAACGA AGTGACCA | 4497 |
| 4545 | CACUGCCU G CUGGGGGC | 2171 | GCCCCCAG GGCTAGCTACAACGA AGGCAGTG | 4498 |
| 4552 | UGCUGGGG G CGUCAGAU | 2172 | ATCTGACG GGCTAGCTACAACGA CCCCAGCA | 4499 |
| 4554 | CUGGGGGC G UCAGAUGC | 2173 | GCATCTGA GGCTAGCTACAACGA GCCCCCAG | 4500 |
| 4559 | GGCGUCAG A UGCAGGUG | 2174 | CACCTGCA GGCTAGCTACAACGA CTGACGCC | 4501 |
| 4561 | CGUCAGAU G CAGGUGAC | 2175 | GTCACCTG GGCTAGCTACAACGA ATCTGACG | 4502 |
| 4565 | AGAUGCAG G UGACCCUG | 2176 | CAGGGTCA GGCTAGCTACAACGA CTGCATCT | 4503 |
| 4568 | UGCAGGUG A CCCUGUGC | 2177 | GCACAGGG GGCTAGCTACAACGA CACCTGCA | 4504 |
| 4573 | GUGACCCU G UGCAGGAG | 2178 | CTCCTGCA GGCTAGCTACAACGA AGGGTCAC | 4505 |
| 4575 | GACCCUGU G CAGGAGGU | 2179 | ACCTCCTG GGCTAGCTACAACGA ACAGGGTC | 4506 |
| 4582 | UGCAGGAG G UAUCUCUG | 2180 | CAGAGATA GGCTAGCTACAACGA CTCCTGCA | 4507 |
| 4584 | CAGGAGGU A UCUCUGGA | 2181 | TCCAGAGA GGCTAGCTACAACGA ACCTCCTG | 4508 |
| 4592 | AUCUCUGG A CCUGCCUC | 2182 | GAGGCAGG GGCTAGCTACAACGA CCAGAGAT | 4509 |
| 4596 | CUGGACCU G CCUCUUGG | 2183 | CCAAGAGG GGCTAGCTACAACGA AGGTCCAG | 4510 |
| 4604 | GCCUCUUG G UCAUUACG | 2184 | CGTAATGA GGCTAGCTACAACGA CAAGAGGC | 4511 |
| 4607 | UCUUGGUC A UUACGGGG | 2185 | CCCCGTAA GGCTAGCTACAACGA GACCAAGA | 4512 |
| 4610 | UGGUCAUU A CGGGGCUG | 2186 | CAGCCCCG GGCTAGCTACAACGA AATGACCA | 4513 |
| 4615 | AUUACGGG G CUGGGCAG | 2187 | CTGCCCAG GGCTAGCTACAACGA CCCGTAAT | 4514 |
| 4620 | GGGGCUGG G CAGGGCCU | 2188 | AGGCCCTG GGCTAGCTACAACGA CCAGCCCC | 4515 |
| 4625 | UGGGCAGG G CCUGGUAU | 2189 | ATACCAGG GGCTAGCTACAACGA CCTGCCCA | 4516 |
| 4630 | AGGGCCUG G UAUCAGGG | 2190 | CCCTGATA GGCTAGCTACAACGA CAGGCCCT | 4517 |
| 4632 | GGCCUGGU A UCAGGGCC | 2191 | GGCCCTGA GGCTAGCTACAACGA ACCAGGCC | 4518 |
| 4638 | GUAUCAGG G CCCCGCUG | 2192 | CAGCGGGG GGCTAGCTACAACGA CCTGATAC | 4519 |
| 4643 | AGGGCCCC G CUGGGGUU | 2193 | AACCCCAG GGCTAGCTACAACGA GGGGCCCT | 4520 |
| 4649 | CCGCUGGG G UUGCAGGG | 2194 | CCCTGCAA GGCTAGCTACAACGA CCCAGCGG | 4521 |
| 4652 | CUGGGGUU G CAGGGCUG | 2195 | CAGCCCTG GGCTAGCTACAACGA AACCCCAG | 4522 |
| 4657 | GUUGCAGG U CUGGGCCU | 2196 | AGGCCCAG GGCTAGCTACAACGA CCTGCAAC | 4523 |
| 4662 | AGGGCUGG G CCUGUGCU | 2197 | AGCACAGG GGCTAGCTACAACGA CCAGCCCT | 4524 |
| 4666 | CUGGGCCU G UGCUGUGG | 2198 | CCACAGCA GGCTAGCTACAACGA AGGCCCAG | 4525 |
| 4668 | GGGCCUGU G CUGUGGUC | 2199 | GACCACAG GGCTAGCTACAACGA ACAGGCCC | 4526 |
| 4671 | CCUGUGCU G UGGUCCUG | 2200 | CAGGACCA GGCTAGCTACAACGA AGCACAGG | 4527 |
| 4674 | GUGCUGUG G UCCUGGGG | 2201 | CCCCAGGA GGCTAGCTACAACGA CACAGCAC | 4528 |
| 4682 | GUCCUGUG G UGUCCAGG | 2202 | CCTGGACA GGCTAGCTACAACGA CCCAGGAC | 4529 |
| 4684 | CCUGGGGU G UCCAGGAC | 2203 | GTCCTGGA GGCTAGCTACAACGA ACCCCAGG | 4530 |
| 4691 | UGUCCAGG A CAGACGUG | 2204 | CACGTCTG GGCTAGCTACAACGA CCTGGACA | 4531 |
| 4695 | CAGGACAG A CGUGGAGG | 2205 | CCTCCACG GGCTAGCTACAACGA CTGTCCTG | 4532 |
| 4697 | GGACAGAC G UGGAGGGG | 2206 | CCCCTCCA GGCTAGCTACAACGA GTCTGTCC | 4533 |
| 4705 | GUGGAGGG G UCAGGGCC | 2207 | GGCCCTGA GGCTAGCTACAACGA CCCTCCAC | 4534 |
| 4711 | GGGUCAGG G CCCAGCAC | 2208 | GTGCTGGG GGCTAGCTACAACGA CCTGACCC | 4535 |
| 4716 | AGGGCCCA G CACCCCUG | 2209 | CAGGGGTG GGCTAGCTACAACGA TGGGCCCT | 4536 |
| 4718 | GGCCCAGC A CCCCUGCU | 2210 | AGCAGGGG GGCTAGCTACAACGA GCTGGGCC | 4537 |
| 4724 | GCACCCCU G CUCCAUGC | 2211 | GCATGGAG GGCTAGCTACAACGA AGGGGTGC | 4538 |
| 4729 | CCUGCUCC A UGCUGAAC | 2212 | GTTCAGCA GGCTAGCTACAACGA GGAGCAGG | 4539 |
| 4731 | UGCUCCAU G CUGAACUG | 2213 | CAGTTCAG GGCTAGCTACAACGA ATGGAGCA | 4540 |
| 4736 | CAUGCUGA A CUGUGGGA | 2214 | TCCCACAG GGCTAGCTACAACGA TCAGCATG | 4541 |
| 4739 | GCUGAACU G UGGGAAGC | 2215 | GCTTCCCA GGCTAGCTACAACGA AGTTCAGC | 4542 |
| 4746 | UGUGGGAA G CAUCCAGG | 2216 | CCTGGATG GGCTAGCTACAACGA TTCCCACA | 4543 |
| 4748 | UGGGAAGC A UCCAGGUC | 2217 | GACCTGGA GGCTAGCTACAACGA GCTTCCCA | 4544 |
| 4754 | GCAUCCAG G UCCCUGGG | 2218 | CCCAGGGA GGCTAGCTACAACGA CTGGATGC | 4545 |
| 4762 | GUCCCUGG G UGGCUUCA | 2219 | TGAAGCCA GGCTAGCTACAACGA CCAGGGAC | 4546 |
| 4765 | CCUGGGUG G CUUCAACA | 2220 | TGTTGAAG GGCTAGCTACAACGA CACCCAGG | 4547 |
| 4771 | UGGCUUCA A CAGGAGUU | 2221 | AACTCCTG GGCTAGCTACAACGA TGAAGCCA | 4548 |
| 4777 | CAACAGGA G UUCCAGCA | 2222 | TGCTGGAA GGCTAGCTACAACGA TCCTGTTG | 4549 |
| 4783 | GAGUUCCA G CACGGGAA | 2223 | TTCCCGTG GGCTAGCTACAACGA TGGAACTC | 4550 |
| 4785 | GUUCCAGC A CGGGAACC | 2224 | GGTTCCCG GGCTAGCTACAACGA GCTGGAAC | 4551 |
| 4791 | GCACGGGA A CCACUGGA | 2225 | TCCAGTGG GGCTAGCTACAACGA TCCCGTGC | 4552 |
| 4794 | CGGGAACC A CUGGACAA | 2226 | TTGTCCAG GGCTAGCTACAACGA GGTTCCCG | 4553 |
| 4799 | ACCACUGG A CAACCUGG | 2227 | CCAGGTTG GGCTAGCTACAACGA CCAGTGGT | 4554 |
| 4802 | ACUGGACA A CCUGGGGU | 2228 | ACCCCAGG GGCTAGCTACAACGA TGTCCAGT | 4555 |
| 4809 | AACCUGGG G UGUGUCCU | 2229 | AGGACACA GGCTAGCTACAACGA CCCAGGTT | 4556 |
| 4811 | CCUGGGGU C UGUCCUGA | 2230 | TCAGGACA GGCTAGCTACAACGA ACCCCAGG | 4557 |
| 4813 | UGGGGUGU G UCCUGAUC | 2231 | GATCAGGA GGCTAGCTACAACGA ACACCCCA | 4558 |
| 4819 | GUGUCCUG A UCUGGGGA | 2232 | TCCCCAGA GGCTAGCTACAACGA CAGGACAC | 4559 |
| 4827 | AUCUGGGG A CAGGCCAG | 2233 | CTGGCCTG GGCTAGCTACAACGA CCCCAGAT | 4560 |
| 4831 | GGGGACAG G CCAGCCAC | 2234 | GTGGCTGG GGCTAGCTACAACGA CTGTCCCC | 4561 |
| 4835 | ACAGGCCA G CCACACCC | 2235 | GGGTGTGG GGCTAGCTACAACGA TGGCCTGT | 4562 |
| 4838 | GGCCAGCC A CACCCCGA | 2236 | TCGGGGTG GGCTAGCTACAACGA GGCTGGCC | 4563 |
| 4840 | CCAGCCAC A CCCCGAGU | 2237 | ACTCGGGG GGCTAGCTACAACGA GTGGCTGG | 4564 |
| 4847 | CACCCCGA G UCCUAGGG | 2238 | CCCTAGGA GGCTAGCTACAACGA TCGGGGTG | 4565 |
| 4856 | UCCUAGGG A CUCCAGAG | 2239 | CTCTGGAG GGCTAGCTACAACGA CCCTAGGA | 4566 |
| 4866 | UCCAGAGA G CAGCCCAC | 2240 | GTGGGCTG GGCTAGCTACAACGA TCTCTGGA | 4567 |
| 4869 | AGAGAGCA G CCCACUGC | 2241 | GCAGTGGG GGCTAGCTACAACGA TGCTCTCT | 4568 |
| 4873 | AGCAGCCC A CUGCCCUG | 2242 | CAGGGCAG GGCTAGCTACAACGA GGGCTGCT | 4569 |
| 4876 | AGCCCACU G CCCUGGGC | 2243 | GCCCAGGG GGCTAGCTACAACGA AGTGGGCT | 4570 |
| 4883 | UGCCCUGG G CUCCACGG | 2244 | CCGTGGAG GGCTAGCTACAACGA CCAGGGCA | 4571 |
| 4888 | UGGGCUCC A CGGAAGCC | 2245 | GGCTTCCG GGCTAGCTACAACGA GGAGCCCA | 4572 |
| 4894 | CCACGGAA G CCCCCUCA | 2246 | TGAGGGGG GGCTAGCTACAACGA TTCCGTGG | 4573 |
| 4902 | GCCCCCUC A UGCCGCUA | 2247 | TAGCGGCA GGCTAGCTACAACGA GAGGGGGC | 4574 |
| 4904 | CCCCUCAU G CCGCUAGG | 2248 | CCTAGCGG GGCTAGCTACAACGA ATGAGGGG | 4575 |
| 4907 | CUCAUGCC G CUAGGCCU | 2249 | AGGCCTAG GGCTAGCTACAACGA GGCATGAG | 4576 |
| 4912 | GCCGCUAG G CCUUGGCC | 2250 | GGCCAAGG GGCTAGCTACAACGA CTAGCGGC | 4577 |
| 4918 | AGGCCUUG G CCUCGGGG | 2251 | CCCCGAGG GGCTAGCTACAACGA CAAGGCCT | 4578 |
| 4927 | CCUCGGGG A CAGCCCAG | 2252 | CTGGGCTG GGCTAGCTACAACGA CCCCGAGG | 4579 |
| 4930 | CGGGGACA G CCCAGCUA | 2253 | TAGCTGGG GGCTAGCTACAACGA TGTCCCCG | 4580 |
| 4935 | ACAGCCCA G CUAGGCCA | 2254 | TGGCCTAG GGCTAGCTACAACGA TGGGCTGT | 4581 |
| 4940 | CCAGCUAG G CCAGUGUG | 2255 | CACACTGG GGCTAGCTACAACGA CTAGCTGG | 4582 |
| 4944 | CUAGGCCA G UGUGUGGC | 2256 | GCCACACA GGCTAGCTACAACGA TGGCCTAG | 4583 |
| 4946 | AGGCCAGU G UGUGGCAG | 2257 | CTGCCACA GGCTAGCTACAACGA ACTGGCCT | 4584 |
| 4948 | GCCAGUGU G UGGCAGGA | 2258 | TCCTGCCA GGCTAGCTACAACGA ACACTGGC | 4585 |
| 4951 | AGUGUGUG G CAGGACCA | 2259 | TGGTCCTG GGCTAGCTACAACGA CACACACT | 4586 |
| 4956 | GUGGCAGG A CCAGGCCC | 2260 | GGGCCTGG GGCTAGCTACAACGA CCTGCCAC | 4587 |
| 4961 | AGGACCAG G CCCCCAUG | 2261 | CATGGGGG GGCTAGCTACAACGA CTGGTCCT | 4588 |
| 4967 | AGGCCCCC A UGUGGGAG | 2262 | CTCCCACA GGCTAGCTACAACGA GGGGGCCT | 4589 |
| 4969 | GCCCCCAU G UGGGAGCU | 2263 | AGCTCCCA GGCTAGCTACAACGA ATGGGGGC | 4590 |
| 4975 | AUGUGGGA G CUGACCCC | 2264 | GGGGTCAG GGCTAGCTACAACGA TCCCACAT | 4591 |
| 4979 | GGGAGCUG A CCCCUUGG | 2265 | CCAAGGGG GGCTAGCTACAACGA CAGCTCCC | 4592 |
| 4989 | CCCUUGGG A UUCUGGAG | 2266 | CTCCAGAA GGCTAGCTACAACGA CCCAAGGG | 4593 |
| 4997 | AUUCUGGA G CUGUGCUG | 2267 | CAGCACAG GGCTAGCTACAACGA TCCAGAAT | 4594 |
| 5000 | CUGGAGCU G UGCUGAUG | 2268 | CATCAGCA GGCTAGCTACAACGA AGCTCCAG | 4595 |
| 5002 | GGAGCUGU G CUGAUGGG | 2269 | CCCATCAG GGCTAGCTACAACGA ACAGCTCC | 4596 |
| 5006 | CUGUGCUG A UGGGCAGG | 2270 | CCTGCCCA GGCTAGCTACAACGA CAGCACAG | 4597 |
| 5010 | GCUGAUGG G CAGGGGAG | 2271 | CTCCCCTG GGCTAGCTACAACGA CCATCAGC | 4598 |
| 5020 | AGOGGAGA G CCAGCUCC | 2272 | GGAGCTGG GGCTAGCTACAACGA TCTCCCCT | 4599 |
| 5024 | GAGAGCCA G CUCCUCCC | 2273 | GGGAGGAG GGCTAGCTACAACGA TGGCTCTC | 4600 |
| 5044 | GAGGGAGG G UCUUGAUG | 2274 | CATCAAGA GGCTAGCTACAACGA CCTCCCTC | 4601 |
| 5050 | GGGUCUUG A UGCCUGGG | 2275 | CCCAGGCA GGCTAGCTACAACGA CAAGACCC | 4602 |
| 5052 | GUCUUGAU G CCUGGGGU | 2276 | ACCCCAGG GGCTAGCTACAACGA ATCAAGAC | 4603 |
| 5059 | UGCCUGGG G UUACCCGC | 2277 | GCGGGTAA GGCTAGCTACAACGA CCCAGGCA | 4604 |
| 5062 | CUGGGGUU A CCCGCAGA | 2278 | TCTGCGGG GGCTAGCTACAACGA AACCCCAG | 4605 |
| 5066 | GGUUACCC G CAGAGGCC | 2279 | GGCCTCTG GGCTAGCTACAACGA GGGTAACC | 4606 |
| 5072 | CCGCAGAG G CCUGGGUG | 2280 | CACCCAGG GGCTAGCTACAACGA CTCTGCGG | 4607 |
| 5078 | AGGCCUGG G UGCCGGGA | 2281 | TCCCGGCA GGCTAGCTACAACGA CCAGGCCT | 4608 |
| 5080 | GCCUGGGU G CCGGGACG | 2282 | CGTCCCGG GGCTAGCTACAACGA ACCCAGGC | 4609 |
| 5086 | GUGCCGGG A CGCUCCCC | 2283 | GGGGAGCG GGCTAGCTACAACGA CCCGGCAC | 4610 |
| 5088 | GCCGGGAC G CUCCCCGG | 2284 | CCGGGGAG GGCTAGCTACAACGA GTCCCGGC | 4611 |
| 5096 | GCUCCCCG G UUUGGCUG | 2285 | CAGCCAAA GGCTAGCTACAACGA CGGGGAGC | 4612 |
| 5101 | CCGGUUUG G CUGAAAGG | 2286 | CCTTTCAG GGCTAGCTACAACGA CAAACCGG | 4613 |
| 5113 | AAAGGAAA G CAGAUGUG | 2287 | CACATCTG GGCTAGCTACAACGA TTTCCTTT | 4614 |
| 5117 | GAAAGCAG A UGUGGUCA | 2288 | TGACCACA GGCTAGCTACAACGA CTGCTTTC | 4615 |
| 5119 | AAGCAGAU G UGGUCAGC | 2289 | GCTGACCA GGCTAGCTACAACGA ATCTGCTT | 4616 |
| 5122 | CAGAUGUG G UCAGCUUC | 2290 | GAAGCTGA GGCTAGCTACAACGA CACATCTG | 4617 |
| 5126 | UGUGGUCA G CUUCUCCA | 2291 | TGGAGAAG GGCTAGCTACAACGA TGACCACA | 4618 |
| 5134 | GCUUCUCC A CUGAGCCC | 2292 | GGGCTCAG GGCTAGCTACAACGA GGAGAAGC | 4619 |
| 5139 | UCCACUGA G CCCAUCUG | 2293 | CAGATGGG GGCTAGCTACAACGA TCAGTGGA | 4620 |
| 5143 | CUGAGCCC A UCUGGUCU | 2294 | AGACCAGA GGCTAGCTACAACGA GGGCTCAG | 4621 |
| 5148 | CCCAUCUG G UCUUCCCG | 2295 | CGGGAAGA GGCTAGCTACAACGA CAGATGGG | 4622 |
| 5159 | UUCCCGGG G CUGGGCCC | 2296 | GGGCCCAG GGCTAGCTACAACGA CCCGGGAA | 4623 |
| 5164 | GGGGCUGG G CCCCAUAG | 2297 | CTATGGGG GGCTAGCTACAACGA CCAGCCCC | 4624 |
| 5169 | UGGGCCCC A UAGAUCUG | 2298 | CAGATCTA GGCTAGCTACAACGA GGGGCCCA | 4625 |
| 5173 | CCCCAUAG A UCUGGGUC | 2299 | GACCCAGA GGCTAGCTACAACGA CTATGGGG | 4626 |
| 5179 | AGAUCUGG G UCCCUGUG | 2300 | CACAGGGA GGCTAGCTACAACGA CCAGATCT | 4627 |
| 5185 | GGGUCCCU G UGUGGCCC | 2301 | GGGCCACA GGCTAGCTACAACGA AGGGACCC | 4628 |
| 5187 | GUCCCUGU G UGGCCCCC | 2302 | GGGGGCCA GGCTAGCTACAACGA ACAGGGAC | 4629 |
| 5190 | CCUGUGUG G CCCCCCUG | 2303 | CAGGGGGG GGCTAGCTACAACGA CACACAGG | 4630 |
| 5199 | CCCCCCUG G UCUGAUGC | 2304 | GCATCAGA GGCTAGCTACAACGA CAGGGGGG | 4631 |
| 5204 | CUGGUCUG A UGCCGAGG | 2305 | CCTCGGCA GGCTAGCTACAACGA CAGACCAG | 4632 |
| 5206 | GGUCUGAU G CCGAGGAU | 2306 | ATCCTCGG GGCTAGCTACAACGA ATCAGACC | 4633 |
| 5213 | UGCCGAGG A UACCCCUG | 2307 | CAGGGGTA GGCTAGCTACAACGA CCTCGGCA | 4634 |
| 5215 | CCGAGGAU A CCCCUGCA | 2308 | TGCAGGGG GGCTAGCTACAACGA ATCCTCGG | 4635 |
| 5221 | AUACCCCU G CAAACUGC | 2309 | GCAGTTTG GGCTAGCTACAACGA AGGGGTAT | 4636 |
| 5225 | CCCUGCAA A CUGCCAAU | 2310 | ATTGGCAG GGCTAGCTACAACGA TTGCAGGG | 4637 |
| 5228 | UGCAAACU G CCAAUCCC | 2311 | GGGATTGG GGCTAGCTACAACGA AGTTTGCA | 4638 |
| 5232 | AACUGCCA A UCCCAGAG | 2312 | CTCTGGGA GGCTAGCTACAACGA TGGCAGTT | 4639 |
| 5242 | CCCAGAGG A CAAGACUG | 2313 | CAGTCTTG GGCTAGCTACAACGA CCTCTGGG | 4640 |
| 5247 | AGGACAAG A CUGGGAAG | 2314 | CTTCCCAG GGCTAGCTACAACGA CTTGTCCT | 4641 |
| 5255 | ACUGGGAA G UCCCUGCA | 2315 | TGCAGGGA GGCTAGCTACAACGA TTCCCAGT | 4642 |
| 5261 | AAGUCCCU G CAGGGAGA | 2316 | TCTCCCTG GGCTAGCTACAACGA AGGGACTT | 4643 |
| 5270 | CAGGGAGA G CCCAUCCC | 2317 | GGGATGGG GGCTAGCTACAACGA TCTCCCTG | 4644 |
| 5274 | GAGAGCCC A UCCCCGCA | 2318 | TGCGGGGA GGCTAGCTACAACGA GGGCTCTC | 4645 |
| 5280 | CCAUCCCC G CACCCUGA | 2319 | TCAGGGTG GGCTAGCTACAACGA GGGGATGG | 4646 |
| 5282 | AUCCCCGC A CCCUGACC | 2320 | GGTCAGGG GGCTAGCTACAACGA GCGGGGAT | 4647 |
| 5288 | GCACCCUG A CCCACAAG | 2321 | CTTGTGGG GGCTAGCTACAACGA CAGGGTGC | 4648 |
| 5292 | CCUGACCC A CAAGAGGG | 2322 | CCCTCTTG GGCTAGCTACAACGA GGGTCAGG | 4649 |
| 5301 | CAAGAGGG A CUCCUGCU | 2323 | AGCAGGAG GGCTAGCTACAACGA CCCTCTTG | 4650 |
| 5307 | GGACUCCU G CUGCCCAC | 2324 | GTGGGCAG GGCTAGCTACAACGA AGGAGTCC | 4651 |
| 5310 | CUCCUGCU G CCCACCAG | 2325 | CTGGTGGG GGCTAGCTACAACGA AGCAGGAG | 4652 |
| 5314 | UGCUGCCC A CCAGGCAU | 2326 | ATGCCTGG GGCTAGCTACAACGA GGGCAGCA | 4653 |
| 5319 | CCCACCAG G CAUCCCUC | 2327 | GAGGGATG GGCTAGCTACAACGA CTGGTGGG | 4654 |
| 5321 | CACCAGGC A UCCCUCCA | 2328 | TGGAGGGA GGCTAGCTACAACGA GCCTGGTG | 4655 |

Input Sequence = HUMRasH_mRNA.
Cut Site = R/Y
Arm Length = 8.
Core Sequence = GGCTAGCTACAACGA
HUMRasH_mRNA (Human c-Ha-ras1 proto-oncogene, spliced mRNA sequence; 5336 nt)

TABLE IV
Human HER2 DNAzyme and Substrate Sequence

| | | | | |
|---|---|---|---|---|
| | | Seq | | Seq |
| Pos | Substrate | ID | DNAzyme | ID |

| | | | | |
|---|---|---|---|---|
| 9 | AAGGGGAG G UAACCCUG | 4656 | CAGGGTTA GGCTAGCTACAACGA CTCCCCTT | 5644 |
| 12 | GGGAGGUA A CCCUGGCC | 4657 | GGCCAGGG GGCTAGCTACAACGA TACCTCCC | 5645 |
| 18 | UAACCCUG G CCCCUUUG | 4658 | CAAAGGGG GGCTAGCTACAACGA CAGGGTTA | 5646 |
| 27 | CCCCUUUG G UCGGGGCC | 4659 | GGCCCCGA GGCTAGCTACAACGA CAAAGGGG | 5647 |
| 33 | UGGUCGGG G CCCCGGGC | 4660 | GCCCGGGG GGCTAGCTACAACGA CCCGACCA | 5648 |
| 40 | GGCCCCGG G CAGCCGCG | 4661 | CGCGGCTG GGCTAGCTACAACGA CCGGGGCC | 5649 |
| 43 | CCCGGGCA G CCGCGCGC | 4662 | GCGCGCGG GGCTAGCTACAACGA TGCCCGGG | 5650 |
| 46 | GGGCAGCC G CGCGCCCC | 4663 | GGGGCGCG GGCTAGCTACAACGA GGCTGCCC | 5651 |
| 48 | GCAGCCGC G CGCCCCUU | 4664 | AAGGGGCG GGCTAGCTACAACGA GCGGCTGC | 5652 |
| 50 | AGCCGCGC G CCCCUUCC | 4665 | GGAAGGGG GGCTAGCTACAACGA GCGCGGCT | 5653 |
| 60 | CCCUUCCC A CGGGGCCC | 4666 | GGGCCCCG GGCTAGCTACAACGA GGGAAGGG | 5654 |
| 65 | CCCACGGG G CCCUUUAC | 4667 | GTAAAGGG GGCTAGCTACAACGA CCCGTGGG | 5655 |
| 72 | GGCCCUUU A CUGCGCCG | 4668 | CGGCGCAG GGCTAGCTACAACGA AAAGGGCC | 5656 |
| 75 | CCUUUACU G CGCCGCGC | 4669 | GCGCGGCG GGCTAGCTACAACGA AGTAAAGG | 5657 |
| 77 | UUUACUGC G CCGCGCGC | 4670 | GCGCGCGG GGCTAGCTACAACGA GCAGTAAA | 5658 |
| 80 | ACUGCGCC G CGCGCCCG | 4671 | CGGGCGCG GGCTAGCTACAACGA GGCGCAGT | 5659 |
| 82 | UGCGCCGC G CGCCCGGC | 4672 | GCCGGGCG GGCTAGCTACAACGA GCGGCGCA | 5660 |
| 84 | CGCCGCGC G CCCGGCCC | 4673 | GGGCCGGG GGCTAGCTACAACGA GCGCGGCG | 5661 |
| 89 | CGCGCCCG G CCCCCACC | 4674 | GGTGGGGG GGCTAGCTACAACGA CGGGCGCG | 5662 |
| 95 | CGGCCCCC A CCCCUCGC | 4675 | GCGAGGGG GGCTAGCTACAACGA GGGGGCCG | 5663 |
| 102 | CACCCCUC G CAGCACCC | 4676 | GGGTGCTG GGCTAGCTACAACGA GAGGGGTG | 5664 |
| 105 | CCCUCGCA G CACCCCGC | 4677 | GCGGGGTG GGCTAGCTACAACGA TGCGAGGG | 5665 |
| 107 | CUCGCAGC A CCCCGCGC | 4678 | GCGCGGGG GGCTAGCTACAACGA GCTGCGAG | 5666 |
| 112 | AGCACCCC G CGCCCCGC | 4679 | GCGGGGCG GGCTAGCTACAACGA GGGGTGCT | 5667 |
| 114 | CACCCCGC G CCCCGCGC | 4680 | GCGCGGGG GGCTAGCTACAACGA GCGGGGTG | 5668 |
| 119 | CGCGCCCC G CGCCCUCC | 4681 | GGAGGGCG GGCTAGCTACAACGA GGGGCGCG | 5669 |
| 121 | CGCCCCGC G CCCUCCCA | 4682 | TGGGAGGG GGCTAGCTACAACGA GCGGGGCG | 5670 |
| 130 | CCCUCCCA G CCGGGUCC | 4683 | GGACCCGG GGCTAGCTACAACGA TGGGAGGG | 5671 |
| 135 | CCAGCCGG G UCCAGCCG | 4684 | CGGCTGGA GGCTAGCTACAACGA CCGGCTGG | 5672 |
| 140 | CGGGUCCA G CCGGAGCC | 4685 | GGCTCCGG GGCTAGCTACAACGA TGGACCCG | 5673 |
| 146 | CAGCCGGA G CCAUGGGG | 4686 | CCCCATGG GGCTAGCTACAACGA TCCGGCTG | 5674 |
| 149 | CCGGAGCC A UGGGGCCG | 4687 | CGGCCCCA GGCTAGCTACAACGA GGCTCCGG | 5675 |
| 154 | GCCAUGGG G CCGGAGCC | 4688 | GGCTCCGG GGCTAGCTACAACGA CCCATGGC | 5676 |
| 160 | GGGCCGGA G CCGCAGUG | 4689 | CACTGCGG GGCTAGCTACAACGA TCCGGCCC | 5677 |
| 163 | CCGGAGCC G CAGUGAGC | 4690 | GCTCACTG GGCTAGCTACAACGA GGCTCCGG | 5678 |
| 166 | GAGCCGCA G UGAGCACC | 4691 | GGTGCTCA GGCTAGCTACAACGA TGCGGCTC | 5679 |
| 170 | CGCAGUGA G CACCAUGG | 4692 | CCATGGTG GGCTAGCTACAACGA TCACTGCG | 5680 |
| 172 | CAGUGAGC A CCAUGGAG | 4693 | CTCCATGG GGCTAGCTACAACGA GCTCACTG | 5681 |
| 175 | UGAGCACC A UGGAGCUG | 4694 | CAGCTCCA GGCTAGCTACAACGA GGTGCTCA | 5682 |
| 180 | ACCAUGGA G CUGGCGGC | 4695 | GCCGCCAG GGCTAGCTACAACGA TCCATGGT | 5683 |
| 184 | UGGAGCUG G CGGCCUUG | 4696 | CAAGGCCG GGCTAGCTACAACGA CAGCTCCA | 5684 |
| 187 | AGCUGGCG G CCUUGUGC | 4697 | GCACAAGG GGCTAGCTACAACGA CGCCAGCT | 5685 |
| 192 | GCGGCCUU G UGCCGCUG | 4698 | CAGCGGCA GGCTAGCTACAACGA AAGGCCGC | 5686 |
| 194 | GGCCUUGU G CCGCUGGG | 4699 | CCCAGCGG GGCTAGCTACAACGA ACAAGGCC | 5687 |
| 197 | CUUGUGCC G CUGGGGGC | 4700 | GCCCCCAG GGCTAGCTACAACGA GGCACAAG | 5688 |
| 204 | CGCUGGGG G CUCCUCCU | 4701 | AGGAGGAG GGCTAGCTACAACGA CCCCAGCG | 5689 |
| 214 | UCCUCCUC G CCCUCUUG | 4702 | CAAGAGGG GGCTAGCTACAACGA GAGGAGGA | 5690 |
| 222 | GCCCUCUU G CCCCCCGG | 4703 | CCGGGGGG GGCTAGCTACAACGA AAGAGGGC | 5691 |
| 232 | CCCCCGGA G CCGCGAGC | 4704 | GCTCGCGG GGCTAGCTACAACGA TCCGGGGG | 5692 |
| 235 | CCGGAGCC G CGAGCACC | 4705 | GGTGCTCG GGCTAGCTACAACGA GGCTCCGG | 5693 |
| 239 | AGCCGCGA G CACCCAAG | 4706 | CTTGGGTG GGCTAGCTACAACGA TCGCGGCT | 5694 |
| 241 | CCGCGAGC A CCCAAGUG | 4707 | CACTTGGG GGCTAGCTACAACGA GCTCGCGG | 5695 |
| 247 | GCACCCAA G UGUGCACC | 4708 | GGTGCACA GGCTAGCTACAACGA TTGGGTGC | 5696 |
| 249 | ACCCAAGU G UGCACCGG | 4709 | CCGGTGCA GGCTAGCTACAACGA ACTTGGGT | 5697 |
| 251 | CCAAGUGU G CACCGGCA | 4710 | TGCCGGTG GGCTAGCTACAACGA ACACTTGG | 5698 |
| 253 | AAGUGUGC A CCGGCACA | 4711 | TGTGCCGG GGCTAGCTACAACGA GCACACTT | 5699 |
| 257 | GUGCACCG G CACAGACA | 4712 | TGTCTGTG GGCTAGCTACAACGA CGGTGCAC | 5700 |
| 259 | GCACCGGC A CAGACAUG | 4713 | CATGTCTG GGCTAGCTACAACGA GCCGGTGC | 5701 |
| 263 | CGGCACAG A CAUGAAGC | 4714 | GCTTCATG GGCTAGCTACAACGA CTGTGCCG | 5702 |
| 265 | GCACAGAC A UGAAGCUG | 4715 | CAGCTTCA GGCTAGCTACAACGA GTCTGTGC | 5703 |
| 270 | GACAUGAA G CUGCGGCU | 4716 | AGCCGCAG GGCTAGCTACAACGA TTCATGTC | 5704 |
| 273 | AUGAAGCU G CGGCUCCC | 4717 | GGGAGCCG GGCTAGCTACAACGA AGCTTCAT | 5705 |
| 276 | AAGCUGCG G CUCCCUGC | 4718 | GCAGGGAG GGCTAGCTACAACGA CGCAGCTT | 5706 |
| 283 | GGCUCCCU G CCAGUCCC | 4719 | GGGACTGG GGCTAGCTACAACGA AGGGAGCC | 5707 |
| 287 | CCCUGCCA G UCCCGAGA | 4720 | TCTCGGGA GGCTAGCTACAACGA TGGCAGGG | 5708 |
| 295 | GUCCCGAG A CCCACCUG | 4721 | CAGGTGGG GGCTAGCTACAACGA CTCGGGAC | 5709 |
| 299 | CGAGACCC A CCUGGACA | 4722 | TGTCCAGG GGCTAGCTACAACGA GGGTCTCG | 5710 |
| 305 | CCACCUGG A CAUGCUCC | 4723 | GGAGCATG GGCTAGCTACAACGA CCAGGTGG | 5711 |
| 307 | ACCUGGAC A UGCUCCGC | 4724 | GCGGAGCA GGCTAGCTACAACGA GTCCAGGT | 5712 |
| 309 | CUGGACAU G CUCCGCCA | 4725 | TGGCGGAG GGCTAGCTACAACGA ATGTCCAG | 5713 |
| 314 | CAUGCUCC G CCACCUCU | 4726 | AGAGGTGG GGCTAGCTACAACGA GGAGCATG | 5714 |
| 317 | GCUCCGCC A CCUCUACC | 4727 | GGTAGAGG GGCTAGCTACAACGA GGCGGAGC | 5715 |
| 323 | CCACCUCU A CCAGGGCU | 4728 | AGCCCTGG GGCTAGCTACAACGA AGAGGTGG | 5716 |
| 329 | CUACCAGG G CUGCCAGG | 4729 | CCTGGCAG GGCTAGCTACAACGA CCTGGTAG | 5717 |
| 332 | CCAGGGCU G CCAGGUGG | 4730 | CCACCTGG GGCTAGCTACAACGA AGCCCTGG | 5718 |
| 337 | GCUGCCAG G UGGUGCAG | 4731 | CTGCACCA GGCTAGCTACAACGA CTGGCAGC | 5719 |
| 340 | GCCAGGUG G UGCAGGGA | 4732 | TCCCTGCA GGCTAGCTACAACGA CACCTGGC | 5720 |
| 342 | CAGGUGGU G CAGGGAAA | 4733 | TTTCCCTG GGCTAGCTACAACGA ACCACCTG | 5721 |
| 350 | GCAGGGAA A CCUGGAAC | 4734 | GTTCCAGG GGCTAGCTACAACGA TTCCCTGC | 5722 |
| 357 | AACCUGGA A CUCACCUA | 4735 | TAGGTGAG GGCTAGCTACAACGA TCCAGGTT | 5723 |
| 361 | UGGAACUC A CCUACCUG | 4736 | CAGGTAGG GGCTAGCTACAACGA GAGTTCCA | 5724 |
| 365 | ACUCACCU A CCUGCCCA | 4737 | TGGGCAGG GGCTAGCTACAACGA AGGTGAGT | 5725 |
| 369 | ACCUACCU G CCCACCAA | 4738 | TTGGTGGG GGCTAGCTACAACGA AGGTAGGT | 5726 |
| 373 | ACCUGCCC A CCAAUGCC | 4739 | GGCATTGG GGCTAGCTACAACGA GGGCAGGT | 5727 |
| 377 | GCCCACCA A UGCCAGCC | 4740 | GGCTGGCA GGCTAGCTACAACGA TGGTGGGC | 5728 |
| 379 | CCACCAAU G CCAGCCUG | 4741 | CAGGCTGG GGCTAGCTACAACGA ATTGGTGG | 5729 |
| 383 | CAAUGCCA G CCUGUCCU | 4742 | AGGACAGG GGCTAGCTACAACGA TGGCATTG | 5730 |
| 387 | GCCAGCCU G UCCUUCCU | 4743 | AGGAAGGA GGCTAGCTACAACGA AGGCTGGC | 5731 |
| 396 | UCCUUCCU G CAGGAUAU | 4744 | ATATCCTG GGCTAGCTACAACGA AGGAAGGA | 5732 |
| 401 | CCUGCAGG A UAUCCAGG | 4745 | CCTGGATA GGCTAGCTACAACGA CCTGCAGG | 5733 |
| 403 | UGCAGGAU A UCCAGGAG | 4746 | CTCCTGGA GGCTAGCTACAACGA ATCCTGCA | 5734 |
| 412 | UCCAGGAG G UGCAGGGC | 4747 | GCCCTGCA GGCTAGCTACAACGA CTCCTGGA | 5735 |
| 414 | CAGGAGGU G CAGGGCUA | 4748 | TAGCCCTG GGCTAGCTACAACGA ACCTCCTG | 5736 |
| 419 | GGUGCAGG G CUACGUGC | 4749 | GCACGTAG GGCTAGCTACAACGA CCTGCACC | 5737 |
| 422 | GCAGGGCU A CGUGCUCA | 4750 | TGAGCACG GGCTAGCTACAACGA AGCCCTGC | 5738 |
| 424 | AGGGCUAC G UGCUCAUC | 4751 | GATGAGCA GGCTAGCTACAACGA GTAGCCCT | 5739 |
| 426 | GGCUACGU G CUCAUCGC | 4752 | GCGATGAG GGCTAGCTACAACGA ACGTAGCC | 5740 |
| 430 | ACGUGCUC A UCGCUCAC | 4753 | GTGAGCGA GGCTAGCTACAACGA GAGCACGT | 5741 |
| 433 | UGCUCAUC G CUCACAAC | 4754 | GTTGTGAG GGCTAGCTACAACGA GATGAGCA | 5742 |
| 437 | CAUCGCUC A CAACCAAG | 4755 | CTTGGTTG GGCTAGCTACAACGA GAGCGATG | 5743 |
| 440 | CGCUCACA A CCAAGUGA | 4756 | TCACTTGG GGCTAGCTACAACGA TGTGAGCG | 5744 |
| 445 | ACAACCAA G UGAGGCAG | 4757 | CTGCCTCA GGCTAGCTACAACGA TTGGTTGT | 5745 |
| 450 | CAAGUGAG G CAGGUCCC | 4758 | GGGACCTG GGCTAGCTACAACGA CTCACTTG | 5746 |
| 454 | UGAGGCAG G UCCCACUG | 4759 | CAGTGGGA GGCTAGCTACAACGA CTGCCTCA | 5747 |
| 459 | CAGGUCCC A CUGCAGAG | 4760 | CTCTGCAG GGCTAGCTACAACGA GGGACCTG | 5748 |
| 462 | GUCCCACU G CAGAGGCU | 4761 | AGCCTCTG GGCTAGCTACAACGA AGTGGGAC | 5749 |
| 468 | CUGCAGAG G CUGCGGAU | 4762 | ATCCGCAG GGCTAGCTACAACGA CTCTGCAG | 5750 |
| 471 | CAGAGGCU G CGGAUUGU | 4763 | ACAATCCG GGCTAGCTACAACGA AGCCTCTG | 5751 |
| 475 | GGCUGCGG A UUGUGCGA | 4764 | TCGCACAA GGCTAGCTACAACGA CCGCAGCC | 5752 |
| 478 | UGCGGAUU G UGCGAGGC | 4765 | GCCTCGCA GGCTAGCTACAACGA AATCCGCA | 5753 |
| 480 | CGGAUUGU G CGAGGCAC | 4766 | GTGCCTCG GGCTAGCTACAACGA ACAATCCG | 5754 |
| 485 | UGUGCGAG G CACCCAGC | 4767 | GCTGGGTG GGCTAGCTACAACGA CTCGCACA | 5755 |
| 487 | UGCGAGGC A CCCAGCUC | 4768 | GAGCTGGG GGCTAGCTACAACGA GCCTCGCA | 5756 |
| 492 | GGCACCCA G CUCUUUGA | 4769 | TCAAAGAG GGCTAGCTACAACGA TGGGTGCC | 5757 |
| 503 | CUUUGAGG A CAACUAUG | 4770 | CATAGTTG GGCTAGCTACAACGA CCTCAAAG | 5758 |
| 506 | UGAGGACA A CUAUGCCC | 4771 | GGGCATAG GGCTAGCTACAACGA TGTCCTCA | 5759 |
| 509 | GGACAACU A UGCCCUGG | 4772 | CCAGGGCA GGCTAGCTACAACGA AGTTGTCC | 5760 |
| 511 | ACAACUAU G CCCUGGCC | 4773 | GGCCAGGG GGCTAGCTACAACGA ATAGTTGT | 5761 |
| 517 | AUGCCCUG G CCGUGCUA | 4774 | TAGCACGG GGCTAGCTACAACGA CAGGGCAT | 5762 |
| 520 | CCCUGGCC G UGCUAGAC | 4775 | GTCTAGCA GGCTAGCTACAACGA GGCCAGGG | 5763 |
| 522 | CUGGCCGU G CUAGACAA | 4776 | TTGTCTAG GGCTAGCTACAACGA ACGGCCAG | 5764 |
| 527 | CGUGCUAG A CAAUGGAG | 4777 | CTCCATTG GGCTAGCTACAACGA CTAGCACG | 5765 |
| 530 | GCUAGACA A UGGAGACC | 4778 | GGTCTCCA GGCTAGCTACAACGA TGTCTAGC | 5766 |
| 536 | CAAUGGAG A CCCGCUGA | 4779 | TCAGCGGG GGCTAGCTACAACGA CTCCATTG | 5767 |
| 540 | GGAGACCC G CUGAACAA | 4780 | TTGTTCAG GGCTAGCTACAACGA GGGTCTCC | 5768 |
| 545 | CCCGCUGA A CAAUACCA | 4781 | TGGTATTG GGCTAGCTACAACGA TCAGCGGG | 5769 |
| 548 | GCUGAACA A UACCACCC | 4782 | GGGTGGTA GGCTAGCTACAACGA TGTTCAGC | 5770 |
| 550 | UGAACAAU A CCACCCCU | 4783 | AGGGGTGG GGCTAGCTACAACGA ATTGTTCA | 5771 |
| 553 | ACAAUACC A CCCCUGUC | 4784 | GACAGGGG GGCTAGCTACAACGA GGTATTGT | 5772 |
| 559 | CCACCCCU G UCACAGGG | 4785 | CCCTGTGA GGCTAGCTACAACGA AGGGGTGG | 5773 |
| 562 | CCCCUGUC A CAGGGGCC | 4786 | GGCCCCTG GGCTAGCTACAACGA GACAGGGG | 5774 |
| 568 | UCACAGGG G CCUCCCCA | 4787 | TGGGGAGG GGCTAGCTACAACGA CCCTGTGA | 5775 |
| 581 | CCCAGGAG G CCUGCGGG | 4788 | CCCGCAGG GGCTAGCTACAACGA CTCCTGGG | 5776 |
| 585 | GGAGGCCU G CGGGAGCU | 4789 | AGCTCCCG GGCTAGCTACAACGA AGGCCTCC | 5777 |
| 591 | CUGCGGGA G CUGCAGCU | 4790 | AGCTGCAG GGCTAGCTACAACGA TCCCGCAG | 5778 |
| 594 | CGGGAGCU G CAGCUUCG | 4791 | CGAAGCTG GGCTAGCTACAACGA AGCTCCCG | 5779 |
| 597 | GAGCUGCA G CUUCGAAG | 4792 | CTTCGAAG GGCTAGCTACAACGA TGCAGCTC | 5780 |
| 605 | GCUUCGAA G CCUCACAG | 4793 | CTGTGAGG GGCTAGCTACAACGA TTCGAAGC | 5781 |
| 610 | GAAGCCUC A CAGAGAUC | 4794 | GATCTCTG GGCTAGCTACAACGA GAGGCTTC | 5782 |
| 616 | UCACAGAG A UCUUGAAA | 4795 | TTTCAAGA GGCTAGCTACAACGA CTCTGTGA | 5783 |
| 631 | AAGGAGGG G UCUUGAUC | 4796 | GATCAAGA GGCTAGCTACAACGA CCCTCCTT | 5784 |
| 637 | GGGUCUUG A UCCAGCGG | 4797 | CCGCTGGA GGCTAGCTACAACGA CAAGACCC | 5785 |
| 642 | UUGAUCCA G CGGAACCC | 4798 | GGGTTCCG GGCTAGCTACAACGA TGGATCAA | 5786 |
| 647 | CCAGCGGA A CCCCCAGC | 4799 | GCTGGGGG GGCTAGCTACAACGA TCCGCTGG | 5787 |
| 654 | AACCCCCA G CUCUGCUA | 4800 | TAGCAGAG GGCTAGCTACAACGA TGGGGGTT | 5788 |
| 659 | CCAGCUCU G CUACCAGG | 4801 | CCTGGTAG GGCTAGCTACAACGA AGAGCTGG | 5789 |
| 662 | GCUCUGCU A CCAGGACA | 4802 | TGTCCTGG GGCTAGCTACAACGA AGCAGAGC | 5790 |
| 668 | CUACCAGG A CACGAUUU | 4803 | AAATCGTG GGCTAGCTACAACGA CCTGGTAG | 5791 |
| 670 | ACCAGGAC A CGAUUUUG | 4804 | CAAAATCG GGCTAGCTACAACGA GTCCTGGT | 5792 |
| 673 | AGGACACG A UUUUGUGG | 4805 | CCACAAAA GGCTAGCTACAACGA CGTGTCCT | 5793 |
| 678 | ACGAUUUU G UGGAAGGA | 4806 | TCCTTCCA GGCTAGCTACAACGA AAAATCGT | 5794 |
| 686 | GUGGAAGG A CAUCUUCC | 4807 | GGAAGATG GGCTAGCTACAACGA CCTTCCAC | 5795 |
| 688 | GGAAGGAC A UCUUCCAC | 4808 | GTGGAAGA GGCTAGCTACAACGA GTCCTTCC | 5796 |
| 695 | CAUCUUCC A CAAGAACA | 4809 | TGTTCTTG GGCTAGCTACAACGA GGAAGATG | 5797 |
| 701 | CCACAAGA A CAACCAGC | 4810 | GCTGGTTG GGCTAGCTACAACGA TCTTGTGG | 5798 |
| 704 | CAAGAACA A CCAGCUGG | 4811 | CCAGCTGG GGCTAGCTACAACGA TGTTCTTG | 5799 |
| 708 | AACAACCA G CUGGCUCU | 4812 | AGAGCCAG GGCTAGCTACAACGA TGGTTGTT | 5800 |
| 712 | ACCAGCUG G CUCUCACA | 4813 | TGTGAGAG GGCTAGCTACAACGA CAGCTGGT | 5801 |
| 718 | UGGCUCUC A CACUGAUA | 4814 | TATCAGTG GGCTAGCTACAACGA GAGAGCCA | 5802 |
| 720 | GCUCUCAC A CUGAUAGA | 4815 | TCTATCAG GGCTAGCTACAACGA GTGAGAGC | 5803 |
| 724 | UCACACUG A UAGACACC | 4816 | GGTGTCTA GGCTAGCTACAACGA CAGTGTGA | 5804 |
| 728 | ACUGAUAG A CACCAACC | 4817 | GGTTGGTG GGCTAGCTACAACGA CTATCAGT | 5805 |
| 730 | UGAUAGAC A CCAACCGC | 4818 | GCGGTTGG GGCTAGCTACAACGA GTCTATCA | 5806 |
| 734 | AGACACCA A CCGCUCUC | 4819 | GAGAGCGG GGCTAGCTACAACGA TGGTGTCT | 5807 |
| 737 | CACCAACC G CUCUCGGG | 4820 | CCCGAGAG GGCTAGCTACAACGA GGTTGGTG | 5808 |
| 745 | GCUCUCGG G CCUGCCAC | 4821 | GTGGCAGG GGCTAGCTACAACGA CCGAGAGC | 5809 |
| 749 | UCGGGCCU G CCACCCCU | 4822 | AGGGGTGG GGCTAGCTACAACGA AGGCCCGA | 5810 |
| 752 | GGCCUGCC A CCCCUGUU | 4823 | AACAGGGG GGCTAGCTACAACGA GGCAGGCC | 5811 |
| 758 | CCACCCCU G UUCUCCGA | 4824 | TCGGAGAA GGCTAGCTACAACGA AGGGGTGG | 5812 |
| 766 | GUUCUCCG A UGUGUAAG | 4825 | CTTACACA GGCTAGCTACAACGA CGGAGAAC | 5813 |
| 768 | UCUCCGAU G UGUAAGGG | 4826 | CCCTTACA GGCTAGCTACAACGA ATCGGAGA | 5814 |
| 770 | UCCGAUGU G UAAGGGCU | 4827 | AGCCCTTA GGCTAGCTACAACGA ACATCGGA | 5815 |
| 776 | GUGUAAGG G CUCCCGCU | 4828 | AGCGGGAG GGCTAGCTACAACGA CCTTACAC | 5816 |
| 782 | GGGCUCCC G CUGCUGGG | 4829 | CCCAGCAG GGCTAGCTACAACGA GGGAGCCC | 5817 |
| 785 | CUCCCGCU G CUGGGGAG | 4830 | CTCCCCAG GGCTAGCTACAACGA AGCGGGAG | 5818 |
| 797 | GGGAGAGA G UUCUGAGG | 4831 | CCTCAGAA GGCTAGCTACAACGA TCTCTCCC | 5819 |
| 806 | UUCUGAGG A UUGUCAGA | 4832 | TCTGACAA GGCTAGCTACAACGA CCTCAGAA | 5820 |
| 809 | UGAGGAUU G UCAGAGCC | 4833 | GGCTCTGA GGCTAGCTACAACGA AATCCTCA | 5821 |
| 815 | UUGUCAGA G CCUGACGC | 4834 | GCGTCAGG GGCTAGCTACAACGA TCTGACAA | 5822 |
| 820 | AGAGCCUG A CGCGCACU | 4835 | AGTGCGCG GGCTAGCTACAACGA CAGGCTCT | 5823 |
| 822 | AGCCUGAC G CGCACUGU | 4836 | ACAGTGCG GGCTAGCTACAACGA GTCAGGCT | 5824 |
| 824 | CCUGACGC G CACUGUCU | 4837 | AGACAGTG GGCTAGCTACAACGA GCGTCAGG | 5825 |
| 826 | UGACGCGC A CUGUCUGU | 4838 | ACAGACAG GGCTAGCTACAACGA GCGCGTCA | 5826 |
| 829 | CGCGCACU G UCUGUGCC | 4839 | GGCACAGA GGCTAGCTACAACGA AGTGCGCG | 5827 |
| 833 | CACUGUCU G UGCCGGUG | 4840 | CACCGGCA GGCTAGCTACAACGA AGACAGTG | 5828 |
| 835 | CUGUCUGU G CCGGUGGC | 4841 | GCCACCGG GGCTAGCTACAACGA ACAGACAG | 5829 |
| 839 | CUGUGCCG G UGGCUGUG | 4842 | CACAGCCA GGCTAGCTACAACGA CGGCACAG | 5830 |
| 842 | UGCCGGUG G CUGUGCCC | 4843 | GGGCACAG GGCTAGCTACAACGA CACCGGCA | 5831 |
| 845 | CGGUGGCU G UGCCCGCU | 4844 | AGCGGGCA GGCTAGCTACAACGA AGCCACCG | 5832 |
| 847 | GUGGCUGU G CCCGCUGC | 4845 | GCAGCGGG GGCTAGCTACAACGA ACAGCCAC | 5833 |
| 851 | CUGUGCCC G CUCCAAGG | 4846 | CCTTGCAG GGCTAGCTACAACGA GGGCACAG | 5834 |
| 854 | UGCCCGCU G CAAGGGGC | 4847 | GCCCCTTG GGCTAGCTACAACGA AGCGGGCA | 5835 |
| 861 | UGCAAGGG G CCACUGCC | 4848 | GGCAGTGG GGCTAGCTACAACGA CCCTTGCA | 5836 |
| 864 | AAGGGGCC A CUGCCCAC | 4849 | GTGGGCAG GGCTAGCTACAACGA GGCCCCTT | 5837 |
| 867 | GGGCCACU G CCCACUGA | 4850 | TCAGTGGG GGCTAGCTACAACGA AGTGGCCC | 5838 |
| 871 | CACUGCCC A CUCACUGC | 4851 | GCAGTCAG GGCTAGCTACAACGA GGGCAGTG | 5839 |
| 875 | GCCCACUG A CUGCUGCC | 4852 | GGCACCAG GGCTAGCTACAACGA CAGTGGGC | 5840 |
| 878 | CACUGACU G CUGCCAUG | 4853 | CATGGCAG GGCTAGCTACAACGA AGTCACTG | 5841 |
| 881 | UGACUGCU G CCAUGAGC | 4854 | GCTCATGG GGCTAGCTACAACGA AGCAGTCA | 5842 |
| 884 | CUGCUGCC A UGAGCAGU | 4855 | ACTGCTCA GGCTAGCTACAACGA GGCAGCAG | 5843 |
| 888 | UGCCAUGA G CAGUGUGC | 4856 | GCACACTG GGCTAGCTACAACGA TCATGGCA | 5844 |
| 891 | CAUGAGCA G UGUGCUGC | 4857 | GCAGCACA GGCTAGCTACAACGA TGCTCATG | 5845 |
| 893 | UGAGCAGU G UGCUGCCG | 4858 | CGGCAGCA GGCTAGCTACAACGA ACTGCTCA | 5846 |
| 895 | AGCAGUGU G CUGCCGGC | 4859 | GCCGGCAG GGCTAGCTACAACGA ACACTGCT | 5847 |
| 898 | AGUGUGCU G CCGGCUGC | 4860 | GCAGCCGG GGCTAGCTACAACGA AGCACACT | 5848 |
| 902 | UGCUGCCG G CUGCACGG | 4861 | CCGTGCAG GGCTAGCTACAACGA CGGCAGCA | 5849 |
| 905 | UGCCGGCU G CACGGGCC | 4862 | GGCCCGTG GGCTAGCTACAACGA AGCCGGCA | 5850 |
| 907 | CCGGCUGC A CGGGCCCC | 4863 | GGGGCCCG GGCTAGCTACAACGA GCAGCCGG | 5851 |
| 911 | CUGCACGG G CCCCAAGC | 4864 | GCTTGGGG GGCTAGCTACAACGA CCGTGCAG | 5852 |
| 918 | GGCCCCAA G CACUCUGA | 4865 | TCAGAGTG GGCTAGCTACAACGA TTGGGGCC | 5853 |
| 920 | CCCCAAGC A CUCUGACU | 4866 | AGTCAGAG GGCTAGCTACAACGA GCTTGGGG | 5854 |
| 926 | GCACUCUG A CUGCCUGG | 4867 | CCAGGCAG GGCTAGCTACAACGA CAGAGTGC | 5855 |
| 929 | CUCUGACU G CCUGGCCU | 4868 | AGGCCAGG GGCTAGCTACAACGA AGTCAGAG | 5856 |
| 934 | ACUGCCUG G CCUGCCUC | 4869 | GAGGCAGG GGCTAGCTACAACGA CAGGCAGT | 5857 |
| 938 | CCUGGCCU G CCUCCACU | 4870 | AGTGGAGG GGCTAGCTACAACGA AGGCCAGG | 5858 |
| 944 | CUGCCUCC A CUUCAACC | 4871 | GGTTGAAG GGCTAGCTACAACGA GGAGGCAG | 5859 |
| 950 | CCACUUCA A CCACAGUG | 4872 | CACTGTGG GGCTAGCTACAACGA TGAAGTGG | 5860 |
| 953 | CUUCAACC A CAGUGGCA | 4873 | TGCCACTG GGCTAGCTACAACGA GGTTGAAG | 5861 |
| 956 | CAACCACA G UGGCAUCU | 4874 | AGATGCCA GGCTAGCTACAACGA TGTGGTTG | 5862 |
| 959 | CCACAGUG G CAUCUGUG | 4875 | CACAGATG GGCTAGCTACAACGA CACTGTGG | 5863 |
| 961 | ACAGUGGC A UCUGUGAG | 4876 | CTCACAGA GGCTAGCTACAACGA GCCACTGT | 5864 |
| 965 | UGGCAUCU G UGAGCUGC | 4877 | GCAGCTCA GGCTAGCTACAACGA AGATGCCA | 5865 |
| 969 | AUCUGUGA G CUGCACUG | 4878 | CAGTGCAG GGCTAGCTACAACGA TCACAGAT | 5866 |
| 972 | UGUGAGCU G CACUGCCC | 4879 | GGGCAGTG GGCTAGCTACAACGA AGCTCACA | 5867 |
| 974 | UGAGCUGC A CUGCCCAG | 4880 | CTGGGCAG GGCTAGCTACAACGA GCAGCTCA | 5868 |
| 977 | GCUGCACU G CCCAGCCC | 4881 | GGGCTGGG GGCTAGCTACAACGA AGTGCAGC | 5869 |
| 982 | ACUGCCCA G CCCUGGUC | 4882 | GACCAGGG GGCTAGCTACAACGA TGGGCAGT | 5870 |
| 988 | CAGCCCUG G UCACCUAC | 4883 | GTAGGTGA GGCTAGCTACAACGA CAGGGCTG | 5871 |
| 991 | CCCUGGUC A CCUACAAC | 4884 | GTTGTAGG GGCTAGCTACAACGA GACCAGGG | 5872 |
| 995 | GGUCACCU A CAACACAG | 4885 | CTGTGTTG GGCTAGCTACAACGA AGGTGACC | 5873 |
| 998 | CACCUACA A CACAGACA | 4886 | TGTCTGTG GGCTAGCTACAACGA TGTAGGTG | 5874 |
| 1000 | CCUACAAC A CAGACACG | 4887 | CGTGTCTG GGCTAGCTACAACGA GTTGTAGG | 5875 |
| 1004 | CAACACAG A CACGUUUG | 4888 | CAAACGTG GGCTAGCTACAACGA CTGTGTTG | 5876 |
| 1006 | ACACAGAC A CGUUUGAG | 4889 | CTCAAACG GGCTAGCTACAACGA GTCTGTGT | 5877 |
| 1008 | ACAGACAC G UUUGAGUC | 4890 | GACTCAAA GGCTAGCTACAACGA GTGTCTGT | 5878 |
| 1014 | ACGUUUGA G UCCAUGCC | 4891 | GGCATGGA GGCTAGCTACAACGA TCAAACGT | 5879 |
| 1018 | UUGAGUCC A UGCCCAAU | 4892 | ATTGGGCA GGCTAGCTACAACGA GGACTCAA | 5880 |
| 1020 | GAGUCCAU G CCCAAUCC | 4893 | GGATTGGG GGCTAGCTACAACGA ATGGACTC | 5881 |
| 1025 | CAUGCCCA A UCCCGAGG | 4894 | CCTCGGGA GGCTAGCTACAACGA TGGGCATG | 5882 |
| 1034 | UCCCGAGG G CCGGUAUA | 4895 | TATACCGG GGCTAGCTACAACGA CCTCGGGA | 5883 |
| 1038 | GAGGGCCG G UAUACAUU | 4896 | AATGTATA GGCTAGCTACAACGA CGGCCCTC | 5884 |
| 1040 | GGGCCGGU A UACAUUCG | 4897 | CGAATGTA GGCTAGCTACAACGA ACCGGCCC | 5885 |
| 1042 | GCCGGUAU A CAUUCGGC | 4898 | GCCGAATG GGCTAGCTACAACGA ATACCGGC | 5886 |
| 1044 | CGGUAUAC A UUCGGCGC | 4899 | GCGCCGAA GGCTAGCTACAACGA GTATACCG | 5887 |
| 1049 | UACAUUCG G CGCCAGCU | 4900 | AGCTGGCG GGCTAGCTACAACGA CGAATGTA | 5888 |
| 1051 | CAUUCGGC G CCAGCUGU | 4901 | ACAGCTGG GGCTAGCTACAACGA GCCGAATG | 5889 |
| 1055 | CGGCGCCA G CUGUGUGA | 4902 | TCACACAG GGCTAGCTACAACGA TGGCGCCG | 5890 |
| 1058 | CGCCAGCU G UGUGACUG | 4903 | CAGTCACA GGCTAGCTACAACGA AGCTGGCG | 5891 |
| 1060 | CCAGCUGU G UGACUGCC | 4904 | GGCAGTCA GGCTAGCTACAACGA ACAGCTGG | 5892 |
| 1063 | GCUGUGUG A CUGCCUGU | 4905 | ACAGGCAG GGCTAGCTACAACGA CACACAGC | 5893 |
| 1066 | GUGUGACU G CCUGUCCC | 4906 | GGGACAGG GGCTAGCTACAACGA AGTCACAC | 5894 |
| 1070 | GACUGCCU G UCCCUACA | 4907 | TGTAGGGA GGCTAGCTACAACGA AGGCAGTC | 5895 |
| 1076 | CUGUCCCU A CAACUACC | 4908 | GGTAGTTG GGCTAGCTACAACGA AGGGACAG | 5896 |
| 1079 | UCCCUACA A CUACCUUU | 4909 | AAAGGTAG GGCTAGCTACAACGA TGTAGGGA | 5897 |
| 1082 | CUACAACU A CCUUUCUA | 4910 | TAGAAAGG GGCTAGCTACAACGA AGTTGTAG | 5898 |
| 1090 | ACCUUUCU A CGGACGUG | 4911 | CACGTCCG GGCTAGCTACAACGA AGAAAGGT | 5899 |
| 1094 | UUCUACGG A CGUGGGAU | 4912 | ATCCCACG GGCTAGCTACAACGA CCGTAGAA | 5900 |
| 1096 | CUACGGAC G UGGGAUCC | 4913 | GGATCCCA GGCTAGCTACAACGA GTCCGTAG | 5901 |
| 1101 | GACGUGGG A UCCUGCAC | 4914 | GTGCAGGA GGCTAGCTACAACGA CCCACGTC | 5902 |
| 1106 | GGGAUCCU G CACCCUCG | 4915 | CGAGGGTG GGCTAGCTACAACGA AGGATCCC | 5903 |
| 1108 | GAUCCUGC A CCCUCGUC | 4916 | GACGAGGG GGCTAGCTACAACGA GCAGGATC | 5904 |
| 1114 | GCACCCUC G UCUGCCCC | 4917 | GGGGCAGA GGCTAGCTACAACGA GAGGGTGC | 5905 |
| 1118 | CCUCGUCU G CCCCCUGC | 4918 | GCAGGGGG GGCTAGCTACAACGA AGACGAGG | 5906 |
| 1125 | UGCCCCCU G CACAACCA | 4919 | TGGTTGTG GGCTAGCTACAACGA AGGGGGCA | 5907 |
| 1127 | CCCCCUGC A CAACCAAG | 4920 | CTTGGTTG GGCTAGCTACAACGA GCAGGGGG | 5908 |
| 1130 | CCUGCACA A CCAAGAGG | 4921 | CCTCTTGG GGCTAGCTACAACGA TGTGCAGG | 5909 |
| 1138 | ACCAAGAG G UGACAGCA | 4922 | TGCTGTCA GGCTAGCTACAACGA CTCTTGGT | 5910 |
| 1141 | AAGAGGUG A CAGCAGAG | 4923 | CTCTGCTG GGCTAGCTACAACGA CACCTCTT | 5911 |
| 1144 | AGGUGACA G CAGAGGAU | 4924 | ATCCTCTG GGCTAGCTACAACGA TGTCACCT | 5912 |
| 1151 | AGCAGAGG A UGGAACAC | 4925 | GTGTTCCA GGCTAGCTACAACGA CCTCTGCT | 5913 |
| 1156 | AGGAUGGA A CACAGCGG | 4926 | CCGCTGTG GGCTAGCTACAACGA TCCATCCT | 5914 |
| 1158 | GAUGGAAC A CAGCGGUG | 4927 | CACCGCTG GGCTAGCTACAACGA GTTCCATC | 5915 |
| 1161 | GGAACACA G CGGUGUGA | 4928 | TCACACCG GGCTAGCTACAACGA TGTGTTCC | 5916 |
| 1164 | ACACAGCG G UGUGAGAA | 4929 | TTCTCACA GGCTAGCTACAACGA CGCTGTGT | 5917 |
| 1166 | ACAGCGGU G UGAGAAGU | 4930 | ACTTCTCA GGCTAGCTACAACGA ACCGCTGT | 5918 |
| 1173 | UGUGAGAA G UGCAGCAA | 4931 | TTGCTGCA GGCTAGCTACAACGA TTCTCACA | 5919 |
| 1175 | UGAGAAGU G CAGCAAGC | 4932 | GCTTGCTG GGCTAGCTACAACGA ACTTCTCA | 5920 |
| 1178 | GAAGUGCA G CAAGCCCU | 4935 | AGGGCTTG GGCTAGCTACAACGA TGCACTTC | 5921 |
| 1182 | UGCAGCAA G CCCUGUGC | 4934 | GCACAGGG GGCTAGCTACAACGA TTGCTGCA | 5922 |
| 1187 | CAAGCCCU G UGCCCGAG | 4935 | CTCGGGCA GGCTAGCTACAACGA AGGGCTTG | 5923 |
| 1189 | AGCCCUGU G CCCGAGUG | 4936 | CACTCGGG GGCTAGCTACAACGA ACAGGGCT | 5924 |
| 1195 | GUGCCCGA G UGUGCUAU | 4937 | ATAGCACA GGCTAGCTACAACGA TCGGGCAC | 5925 |
| 1197 | GCCCGAGU G UGCUAUGG | 4938 | CCATAGCA GGCTAGCTACAACGA ACTCGGGC | 5926 |
| 1199 | CCGAGUGU G CUAUGGUC | 4939 | GACCATAG GGCTAGCTACAACGA ACACTCGG | 5927 |
| 1202 | AGUGUGCU A UGGUCUGG | 4940 | CCAGACCA GGCTAGCTACAACGA AGCACACT | 5928 |
| 1205 | GUGCUAUG G UCUGGGCA | 4941 | TGCCCAGA GGCTAGCTACAACGA CATAGCAC | 5929 |
| 1211 | UGGUCUGG G CAUGGAGC | 4942 | GCTCCATG GGCTAGCTACAACGA CCAGACCA | 5930 |
| 1213 | GUCUGGGC A UGGAGCAC | 4943 | GTGCTCCA GGCTAGCTACAACGA GCCCAGAC | 5931 |
| 1218 | GGCAUGGA G CACUUGCG | 4944 | CGCAAGTG GGCTAGCTACAACGA TCCATGCC | 5932 |
| 1220 | CAUGGAGC A CUUGCGAG | 4945 | CTCGCAAG GGCTAGCTACAACGA GCTCCATG | 5933 |
| 1224 | GAGCACUU G CGAGAGGU | 4946 | ACCTCTCG GGCTAGCTACAACGA AAGTGCTC | 5934 |
| 1231 | UGCGAGAG G UGAGGGCA | 4947 | TGCCCTCA GGCTAGCTACAACGA CTCTCGCA | 5935 |
| 1237 | AGGUGAGG G CAGUUACC | 4948 | GGTAACTG GGCTAGCTACAACGA CCTCACCT | 5936 |
| 1240 | UGAGGGCA G UUACCAGU | 4949 | ACTGGTAA GGCTAGCTACAACGA TGCCCTCA | 5937 |
| 1243 | GGGCAGUU A CCAGUGCC | 4950 | GGCACTGG GGCTAGCTACAACGA AACTGCCC | 5938 |
| 1247 | AGUUACCA G UGCCAAUA | 4951 | TATTGGCA GGCTAGCTACAACGA TGGTAACT | 5939 |
| 1249 | UUACCAGU G CCAAUAUC | 4952 | GATATTGG GGCTAGCTACAACGA ACTGGTAA | 5940 |
| 1253 | CAGUGCCA A UAUCCAGG | 4953 | CCTGGATA GGCTAGCTACAACGA TGGCACTG | 5941 |
| 1255 | GUGCCAAU A UCCAGGAG | 4954 | CTCCTGGA GGCTAGCTACAACGA ATTGGCAC | 5942 |
| 1263 | AUCCAGGA G UUUGCUGG | 4955 | CCAGCAAA GGCTAGCTACAACGA TCCTGGAT | 5943 |
| 1267 | AGGAGUUU G CUGGCUGC | 4956 | GCAGCCAG GGCTAGCTACAACGA AAACTCCT | 5944 |
| 1271 | GUUUGCUG G CUGCAAGA | 4957 | TCTTGCAG GGCTAGCTACAACGA CAGCAAAC | 5945 |
| 1274 | UGCUGGCU G CAAGAAGA | 4958 | TCTTCTTG GGCTAGCTACAACGA AGCCAGCA | 5946 |
| 1282 | GCAAGAAG A UCUUUGGG | 4959 | CCCAAAGA GGCTAGCTACAACGA CTTCTTGC | 5947 |
| 1292 | CUUUGGGA G CCUGGCAU | 4960 | ATGCCAGG GGCTAGCTACAACGA TCCCAAAG | 5948 |
| 1297 | GGAGCCUG G CAUUUCUG | 4961 | CAGAAATG GGCTAGCTACAACGA CAGGCTCC | 5949 |
| 1299 | AGCCUGGC A UUUCUGCC | 4962 | GGCAGAAA GGCTAGCTACAACGA GCCAGGCT | 5950 |
| 1305 | GCAUUUCU G CCGGAGAG | 4963 | CTCTCCGG GGCTAGCTACAACGA AGAAATGC | 5951 |
| 1313 | GCCGGAGA G CUUUGAUG | 4964 | CATCAAAG GGCTAGCTACAACGA TCTCCGGC | 5952 |
| 1319 | GAGCUUUG A UGGGGACC | 4965 | GGTCCCCA GGCTAGCTACAACGA CAAAGCTC | 5953 |
| 1325 | UGAUGGGG A CCCAGCCU | 4966 | AGGCTGGG GGCTAGCTACAACGA CCCCATCA | 5954 |
| 1330 | GGGACCCA G CCUCCAAC | 4967 | GTTGGAGG GGCTAGCTACAACGA TGGGTCCC | 5955 |
| 1337 | AGCCUCCA A CACUGCCC | 4968 | GGGCAGTG GGCTAGCTACAACGA TGGAGGCT | 5956 |
| 1339 | CCUCCAAC A CUGCCCCG | 4969 | CGGGGCAG GGCTAGCTACAACGA GTTGGAGG | 5957 |
| 1342 | CCAACACU G CCCCGCUC | 4970 | GAGCGGGG GGCTAGCTACAACGA AGTGTTGG | 5958 |
| 1347 | ACUGCCCC G CUCCAGCC | 4971 | GGCTGGAG GGCTAGCTACAACGA GGGGCAGT | 5959 |
| 1353 | CCGCUCCA G CCAGAGCA | 4972 | TGCTCTGG GGCTAGCTACAACGA TGGAGCGG | 5960 |
| 1359 | CAGCCAGA G CAGCUCCA | 4973 | TGGAGCTG GGCTAGCTACAACGA TCTGGCTG | 5961 |
| 1362 | CCAGAGCA G CUCCAAGU | 4974 | ACTTGGAG GGCTAGCTACAACGA TGCTCTGG | 5962 |
| 1369 | AGCUCCAA G UGUUUGAG | 4975 | CTCAAACA GGCTAGCTACAACGA TTGGAGCT | 5963 |
| 1371 | CUCCAAGU G UUUGAGAC | 4976 | GTCTCAAA GGCTAGCTACAACGA ACTTGGAG | 5964 |
| 1378 | UGUUUGAG A CUCUGGAA | 4977 | TTCCAGAG GGCTAGCTACAACGA CTCAAACA | 5965 |
| 1390 | UGGAAGAG A UCACAGGU | 4978 | ACCTGTGA GGCTAGCTACAACGA CTCTTCCA | 5966 |
| 1393 | AAGAGAUC A CAGGUUAC | 4979 | GTAACCTG GGCTAGCTACAACGA GATCTCTT | 5967 |
| 1397 | GAUCACAG G UUACCUAU | 4980 | ATAGGTAA GGCTAGCTACAACGA CTGTGATC | 5968 |
| 1400 | CACAGGUU A CCUAUACA | 4981 | TGTATAGG GGCTAGCTACAACGA AACCTGTG | 5969 |
| 1404 | GGUUACCU A UACAUCUC | 4982 | GAGATGTA GGCTAGCTACAACGA AGGTAACC | 5970 |
| 1406 | UUACCUAU A CAUCUCAG | 4983 | CTGAGATG GGCTAGCTACAACGA ATAGGTAA | 5971 |
| 1408 | ACCUAUAC A UCUCAGCA | 4984 | TGCTGAGA GGCTAGCTACAACGA GTATAGGT | 5972 |
| 1414 | ACAUCUCA G CAUGGCCG | 4985 | CGGCCATG GGCTAGCTACAACGA TGAGATGT | 5973 |
| 1416 | AUCUCAGC A UGGCCGGA | 4986 | TCCGGCCA GGCTAGCTACAACGA GCTGAGAT | 5974 |
| 1419 | UCAGCAUG G CCGGACAG | 4987 | CTGTCCGG GGCTAGCTACAACGA CATGCTGA | 5975 |
| 1424 | AUGGCCGG A CAGCCUGC | 4988 | GCAGGCTG GGCTAGCTACAACGA CCGGCCAT | 5976 |
| 1427 | GCCGGACA G CCUGCCUG | 4989 | CAGGCAGG GGCTAGCTACAACGA TGTCCGGC | 5977 |
| 1431 | GACAGCCU G CCUGACCU | 4990 | AGGTCAGG GGCTAGCTACAACGA AGGCTGTC | 5978 |
| 1436 | CCUGCCUG A CCUCAGCG | 4991 | CGCTGAGG GGCTAGCTACAACGA CAGGCAGG | 5979 |
| 1442 | UGACCUCA G CGUCUUCC | 4992 | GGAAGACG GGCTAGCTACAACGA TGAGGTCA | 5980 |
| 1444 | ACCUCAGC G UCUUCCAG | 4993 | CTGGAAGA GGCTAGCTACAACGA GCTGAGGT | 5981 |
| 1454 | CUUCCAGA A CCUGCAAG | 4994 | CTTGCAGG GGCTAGCTACAACGA TCTGGAAG | 5982 |
| 1458 | CAGAACCU G CAAGUAAU | 4995 | ATTACTTG GGCTAGCTACAACGA AGGTTCTG | 5983 |
| 1462 | ACCUGCAA G UAAUCCGG | 4996 | CCGGATTA GGCTAGCTACAACGA TTGCAGGT | 5984 |
| 1465 | UGCAAGUA A UCCGGGGA | 4997 | TCCCCGGA GGCTAGCTACAACGA TACTTGCA | 5985 |
| 1473 | AUCCGGGG A CGAAUUCU | 4998 | AGAATTCG GGCTAGCTACAACGA CCCCGGAT | 5986 |
| 1477 | GGGGACGA A UUCUGCAC | 4999 | GTGCAGAA GGCTAGCTACAACGA TCGTCCCC | 5987 |
| 1482 | CGAAUUCU G CACAAUGG | 5000 | CCATTGTG GGCTAGCTACAACGA AGAATTCG | 5988 |
| 1484 | AAUUCUGC A CAAUGGCG | 5001 | CGCCATTG GGCTAGCTACAACGA GCAGAATT | 5989 |
| 1487 | UCUGCACA A UGGCGCCU | 5002 | AGGCGCCA GGCTAGCTACAACGA TGTGCAGA | 5990 |
| 1490 | GCACAAUG G CGCCUACU | 5003 | AGTAGGCG GGCTAGCTACAACGA CATTGTGC | 5991 |
| 1492 | ACAAUGGC G CCUACUCG | 5004 | CGAGTAGG GGCTAGCTACAACGA GCCATTGT | 5992 |
| 1496 | UGGCGCCU A CUCGCUGA | 5005 | TCAGCGAG GGCTAGCTACAACGA AGGCGCCA | 5993 |
| 1500 | GCCUACUC G CUGACCCU | 5006 | AGGGTCAG GGCTAGCTACAACGA GAGTAGGC | 5994 |
| 1504 | ACUCGCUG A CCCUGCAA | 5007 | TTGCAGGG GGCTAGCTACAACGA CAGCGAGT | 5995 |
| 1509 | CUGACCCU G CAAGGGCU | 5008 | AGCCCTTG GGCTAGCTACAACGA AGGGTCAG | 5996 |
| 1515 | CUGCAAGG G CUGGGCAU | 5009 | ATGCCCAG GGCTAGCTACAACGA CCTTGCAG | 5997 |
| 1520 | AGGGCUGG G CAUCAGCU | 5010 | AGCTGATG GGCTAGCTACAACGA CCAGCCCT | 5998 |
| 1522 | GGCUGGGC A UCAGCUGG | 5011 | CCAGCTGA GGCTAGCTACAACGA GCCCAGCC | 5999 |
| 1526 | GGGCAUCA G CUGGCUGG | 5012 | CCAGCCAG GGCTAGCTACAACGA TGATGCCC | 6000 |
| 1530 | AUCAGCUG G CUGGGGCU | 5013 | AGCCCCAG GGCTAGCTACAACGA CAGCTGAT | 6001 |
| 1536 | UGGCUGGG G CUGCGCUC | 5014 | GAGCGCAG GGCTAGCTACAACGA CCCAGCCA | 6002 |
| 1539 | CUGGGGCU G CGCUCACU | 5015 | AGTGAGCG GGCTAGCTACAACGA AGCCCCAG | 6003 |
| 1541 | GGGGCUGC G CUCACUGA | 5016 | TCAGTGAG GGCTAGCTACAACGA GCAGCCCC | 6004 |
| 1545 | CUGCGCUC A CUGAGGGA | 5017 | TCCCTCAG GGCTAGCTACAACGA GAGCGCAG | 6005 |
| 1554 | CUGAGGGA A CUGGGCAG | 5018 | CTGCCCAG GGCTAGCTACAACGA TCCCTCAG | 6006 |
| 1559 | GGAACUGG G CAGUGGAC | 5019 | GTCCACTG GGCTAGCTACAACGA CCAGTTCC | 6007 |
| 1562 | ACUGGGCA G UGGACUGG | 5020 | CCAGTCCA GGCTAGCTACAACGA TGCCCAGT | 6008 |
| 1566 | GGCAGUGG A CUGGCCCU | 5021 | AGGGCCAG GGCTAGCTACAACGA CCACTGCC | 6009 |
| 1570 | GUGGACUG G CCCUCAUC | 5022 | GATGAGGG GGCTAGCTACAACGA CAGTCCAC | 6010 |
| 1576 | UGGCCCUC A UCCACCAU | 5023 | ATGGTGGA GGCTAGCTACAACGA GAGGGCCA | 6011 |
| 1580 | CCUCAUCC A CCAUAACA | 5024 | TGTTATGG GGCTAGCTACAACGA GGATGAGG | 6012 |
| 1583 | CAUCCACC A UAACACCC | 5025 | GGGTGTTA GGCTAGCTACAACGA GGTGGATG | 6013 |
| 1586 | CCACCAUA A CACCCACC | 5026 | GGTGGGTG GGCTAGCTACAACGA TATGGTGG | 6014 |
| 1588 | ACCAUAAC A CCCACCUC | 5027 | GAGGTGGG GGCTAGCTACAACGA GTTATGGT | 6015 |
| 1592 | UAACACCC A CCUCUGCU | 5028 | AGCAGAGG GGCTAGCTACAACGA GGGTGTTA | 6016 |
| 1598 | CCACCUCU G CUUCGUGC | 5029 | GCACGAAG GGCTAGCTACAACGA AGAGGTGG | 6017 |
| 1603 | UCUGCUUC G UGCACACG | 5030 | CGTGTGCA GGCTAGCTACAACGA GAAGCAGA | 6018 |
| 1605 | UGCUUCGU G CACACGGU | 5031 | ACCGTGTG GGCTAGCTACAACGA ACGAAGCA | 6019 |
| 1607 | CUUCGUGC A CACGGUGC | 5032 | GCACCGTG GGCTAGCTACAACGA GCACGAAG | 6020 |
| 1609 | UCGUGCAC A CGGUGCCC | 5033 | GGGCACCG GGCTAGCTACAACGA GTGCACGA | 6021 |
| 1612 | UGCACACG G UGCCCUGG | 5034 | CCAGGGCA GGCTAGCTACAACGA CGTGTGCA | 6022 |
| 1614 | CACACGGU G CCCUGGGA | 5035 | TCCCAGGG GGCTAGCTACAACGA ACCGTGTG | 6023 |
| 1622 | GCCCUGGG A CCAGCUCU | 5036 | AGAGCTGG GGCTAGCTACAACGA CCCAGGGC | 6024 |
| 1626 | UGGGACCA G CUCUUUCG | 5037 | CGAAAGAG GGCTAGCTACAACGA TGGTCCCA | 6025 |
| 1637 | CUUUCGGA A CCCGCACC | 5038 | GGTGCGGG GGCTAGCTACAACGA TCCGAAAG | 6026 |
| 1641 | CGGAACCC G CACCAAGC | 5039 | GCTTGGTG GGCTAGCTACAACGA GGGTTCCG | 6027 |
| 1643 | GAACCCGC A CCAAGCUC | 5040 | GAGCTTGG GGCTAGCTACAACGA GCGGGTTC | 6028 |
| 1648 | CGCACCAA G CUCUGCUC | 5041 | GAGCAGAG GGCTAGCTACAACGA TTGGTGCG | 6029 |
| 1653 | CAAGCUCU G CUCCACAC | 5042 | GTGTGGAG GGCTAGCTACAACGA AGAGCTTG | 6030 |
| 1658 | UCUGCUCC A CACUGCCA | 5043 | TGGCAGTG GGCTAGCTACAACGA GGAGCAGA | 6031 |
| 1660 | UGCUCCAC A CUGCCAAC | 5044 | GTTGGCAG GGCTAGCTACAACGA GTGGAGCA | 6032 |
| 1663 | UCCACACU G CCAACCGG | 5045 | CCGGTTGG GGCTAGCTACAACGA AGTGTGGA | 6033 |
| 1667 | CACUGCCA A CCGGCCAG | 5046 | CTGGCCGG GGCTAGCTACAACGA TGGCAGTG | 6034 |
| 1671 | GCCAACCG G CCAGAGGA | 5047 | TCCTCTGG GGCTAGCTACAACGA CGGTTGGC | 6035 |
| 1679 | GCCAGAGG A CGAGUGUG | 5048 | CACACTCG GGCTAGCTACAACGA CCTCTGGC | 6036 |
| 1683 | GAGGACGA G UGUGUGGG | 5049 | CCCACACA GGCTAGCTACAACGA TCGTCCTC | 6037 |
| 1685 | GGACGAGU G UGUGGGCG | 5050 | CGCCCACA GGCTAGCTACAACGA ACTCGTCC | 6038 |
| 1687 | ACGAGUGU G UGGGCGAG | 5051 | CTCGCCCA GGCTAGCTACAACGA ACACTCGT | 6039 |
| 1691 | GUGUGUGG G CGAGGGCC | 5052 | GGCCCTCG GGCTAGCTACAACGA CCACACAC | 6040 |
| 1697 | GGGCGAGG G CCUGGCCU | 5053 | AGGCCAGG GGCTAGCTACAACGA CCTCGCCC | 6041 |
| 1702 | AGGGCCUG G CCUGCCAC | 5054 | GTGGCAGG GGCTAGCTACAACGA CAGGCCCT | 6042 |
| 1706 | CCUGGCCU G CCACCAGC | 5055 | GCTGGTGG GGCTAGCTACAACGA AGGCCAGG | 6043 |
| 1709 | GGCCUGCC A CCAGCUGU | 5056 | ACAGCTGG GGCTAGCTACAACGA GGCAGGCC | 6044 |
| 1713 | UGCCACCA G CUGUGCGC | 5057 | GCGCACAG GGCTAGCTACAACGA TGGTGGCA | 6045 |
| 1716 | CACCAGCU G UGCGCCCG | 5058 | CGGGCGCA GGCTAGCTACAACGA AGCTGGTC | 6046 |
| 1718 | CCACCUGU G CGCCCGAG | 5059 | CTCGGGCG GGCTAGCTACAACGA ACAGCTGG | 6047 |
| 1720 | AGCUGUGC G CCCGAGGG | 5060 | CCCTCGGG GGCTAGCTACAACGA GCACACCT | 6048 |
| 1728 | GCCCGAGG G CACUCCUG | 5061 | CAGCAGTG GGCTAGCTACAACGA CCTCGGGC | 6049 |
| 1730 | CCGAGGGC A CUGCUGGG | 5062 | CCCACCAG GGCTAGCTACAACGA GCCCTCGG | 6050 |
| 1733 | AGGGCACU G CUGGGCUC | 5063 | GACCCCAG GGCTAGCTACAACGA AGTGCCCT | 6051 |
| 1739 | CUGCUGGG G UCCAGGGC | 5064 | GCCCTGGA GGCTAGCTACAACGA CCCAGCAG | 6052 |
| 1746 | GGUCCAGG G CCCACCCA | 5065 | TGCGTGGG GGCTAGCTACAACGA CCTCGACC | 6053 |
| 1750 | CAGGGCCC A CCCAGUGU | 5066 | ACACTGGG GGCTAGCTACAACGA GGCCCCTG | 6054 |
| 1755 | CCCACCCA G UGUGUCAA | 5067 | TTGACACA GGCTAGCTACAACGA TGGGTGGG | 6055 |
| 1757 | CACCCAGU G UGUCAACU | 5068 | AGTTGACA GGCTAGCTACAACGA ACTGGGTC | 6056 |
| 1759 | CCCAGUGU G UCAACUGC | 5069 | GCAGTTGA GGCTAGCTACAACGA ACACTGGG | 6057 |
| 1763 | GUGUGUCA A CUGCAGCC | 5070 | GGCTGCAG GGCTAGCTACAACGA TGACACAC | 6058 |
| 1766 | UGUCAACU G CAGCCAGU | 5071 | ACTGGCTG GGCTAGCTACAACGA AGTTGACA | 6059 |
| 1769 | CAACUGCA G CCAGUUCC | 5072 | GGAACTGC GGCTAGCTACAACGA TGCAGTTG | 6060 |
| 1773 | UGCAGCCA G UUCCUUCG | 5073 | CGAACCAA GGCTAGCTACAACGA TGCCTGCA | 6061 |
| 1784 | CCUUCGGG G CCAGGAGU | 5074 | ACTCCTGC GGCTAGCTACAACGA CCCGAAGG | 6062 |
| 1791 | GGCCAGGA G UGCGUGGA | 5075 | TCCACGCA GGCTAGCTACAACGA TCCTGGCC | 6063 |
| 1793 | CCAGGAGU G CGUGGAGG | 5076 | CCTCCACG GGCTAGCTACAACGA ACTCCTGG | 6064 |
| 1795 | AGGAGUGC G UGGAGGAA | 5077 | TTCCTCCA GGCTAGCTACAACGA GCACTCCT | 6065 |
| 1803 | GUGGAGGA A UGCCGAGU | 5078 | ACTCGGCA GGCTAGCTACAACGA TCCTCCAC | 6066 |
| 1805 | GGAGGAAU G CCGAGUAC | 5079 | GTACTCGG GGCTAGCTACAACGA ATTCCTCC | 6067 |
| 1810 | AAUGCCGA G UACUGCAG | 5080 | CTGCAGTA GGCTAGCTACAACGA TCGGCATT | 6068 |
| 1812 | UGCCGAGU A CUGCAGGG | 5081 | CCCTGCAG GGCTAGCTACAACGA ACTCGGCA | 6069 |
| 1815 | CGAGUACU G CAGGGGCU | 5082 | AGCCCCTG GGCTAGCTACAACGA AGTACTCG | 6070 |
| 1821 | CUGCAGGG G CUCCCCAG | 5083 | CTGGGGAG GGCTAGCTACAACGA CCCTGCAG | 6071 |
| 1833 | CCCAGGGA G UAUGUGAA | 5084 | TTCACATA GGCTAGCTACAACGA TCCCTGGG | 6072 |
| 1835 | CAGGGAGU A UGUGAAUG | 5085 | CATTCACA GGCTAGCTACAACGA ACTCCCTG | 6073 |
| 1837 | GGGAGUAU G UGAAUGCC | 5086 | GGCATTCA GGCTAGCTACAACGA ATACTCCC | 6074 |
| 1841 | GUAUGUGA A UGCCAGGC | 5087 | GCCTGGCA GGCTAGCTACAACGA TCACATAC | 6075 |
| 1843 | AUGUGAAU G CCAGGCAC | 5088 | GTGCCTGG GGCTAGCTACAACGA ATTCACAT | 6076 |
| 1848 | AAUGCCAG G CACUGUUU | 5089 | AAACAGTG GGCTAGCTACAACGA CTGGCATT | 6077 |
| 1850 | UGCCAGGC A CUGUUUGC | 5090 | GCAAACAG GGCTAGCTACAACGA GCCTGGCA | 6078 |
| 1853 | CAGGCACU G UUUGCCGU | 5091 | ACGGCAAA GGCTAGCTACAACGA AGTGCCTG | 6079 |
| 1857 | CACUGUUU G CCGUGCCA | 5092 | TGGCACGG GGCTAGCTACAACGA AAACAGTG | 6080 |
| 1860 | UGUUUGCC G UGCCACCC | 5093 | GGGTGGCA GGCTAGCTACAACGA GGCAAACA | 6081 |
| 1862 | UUUGCCGU G CCACCCUG | 5094 | CAGGGTGG GGCTAGCTACAACGA ACGGCAAA | 6082 |
| 1865 | GCCGUGCC A CCCUGAGU | 5095 | ACTCAGGG GGCTAGCTACAACGA GGCACGGC | 6083 |
| 1872 | CACCCUGA G UGUCAGCC | 5096 | GGCTGACA GGCTAGCTACAACGA TCAGGGTG | 6084 |
| 1874 | CCCUGAGU G UCAGCCCC | 5097 | GGGGCTGA GGCTAGCTACAACGA ACTCAGGG | 6085 |
| 1878 | GAGUGUCA G CCCCAGAA | 5098 | TTCTGGGG GGCTAGCTACAACGA TGACACTC | 6086 |
| 1886 | GCCCCAGA A UGGCUCAG | 5099 | CTGAGCCA GGCTAGCTACAACGA TCTGGGGC | 6087 |
| 1889 | CCAGAAUG G CUCAGUGA | 5100 | TCACTGAG GGCTAGCTACAACGA CATTCTGG | 6088 |
| 1894 | AUGGCUCA G UGACCUGU | 5101 | ACAGGTCA GGCTAGCTACAACGA TGAGCCAT | 6089 |
| 1897 | GCUCAGUG A CCUGUUUU | 5102 | AAAACAGG GGCTAGCTACAACGA CACTGAGC | 6090 |
| 1901 | AGUGACCU G UUUUGGAC | 5103 | GTCCAAAA GGCTAGCTACAACGA AGGTCACT | 6091 |
| 1908 | UGUUUUGG A CCGGAGGC | 5104 | GCCTCCGG GGCTAGCTACAACGA CCAAAACA | 6092 |
| 1915 | GACCGGAG G CUGACCAG | 5105 | CTGGTCAG GGCTAGCTACAACGA CTCCGGTC | 6093 |
| 1919 | GGAGGCUG A CCAGUGUG | 5106 | CACACTGG GGCTAGCTACAACGA CAGCCTCC | 6094 |
| 1923 | GCUGACCA G UGUGUGGC | 5107 | GCCACACA GGCTAGCTACAACGA TGGTCAGC | 6095 |
| 1925 | UGACCAGU G UGUGGCCU | 5108 | AGGCCACA GGCTAGCTACAACGA ACTGGTCA | 6096 |
| 1927 | ACCAGUGU G UGGCCUGU | 5109 | ACAGGCCA GGCTAGCTACAACGA ACACTGGT | 6097 |
| 1930 | AGUGUGUG G CCUGUGCC | 5110 | GGCACAGG GGCTAGCTACAACGA CACACACT | 6098 |
| 1934 | UGUGGCCU G UGCCCACU | 5111 | AGTGGGCA GGCTAGCTACAACGA AGGCCACA | 6099 |
| 1936 | UGGCCUGU G CCCACUAU | 5112 | ATAGTGGG GGCTAGCTACAACGA ACAGGCCA | 6100 |
| 1940 | CUGUGCCC A CUAUAAGG | 5113 | CCTTATAG GGCTAGCTACAACGA GGGCACAG | 6101 |
| 1943 | UGCCCACU A UAAGGACC | 5114 | GGTCCTTA GGCTAGCTACAACGA AGTGGGCA | 6102 |
| 1949 | CUAUAAGG A CCCUCCCU | 5115 | AGGGAGGG GGCTAGCTACAACGA CCTTATAG | 6103 |
| 1961 | UCCCUUCU G CGUGGCCC | 5116 | GGGCCACG GGCTAGCTACAACGA AGAAGGGA | 6104 |
| 1963 | CCUUCUGC G UGGCCCGC | 5117 | GCGGGCCA GGCTAGCTACAACGA GCAGAAGG | 6105 |
| 1966 | UCUGCGUG G CCCGCUGC | 5118 | GCAGCGGG GGCTAGCTACAACGA CACGCAGA | 6106 |
| 1970 | CGUGGCCC G CUGCCCCA | 5119 | TGGGGCAG GGCTAGCTACAACGA GGGCCACG | 6107 |
| 1973 | GGCCCGCU G CCCCAGCG | 5120 | CGCTGGGG GGCTAGCTACAACGA AGCGGGCC | 6108 |
| 1979 | CUGCCCCA G CGGUGUGA | 5121 | TCACACCG GGCTAGCTACAACGA TGGGGCAG | 6109 |
| 1982 | CCCCAGCG G UGUGAAAC | 5122 | GTTTCACA GGCTAGCTACAACGA CGCTGGGG | 6110 |
| 1984 | CCAGCGGU G UGAAACCU | 5123 | AGGTTTCA GGCTAGCTACAACGA ACCGCTGG | 6111 |
| 1989 | GGUGUGAA A CCUGACCU | 5124 | AGGTCAGG GGCTAGCTACAACGA TTCACACC | 6112 |
| 1994 | GAAACCUG A CCUCUCCU | 5125 | AGGAGAGG GGCTAGCTACAACGA CAGGTTTC | 6113 |
| 2003 | CCUCUCCU A CAUGCCCA | 5126 | TGGGCATG GGCTAGCTACAACGA AGGAGAGG | 6114 |
| 2005 | UCUCCUAC A UGCCCAUC | 5127 | GATGGGCA GGCTAGCTACAACGA GTAGGAGA | 6115 |
| 2007 | UCCUACAU G CCCAUCUG | 5128 | CAGATGGG GGCTAGCTACAACGA ATGTAGGA | 6116 |
| 2011 | ACAUGCCC A UCUGGAAG | 5129 | CTTCCAGA GGCTAGCTACAACGA GGGCATGT | 6117 |
| 2019 | AUCUGGAA G UUUCCAGA | 5130 | TCTGGAAA GGCTAGCTACAACGA TTCCAGAT | 6118 |
| 2027 | GUUUCCAG A UGAGGAGG | 5131 | CCTCCTCA GGCTAGCTACAACGA CTGGAAAC | 6119 |
| 2036 | UGAGGAGG G CGCAUGCC | 5132 | GGCATGCG GGCTAGCTACAACGA CCTCCTCA | 6120 |
| 2038 | AGGAGGGC G CAUGCCAG | 5133 | CTGGCATG GGCTAGCTACAACGA GCCCTCCT | 6121 |
| 2040 | GAGGGCGC A UGCCAGCC | 5134 | GGCTGGCA GGCTAGCTACAACGA GCGCCCTC | 6122 |
| 2042 | GGGCGCAU G CCAGCCUU | 5135 | AAGGCTGG GGCTAGCTACAACGA ATGCGCCC | 6123 |
| 2046 | GCAUGCCA G CCUUGCCC | 5136 | GGGCAAGG GGCTAGCTACAACGA TGGCATGC | 6124 |
| 2051 | CCAGCCUU G CCCCAUCA | 5137 | TGATGGGG GGCTAGCTACAACGA AAGGCTGG | 6125 |
| 2056 | CUUGCCCC A UCAACUGC | 5138 | GCAGTTGA GGCTAGCTACAACGA GGGGCAAG | 6126 |
| 2060 | CCCCAUCA A CUGCACCC | 5139 | GGGTGCAG GGCTAGCTACAACGA TGATGGGG | 6127 |
| 2063 | CAUCAACU G CACCCACU | 5140 | AGTGGGTG GGCTAGCTACAACGA AGTTGATG | 6128 |
| 2065 | UCAACUGC A CCCACUCC | 5141 | GGAGTGGG GGCTAGCTACAACGA GCAGTTGA | 6129 |
| 2069 | CUGCACCC A CUCCUGUG | 5142 | CACAGGAG GGCTAGCTACAACGA GGGTGCAG | 6130 |
| 2075 | CCACUCCU G UGUGGACC | 5143 | GGTCCACA GGCTAGCTACAACGA AGGAGTGG | 6131 |
| 2077 | ACUCCUGU G UGGACCUG | 5144 | CAGGTCCA GGCTAGCTACAACGA ACAGGAGT | 6132 |
| 2081 | CUGUGUGG A CCUGGAUG | 5145 | CATCCAGG GGCTAGCTACAACGA CCACACAG | 6133 |
| 2087 | GGACCUGG A UGACAAGG | 5146 | CCTTGTCA GGCTAGCTACAACGA CCAGGTCC | 6134 |
| 2090 | CCUGGAUG A CAAGGGCU | 5147 | AGCCCTTG GGCTAGCTACAACGA CATCCAGG | 6135 |
| 2096 | UGACAAGG G CUGCCCCG | 5148 | CGGGGCAG GGCTAGCTACAACGA CCTTGTCA | 6136 |
| 2099 | CAAGGGCU G CCCCGCCG | 5149 | CGGCGGGG GGCTAGCTACAACGA AGCCCTTG | 6137 |
| 2104 | GCUGCCCC G CCGAGCAG | 5150 | CTGCTCGG GGCTAGCTACAACGA GGGGCAGC | 6138 |
| 2109 | CCCGCCGA G CAGAGAGC | 5151 | GCTCTCTG GGCTAGCTACAACGA TCGGCGGG | 6139 |
| 2116 | AGCAGAGA G CCAGCCCU | 5152 | AGGGCTGG GGCTAGCTACAACGA TCTCTGCT | 6140 |
| 2120 | GAGAGCCA G CCCUCUGA | 5153 | TCAGAGGG GGCTAGCTACAACGA TGGCTCTC | 6141 |
| 2128 | GCCCUCUG A CGUCCAUC | 5154 | GATGGACG GGCTAGCTACAACGA CAGAGGGC | 6142 |
| 2130 | CCUCUGAC G UCCAUCAU | 5155 | ATGATGGA GGCTAGCTACAACGA GTCAGAGG | 6143 |
| 2134 | UGACGUCC A UCAUCUCU | 5156 | AGAGATGA GGCTAGCTACAACGA GGACGTCA | 6144 |
| 2137 | CGUCCAUC A UCUCUGCG | 5157 | CGCAGAGA GGCTAGCTACAACGA GATGGACG | 6145 |
| 2143 | UCAUCUCU G CGGUGGUU | 5158 | AACCACCG GGCTAGCTACAACGA AGAGATGA | 6146 |
| 2146 | UCUCUGCG G UGGUUGGC | 5159 | GCCAACCA GGCTAGCTACAACGA CGCAGAGA | 6147 |
| 2149 | CUGCGGUG G UUGGCAUU | 5160 | AATGCCAA GGCTAGCTACAACGA CACCGCAG | 6148 |
| 2153 | GGUGGUUG G CAUUCUGC | 5161 | GCAGAATG GGCTAGCTACAACGA CAACCACC | 6149 |
| 2155 | UGGUUGGC A UUCUGCUG | 5162 | CAGCAGAA GGCTAGCTACAACGA GCCAACCA | 6150 |
| 2160 | GGCAUUCU G CUGGUCGU | 5163 | ACGACCAG GGCTAGCTACAACGA AGAATGCC | 6151 |
| 2164 | UUCUGCUG G UCGUGGUC | 5164 | GACCACGA GGCTAGCTACAACGA CAGCAGAA | 6152 |
| 2167 | UGCUGGUC G UGGUCUUG | 5165 | CAAGACCA GGCTAGCTACAACGA GACCAGCA | 6153 |
| 2170 | UGGUCGUG G UCUUGGGG | 5166 | CCCCAAGA GGCTAGCTACAACGA CACGACCA | 6154 |
| 2179 | UCUUGGGG G UGGUCUUU | 5167 | AAAGACCA GGCTAGCTACAACGA CCCCAAGA | 6155 |
| 2182 | UGGGGGUG G UCUUUGGG | 5168 | CCCAAAGA GGCTAGCTACAACGA CACCCCCA | 6156 |
| 2191 | UCUUUGGG A UCCUCAUC | 5169 | GATGAGGA GGCTAGCTACAACGA CCCAAAGA | 6157 |
| 2197 | GGAUCCUC A UCAAGCGA | 5170 | TCGCTTGA GGCTAGCTACAACGA GAGGATCC | 6158 |
| 2202 | CUCAUCAA G CGACGGCA | 5171 | TGCCGTCG GGCTAGCTACAACGA TTGATGAG | 6159 |
| 2205 | AUCAAGCG A CGGCAGCA | 5172 | TGCTGCCG GGCTAGCTACAACGA CGCTTGAT | 6160 |
| 2208 | AAGCGACG G CAGCAGAA | 5173 | TTCTGCTG GGCTAGCTACAACGA CGTCGCTT | 6161 |
| 2211 | CGACGGCA G CAGAAGAU | 5174 | ATCTTCTG GGCTAGCTACAACGA TGCCGTCG | 6162 |
| 2218 | AGCAGAAG A UCCGGAAG | 5175 | CTTCCGGA GGCTAGCTACAACGA CTTCTGCT | 6163 |
| 2226 | AUCCGGAA G UACACGAU | 5176 | ATCGTGTA GGCTAGCTACAACGA TTCCGGAT | 6164 |
| 2228 | CCGGAAGU A CACGAUGC | 5177 | GCATCGTG GGCTAGCTACAACGA ACTTCCGG | 6165 |
| 2230 | GGAAGUAC A CGAUGCGG | 5178 | CCGCATCG GGCTAGCTACAACGA GTACTTCC | 6166 |
| 2233 | AGUACACG A UGCGGAGA | 5179 | TCTCCGCA GGCTAGCTACAACGA CGTGTACT | 6167 |
| 2235 | UACACGAU G CGGAGACU | 5180 | AGTCTCCG GGCTAGCTACAACGA ATCGTGTA | 6168 |
| 2241 | AUGCGGAG A CUGCUGCA | 5181 | TGCAGCAG GGCTAGCTACAACGA CTCCGCAT | 6169 |
| 2244 | CGGAGACU G CUGCAGGA | 5182 | TCCTGCAG GGCTAGCTACAACGA AGTCTCCG | 6170 |
| 2247 | AGACUGCU G CAGGAAAC | 5183 | GTTTCCTG GGCTAGCTACAACGA AGCAGTCT | 6171 |
| 2254 | UGCAGGAA A CGGAGCUGC 5184 | CAGCTCCG GGCTAGCTACAACGA TTCCTGCA | 6172 | |
| 2259 | GAAACGGA G CUGGUGGA | 5185 | TCCACCAG GGCTAGCTACAACGA TCCGTTTC | 6173 |
| 2263 | CGGAGCUG G UGGAGCCG | 5186 | CGGCTCCA GGCTAGCTACAACGA CAGCTCCG | 6174 |
| 2268 | CUGGUGGA G CCGCUGAC | 5187 | GTCAGCGG GGCTAGCTACAACGA TCCACCAG | 6175 |
| 2271 | GUGGAGCC G CUGACACC | 5188 | GGTGTCAG GGCTAGCTACAACGA GGCTCCAC | 6176 |
| 2275 | AGCCGCUG A CACCUAGC | 5189 | GCTAGGTG GGCTAGCTACAACGA CAGCGGCT | 6177 |
| 2277 | CCGCUGAC A CCUAGCGG | 5190 | CCGCTAGG GGCTAGCTACAACGA GTCAGCGG | 6178 |
| 2282 | GACACCUA G CGGAGCGA | 5191 | TCGCTCCG GGCTAGCTACAACGA TAGGTGTC | 6179 |
| 2287 | CUAGCGGA G CGAUGCCC | 5192 | GGGCATCG GGCTAGCTACAACGA TCCGCTAG | 6180 |
| 2290 | GCGGAGCG A UGCCCAAC | 5193 | GTTGGGCA GGCTAGCTACAACGA CGCTCCGC | 6181 |
| 2292 | GGAGCGAU G CCCAACCA | 5194 | TGGTTGGG GGCTAGCTACAACGA ATCGCTCC | 6182 |
| 2297 | GAUGCCCA A CCAGGCGC | 5195 | GCGCCTGG GGCTAGCTACAACGA TGGGCATC | 6183 |
| 2302 | CCAACCAG G CGCAGAUG | 5196 | CATCTGCG GGCTAGCTACAACGA CTGGTTGG | 6184 |
| 2304 | AACCAGGC G CAGAUGCG | 5197 | CGCATCTG GGCTAGCTACAACGA GCCTGGTT | 6185 |
| 2308 | AGGCGCAG A UGCGGAUC | 5198 | GATCCGCA GGCTAGCTACAACGA CTGCGCCT | 6186 |
| 2310 | GCGCAGAU G CGGAUCCU | 5199 | AGGATCCG GGCTAGCTACAACGA ATCTGCGC | 6187 |
| 2314 | AGAUGCGG A UCCUGAAA | 5200 | TTTCAGGA GGCTAGCTACAACGA CCGCATCT | 6188 |
| 2326 | UGAAAGAG A CGGAGCUG | 5201 | CAGCTCCG GGCTAGCTACAACGA CTCTTTCA | 6189 |
| 2331 | GAGACGGA G CUGAGGAA | 5202 | TTCCTCAG GGCTAGCTACAACGA TCCGTCTC | 6190 |
| 2341 | UGAGGAAG G UGAAGGUG | 5203 | CACCTTCA GGCTAGCTACAACGA CTTCCTCA | 6191 |
| 2347 | AGGUGAAG G UGCUUGGA | 5204 | TCCAAGCA GGCTAGCTACAACGA CTTCACCT | 6192 |
| 2349 | GUGAAGGU G CUUGGAUC | 5205 | GATCCAAG GGCTAGCTACAACGA ACCTTCAC | 6193 |
| 2355 | GUGCUUGG A UCUGGCGC | 5206 | GCGCCAGA GGCTAGCTACAACGA CCAAGCAC | 6194 |
| 2360 | UGGAUCUG G CGCUUUUG | 5207 | CAAAAGCG GGCTAGCTACAACGA CAGATCCA | 6195 |
| 2362 | GAUCUGGC G CUUUUGGC | 5208 | GCCAAAAG GGCTAGCTACAACGA GCCAGATC | 6196 |
| 2369 | CGCUUUUG G CACAGUCU | 5209 | AGACTGTG GGCTAGCTACAACGA CAAAAGCG | 6197 |
| 2371 | CUUUUGGC A CAGUCUAC | 5210 | GTAGACTG GGCTAGCTACAACGA GCCAAAAG | 6198 |
| 2374 | UUGGCACA G UCUACAAG | 5211 | CTTGTAGA GGCTAGCTACAACGA TGTGCCAA | 6199 |
| 2378 | CACAGUCU A CAAGGGCA | 5212 | TGCCCTTG GGCTAGCTACAACGA AGACTGTG | 6200 |
| 2384 | CUACAAGG G CAUCUGGA | 5213 | TCCAGATG GGCTAGCTACAACGA CCTTGTAG | 6201 |
| 2386 | ACAAGGGC A UCUGGAUC | 5214 | GATCCAGA GGCTAGCTACAACGA GCCCTTGT | 6202 |
| 2392 | GCAUCUGG A UCCCUGAU | 5215 | ATCAGGGA GGCTAGCTACAACGA CCAGATGC | 6203 |
| 2399 | GAUCCCUG A UGGGGAGA | 5216 | TCTCCCCA GGCTAGCTACAACGA CAGGGATC | 6204 |
| 2408 | UGGGGAGA A UGUGAAAA | 5217 | TTTTCACA GGCTAGCTACAACGA TCTCCCCA | 6205 |
| 2410 | GGGAGAAU G UGAAAAUU | 5218 | AATTTTCA GGCTAGCTACAACGA ATTCTCCC | 6206 |
| 2416 | AUGUGAAA A UUCCAGUG | 5219 | CACTGGAA GGCTAGCTACAACGA TTTCACAT | 6207 |
| 2422 | AAAUUCCA G UGGCCAUC | 5220 | GATGGCCA GGCTAGCTACAACGA TGGAATTT | 6208 |
| 2425 | UUCCAGUG G CCAUCAAA | 5221 | TTTGATGG GGCTAGCTACAACGA CACTGGAA | 6209 |
| 2428 | CAGUGGCC A UCAAAGUG | 5222 | CACTTTGA GGCTAGCTACAACGA GGCCACTG | 6210 |
| 2434 | CCAUCAAA G UGUUGAGG | 5223 | CCTCAACA GGCTAGCTACAACGA TTTGATGG | 6211 |
| 2436 | AUCAAAGU G UUGAGGGA | 5224 | TCCCTCAA GGCTAGCTACAACGA ACTTTGAT | 6212 |
| 2447 | GAGGGAAA A CACAUCCC | 5225 | GGGATGTG GGCTAGCTACAACGA TTTCCCTC | 6213 |
| 2449 | GGGAAAAC A CAUCCCCC | 5226 | GGGGGATG GGCTAGCTACAACGA GTTTTCCC | 6214 |
| 2451 | GAAAACAC A UCCCCCAA | 5227 | TTGGGGGA GGCTAGCTACAACGA GTGTTTTC | 6215 |
| 2461 | CCCCCAAA G CCAACAAA | 5228 | TTTGTTGG GGCTAGCTACAACGA TTTGGGGG | 6216 |
| 2465 | CAAAGCCA A CAAAGAAA | 5229 | TTTCTTTG GGCTAGCTACAACGA TGGCTTTG | 6217 |
| 2473 | ACAAAGAA A UCUUAGAC | 5230 | GTCTAAGA GGCTAGCTACAACGA TTCTTTGT | 6218 |
| 2480 | AAUCUUAG A CGAAGCAU | 5231 | ATGCTTCG GGCTAGCTACAACGA CTAAGATT | 6219 |
| 2485 | UAGACGAA G CAUACGUG | 5232 | CACGTATG GGCTAGCTACAACGA TTCGTCTA | 6220 |
| 2487 | GACGAAGC A UACGUGAU | 5233 | ATCACGTA GGCTAGCTACAACGA GCTTCGTC | 6221 |
| 2489 | CGAAGCAU A CGUGAUGG | 5234 | CCATCACG GGCTAGCTACAACGA ATGCTTCG | 6222 |
| 2491 | AAGCAUAC G UGAUGGCU | 5235 | AGCCATCA GGCTAGCTACAACGA GTATGCTT | 6223 |
| 2494 | CAUACGUG A UGGCUGGU | 5236 | ACCAGCCA GGCTAGCTACAACGA CACGTATG | 6224 |
| 2497 | ACGUGAUG G CUGGUGUG | 5237 | CACACCAG GGCTAGCTACAACGA CATCACGT | 6225 |
| 2501 | GAUGGCUG G UGUGGGCU | 5238 | AGCCCACA GGCTAGCTACAACGA CAGCCATC | 6226 |
| 2503 | UGGCUGGU G UGGGCUCC | 5239 | GGAGCCCA GGCTAGCTACAACGA ACCAGCCA | 6227 |
| 2507 | UGGUGUGG G CUCCCCAU | 5240 | ATGGGGAG GGCTAGCTACAACGA CCACACCA | 6228 |
| 2514 | GGCUCCCC A UAUGUCUC | 5241 | GAGACATA GGCTAGCTACAACGA GGGGAGCC | 6229 |
| 2516 | CUCCCCAU A UGUCUCCC | 5242 | GGGAGACA GGCTAGCTACAACGA ATGGGGAG | 6230 |
| 2518 | CCCCAUAU G UCUCCCGC | 5243 | GCGGGAGA GGCTAGCTACAACGA ATATGGGG | 6231 |
| 2525 | UGUCUCCC G CCUUCUGG | 5244 | CCAGAAGG GGCTAGCTACAACGA GGGAGACA | 6232 |
| 2534 | CCUUCUGG G CAUCUGCC | 5245 | GGCAGATG GGCTAGCTACAACGA CCAGAAGG | 6233 |
| 2536 | UUCUGGGC A UCUOCCUG | 5246 | CAGGCAGA GGCTAGCTACAACGA GCCCAGAA | 6234 |
| 2540 | GGGCAUCU G CCUGACAU | 5247 | ATGTCAGG GGCTAGCTACAACGA AGATGCCC | 6235 |
| 2545 | UCUGCCUG A CAUCCACG | 5248 | CGTGGATG GGCTAGCTACAACGA CAGGCAGA | 6236 |
| 2547 | UGCCUGAC A UCCACGGU | 5249 | ACCGTGGA GGCTAGCTACAACGA GTCAGGCA | 6237 |
| 2551 | UGACAUCC A CGGUGCAG | 5250 | CTGCACCG GGCTAGCTACAACGA GGATGTCA | 6238 |
| 2554 | CAUCCACG G UGCAGCUG | 5251 | CAGCTGCA GGCTAGCTACAACGA CGTGGATG | 6239 |
| 2556 | UCCACGGU G CAGCUGGU | 5252 | ACCAGCTG GGCTAGCTACAACGA ACCGTGGA | 6240 |
| 2559 | ACGGUGCA G CUGGUGAC | 5253 | GTCACCAG GGCTAGCTACAACGA TGCACCGT | 6241 |
| 2563 | UGCAGCUG G UGACACAG | 5254 | CTGTGTCA GGCTAGCTACAACGA CAGCTGCA | 6242 |
| 2566 | AGCUGGUG A CACAGCUU | 5255 | AAGCTGTG GGCTAGCTACAACGA CACCAGCT | 6243 |
| 2568 | CUGGUGAC A CAGCUUAU | 5256 | ATAAGCTG GGCTAGCTACAACGA GTCACCAG | 6244 |
| 2571 | GUGACACA G CUUAUGCC | 5257 | GGCATAAG GGCTAGCTACAACGA TGTGTCAC | 6245 |
| 2575 | CACAGCUU A UGCCCUAU | 5258 | ATAGGGCA GGCTAGCTACAACGA AAGCTGTG | 6246 |
| 2577 | CAGCUUAU G CCCUAUGG | 5259 | CCATAGGG GGCTAGCTACAACGA ATAAGCTG | 6247 |
| 2582 | UAUGCCCU A UGGCUGCC | 5260 | GGCAGCCA GGCTAGCTACAACGA AGGGCATA | 6248 |
| 2585 | GCCCUAUG G CUGCCUCU | 5261 | AGAGGCAG GGCTAGCTACAACGA CATAGGGC | 6249 |
| 2588 | CUAUGGCU G CCUCUUAG | 5262 | CTAAGAGG GGCTAGCTACAACGA AGCCATAG | 6250 |
| 2597 | CCUCUUAG A CCAUGUCC | 5263 | GGACATGG GGCTAGCTACAACGA CTAAGAGG | 6251 |
| 2600 | CUUAGACC A UGUCCGGG | 5264 | CCCGGACA GGCTAGCTACAACGA GGTCTAAG | 6252 |
| 2602 | UAGACCAU G UCCGGGAA | 5265 | TTCCCGGA GGCTAGCTACAACGA ATGGTCTA | 6253 |
| 2612 | CCGGGAAA A CCGCGGAC | 5266 | GTCCGCGG GGCTAGCTACAACGA TTTCCCGG | 6254 |
| 2615 | GGAAAACC G CGGACGCC | 5267 | GGCGTCCG GGCTAGCTACAACGA GGTTTTCC | 6255 |
| 2619 | AACCGCGG A CGCCUGGG | 5268 | CCCAGGCG GGCTAGCTACAACGA CCGCGGTT | 6256 |
| 2621 | CCGCGGAC G CCUGGGCU | 5269 | AGCCCAGG GGCTAGCTACAACGA GTCCGCGG | 6257 |
| 2627 | ACGCCUGG G CUCCCAGG | 5270 | CCTGGGAG GGCTAGCTACAACGA CCAGGCGT | 6258 |
| 2636 | CUCCCAGG A CCUGCUGA | 5271 | TCAGCAGG GGCTAGCTACAACGA CCTGGGAG | 6259 |
| 2640 | CAGGACCU G CUGAACUG | 5272 | CAGTTCAG GGCTAGCTACAACGA AGGTCCTG | 6260 |
| 2645 | CCUGCUGA A CUGGUGUA | 5273 | TACACCAG GGCTAGCTACAACGA TCAGCAGG | 6261 |
| 2649 | CUGAACUG G UGUAUGCA | 5274 | TGCATACA GGCTAGCTACAACGA CAGTTCAG | 6262 |
| 2651 | GAACUGGU G UAUGCAGA | 5275 | TCTGCATA GGCTAGCTACAACGA ACCAGTTC | 6263 |
| 2653 | ACUGGUGU A UGCAGAUU | 5276 | AATCTGCA GGCTAGCTACAACGA ACACCAGT | 6264 |
| 2655 | UGGUGUAU G CAGAUUGC | 5277 | GCAATCTG GGCTAGCTACAACGA ATACACCA | 6265 |
| 2659 | GUAUGCAG A UUGCCAAG | 5278 | CTTGGCAA GGCTAGCTACAACGA CTGCATAC | 6266 |
| 2662 | UGCAGAUU G CCAAGGGG | 5279 | CCCCTTGG GGCTAGCTACAACGA AATCTGCA | 6267 |
| 2671 | CCAAGGGG A UGAGCUAC | 5280 | GTAGCTCA GGCTAGCTACAACGA CCCCTTGG | 6268 |
| 2675 | GGGGAUGA G CUACCUGG | 5281 | CCAGGTAG GGCTAGCTACAACGA TCATCCCC | 6269 |
| 2678 | GAUGAGCU A CCUGGAGG | 5282 | CCTCCAGG GGCTAGCTACAACGA AGCTCATC | 6270 |
| 2687 | CCUGGAGG A UGUGCGGC | 5283 | GCCGCACA GGCTAGCTACAACGA CCTCCAGG | 6271 |
| 2689 | UGGAGGAU G UGCGGCUC | 5284 | GAGCCGCA GGCTAGCTACAACGA ATCCTCCA | 6272 |
| 2691 | GAGGAUGU G CGGCUCGU | 5285 | ACGAGCCG GGCTAGCTACAACGA ACATCCTC | 6273 |
| 2694 | GAUGUGCG G CUCGUACA | 5286 | TGTACGAG GGCTAGCTACAACGA CGCACATC | 6274 |
| 2698 | UGCGGCUC G UACACAGG | 5287 | CCTGTGTA GGCTAGCTACAACGA GAGCCGCA | 6275 |
| 2700 | CGGCUCGU A CACAGGGA | 5288 | TCCCTGTG GGCTAGCTACAACGA ACGAGCCG | 6276 |
| 2702 | GCUCGUAC A CAGGGACU | 5289 | AGTCCCTG GGCTAGCTACAACGA GTACGAGC | 6277 |
| 2708 | ACACAGGG A CUUGGCCG | 5290 | CGGCCAAG GGCTAGCTACAACGA CCCTGTGT | 6278 |
| 2713 | GGGACUUG G CCGCUCGG | 5291 | CCGAGCGG GGCTAGCTACAACGA CAAGTCCC | 6279 |
| 2716 | ACUUGGCC G CUCGGAAC | 5292 | GTTCCGAG GGCTAGCTACAACGA GGCCAAGT | 6280 |
| 2723 | CGCUCGGA A CGUGCUGG | 5293 | CCAGCACG GGCTAGCTACAACGA TCCGAGCG | 6281 |
| 2725 | CUCGGAAC G UGCUGGUC | 5294 | GACCAGCA GGCTAGCTACAACGA GTTCCGAG | 6282 |
| 2727 | CGGAACGU G CUGGUCAA | 5295 | TTGACCAG GGCTAGCTACAACGA ACGTTCCG | 6283 |
| 2731 | ACGUGCUG G UCAAGAGU | 5296 | ACTCTTGA GGCTAGCTACAACGA CAGCACGT | 6284 |
| 2738 | GGUCAAGA G UCCCAACC | 5297 | GGTTGGGA GGCTAGCTACAACGA TCTTGACC | 6285 |
| 2744 | GAGUCCCA A CCAUGUCA | 5298 | TGACATGG GGCTAGCTACAACGA TGGGACTC | 6286 |
| 2747 | UCCCAACC A UGUCAAAA | 5299 | TTTTGACA GGCTAGCTACAACGA GGTTGGGA | 6287 |
| 2749 | CCAACCAU G UCAAAAUU | 5300 | AATTTTGA GGCTAGCTACAACGA ATGGTTGG | 6288 |
| 2755 | AUGUCAAA A UUACAGAC | 5301 | GTCTGTAA GGCTAGCTACAACGA TTTGACAT | 6289 |
| 2758 | UCAAAAUU A CAGACUUC | 5302 | GAAGTCTG GGCTAGCTACAACGA AATTTTGA | 6290 |
| 2762 | AAUUACAG A CUUCGGGC | 5303 | GCCCGAAG GGCTAGCTACAACGA CTGTAATT | 6291 |
| 2769 | GACUUCGG G CUGGCUCG | 5304 | CGAGCCAG GGCTAGCTACAACGA CCGAAGTC | 6292 |
| 2773 | UCGGGCUG G CUCGGCUG | 5305 | CAGCCGAG GGCTAGCTACAACGA CAGCCCGA | 6293 |
| 2778 | CUGGCUCG G CUGCUCGA | 5306 | TCCAGCAG GGCTAGCTACAACGA CGAGCCAG | 6294 |
| 2781 | GCUCGGCU G CUGGACAU | 5307 | ATGTCCAG GGCTAGCTACAACGA AGCCGAGC | 6295 |
| 2786 | GCUGCUGG A CAUUGACG | 5308 | CGTCAATG GGCTAGCTACAACGA CCAGCAGC | 6296 |
| 2788 | UGCUGGAC A UUGACGAG | 5309 | CTCGTCAA GGCTAGCTACAACGA GTCCAGCA | 6297 |
| 2792 | GGACAUUG A CGAGACAG | 5310 | CTGTCTCG GGCTAGCTACAACGA CAATGTCC | 6298 |
| 2797 | UUGACGAG A CAGAGUAC | 5311 | GTACTCTG GGCTAGCTACAACGA CTCGTCAA | 6299 |
| 2802 | GAGACAGA G UACCAUGC | 5312 | GCATGGTA GGCTAGCTACAACGA TCTGTCTC | 6300 |
| 2804 | GACAGAGU A CCAUGCAG | 5313 | CTGCATGG GGCTAGCTACAACGA ACTCTGTC | 6301 |
| 2807 | AGAGUACC A UGCAGAUG | 5314 | CATCTGCA GGCTAGCTACAACGA GGTACTCT | 6302 |
| 2809 | AGUACCAU G CAGAUGGG | 5315 | CCCATCTG GGCTAGCTACAACGA ATGGTACT | 6303 |
| 2813 | CCAUGCAG A UGGGGGCA | 5316 | TGCCCCCA GGCTAGCTACAACGA CTGCATGG | 6304 |
| 2819 | AGAUGGGG G CAAGGUGC | 5317 | GCACCTTG GGCTAGCTACAACGA CCCCATCT | 6305 |
| 2824 | GGGGCAAG G UGCCCAUC | 5318 | GATGGGCA GGCTAGCTACAACGA CTTGCCCC | 6306 |
| 2826 | GGCAAGGU G CCCAUCAA | 5319 | TTGATGGG GGCTAGCTACAACGA ACCTTGCC | 6307 |
| 2830 | AGGUGCCC A UCAAGUGG | 5320 | CCACTTGA GGCTAGCTACAACGA GGGCACCT | 6308 |
| 2835 | CCCAUCAA G UGGAUGGC | 5321 | GCCATCCA GGCTAGCTACAACGA TTGATGGG | 6309 |
| 2839 | UCAAGUGG A UGGCGCUG | 5322 | CAGCGCCA GGCTAGCTACAACGA CCACTTGA | 6310 |
| 2842 | AGUGGAUG G CGCUGGAG | 5323 | CTCCAGCG GGCTAGCTACAACGA CATCCACT | 6311 |
| 2844 | UGGAUGGC G CUGGAGUC | 5324 | GACTCCAG GGCTAGCTACAACGA GCCATCCA | 6312 |
| 2850 | GCGCUGGA G UCCAUUCU | 5325 | AGAATGGA GGCTAGCTACAACGA TCCAGCGC | 6313 |
| 2854 | UGGAGUCC A UUCUCCGC | 5326 | GCGGAGAA GGCTAGCTACAACGA GGACTCCA | 6314 |
| 2861 | CAUUCUCC G CCGGCGGU | 5327 | ACCGCCGG GGCTAGCTACAACGA GGAGAATG | 6315 |
| 2865 | CUCCGCCG G CGGUUCAC | 5328 | GTGAACCG GGCTAGCTACAACGA CGGCGGAG | 6316 |
| 2868 | CGCCGGCG G UUCACCCA | 5329 | TGGGTGAA GGCTAGCTACAACGA CGCCGGCG | 6317 |
| 2872 | GGCGGUUC A CCCACCAG | 5330 | CTGGTGGG GGCTAGCTACAACGA GAACCGCC | 6318 |
| 2876 | GUUCACCC A CCAGAGUG | 5331 | CACTCTGG GGCTAGCTACAACGA GGGTGAAC | 6319 |
| 2882 | CCACCAGA G UGAUGUGU | 5332 | ACACATCA GGCTAGCTACAACGA TCTGGTGG | 6320 |
| 2885 | CCAGAGUG A UGUGUGGA | 5333 | TCCACACA GGCTAGCTACAACGA CACTCTGG | 6321 |
| 2887 | AGAGUGAU G UGUGGAGU | 5334 | ACTCCACA GGCTAGCTACAACGA ATCACTCT | 6322 |
| 2889 | AGUGAUGU G UGGAGUUA | 5335 | TAACTCCA GGCTAGCTACAACGA ACATCACT | 6323 |
| 2894 | UGUGUGGA G UUAUGGUG | 5336 | CACCATAA GGCTAGCTACAACGA TCCACACA | 6324 |
| 2897 | GUGGAGUU A UGGUGUGA | 5337 | TCACACCA GGCTAGCTACAACGA AACTCCAC | 6325 |
| 2900 | GAGUUAUG G UGUGACUG | 5338 | CAGTCACA GGCTAGCTACAACGA CATAACTC | 6326 |
| 2902 | GUUAUGGU G UGACUGUG | 5339 | CACAGTCA GGCTAGCTACAACGA ACCATAAC | 6327 |
| 2905 | AUGGUGUG A CUGUGUGG | 5340 | CCACACAG GGCTAGCTACAACGA CACACCAT | 6328 |
| 2908 | GUGUGACU G UGUGGGAG | 5341 | CTCCCACA GGCTAGCTACAACGA AGTCACAC | 6329 |
| 2910 | GUGACUGU G UGGGAGCU | 5342 | AGCTCCCA GGCTAGCTACAACGA ACAGTCAC | 6330 |
| 2916 | GUGUGGGA G CUGAUGAC | 5343 | GTCATCAG GGCTAGCTACAACGA TCCCACAC | 6331 |
| 2920 | GGGAGCUG A UGACUUUU | 5344 | AAAAGTCA GGCTAGCTACAACGA CAGCTCCC | 6332 |
| 2923 | AGCUGAUG A CUUUUGGG | 5345 | CCCAAAAG GGCTAGCTACAACGA CATCAGCT | 6333 |
| 2932 | CUUUUGGG G CCAAACCU | 5346 | AGGTTTGG GGCTAGCTACAACGA CCCAAAAG | 6334 |
| 2937 | GGGGCCAA A CCUUACGA | 5347 | TCGTAAGG GGCTAGCTACAACGA TTGGCCCC | 6335 |
| 2942 | CAAACCUU A CGAUGGGA | 5348 | TCCCATCG GGCTAGCTACAACGA AAGGTTTG | 6336 |
| 2945 | ACCUUACG A UGGGAUCC | 5349 | GGATCCCA GGCTAGCTACAACGA CGTAAGGT | 6337 |
| 2950 | ACGAUGGG A UCCCAGCC | 5350 | GGCTGGGA GGCTAGCTACAACGA CCCATCGT | 6338 |
| 2956 | GGAUCCCA G CCCGGGAG | 5351 | CTCCCGGG GGCTAGCTACAACGA TGGGATCC | 6339 |
| 2965 | CCCGGGAG A UCCCUGAC | 5352 | GTCAGGGA GGCTAGCTACAACGA CTCCCGGG | 6340 |
| 2972 | GAUCCCUG A CCUGCUGG | 5353 | CCAGCAGG GGCTAGCTACAACGA CAGGGATC | 6341 |
| 2976 | CCUGACCU G CUGGAAAA | 5354 | TTTTCCAG GGCTAGCTACAACGA AGGTCAGG | 6342 |
| 2991 | AAGGGGGA G CGGCUGCC | 5355 | GGCAGCCG GGCTAGCTACAACGA TCCCCCTT | 6343 |
| 2994 | GGGGAGCG G CUGCCCCA | 5356 | TGGGGCAG GGCTAGCTACAACGA CGCTCCCC | 6344 |
| 2997 | GAGCGGCU G CCCCAGCC | 5357 | GGCTGGGG GGCTAGCTACAACGA AGCCGCTC | 6345 |
| 3003 | CUGCCCCA G CCCCCCAU | 5358 | ATGGGGGG GGCTAGCTACAACGA TGGGGCAG | 6346 |
| 3010 | AGCCCCCC A UCUGCACC | 5359 | GGTGCAGA GGCTAGCTACAACGA GGGGGGCT | 6347 |
| 3014 | CCCCAUCU G CACCAUUG | 5360 | CAATGGTG GGCTAGCTACAACGA AGATGGGG | 6348 |
| 3016 | CCAUCUGC A CCAUUGAU | 5361 | ATCAATGG GGCTAGCTACAACGA GCAGATGG | 6349 |
| 3019 | UCUGCACC A UUGAUGUC | 5362 | GACATCAA GGCTAGCTACAACGA GGTGCAGA | 6350 |
| 3023 | CACCAUUG A UGUCUACA | 5363 | TGTAGACA GGCTAGCTACAACGA CAATGGTG | 6351 |
| 3025 | CCAUUGAU G UCUACAUG | 5364 | CATGTAGA GGCTAGCTACAACGA ATCAATGG | 6352 |
| 3029 | UGAUGUCU A CAUGAUCA | 5365 | TGATCATG GGCTAGCTACAACGA AGACATCA | 6353 |
| 3031 | AUGUCUAC A UGAUCAUG | 5366 | CATGATCA GGCTAGCTACAACGA GTAGACAT | 6354 |
| 3034 | UCUACAUG A UCAUGGUC | 5367 | GACCATGA GGCTAGCTACAACGA CATGTAGA | 6355 |
| 3037 | ACAUGAUC A UGGUCAAA | 5368 | TTTGACCA GGCTAGCTACAACGA GATCATGT | 6356 |
| 3040 | UGAUCAUG G UCAAAUGU | 5369 | ACATTTGA GGCTAGCTACAACGA CATGATCA | 6357 |
| 3045 | AUGGUCAA A UGUUGGAU | 5370 | ATCCAACA GGCTAGCTACAACGA TTGACCAT | 6358 |
| 3047 | GGUCAAAU G UUGGAUGA | 5371 | TCATCCAA GGCTAGCTACAACGA ATTTGACC | 6359 |
| 3052 | AAUGUUGG A UGAUUGAC | 5372 | GTCAATCA GGCTAGCTACAACGA CCAACATT | 6360 |
| 3055 | GUUGGAUG A UUGACUCU | 5373 | AGAGTCAA GGCTAGCTACAACGA CATCCAAC | 6361 |
| 3059 | GAUGAUUG A CUCUGAAU | 5374 | ATTCAGAG GGCTAGCTACAACGA CAATCATC | 6362 |
| 3066 | GACUCUGA A UGUCGGCC | 5375 | GGCCGACA GGCTAGCTACAACGA TCAGAGTC | 6363 |
| 3068 | CUCUGAAU G UCGGCCAA | 5376 | TTGGCCGA GGCTAGCTACAACGA ATTCAGAG | 6364 |
| 3072 | GAAUGUCG G CCAAGAUU | 5377 | AATCTTGG GGCTAGCTACAACGA CGACATTC | 6365 |
| 3078 | CGGCCAAG A UUCCGGGA | 5378 | TCCCGGAA GGCTAGCTACAACGA CTTGGCCG | 6366 |
| 3087 | UUCCGGGA G UUGGUGUC | 5379 | GACACCAA GGCTAGCTACAACGA TCCCGGAA | 6367 |
| 3091 | GGGAGUUG G UGUCUGAA | 5380 | TTCAGACA GGCTAGCTACAACGA CAACTCCC | 6368 |
| 3093 | GAGUUGGU G UCUGAAUU | 5381 | AATTCAGA GGCTAGCTACAACGA ACCAACTC | 6369 |
| 3099 | GUGUCUGA A UUCUCCCG | 5382 | CGGGAGAA GGCTAGCTACAACGA TCAGACAC | 6370 |
| 3107 | AUUCUCCC G CAUGGCCA | 5383 | TGGCCATG GGCTAGCTACAACGA GGGAGAAT | 6371 |
| 3109 | UCUCCCGC A UGGCCAGG | 5384 | CCTGGCCA GGCTAGCTACAACGA GCGGGAGA | 6372 |
| 3112 | CCCGCAUG G CCAGGGAC | 5385 | GTCCCTGG GGCTAGCTACAACGA CATGCGGG | 6373 |
| 3119 | GGCCAGGG A CCCCCAGC | 5386 | GCTGGGGG GGCTAGCTACAACGA CCCTGGCC | 6374 |
| 3126 | GACCCCCA G CGCUUUGU | 5387 | ACAAAGCG GGCTAGCTACAACGA TGGGGGTC | 6375 |
| 3128 | CCCCCAGC G CUUUGUGG | 5388 | CCACAAAG GGCTAGCTACAACGA GCTGGGGG | 6376 |
| 3133 | AGCGCUUU G UGGUCAUC | 5389 | GATGACCA GGCTAGCTACAACGA AAAGCGCT | 6377 |
| 3136 | GCUUUGUG G UCAUCCAG | 5390 | CTGGATGA GGCTAGCTACAACGA CACAAAGC | 6378 |
| 3139 | UUGUGGUC A UCCAGAAU | 5391 | ATTCTGGA GGCTAGCTACAACGA GACCACAA | 6379 |
| 3146 | CAUCCAGA A UGAGGACU | 5392 | AGTCCTCA GGCTAGCTACAACGA TCTGGATG | 6380 |
| 3152 | GAAUGAGG A CUUGGGCC | 5393 | GGCCCAAG GGCTAGCTACAACGA CCTCATTC | 6381 |
| 3158 | GGACUUGG G CCCAGCCA | 5394 | TGGCTGGG GGCTAGCTACAACGA CCAAGTCC | 6382 |
| 3163 | UGGGCCCA G CCAGUCCC | 5395 | GGGACTGG GGCTAGCTACAACGA TGGGCCCA | 6383 |
| 3167 | CCCAGCCA G UCCCUUGG | 5396 | CCAAGGGA GGCTAGCTACAACGA TGGCTGGG | 6384 |
| 3176 | UCCCUUGG A CAGCACCU | 5397 | AGGTGCTG GGCTAGCTACAACGA CCAAGGGA | 6385 |
| 3179 | CUUGGACA G CACCUUCU | 5398 | AGAAGGTG GGCTAGCTACAACGA TGTCCAAG | 6386 |
| 3181 | UGGACAGC A CCUUCUAC | 5399 | GTAGAAGG GGCTAGCTACAACGA GCTGTCCA | 6387 |
| 3188 | CACCUUCU A CCGCUCAC | 5400 | GTGAGCGG GGCTAGCTACAACGA AGAAGGTG | 6388 |
| 3191 | CUUCUACC G CUCACUGC | 5401 | GCAGTGAG GGCTAGCTACAACGA GGTAGAAG | 6389 |
| 3195 | UACCGCUC A CUGCUGGA | 5402 | TCCAGCAG GGCTAGCTACAACGA GAGCGGTA | 6390 |
| 3198 | CGCUCACU G CUGGAGGA | 5403 | TCCTCCAG GGCTAGCTACAACGA AGTGAGCG | 6391 |
| 3206 | GCUGGAGG A CGAUGACA | 5404 | TGTCATCG GGCTAGCTACAACGA CCTCCAGC | 6392 |
| 3209 | GGAGGACG A UGACAUGG | 5405 | CCATGTCA GGCTAGCTACAACGA CGTCCTCC | 6393 |
| 3212 | GGACGAUG A CAUGGGGG | 5406 | CCCCCATG GGCTAGCTACAACGA CATCGTCC | 6394 |
| 3214 | ACGAUGAC A UGGGGGAC | 5407 | GTCCCCCA GGCTAGCTACAACGA GTCATCGT | 6395 |
| 3221 | CAUGGGGG A CCUGGUGG | 5408 | CCACCAGG GGCTAGCTACAACGA CCCCCATG | 6396 |
| 3226 | GGGACCUG G UGGAUGCU | 5409 | AGCATCCA GGCTAGCTACAACGA CAGGTCCC | 6397 |
| 3230 | CCUGGUGG A UGCUGAGG | 5410 | CCTCAGCA GGCTAGCTACAACGA CCACCAGG | 6398 |
| 3232 | UGGUGGAU G CUGAGGAG | 5411 | CTCCTCAG GGCTAGCTACAACGA ATCCACCA | 6399 |
| 3240 | GCUGAGGA G UAUCUGGU | 5412 | ACCAGATA GGCTAGCTACAACGA TCCTCAGC | 6400 |
| 3242 | UGAGGAGU A UCUGGUAC | 5413 | GTACCAGA GGCTAGCTACAACGA ACTCCTCA | 6401 |
| 3247 | AGUAUCUG G UACCCCAG | 5414 | CTGGGGTA GGCTAGCTACAACGA CAGATACT | 6402 |
| 3249 | UAUCUGGU A CCCCAGCA | 5415 | TGCTGGGG GGCTAGCTACAACGA ACCAGATA | 6403 |
| 3255 | GUACCCCA G CAGGGCUU | 5416 | AAGCCCTG GGCTAGCTACAACGA TGGGGTAC | 6404 |
| 3260 | CCAGCAGG G CUUCUUCU | 5417 | AGAAGAAG GGCTAGCTACAACGA CCTGCTGG | 6405 |
| 3269 | CUUCUUCU G UCCAGACC | 5418 | GGTCTGGA GGCTAGCTACAACGA AGAAGAAG | 6406 |
| 3275 | CUGUCCAG A CCCUGCCC | 5419 | GGGCAGGG GGCTAGCTACAACGA CTGGACAG | 6407 |
| 3280 | CAGACCCU G CCCCGGGC | 5420 | GCCCGGGG GGCTAGCTACAACGA AGGGTCTG | 6408 |
| 3287 | UGCCCCGG G CGCUGGGG | 5421 | CCCCAGCG GGCTAGCTACAACGA CCGGGGCA | 6409 |
| 3289 | CCCCGGGC G CUGGGGGC | 5422 | GCCCCCAG GGCTAGCTACAACGA GCCCGGGG | 6410 |
| 3296 | CGCUGGGG G CAUGGUCC | 5423 | GGACCATG GGCTAGCTACAACGA CCCCAGCG | 6411 |
| 3298 | CUGGGGGC A UGGUCCAC | 5424 | GTGGACCA GGCTAGCTACAACGA GCCCCCAG | 6412 |
| 3301 | GGGGCAUG G UCCACCAC | 5425 | GTGGTGGA GGCTAGCTACAACGA CATGCCCC | 6413 |
| 3305 | CAUGGUCC A CCACAGGC | 5426 | GCCTGTGG GGCTAGCTACAACGA GGACCATG | 6414 |
| 3308 | GGUCCACC A CAGGCACC | 5427 | GGTGCCTG GGCTAGCTACAACGA GGTGGACC | 6415 |
| 3312 | CACCACAG G CACCGCAG | 5428 | CTGCGGTG GGCTAGCTACAACGA CTGTGGTG | 6416 |
| 3314 | CCACAGGC A CCGCAGCU | 5429 | AGCTGCGG GGCTAGCTACAACGA GCCTGTGG | 6417 |
| 3317 | CAGGCACC G CAGCUCAU | 5430 | ATGAGCTG GGCTAGCTACAACGA GGTGCCTG | 6418 |
| 3320 | GCACCGCA G CUCAUCUA | 5431 | TAGATGAG GGCTAGCTACAACGA TGCGGTGC | 6419 |
| 3324 | CGCAGCUC A UCUACCAG | 5432 | CTGGTAGA GGCTAGCTACAACGA GAGCTGCG | 6420 |
| 3328 | GCUCAUCU A CCAGGAGU | 5433 | ACTCCTGG GGCTAGCTACAACGA AGATGAGC | 6421 |
| 3335 | UACCAGGA G UGGCGGUG | 5434 | CACCGCCA GGCTAGCTACAACGA TCCTGGTA | 6422 |
| 3338 | CAGGAGUG G CGGUGGGG | 5435 | CCCCACCG GGCTAGCTACAACGA CACTCCTG | 6423 |
| 3341 | GAGUGGCG G UGGGGACC | 5436 | GGTCCCCA GGCTAGCTACAACGA CGCCACTC | 6424 |
| 3347 | CGGUGGGG A CCUGACAC | 5437 | GTGTCAGG GGCTAGCTACAACGA CCCCACCG | 6425 |
| 3352 | GGGACCUG A CACUAGGG | 5438 | CCCTAGTG GGCTAGCTACAACGA CAGGTCCC | 6426 |
| 3354 | GACCUGAC A CUAGGGCU | 5439 | AGCCCTAG GGCTAGCTACAACGA GTCAGGTC | 6427 |
| 3360 | ACACUAGG G CUGGAGCC | 5440 | GGCTCCAG GGCTAGCTACAACGA CCTAGTGT | 6428 |
| 3366 | GGGCUGGA G CCCUCUGA | 5441 | TCAGAGGG GGCTAGCTACAACGA TCCAGCCC | 6429 |
| 3382 | AAGAGGAG G CCCCCAGG | 5442 | CCTGGGGG GGCTAGCTACAACGA CTCCTCTT | 6430 |
| 3390 | GCCCCCAG G UCUCCACU | 5443 | AGTGGAGA GGCTAGCTACAACGA CTGGGGGC | 6431 |
| 3396 | AGGUCUCC A CUGGCACC | 5444 | GGTGCCAG GGCTAGCTACAACGA GGAGACCT | 6432 |
| 3400 | CUCCACUG G CACCCUCC | 5445 | GGAGGGTG GGCTAGCTACAACGA CAGTGGAG | 6433 |
| 3402 | CCACUGGC A CCCUCCGA | 5446 | TCGGAGGG GGCTAGCTACAACGA GCCAGTGG | 6434 |
| 3415 | CCGAAGGG G CUGGCUCC | 5447 | GGAGCCAG GGCTAGCTACAACGA CCCTTCGG | 6435 |
| 3419 | AGGGGCUG G CUCCGAUG | 5448 | CATCGGAG GGCTAGCTACAACGA CAGCCCCT | 6436 |
| 3425 | UGGCUCCG A UGUAUUUG | 5449 | CAAATACA GGCTAGCTACAACGA CGGAGCCA | 6437 |
| 3427 | GCUCCGAU G UAUUUGAU | 5450 | ATCAAATA GGCTAGCTACAACGA ATCGGAGC | 6438 |
| 3429 | UCCGAUGU A UUUGAUGG | 5451 | CCATCAAA GGCTAGCTACAACGA ACATCGGA | 6439 |
| 3434 | UGUAUUUG A UGGUGACC | 5452 | GGTCACCA GGCTAGCTACAACGA CAAATACA | 6440 |
| 3437 | AUUUGAUG G UGACCUGG | 5453 | CCAGGTCA GGCTAGCTACAACGA CATCAAAT | 6441 |
| 3440 | UGAUGGUG A CCUGGGAA | 5454 | TTCCCAGG GGCTAGCTACAACGA CACCATCA | 6442 |
| 3448 | ACCUGGGA A UGGGGGCA | 5455 | TGCCCCCA GGCTAGCTACAACGA TCCCAGGT | 6443 |
| 3454 | GAAUGGGG G CAGCCAAG | 5456 | CTTGGCTG GGCTAGCTACAACGA CCCCATTC | 6444 |
| 3457 | UGGGGGCA G CCAAGGGG | 5457 | CCCCTTGG GGCTAGCTACAACGA TGCCCCCA | 6445 |
| 3465 | GCCAAGGG G CUGCAAAG | 5458 | CTTTGCAG GGCTAGCTACAACGA CCCTTGGC | 6446 |
| 3468 | AAGGGGCU G CAAAGCCU | 5459 | AGGCTTTG GGCTAGCTACAACGA AGCCCCTT | 6447 |
| 3473 | GCUGCAAA G CCUCCCCA | 5460 | TGGGGAGG GGCTAGCTACAACGA TTTGCAGC | 6448 |
| 3481 | GCCUCCCC A CACAUGAC | 5461 | GTCATGTG GGCTAGCTACAACGA GGGGAGGC | 6449 |
| 3483 | CUCCCCAC A CAUGACCC | 5462 | GGGTCATG GGCTAGCTACAACGA GTGGGGAG | 6450 |
| 3485 | CCCCACAC A UGACCCCA | 5463 | TGGGGTCA GGCTAGCTACAACGA GTGTGGGG | 6451 |
| 3488 | CACACAUG A CCCCAGCC | 5464 | GGCTGGGG GGCTAGCTACAACGA CATGTGTG | 6452 |
| 3494 | UGACCCCA G CCCUCUAC | 5465 | GTAGAGGG GGCTAGCTACAACGA TGGGGTCA | 6453 |
| 3501 | AGCCCUCU A CAGCGGUA | 5466 | TACCGCTG GGCTAGCTACAACGA AGAGGGCT | 6454 |
| 3504 | CCUCUACA G CGGUACAG | 5467 | CTGTACCG GGCTAGCTACAACGA TGTAGAGG | 6455 |
| 3507 | CUACAGCG G UACAGUGA | 5468 | TCACTGTA GGCTAGCTACAACGA CGCTGTAG | 6456 |
| 3509 | ACAGCGGU A CACUGAGG | 5469 | CCTCACTG GGCTAGCTACAACGA ACCGCTGT | 6457 |
| 3512 | GCGGUACA G UGAGGACC | 5470 | GGTCCTCA GGCTAGCTACAACGA TGTACCGC | 6458 |
| 3518 | CAGUGAGG A CCCCACAG | 5471 | CTGTGGGG GGCTAGCTACAACGA CCTCACTG | 6459 |
| 3523 | AGGACCCC A CAGUACCC | 5472 | GGGTACTG GGCTAGCTACAACGA GGGGTCCT | 6460 |
| 3526 | ACCCCACA G UACCCCUG | 5473 | CAGGGGTA GGCTAGCTACAACGA TGTGGGGT | 6461 |
| 3528 | CCCACAGU A CCCCUGCC | 5474 | GGCAGGGG GGCTAGCTACAACGA ACTGTGGG | 6462 |
| 3534 | GUACCCCU G CCCUCUGA | 5475 | TCAGAGGG GGCTAGCTACAACGA AGGGGTAC | 6463 |
| 3544 | CCUCUGAG A CUCAUGGC | 5476 | GCCATCAG GGCTAGCTACAACGA CTCAGAGG | 6464 |
| 3548 | UGAGACUG A UGGCUACG | 5477 | CGTAGCCA GGCTAGCTACAACGA CAGTCTCA | 6465 |
| 3551 | GACUCAUG G CUACGUUG | 5478 | CAACGTAG GGCTAGCTACAACGA CATCAGTC | 6466 |
| 3554 | UGAUGGCU A CGUUGCCC | 5479 | GGGCAACG GGCTAGCTACAACGA AGCCATCA | 6467 |
| 3556 | AUGGCUAC G UUGCCCCC | 5480 | GGGGGCAA GGCTAGCTACAACGA GTAGCCAT | 6468 |
| 3559 | GCUACGUU G CCCCCCUG | 5481 | CAGGGGGG GGCTAGCTACAACGA AACGTAGC | 6469 |
| 3568 | CCCCCCUG A CCUGCAGC | 5482 | GCTGCAGG GGCTAGCTACAACGA CAGGGGGG | 6470 |
| 3572 | CCUGACCU G CAGCCCCC | 5483 | GGGGGCTG GGCTAGCTACAACGA AGGTCAGG | 6471 |
| 3575 | GACCUGCA G CCCCCAGC | 5484 | GCTGGGGG GGCTAGCTACAACGA TGCAGGTC | 6472 |
| 3582 | AGCCCCCA G CCUGAAUA | 5485 | TATTCAGG GGCTAGCTACAACGA TGGGGGCT | 6473 |
| 3588 | CAGCCUGA A UAUGUCAA | 5486 | TTCACATA GGCTAGCTACAACGA TCAGGCTG | 6474 |
| 3590 | GCCUGAAU A UGUGAACC | 5487 | GGTTCACA GGCTAGCTACAACGA ATTCAGGC | 6475 |
| 3592 | CUGAAUAU G UGAACCAG | 5488 | CTGGTTCA GGCTAGCTACAACGA ATATTCAG | 6476 |
| 3596 | AUAUGUGA A CCAGCCAG | 5489 | CTGGCTGG GGCTAGCTACAACGA TCACATAT | 6477 |
| 3600 | GUGAACCA G CCAGAUGU | 5490 | ACATCTGG GGCTAGCTACAACGA TGGTTCAC | 6478 |
| 3605 | CCAGCCAG A UGUUCGGC | 5491 | GCCGAACA GGCTAGCTACAACGA CTGGCTGG | 6479 |
| 3607 | AGCCAGAU G UUCGGCCC | 5492 | GGGCCGAA GGCTAGCTACAACGA ATCTGGCT | 6480 |
| 3612 | GAUGUUCG G CCCCAGCC | 5493 | GGCTGGGG GGCTAGCTACAACGA CGAACATC | 6481 |
| 3618 | CGGCCCCA G CCCCCUUC | 5494 | GAAGGGGG GGCTAGCTACAACGA TGGGGCCG | 6482 |
| 3627 | CCCCCUUC G CCCCGAGA | 5495 | TCTCGGGG GGCTAGCTACAACGA GAAGGGGG | 6483 |
| 3638 | CCGAGAGG G CCCUCUGC | 5496 | GCAGAGGG GGCTAGCTACAACGA CCTCTCGG | 6484 |
| 3645 | GGCCCUCU G CCUGCUGC | 5497 | GCAGCAGG GGCTAGCTACAACGA AGAGGGCC | 6485 |
| 3649 | CUCUGCCU G CUGCCCGA | 5498 | TCGGGCAG GGCTAGCTACAACGA AGGCAGAG | 6486 |
| 3652 | UGCCUGCU G CCCGACCU | 5499 | AGGTCGGG GGCTAGCTACAACGA AGCAGGCA | 6487 |
| 3657 | GCUGCCCG A CCUGCUGG | 5500 | CCAGCAGG GGCTAGCTACAACGA CGGGCAGC | 6488 |
| 3661 | CCCGACCU G CUGGUGCC | 5501 | GGCACCAG GGCTAGCTACAACGA AGGTCGGG | 6489 |
| 3665 | ACCUGCUG G UGCCACUC | 5502 | GAGTGGCA GGCTAGCTACAACGA CAGCAGGT | 6490 |
| 3667 | CUGCUGGU G CCACUCUG | 5503 | CAGAGTGG GGCTAGCTACAACGA ACCAGCAG | 6491 |
| 3670 | CUGGUGCC A CUCUGGAA | 5504 | TTCCAGAG GGCTAGCTACAACGA GGCACCAG | 6492 |
| 3681 | CUGGAAAG G CCCAAGAC | 5505 | GTCTTGGG GGCTAGCTACAACGA CTTTCCAG | 6493 |
| 3688 | GGCCCAAG A CUCUCUCC | 5506 | GGAGAGAG GGCTAGCTACAACGA CTTGGGCC | 6494 |
| 3707 | AGGGAAGA A UGGGGUCG | 5507 | CGACCCCA GGCTAGCTACAACGA TCTTCCCT | 6495 |
| 3712 | AGAAUGGG G UCGUCAAA | 5508 | TTTGACGA GGCTAGCTACAACGA CCCATTCT | 6496 |
| 3715 | AUGGGGUC G UCAAAGAC | 5509 | GTCTTTGA GGCTAGCTACAACGA GACCCCAT | 6497 |
| 3722 | CGUCAAAG A CGUUUUUG | 5510 | CAAAAACG GGCTAGCTACAACGA CTTTGACG | 6498 |
| 3724 | UCAAAGAC G UUUUUGCC | 5511 | GGCAAAAA GGCTAGCTACAACGA GTCTTTGA | 6499 |
| 3730 | ACGUUUUU G CCUUUGGG | 5512 | CCCAAAGG GGCTAGCTACAACGA AAAAACGT | 6500 |
| 3740 | CUUUGGGG G UGCCGUGG | 5513 | CCACGGCA GGCTAGCTACAACGA CCCCAAAG | 6501 |
| 3742 | UUGGGGGU G CCGUGGAG | 5514 | CTCCACGG GGCTAGCTACAACGA ACCCCCAA | 6502 |
| 3745 | GGGGUGCC G UGGAGAAC | 5515 | GTTCTCCA GGCTAGCTACAACGA GGCACCCC | 6503 |
| 3752 | CGUGGAGA A CCCCGAGU | 5516 | ACTCGGGG GGCTAGCTACAACGA TCTCCACG | 6504 |
| 3759 | AACCCCGA G UACUUGAC | 5517 | GTCAAGTA GGCTAGCTACAACGA TCGGGGTT | 6505 |
| 3761 | CCCCGAGU A CUUGACAC | 5518 | GTGTCAAG GGCTAGCTACAACGA ACTCGGGG | 6506 |
| 3766 | AGUACUUG A CACCCCAG | 5519 | CTGGGGTG GGCTAGCTACAACGA CAAGTACT | 6507 |
| 3768 | UACUUGAC A CCCCAGGG | 5520 | CCCTGGGG GGCTAGCTACAACGA GTCAAGTA | 6508 |
| 3781 | AGGGAGGA G CUGCCCCU | 5521 | AGGGGCAG GGCTAGCTACAACGA TCCTCCCT | 6509 |
| 3784 | GAGGAGCU G CCCCUCAG | 5522 | CTGAGGGG GGCTAGCTACAACGA AGCTCCTC | 6510 |
| 3792 | GCCCCUCA G CCCCACCC | 5523 | GGGTGGGG GGCTAGCTACAACGA TGAGGGGC | 6511 |
| 3797 | UCAGCCCC A CCCUCCUC | 5524 | GAGGAGGG GGCTAGCTACAACGA GGGGCTGA | 6512 |
| 3808 | CUCCUCCU G CCUUCAGC | 5525 | GCTGAAGG GGCTAGCTACAACGA AGGAGGAG | 6513 |
| 3815 | UGCCUUCA G CCCAGCCU | 5526 | AGGCTGGG GGCTAGCTACAACGA TGAAGGCA | 6514 |
| 3820 | UCAGCCCA G CCUUCGAC | 5527 | GTCGAAGG GGCTAGCTACAACGA TGGGCTGA | 6515 |
| 3827 | AGCCUUCG A CAACCUCU | 5528 | AGAGGTTG GGCTAGCTACAACGA CGAAGGCT | 6516 |
| 3830 | CUUCGACA A CCUCUAUU | 5529 | AATAGAGG GGCTAGCTACAACGA TGTCGAAG | 6517 |
| 3836 | CAACCUCU A UUACUGGG | 5530 | CCCAGTAA GGCTAGCTACAACGA AGAGGTTG | 6518 |
| 3839 | CCUCUAUU A CUGGGACC | 5531 | GGTCCCAG GGCTAGCTACAACGA AATAGAGG | 6519 |
| 3845 | UUACUGGG A CCAGGACC | 5532 | GGTCCTGG GGCTAGCTACAACGA CCCAGTAA | 6520 |
| 3851 | GGACCAGG A CCCACCAG | 5533 | CTGGTGGG GGCTAGCTACAACGA CCTGGTCC | 6521 |
| 3855 | CAGGACCC A CCAGAGCG | 5534 | CGCTCTGG GGCTAGCTACAACGA GGGTCCTG | 6522 |
| 3861 | CCACCAGA G CGGGGGGC | 5535 | GCCCCCCG GGCTAGCTACAACGA TCTGGTGG | 6523 |
| 3868 | AGCGGGGG G CUCCACCC | 5536 | GGGTGGAG GGCTAGCTACAACGA CCCCCGCT | 6524 |
| 3873 | GGGGCUCC A CCCAGCAC | 5537 | GTGCTGGG GGCTAGCTACAACGA GGAGCCCC | 6525 |
| 3878 | UCCACCCA G CACCUUCA | 5538 | TGAAGGTG GGCTAGCTACAACGA TGGGTGGA | 6526 |
| 3880 | CACCCAGC A CCUUCAAA | 5539 | TTTGAAGG GGCTAGCTACAACGA GCTGGGTG | 6527 |
| 3892 | UCAAAGGG A CACCUACG | 5540 | CGTAGGTG GGCTAGCTACAACGA CCCTTTGA | 6528 |
| 3894 | AAAGGGAC A CCUACGGC | 5541 | GCCGTAGG GGCTAGCTACAACGA GTCCCTTT | 6529 |
| 3898 | GGACACCU A CGGCAGAG | 5542 | CTCTGCCG GGCTAGCTACAACGA AGGTGTCC | 6530 |
| 3901 | CACCUACG G CAGAGAAC | 5543 | GTTCTCTG GGCTAGCTACAACGA CGTAGGTG | 6531 |
| 3908 | GGCAGAGA A CCCAGAGU | 5544 | ACTCTGGG GGCTAGCTACAACGA TCTCTGCC | 6532 |
| 3915 | AACCCAGA G UACCUGGG | 5545 | CCCAGGTA GGCTAGCTACAACGA TCTGGGTT | 6533 |
| 3917 | CCCAGAGU A CCUGGGUC | 5546 | GACCCAGG GGCTAGCTACAACGA ACTCTGGG | 6534 |
| 3923 | GUACCUGG G UCUGGACG | 5547 | CGTCCAGA GGCTAGCTACAACGA CCAGGTAC | 6535 |
| 3929 | GGGUCUGG A CGUGCCAG | 5548 | CTGGCACG GGCTAGCTACAACGA CCAGACCC | 6536 |
| 3931 | GUCUGGAC G UGCCAGUG | 5549 | CACTGGCA GGCTAGCTACAACGA GTCCAGAC | 6537 |
| 3933 | CUGGACGU G CCAGUGUG | 5550 | CACACTGG GGCTAGCTACAACGA ACGTCCAG | 6538 |
| 3937 | ACGUGCCA G UGUGAACC | 5551 | GGTTCACA GGCTAGCTACAACGA TGGCACGT | 6539 |
| 3939 | GUGCCAGU G UGAACCAG | 5552 | CTGGTTCA GGCTAGCTACAACGA ACTGGCAC | 6540 |
| 3943 | CAGUGUGA A CCAGAAGG | 5553 | CCTTCTGG GGCTAGCTACAACGA TCACACTG | 6541 |
| 3951 | ACCAGAAG G CCAAGUCC | 5554 | GGACTTGG GGCTAGCTACAACGA CTTCTGGT | 6542 |
| 3956 | AAGGCCAA G UCCGCAGA | 5555 | TCTGCGGA GGCTAGCTACAACGA TTGGCCTT | 6543 |
| 3960 | CCAAGUCC G CAGAAGCC | 5556 | GGCTTCTG GGCTAGCTACAACGA GGACTTGG | 6544 |
| 3966 | CCGCAGAA G CCCUGAUG | 5557 | CATCAGGG GGCTAGCTACAACGA TTCTGCGG | 6545 |
| 3972 | AAGCCCUG A UGUGUCCU | 5558 | AGGACACA GGCTAGCTACAACGA CAGGGCTT | 6546 |
| 3974 | GCCCUGAU G UGUCCUCA | 5559 | TGAGGACA GGCTAGCTACAACGA ATCAGGGC | 6547 |
| 3976 | CCUGAUGU G UCCUCAGG | 5560 | CCTGAGGA GGCTAGCTACAACGA ACATCAGG | 6548 |
| 3987 | CUCAGGGA G CAGGGAAG | 5561 | CTTCCCTG GGCTAGCTACAACGA TCCCTGAG | 6549 |
| 3996 | CAGGGAAG G CCUGACUU | 5562 | AAGTCAGG GGCTAGCTACAACGA CTTCCCTG | 6550 |
| 4001 | AAGGCCUG A CUUCUGCU | 5563 | AGCAGAAG GGCTAGCTACAACGA CAGGCCTT | 6551 |
| 4007 | UGACUUCU G CUGGCAUC | 5564 | GATGCCAG GGCTAGCTACAACGA AGAAGTCA | 6552 |
| 4011 | UUCUGCUG G CAUCAAGA | 5565 | TCTTGATG GGCTAGCTACAACGA CAGCAGAA | 6553 |
| 4013 | CUGCUGGC A UCAAGAGG | 5566 | CCTCTTGA GGCTAGCTACAACGA GCCAGCAG | 6554 |
| 4021 | AUCAAGAG G UGGGAGGG | 5567 | CCCTCCCA GGCTAGCTACAACGA CTCTTGAT | 6555 |
| 4029 | GUGGGAGG G CCCUCCGA | 5568 | TCGGAGGG GGCTAGCTACAACGA CCTCCCAC | 6556 |
| 4037 | GCCCUCCG A CCACUUCC | 5569 | GGAAGTGG GGCTAGCTACAACGA CGGAGGGC | 6557 |
| 4040 | CUCCGACC A CUUCCAGG | 5570 | CCTGGAAG GGCTAGCTACAACGA GGTCGGAG | 6558 |
| 4052 | CCAGGGGA A CCUGCCAU | 5571 | ATGGCAGG GGCTAGCTACAACGA TCCCCTGG | 6559 |
| 4056 | GGGAACCU G CCAUGCCA | 5572 | TGGCATGG GGCTAGCTACAACGA AGGTTCCC | 6560 |
| 4059 | AACCUGCC A UGCCAGGA | 5573 | TCCTGGCA GGCTAGCTACAACGA GGCAGGTT | 6561 |
| 4061 | CCUGCCAU G CCAGGAAC | 5574 | GTTCCTGG GGCTAGCTACAACGA ATGGCAGG | 6562 |
| 4068 | UGCCAGGA A CCUGUCCU | 5575 | AGGACAGG GGCTAGCTACAACGA TCCTGGCA | 6563 |
| 4072 | AGGAACCU G UCCUAAGG | 5576 | CCTTAGGA GGCTAGCTACAACGA AGGTTCCT | 6564 |
| 4082 | CCUAAGGA A CCUUCCUU | 5577 | AAGGAAGG GGCTAGCTACAACGA TCCTTAGG | 6565 |
| 4094 | UCCUUCCU G CUUGAGUU | 5578 | AACTCAAG GGCTAGCTACAACGA AGGAAGGA | 6566 |
| 4100 | CUGCUUGA G UUCCCAGA | 5579 | TCTGGGAA GGCTAGCTACAACGA TCAAGCAG | 6567 |
| 4108 | GUUCCCAG A UGGCUGGA | 5580 | TCCAGCCA GGCTAGCTACAACGA CTGGGAAC | 6568 |
| 4111 | CCCAGAUG G CUGGAAGG | 5581 | CCTTCCAG GGCTAGCTACAACGA CATCTGGG | 6569 |
| 4121 | UGGAAGGG G UCCAGCCU | 5582 | AGGCTGGA GGCTAGCTACAACGA CCCTTCCA | 6570 |
| 4126 | GGGGUCCA G CCUCGUUG | 5583 | CAACGAGG GGCTAGCTACAACGA TGGACCCC | 6571 |
| 4131 | CCAGCCUC G UUGGAAGA | 5584 | TCTTCCAA GGCTAGCTACAACGA GAGGCTGG | 6572 |
| 4143 | GAAGAGGA A CAGCACUG | 5585 | CAGTGCTG GGCTAGCTACAACGA TCCTCTTC | 6573 |
| 4146 | GAGGAACA G CACUGGGG | 5586 | CCCCAGTG GGCTAGCTACAACGA TGTTCCTC | 6574 |
| 4148 | GGAACAGC A CUGGGGAG | 5587 | CTCCCCAG GGCTAGCTACAACGA GCTGTTCC | 6575 |
| 4156 | ACUGGGGA G UCUUUGUG | 5588 | CACAAAGA GGCTAGCTACAACGA TCCCCAGT | 6576 |
| 4162 | GAGUCUUU G UGGAUUCU | 5589 | AGAATCCA GGCTAGCTACAACGA AAAGACTC | 6577 |
| 4166 | CUUUGUGG A UUCUGAGG | 5590 | CCTCAGAA GGCTAGCTACAACGA CCACAAAG | 6578 |
| 4174 | AUUCUGAG G CCCUGCCC | 5591 | GGGCAGGG GGCTAGCTACAACGA CTCAGAAT | 6579 |
| 4179 | GAGGCCCU G CCCAAUGA | 5592 | TCATTGGG GGCTAGCTACAACGA AGGGCCTC | 6580 |
| 4184 | CCUGCCCA A UGAGACUC | 5593 | GAGTCTCA GGCTAGCTACAACGA TGGGCAGG | 6581 |
| 4189 | CCAAUGAG A CUCUAGGG | 5594 | CCCTAGAG GGCTAGCTACAACGA CTCATTGG | 6582 |
| 4197 | ACUCUAGG G UCCAGUGG | 5595 | CCACTGGA GGCTAGCTACAACGA CCTAGAGT | 6583 |
| 4202 | AGGGUCCA G UGGAUGCC | 5596 | GGCATCCA GGCTAGCTACAACGA TGGACCCT | 6584 |
| 4206 | UCCAGUGG A UGCCACAG | 5597 | CTGTGGCA GGCTAGCTACAACGA CCACTGGA | 6585 |
| 4208 | CAGUGGAU G CCACAGCC | 5598 | GGCTGTGG GGCTAGCTACAACGA ATCCACTG | 6586 |
| 4211 | UGGAUGCC A CAGCCCAG | 5599 | CTGGGCTG GGCTAGCTACAACGA GGCATCCA | 6587 |
| 4214 | AUGCCACA G CCCAGCUU | 5600 | AAGCTGGG GGCTAGCTACAACGA TGTGGCAT | 6588 |
| 4219 | ACAGCCCA G CUUGGCCC | 5601 | GGGCCAAG GGCTAGCTACAACGA TGGGCTGT | 6589 |
| 4224 | CCAGCUUG G CCCUUUCC | 5602 | GGAAAGGG GGCTAGCTACAACGA CAAGCTGG | 6590 |
| 4239 | CCUUCCAG A UCCUGGGU | 5603 | ACCCAGGA GGCTAGCTACAACGA CTGGAAGG | 6591 |
| 4246 | GAUCCUGG G UACUGAAA | 5604 | TTTCAGTA GGCTAGCTACAACGA CCAGGATC | 6592 |
| 4248 | UCCUGGGU A CUGAAAGC | 5605 | GCTTTCAG GGCTAGCTACAACGA ACCCAGGA | 6593 |
| 4255 | UACUGAAA G CCUUAGGG | 5606 | CCCTAAGG GGCTAGCTACAACGA TTTCAGTA | 6594 |
| 4266 | UUAGGGAA G CUGGCCUG | 5607 | CAGGCCAG GGCTAGCTACAACGA TTCCCTAA | 6595 |
| 4270 | GGAAGCUC G CCUGAGAG | 5608 | CTCTCAGG GGCTAGCTACAACGA CAGCTTCC | 6596 |
| 4284 | GAGGGGAA G CGGCCCUA | 5609 | TAGGGCCG GGCTAGCTACAACGA TTCCCCTC | 6597 |
| 4287 | GGGAAGCG G CCCUAAGG | 5610 | CCTTAGGG GGCTAGCTACAACGA CGCTTCCC | 6598 |
| 4298 | CUAAGGGA G UGUCUAAG | 5611 | CTTAGACA GGCTAGCTACAACGA TCCCTTAG | 6599 |
| 4300 | AAGGGAGU G UCUAAGAA | 5612 | TTCTTAGA GGCTAGCTACAACGA ACTCCCTT | 6600 |
| 4308 | GUCUAAGA A CAAAAGCG | 5613 | CGCTTTTG GGCTAGCTACAACGA TCTTAGAC | 6601 |
| 4314 | GAACAAAA G CGACCCAU | 5614 | ATGGGTCG GGCTAGCTACAACGA TTTTGTTC | 6602 |
| 4317 | CAAAAGCG A CCCAUUCA | 5615 | TGAATGGG GGCTAGCTACAACGA CGCTTTTG | 6603 |
| 4321 | AGCGACCC A UUCAGAGA | 5616 | TCTCTGAA GGCTAGCTACAACGA GGGTCGCT | 6604 |
| 4329 | AUUCAGAG A CUGUCCCU | 5617 | AGGGACAG GGCTAGCTACAACGA CTCTGAAT | 6605 |
| 4332 | CAGAGACU G UCCCUGAA | 5618 | TTCAGGGA GGCTAGCTACAACGA AGTCTCTG | 6606 |
| 4341 | UCCCUGAA A CCUAGUAC | 5619 | GTACTAGG GGCTAGCTACAACGA TTCAGGGA | 6607 |
| 4346 | GAAACCUA G UACUGCCC | 5620 | GGGCAGTA GGCTAGCTACAACGA TAGGTTTC | 6608 |
| 4348 | AACCUAGU A CUGCCCCC | 5621 | GGGGGCAG GGCTAGCTACAACGA ACTAGGTT | 6609 |
| 4351 | CUAGUACU G CCCCCCAU | 5622 | ATGGGGGG GGCTAGCTACAACGA AGTACTAG | 6610 |
| 4358 | UCCCCCCC A UGAGGAAG | 5623 | CTTCCTCA GGCTAGCTACAACGA GGGGGGCA | 6611 |
| 4369 | AGGAAGGA A CAGCAAUG | 5624 | CATTGCTG GGCTAGCTACAACGA TCCTTCCT | 6612 |
| 4372 | AAGGAACA G CAAUGGUG | 5625 | CACCATTG GGCTAGCTACAACGA TGTTCCTT | 6613 |
| 4375 | GAACAGCA A UGGUGUCA | 5626 | TGACACCA GGCTAGCTACAACGA TGCTGTTC | 6614 |
| 4378 | CAGCAAUG G UGUCAGUA | 5627 | TACTGACA GGCTAGCTACAACGA CATTGCTG | 6615 |
| 4380 | GCAAUGGU G UCAGUAUC | 5628 | GATACTGA GGCTAGCTACAACGA ACCATTGC | 6616 |
| 4384 | UGGUGUCA G UAUCCAGG | 5629 | CCTGGATA GGCTAGCTACAACGA TGACACCA | 6617 |
| 4386 | GUGUCAGU A UCCAGGCU | 5630 | AGCCTGGA GGCTAGCTACAACGA ACTGACAC | 6618 |
| 4392 | GUAUCCAG G CUUUGUAC | 5631 | GTACAAAG GGCTAGCTACAACGA CTGGATAC | 6619 |
| 4397 | CAGGCUUU G UACAGAGU | 5632 | ACTCTGTA GGCTAGCTACAACGA AAAGCCTG | 6620 |
| 4399 | GGCUUUGU A CAGAGUGC | 5633 | GCACTCTG GGCTAGCTACAACGA ACAAAGCC | 6621 |
| 4404 | UGUACAGA G UGCUUUUC | 5634 | GAAAAGCA GGCTAGCTACAACGA TCTGTACA | 6622 |
| 4406 | UACAGAGU G CUUUUCUG | 5635 | CAGAAAAG GGCTAGCTACAACGA ACTCTGTA | 6623 |
| 4414 | GCUUUUCU G UUUAGUUU | 5636 | AAACTAAA GGCTAGCTACAACGA AGAAAAGC | 6624 |
| 4419 | UCUGUUUA G UUUUUACU | 5637 | AGTAAAAA GGCTAGCTACAACGA TAAACAGA | 6625 |
| 4425 | UAGUUUUU A CUUUUUUU | 5638 | AAAAAAAG GGCTAGCTACAACGA AAAAACTA | 6626 |
| 4434 | CUUUUUUU G UUUUGUUU | 5639 | AAACAAAA GGCTAGCTACAACGA AAAAAAAG | 6627 |
| 4439 | UUUGUUUU G UUUUUUUA | 5640 | TAAAAAAA GGCTAGCTACAACGA AAAACAAA | 6628 |
| 4451 | UUUUAAAG A UGAAAUAA | 5641 | TTATTTCA GGCTAGCTACAACGA CTTTAAAA | 6629 |
| 4456 | AAGAUGAA A UAAAGACC | 5642 | GGTCTTTA GGCTAGCTACAACGA TTCATCTT | 6630 |
| 4462 | AAAUAAAG A CCCAGGGG | 5643 | CCCCTGGG GGCTAGCTACAACGA CTTTATTT | 6631 |

Input Sequence = HSERB2R.
Cut Site = R/Y
Arm Length = 8.
Core Sequence = GGCTAGCTACAACGA
HSERB2R (Human c-erb-B-2 mRNA; 4473 bp)

TABLE V
Human HER2 Synthetic DNAzyme and Target molecules

| | | | | | | |
|---|---|---|---|---|---|---|
| | | | Seq | | | |
| Gene | Pos | Target | ID | RPI# | DNAzyme | Seq ID |

| | | | | | | |
|---|---|---|---|---|---|---|
| erbB2 | 377 | CCACCA A UGCCAG | 6632 | 24998 | cuggca GGCTAGCTACAACGA uggugg B | 6637 |
| erbB2 | 766 | UUCUCCG A UGUGUAA | 6633 | 24999 | uuacaca GGCTAGCTACAACGA cggagaa B | 6638 |
| erbB2 | 1202 | UGUGCU A UGGUCU | 6634 | 25000 | agacca GGCTAGCTACAACGA agcaca B | 6639 |
| erbB2 | 1444 | CCUCAGC G UCUUCCA | 6635 | 25001 | uggaaga GGCTAGCTACAACGA gcugagg B | 6640 |
| erbB2 | 1583 | AUCCACC A UAACACC | 6636 | 25002 | gguguua GGCTAGCTACAACGA gguggau B | 6641 |

A, G, C, T (italic) = deoxy
lower case = 2′-O-methyl
B = inverted deoxyabasic derivative

TABLE VI
Human HIV Hammerhead Ribozyme and Substrate Sequence

| | Seq | | Seq |
|---|---|---|---|
| Substrate | ID | Hammerhead | ID |
| AUAAAGCU U GCCUUGAG | 6642 | CUCAAGGC CUGAUGAGGCCGUUAGGCCGAA AGCUUUAU | 6727 |
| AGGCUAAU U UUUUAGGG | 6643 | CCCUAAAA CUGAUGAGGCCGUUAGGCCGAA AUUAGCCU | 6728 |
| GGCUAAUU U UUUAGGGA | 6644 | UCCCUAAA CUGAUGAGGCCGUUAGGCCGAA AAUUAGCC | 6729 |
| GCCUCAAU A AAGCUUGC | 6645 | GCAAGCUU CUGAUGAGGCCGUUAGGCCGAA AUUGAGGC | 6730 |
| UUUCGGGU U UAUUACAG | 6646 | CUGUAAUA CUGAUGAGGCCGUUAGGCCGAA ACCCGAAA | 6731 |
| GCAGGACU C GGCUUGCU | 6647 | AGCAAGCC CUGAUGAGGCCGUUAGGCCGAA AGUCCUGC | 6732 |

Input Sequence = HIV1.
Cut Site = UH/.
Arm Length = 8.
Core Sequence = CUGAUGAG GCCGUUAGGC CGAA
HIV1 Consensus
Underlined region can be any X sequence or linker, as described herein.

TABLE VII
Human HIV Inozyme and Substrate Sequence

| | Seq | | Seq |
|---|---|---|---|
| Substrate | ID | Inozyme | ID |
| UGGAAAAC A GAUGGCAG | 6648 | CUGCCAUC CUGAUGAGGCCGUUAGGCCGAA IUUUUCCA | 6733 |
| AAUAAAGC U UGCCUUGA | 6649 | UCAAGGCA CUGAUGAGGCCGUUAGGCCGAA ICUUUAUU | 6734 |
| UCUCUAGC A GUGGCGCC | 6650 | GGCGCCAC CUGAUGAGGCCGUUAGGCCGAA ICUAGAGA | 6735 |
| GGAGCCAC C CCACAAGA | 6651 | UCUUGUGG CUGAUGAGGCCGUUAGGCCGAA IUGGCUCC | 6736 |
| AGUGGCGC C CGAACAGG | 6652 | CCUGUUCG CUGAUGAGGCCGUUAGGCCGAA ICGCCACU | 6737 |
| GUGGCGCC C GAACAGGG | 6653 | CCCUGUUC CUGAUGAGGCCGUUAGGCCGAA IGCGCCAC | 6738 |
| CUCGACGC A GGACUCGG | 6654 | CCGAGUCC CUGAUGAGGCCGUUAGGCCGAA ICGUCGAG | 6739 |
| CGCAGGAC U CGGCUUGC | 6655 | GCAAGCCG CUGAUGAGGCCGUUAGGCCGAA IUCCUGCG | 6740 |

Input Sequence = HIV1.
Cut Site = CH/.
Arm Length = 8.
Core Sequence = CUGAUGAG GCCGUUAGGC CGAA
HIV1 Consensus
Underlined region can be any X sequence or linker, as described herein.
“I” stands for Inosine.

TABLE VIII
Human HIV Zinzyme and Substrate Sequence

| | Seq | | Seq |
|---|---|---|---|
| Substrate | ID | Zinzyme | ID |
| UCAAUAAA G CUUGCCUU | 6656 | AAGGCAAG GCCGAAAGGCGAGUGAGGUCU UUUAUUGA | 6741 |
| AGGACUCG G CUUGCUGA | 6657 | UCAGCAAG GCCGAAAGGCGAGUGAGGUCU CGAGUCCU | 6742 |
| GCAGUGGC G CCCGAACA | 6658 | UGUUCGGG GCCGAAAGGCGAGUGAGGUCU GCCACUGC | 6743 |
| CUCUAGCA G UGGCGCCC | 6659 | GGGCGCCA GCCGAAAGGCGAGUGAGGUCU UGCUAGAG | 6744 |
| UAGCAGUG G CGCCCGAA | 6660 | UUCGGGCG GCCGAAAGGCGAGUGAGGUCU CACUGCUA | 6745 |
| AGAGAUGG G UGCGAGAG | 6661 | CUCUCGCA GCCGAAAGGCGAGUGAGGUCU CCAUCUCU | 6746 |
| AGAUGGGU G CGAGAGCG | 6662 | CGCUCUCG GCCGAAAGGCGAGUGAGGUCU ACCCAUCU | 6747 |
| CUCUCGAC G CAGGACUC | 6663 | GAGUCCUG GCCGAAAGGCGAGUGAGGUCU GUCGAGAG | 6748 |

Input Sequence = HIV1.
Cut Site = G/Y
Arm Length = 8.
Core Sequence = GCcgaaagGCGaGuCaaGGuCu
HIV1 Consensus

TABLE IX
Human HIV DNAzyme and Substrate Sequence

| | Seq | | Seq |
|---|---|---|---|
| Substrate | ID | DNAzyme | ID |
| UCAAUAAA G CUUGCCUU | 6656 | AAGGCAAG GGCTAGCTACAACGA TTTATTGA | 6749 |
| AGGACUCG G CUUGCUGA | 6657 | TCAGCAAG GGCTAGCTACAACGA CGAGTCCT | 6750 |
| GCAGUGGC G CCCGAACA | 6658 | TGTTCGGG GGCTAGCTACAACGA GCCACTGC | 6751 |
| CUCUAGCA G UGGCGCCC | 6659 | GGGCGCCA GGCTAGCTACAACGA TGCTAGAG | 6752 |
| UAGCAGUG G CGCCCGAA | 6660 | TTCGGGCG GGCTAGCTACAACGA CACTGCTA | 6753 |
| AGAGAUGG G UGCGAGAG | 6661 | CTCTCGCA GGCTAGCTACAACGA CCATCTCT | 6754 |
| AGAUGGGU G CGAGAGCG | 6662 | CGCTCTCG GGCTAGCTACAACGA ACCCATCT | 6755 |
| CUCUCGAC G CAGGACUC | 6663 | GAGTCCTG GGCTAGCTACAACGA GTCGAGAG | 6756 |
| UAUGGAAA A CAGAUGGC | 6664 | GCCATCTG GGCTAGCTACAACGA TTTCCATA | 6757 |
| GAAAACAG A UGGCAGGU | 6665 | ACCTGCCA GGCTAGCTACAACGA CTGTTTTC | 6758 |
| AAGCCUCA A UAAAGCUU | 6666 | AAGCTTTA GGCTAGCTACAACGA TGAGGCTT | 6759 |
| GGAGAGAG A UGGGUGCG | 6667 | CGCACCCA GGCTAGCTACAACGA CTCTCTCC | 6760 |
| GACGCAGG A CUCGGCUU | 6668 | AAGCCGAG GGCTAGCTACAACGA CCTGCGTC | 6761 |

Input Sequence = HIV1.
Cut Site = R/Y
Arm Length = 8.
Core Sequence = GGCTAGCTACAACGA
HIV1 Consensus

TABLE X
Human HIV Amberzyme and Substrate Sequence

| | Seq | | Seq |
|---|---|---|---|
| Substrate | ID | Amberzyme | ID |
| UCAAUAAA G CUUGCCUU | 6656 | AAGGCAAG GGAGGAAACUCC CU UCAAGGACAUCGUCCGGG UUUAUUGA | 6762 |
| AGGACUCG G CUUGCUGA | 6657 | UCAGCAAG GGAGGAAACUCC CU UCAAGGACAUCGUCCGGG CGAGUCCU | 6763 |
| GCAGUGGC G CCCGAACA | 6658 | UGUUCGGG GGAGGAAACUCC CU UCAAGGACAUCGUCCGGG GCCACUGC | 6764 |
| CUCUAGCA G UGGCGCCC | 6659 | GGGCGCCA GGAGGAAACUCC CU UCAAGGACAUCGUCCGGG UGCUAGAG | 6765 |
| UAGCAGUG G CGCCCGAA | 6660 | UUCGGGCG GGAGGAAACUCC CU UCAAGGACAUCGUCCGGG CACUGCUA | 6766 |
| AGAGAUGG G UGCGAGAG | 6661 | CUCUCGCA GGAGGAAACUCC CU UCAAGGACAUCGUCCGGG CCAUCUCU | 6767 |
| AGAUGGGU G CGAGAGCG | 6662 | CGCUCUCG GGAGGAAACUCC CU UCAAGGACAUCGUCCGGG ACCCAUCU | 6768 |
| CUCUCGAC G CAGGACUC | 6663 | GAGUCCUG GGAGGAAACUCC CU UCAAGGACAUCGUCCGGG GUCGAGAG | 6769 |
| GGAAAACA G AUGGCAGG | 6669 | CCUGCCAU GGAGGAAACUCC CU UCAAGGACAUCGUCCGGG UGUUUUCC | 6770 |
| AUGGGUGC G AGAGCGUC | 6670 | GACGCUCU GGAGGAAACUCC CU UCAAGGACAUCGUCCGGG GCACCCAU | 6771 |
| AAAAGGGG G GAUUGGGG | 6671 | CCCCAAUC GGAGGAAACUCC CU UCAAGGACAUCGUCCGGG CCCCUUUU | 6772 |
| AGAAAAGG G GGGAUUGG | 6672 | CCAAUCCC GGAGGAAACUCC CU UCAAGGACAUCGUCCGGG CCUUUUCU | 6773 |
| GAAAAGGG G GGAUUGGG | 6673 | CCCAAUCC GGAGGAAACUCC CU UCAAGGACAUCGUCCGGG CCCUUUUC | 6774 |
| GGCUAGAA G GAGAGAGA | 6674 | UCUCUCUC GGAGGAAACUCC CU UCAAGGACAUCGUCCGGG UUCUAGCC | 6775 |
| UUUUAAAA G AAAAGGGG | 6675 | CCCCUUUU GGAGGAAACUCC CU UCAAGGACAUCGUCCGGG UUUUAAAA | 6776 |
| UAUGGCAG G AAGAAGCG | 6676 | CGCUUCUU GGAGGAAACUCC CU UCAAGGACAUCGUCCGGG CUGCCAUA | 6777 |
| UGGCGCCC G AACAGGGA | 6677 | UCCCUGUU GGAGGAAACUCC CU UCAAGGACAUCGUCCGGG GGGCGCCA | 6778 |
| GAGAGAUG G GUGCGAGA | 6678 | UCUCGCAC GGAGGAAACUCC CU UCAAGGACAUCGUCCGGG CAUCUCUC | 6779 |
| CGACGCAG G ACUCGGCU | 6679 | AGCCGAGU GGAGGAAACUCC CU UCAAGGACAUCGUCCGGG CUGCGUCG | 6780 |
| UGACUAGC G GAGGCUAG | 6680 | CUAGCCUC GGAGGAAACUCC CU UCAAGGACAUCGUCCGGG GCUAGUCA | 6781 |
| UAGAAGGA G AGAGAUGG | 6681 | CCAUCUCU GGAGGAAACUCC CU UCAAGGACAUCGUCCGGG UCCUUCUA | 6782 |
| AGGAGAGA G AUGGGUGC | 6682 | GCACCCAU GGAGGAAACUCC CU UCAAGGACAUCGUCCGGG UCUCUCCU | 6783 |
| GAAGGAGA G AGAUGGGU | 6683 | ACCCAUCU GGAGGAAACUCC CU UCAAGGACAUCGUCCGGG UCUCCUUC | 6784 |
| UCGACGCA G GACUCGGC | 6684 | GCCGAGUC GGAGGAAACUCC CU UCAAGGACAUCGUCCGGG UGCGUCGA | 6785 |
| CUAGCAGU G GCGCCCGA | 6685 | UCGGGCGC GGAGGAAACUCC CU UCAAGGACAUCGUCCGGG ACUGCUAG | 6786 |
| GACUAGCG G AGGCUAGA | 6686 | UCUAGCCU GGAGGAAACUCC CU UCAAGGACAUCGUCCGGG CGCUAGUC | 6787 |
| GCUAGAAG G AGAGAGAU | 6687 | AUCUCUCU GGAGGAAACUCC CU UCAAGGACAUCGUCCGGG CUUCUAGC | 6788 |
| AAAGGGGG G AUUGGGGG | 6688 | CCCCCAAU GGAGGAAACUCC CU UCAAGGACAUCGUCCGGG CCCCCUUU | 6789 |

Input Sequence = HIV1.
Cut Site = G/.
Arm Length = 8.
Core Sequence = GGAGGAAACUCC CU UCAAGGACAUCGUCCGGG
HIV1 Consensus

TABLE XI
Human HIV Enzymatic Nucleic Acid and Target molecules

| | | | | |
|---|---|---|---|---|
| Target | Seq ID | RPI# | Enzymatic Nucleic Acid | Seq ID |

| | | | | |
|---|---|---|---|---|
| GAGAUGG G UGCGAGA | 6718 | 25003 | ucucgca GGCTAGCTACAACGA ccaucuc B | 6790 |
| AUGGAAA A CAGAUGG | 6719 | 25004 | ccaucug GGCTAGCTACAACGA uuuccau B | 6791 |
| AAAACAG A UGGCAGG | 6720 | 25005 | ccugcca GGCTAGCTACAACGA cuguuuu B | 6792 |
| AGCCUCA A UAAAGCU | 6721 | 25006 | agcuuua GGCTAGCTACAACGA ugaggcu B | 6793 |
| GAGAGAG A UGGGUGC | 6722 | 25007 | gcaccca GGCTAGCTACAACGA cucucuc B | 6794 |
| CAAUAAA G CUUGCCU | 6723 | 25008 | aggcaag gccgaaaggCgagugaGGuCu uuuauug B | 6795 |
| GGACUCG G CUUGCUG | 6724 | 25009 | cagcaag gccgaaaggCgagugaGGuCu cgagucc B | 6796 |
| GAGAUGG G UGCGAGA | 6718 | 25010 | ucucgca gccgaaaggCgagugaGGuCu ccaucuc B | 6797 |
| GAUGGGU G CGAGAGC | 6725 | 25011 | gcucucg gccgaaaggCgagugaGGuCu acccauc B | 6798 |
| UCUCGAC G CAGGACU | 6726 | 25012 | aguccug gccgaaaggCgagugaGGuCu gucgaga B | 6799 |

G = Guanosine
A, G, C, T (italic) = deoxy
lower case = 2′-O-methyl
s = phosphorothioate 3′-internucleotide linkage
C = 2′-deoxy-2′-Amino cytidine
B = inverted deoxyabasic derivative

TABLE XII
Human HIV-1 Sequences

| Genbank | | | |
|---|---|---|---|
| Acc# | Seq Name(s) | Subtype | Organism |
| A04321 | IIIB LAI | B | HIV-1 |
| AF110962 | 96BW0402 | C | HIV-1 |
| AF110963 | 96BW0407 | C | HIV-1 |
| AF110968 | 96BW0504 | C | HIV-1 |
| AF110965 | 96BW0409 | C | HIV-1 |
| AF110966 | 96BW0410 | C | HIV-1 |
| AF110964 | 96BW0408 | C | HIV-1 |
| AF110975 | 96BW15C05 | C | HIV-1 |
| AF110974 | 96BW15C02 | C | HIV-1 |
| AF110973 | 96BW15B03 | C | HIV-1 |
| AF107771 | UGSE8131 | A | HIV-1 |
| U69585 | WCIPR854 | B | HIV-1 |
| U69588 | WCIPR855 | B | HIV-1 |
| U69589 | WCIPR9011 | B | HIV-1 |
| U69591 | WCIPR9018 | B | HIV-1 |
| U69592 | WCIPR9031 | B | HIV-1 |
| U69593 | WCIPR9032 | B | HIV-1 |
| U69586 | WCIPR8546 | B | HIV-1 |
| AF003888 | NL43WC001 | B | HIV-1 |
| X01762 | REHTLV3 LAI IIIB | B | HIV-1 |
| AF075719 | MNTQ MNcloneTQ | B | HIV-1 |
| AJ239083 | 97CAMP645MO | MO | HIV-1 |
| D86069 | PM213 | B | HIV-1 |
| K02083 | PV22 | B | HIV-1 |
| M93259 | YU10 | B | HIV-1 |
| Z11530 | F12CG | B | HIV-1 |
| AB032740 | TH022 95TNIH022 | CRF01_AE | HIV-1 |
| AF107770 | SE7812 | CRF02_AG | HIV-1 |
| AF070521 | NL43E9 | B | HIV-1 |
| AF033819 | HXB2-copy LAI | B | HIV-1 |
| AF003887 | WC001 | B | HIV-1 |
| AF069140 | DH123 | B | HIV-1 |
| AF110967 | 96BW0502 | C | HIV-1 |
| K03455 | HXB2 HXB2CG | B | HIV-1 |
| M96155 | P896 89.6 | B | HIV-1 |
| X04415 | MAL MALCG | ADK | HIV-1 |
| AF133821 | MB2059 | D | HIV-1 |
| D86068 | MCK1 | B | HIV-1 |
| U69587 | WCIPR8552 | B | HIV-1 |
| U69590 | WCIPR9012 | B | HIV-1 |
| AB032741 | 95TNIH047 TH047 | CRF01_AE | HIV-1 |
| AB023804 | 93IN101 | C | HIV-1 |
| AF193275 | 97BL006 | A | HIV-1 |
| AF197340 | 90CF11697 | CRF01_AE | HIV-1 |
| AF224507 | WK | B | HIV-1 |
| AJ271445 | GB8 GB8-46R | B | HIV-1 |
| AF197338 | 93TH057 | CRF01_AE | HIV-1 |
| AF197339 | 93TH065 | CRF01_AE | HIV-1 |
| AF197341 | 90CF4071 | CRF01_AE | HIV-1 |
| U69584 | 85WCIPR54 | B | HIV-1 |
| L31963 | TH475A LAI | B | HIV-1 |
| U46016 | ETH2220 C2220 | C | HIV-1 |
| U21135 | WEAU160 GHOSH | B | HIV-1 |
| AF042106 | MBCC18R01 | B | HIV-1 |
| K03454 | ELI | D | HIV-1 |
| U51188 | 90CF402 90CR402 | CRF01_AE | HIV-1 |
| U51189 | 93TH253 | CRF01_AE | HIV-1 |
| U34603 | H0320-2A12 | B | HIV-1 |
| M38429 | JRCSF JR-CSF | B | HIV-1 |
| M17451 | RF HAT3 | B | HIV-1 |
| L02317 | BC BCSG3 | B | HIV-1 |
| M93258 | YU2 YU2X | B | HIV-1 |
| M22639 | Z2Z6 Z2 CDC-Z34 | D | HIV-1 |
| AF004394 | AD8, AD87 ADA | B | HIV-1 |
| AF049337 | 94CY032-3 | CRF04_cpx | HIV-1 |
| U34604 | 3202A21 | B | HIV-1 |
| L20587 | ANT70 | O | HIV-1 |
| D10112 | CAM1 | B | HIV-1 |
| U54771 | CM240 | CRF01_AE | HIV-1 |
| U43096 | D31 | B | HIV-1 |
| U37270 | C18MBC | B | HIV-1 |
| U43141 | HAN | B | HIV-1 |
| U23487 | MANC | B | HIV-1 |
| M17449 | MNCG MN | B | HIV-1 |
| L20571 | MVP5180 | O | HIV-1 |
| M27323 | NDK | D | HIV-1 |
| M38431 | NY5CG | B | HIV-1 |
| M26727 | OYI, 397 | B | HIV-1 |
| K02007 | SF2 LAV2 ARV2 | B | HIV-1 |
| M62320 | U455 U455A | A | HIV-1 |
| U26546 | WR27 | B | HIV-1 |
| AF004885 | Q23 | A | HIV-1 |
| AF042100 | MBC200 | B | HIV-1 |
| AF042101 | MBC925 | B | HIV-1 |
| AJ006287 | 89SP061 89ES061 | B | HIV-1 |
| AF067154 | 93IN999 301999 | C | HIV-1 |
| AF067155 | 95IN21068 21068 | C | HIV-1 |
| AJ006022 | YBF30 | N | HIV-1 |
| AF061642 | SE6165 G6165 | G | HIV-1 |
| AF119820 | 97PVCH GR11 | CRF04_cpx | HIV-1 |
| AF119819 | 97PVMY GR84 | CRF04_cpx | HIV-1 |
| K02013 | LAI BRU | B | HIV-1 |
| L39106 | IBNG | CRF02_AG | HIV-1 |
| U12055 | LW123 | B | HIV-1 |
| M19921 | NL43 pNL43 | B | HIV-1 |
| AF061640 | HH8793-1.1 | G | HIV-1 |
| AF061641 | HH8793-12.1 | G | HIV-1 |
| AF063223 | DJ263 | CRF02_AG | HIV-1 |
| AF049495 | NC7 | B | HIV-1 |
| AF049494 | 499JC16 | B | HIV-1 |
| AF086817 | TWCYS LM49 | B | HIV-1 |
| AF064699 | BFP90 | CRF06_cpx | HIV-1 |
| AF084936 | DRCBL | G | HIV-1 |
| AF193253 | VI1310 AF193253 | CRF05_DF | HIV-1 |
| AF190127 | VI991 | H | HIV-1 |
| AF193276 | KAL153-2 | CRF03_AB | HIV-1 |
| AF192135 | BW2117 | AJ | HIV-1 |
| AJ288982 | 95ML127 | CRF06_cpx | HIV-1 |
| AJ288981 | 97SE1078 | CRF06_cpx | HIV-1 |
| AJ271370 | YBF106 | N | HIV-1 |
| AJ237565 | 97NOGIL3 | ADHK | HIV-1 |

1. A double stranded short interfering RNA (siRNA) molecule that comprises a first nucleotide sequence complementary to RNA sequence encoding HER2 or a portion thereof, and a second nucleotide sequence having complementarity to said first sequence, wherein said first sequence and said second sequence independently comprise from about 19 nucleotides to about 23 nucleotides, and wherein said siRNA includes at least one nucleotide that is not a 2′-OH containing ribonucleotide.

2. The siRNA molecule of claim 1, wherein said first sequence and said second sequence of said siRNA molecule each comprise about 21 nucleotides.

3. The siRNA molecule of claim 1, wherein said first sequence and said second sequence of said siRNA molecule each comprise about 19 nucleotides.

4. The siRNA molecule of claim 1, wherein said siRNA comprises a nucleotide overhang at the 3′-end, 5′-end, or both 3′ and 5′ ends of said siRNA.

5. The siRNA molecule of claim 1, wherein said siRNA does not comprise a nucleotide overhang.

6. The siRNA molecule of claim 1, wherein said siRNA molecule is chemically synthesized.

7. The siRNA molecule of claim 1, wherein said at least one nucleotide is a 2′-deoxy (2′-H) nucleotide.

8. The siRNA molecule of claim 1, wherein said at least one nucleotide is a 2′-deoxy--2′-fluoro (2′-F) nucleotide.

9. The siRNA molecule of claim 1, wherein said at least one nucleotide is a 2′-O-alkyl nucleotide.

10. The siRNA molecule of claim 9, wherein said 2′-O-alkyl nucleotide is a 2′-O-methyl nucleotide.

11. The siRNA molecule of claim 9, wherein said 2′-O-alkyl nucleotide is a 2′-O-allyl nucleotide.

12. The siRNA molecule of claim 1, wherein said siRNA molecule comprises at least one nucleic acid base modification.

13. The siRNA molecule of claim 1, wherein said siRNA molecule comprises at least one nucleic acid backbone modification.

14. The siRNA molecule of claim 1, wherein said siRNA molecule comprises at least one non-nucleotide.

15. The siRNA molecule of claim 14, wherein said non-nucleotide comprises an abasic moiety.

16. The siRNA molecule of claim 15, wherein said abasic moiety is present at the 3′-end, 5′-end, or both 3′ and 5′-ends of said first and/or said second sequence.

17. A composition comprising the siRNA of claim 1 in a pharmaceutically acceptable carrier or diluent.
